(19) United States
(12) Patent Application Publication
(10) Pub. No.: US 2007/0031873 A1
(43) Pub. Date: Feb. 8, 2007

(54) Predicting bone relapse of breast cancer

(76) Inventors: Yixin Wang, San Diego, CA (US); Yi Zhang, San Diego, CA (US); David Atkins, Toronto (CA); Anieta Sieuwerts, Rotterdam (NL); Marcel Smid, Rotterdam (NL); Jan G.M. Klijn, Rotterdam (NL); John W.M. Martens, Rotterdam (NL); John A. Foekens, Rotterdam (NL)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 11/497,180

(22) Filed: Aug. 1, 2006

Related U.S. Application Data
(60) Provisional application No. 60/704,740, filed on Aug. 2, 2005.

(51) Int. Cl. C12Q 1/68; G01N 33/574

(52) U.S. Cl. 435/6; 435/7.23; 977/924

(57) ABSTRACT

A method of providing predicting relapse of breast cancer in bone is conducted by analyzing the expression of a group of genes. Gene expression profiles in a variety of medium such as microarrays are included as are kits that contain them.

### CROSS REFERENCE TO RELATED APPLICATION

This application claims the benefit of U.S. National Application Ser. No. 60/704,740, filed Aug. 2, 2005.

### BACKGROUND

This invention relates to breast cancer patient prognosis with respect to relapse to bone and is based on the gene expression profiles of patient biological samples.

The most abundant site of a distant relapse in breast cancer is the bone. Many factors have been implicated in facilitating bone relapse including blood flow in red bone marrow, adhesive molecules in the tumor cells, and immobilized growth factors in the bone matrix such as transforming growth factors-β, bone morphogenetic proteins, platelet derived growth factor, insulin-like growth factors, and fibroblast growth factors. However, gene-based relationships involving the promotion of interactions with bone and cancer cells derived from breast cancers have been largely unknown.

A breast cancer prognostic was recently described for predicting distant recurrence in lymph node negative patients. Wang et. al, PCT/US2005/005711 filed Feb. 18, 2005. Gene expression patterns have also been used to classify breast tumors into different clinically relevant subtypes. Perou et al. (2000); Sørlie et al. (2001); Sørlie et al. (2003); Gruvberger et al. (2001); van't Veer et al. (2002); van de Vijver et al. (2002); Ahr et al. (2002); Huang et al. (2003); Sotiriou et al. (2003); Woelfle et al. (2003); Ma et al. (2003); Ramaswamy et al. (2003); Chang et al. (2003); Sotiriou et al. (2003); and Hedenfalk et al. (2001). Currently, however, there are few diagnostic tools available to identify patients specifically at risk for relapse to bone. There is a need to specifically identify a patient's risk of disease relapse to bone to ensure she receives appropriate therapy.

### SUMMARY OF THE INVENTION

The invention encompasses a method of assessing breast cancer status by obtaining a biological sample from a breast cancer patient and measuring the expression levels of genes via Markers where the gene expression levels above or below pre-determined cut-off levels are indicative of breast cancer status with respect to bone metastasis.

The invention encompasses a method of staging breast cancer by obtaining a biological sample from a breast cancer patient and measuring the expression levels in the sample of genes via Markers where the gene expression levels above or below pre-determined cut-off levels are indicative of the breast cancer stage.

The invention encompasses a method of monitoring breast cancer patient treatment by obtaining a biological sample from a breast cancer patient and measuring the expression levels in the sample of genes via Markers where the gene expression levels above or below pre-determined cut-off levels (as set forth in an algorithm) are sufficiently indicative of risk of metastasis to bone to enable a physician to determine the degree and type of therapy recommended to prevent such metastasis.

The invention encompasses a method of treating a breast cancer patient by obtaining a biological sample from a breast cancer patient; and measuring the expression levels in the sample of genes via Markers where the gene expression levels above or below pre-determined cut-off levels indicate a high risk of bone metastasis and; treating the patient with adjuvant therapy if they are a high risk patient.

The invention encompasses a method of generating a bone relapse probability score to enable prognosis of breast cancer patients by obtaining gene expression data from a statistically significant number of patient biological samples, applying univariate Cox's regression analysis to the data to obtain selected genes; applying weighted expression levels to the selected genes with standard Cox's coefficients to obtain a prediction model that can be applied as a bone relapse probability score.

The invention encompasses a method of generating a breast cancer prognostic patient report by obtaining a biological sample from the patient; measuring gene expression of the sample; applying a bone relapse probability score; and using the results obtained to generate the report and patient reports generated thereby.

The invention encompasses a composition containing Markers.

The invention encompasses a kit for conducting an assay to determine breast cancer prognosis using a biological sample obtained from the patient. The kit contains materials for detecting Markers. Preferably, the kit includes instructions for its use.

The invention encompasses articles for assessing breast cancer status containing Markers.

The invention encompasses a diagnostic/prognostic portfolio containing Markers where the combination is sufficient to characterize breast cancer status or risk of relapse in bone in a biological sample.

The inventive methods can be advantageously used in conjunction with other breast prognostics. This can be done reflexively so that first the prognosis of any relapse is determined followed by the application of the PAM approach presented in this application. Alternatively, these methods can be conducted simultaneously or near-simultaneously to provide the physician and/or patient with information concerning the likelihood of relapse anywhere and, more specifically, relapse to bone.

### DETAILED DESCRIPTION

The invention encompassing a method of assessing breast cancer status determines whether a patient is at high risk of a recurrence of the disease in bone. References to prognosis and prediction throughout this application are drawn to predictions relating to the relapse of breast cancer with its appearance in bone. These methods involve obtaining a biological sample from a breast cancer patient and measuring the expression levels in the sample of certain genes where the gene expression levels above or below pre-determined cut-off levels are indicative of breast cancer status with respect to its relapse in bone.

The inventive methods, compositions, articles, and kits described and claimed in this specification include one or more Markers. “Marker” is used throughout this specification to refer to:
a) genes and gene expression products such as RNA, mRNA and corresponding cDNA, peptides, proteins, fragments and complements of each of the foregoing, and
b) compositions such as probes, antibodies, ligands, haptens, and labels that, through physical or chemical interaction with a) indicate the expression of the gene or presence of the gene expression product and wherein the gene, gene expression product or compositions correspond with:
   i) SEQ ID NO 112,
   ii) a combination of SEQ ID NO 112 and a member of the group consisting of SEQ ID NO 113, SEQ ID NO 114, SEQ ID NO 115, SEQ ID NO 116,
   iii) a combination of SEQ ID NO 112 and all of SEQ ID NO 113, SEQ ID NO 114, SEQ ID NO 115, and SEQ ID NO 116,
   iv) one or more of SEQ ID NO 112-SEQ ID NO 116 and one or more of SEQ ID NO 117-198, or
   v) all of SEQ ID NO 112-SEQ ID NO 198.

A gene corresponds to the sequence designated by a SEQ ID NO when it contains that sequence. A gene segment or fragment corresponds to the sequence of such gene when it contains a portion of the referenced sequence or its complement sufficient to distinguish it as being the sequence of the gene. A gene expression product corresponds to such sequence when its RNA, mRNA, or cDNA hybridizes to the composition having such sequence (e.g. a probe) or, in the case of a peptide or protein, it is encoded by such mRNA. A segment or fragment of a gene expression product corresponds to the sequence of such gene or gene expression product when it contains a portion of the referenced gene expression product or its complement sufficient to distinguish it as being the sequence of the gene or gene expression product.

Markers corresponding to ii and iii are preferred. Markers corresponding to iv and v are most preferred.

While the mere presence or absence of particular nucleic acid sequences (e.g., genes containing SNPs) in a tissue sample has only rarely been found to have diagnostic or prognostic value, information about the expression of various proteins, peptides or mRNA is increasingly viewed as important. The mere presence of nucleic acid sequences having the potential to express proteins, peptides, or mRNA (such sequences referred to as “genes”) within the genome by itself is not determinative of whether a protein, peptide, or mRNA is expressed in a given cell. Whether or not a given gene capable of expressing proteins, peptides, or mRNA does so and to what extent such expression occurs, if at all, is determined by a variety of complex factors. However, relative indications of the degree to which genes are active or inactive can be found in gene expression profiles. Here it is reported that assaying gene expression is useful in identifying and reporting whether a breast cancer patient is likely to experience a relapse to bone. This is important for a number of reasons including insuring that the patient can receive the most beneficial treatment.

Sample preparation is an important aspect of practicing the methods and using the kits and articles of the invention. Sample preparation requires the collection of patient samples. Patient samples used in the inventive method are those that are suspected of containing diseased cells such as epithelial cells taken from the primary tumor in a breast sample. The sample can be any sample that is suspected of having cancer cells present including, without limitation, primary tumor tissue, aspirates of tissue or fluid, ductal fluids, prepared by any method known in the art including bulk tissue preparation and laser capture microdissection. Bulk tissue preparations can be obtained from a biopsy or a surgical specimen. Fluids can be readily obtained with fine needle aspirates, lavages, and other methods of extraction known in the medical arts. Most preferably, the sample is obtained from a primary tumor. Samples taken from surgical margins are also preferred. Laser Capture Microdissection (LCM) technology is one way to select the cells to be studied, minimizing variability caused by cell type heterogeneity. Samples can also comprise circulating epithelial cells extracted from peripheral blood. These can be obtained according to a number of methods but the most preferred method is the magnetic separation technique described in U.S. Pat. No. 6,136,182, incorporated in its entirety in this specification. Once the sample containing the cells of interest has been obtained, genetic material is extracted and used in the methods or with the kits or articles of the inventions. Preferably, RNA is extracted and amplified and a gene expression profile is obtained, preferably via micro-array, for genes in the appropriate portfolios.

Using gene expression microarray data (Affymetrix U133A Chips) of 107 primary breast tumors that were all lymph-node negative at the time of diagnosis and that all had relapsed, panels of genes were found significantly differentially expressed between patients who relapsed to bone versus those who relapsed elsewhere in the body. This panel was arrived at using the SAM approach that is described in this application. The most differentially expressed gene in that panel, TFF1, was confirmed by quantitative RT-PCR in an independent cohort (n=122, p=0.0015). Additionally, a classifier was developed that accurately predicts bone relapse in general. This classifier/panel is referred to as the PAM panel in this application. This classifier can be used as tool to recommend adjuvant therapy particularly suited for treatment of bone metastasis including without limitation, bisphosphonate treatment. These treatments can be recommended in addition to endocrine, chemotherapy, radiation, or other treatments.

The inventive methods of staging breast cancer involve obtaining a biological sample from a breast cancer patient and measuring the expression levels in the sample of genes via Markers where the gene expression levels above or below pre-determined cut-off levels are used as input to indicate breast cancer stage. The information is utilized in any classification known in the art including the TNM system American Joint Committee on Cancer www.cancerstaging.org and comparison to stages corresponding to patients with similar gene expression profiles.

The methods of determining breast cancer patient treatment involve obtaining a biological sample from a breast cancer patient and measuring the expression levels in the sample of genes via Markers where the gene expression levels above or below pre-determined cut-off levels are sufficiently indicative of risk of relapse to bone to enable a physician to determine the degree and type of therapy recommended to prevent such relapse.

The method of treating a breast cancer patient involve obtaining a biological sample from a breast cancer patient; and measuring the expression levels in the sample of genes via Markers where the gene expression levels above or below pre-determined cut-off levels indicate a high risk of relapse to bone and treating the patient with adjuvant therapy if they are a high risk patient.

The above methods can further include measuring the expression level of at least one gene constitutively expressed in the sample.

The above methods preferably have a specificity of at least 40% and a sensitivity of at least at least 80%.

The above methods can be used where the expression pattern of the genes is compared to an expression pattern indicative of a breast cancer patient who has relapsed in bone. The comparison can be by any method known in the art including comparison of expression patterns is conducted with pattern recognition methods. Pattern recognition methods can be any known in the art including PAM analysis and, alternatively, Cox's proportional hazards analysis.

Preferably, levels of up- and down-regulation of the gene markers used in the invention are distinguished based on fold changes of the intensity measurements of hybridized microarray probes. In any event, the inventive methods, kits, portfolios, and measurements and analyses undertaken in them employ pre-determined cut-off levels indicative of at least 1.7-fold over- or under-expression in the sample relative to samples from patients without bone relapse. Preferably, the pre-determined cut-off levels have at least a statistically significant p-value for over-expression in the sample from patients having relapse to bone relative to non-bone relapse patients. More preferably, the p-value is less than 0.05. A 2.0 fold difference is more preferred for making such distinctions. That is, before a gene is said to be differentially expressed in samples from relapsing versus non-relapsing patients, the samples from the relapsing patients are found to yield at least 2 times more, or 2 times less intensity than the those of the non-relapsing patient. The greater the fold difference, the more preferred is use of the gene as a diagnostic or prognostic tool provided that the p-value of the gene is acceptable from a clinical point of view (i.e., closely associated with relapse to bone). Genes selected for the gene expression profiles of the instant invention have expression levels that result in the generation of a signal that is distinguishable from those of the non-relapsing or non-modulated genes by an amount that exceeds background using clinical laboratory instrumentation.

The above methods can be used where gene expression is measured on a microarray or gene chip. Gene chips and microarrays suitable for use herein are also included in the invention. The microarray can be a cDNA array or an oligonucleotide array and can further contain one or more internal control reagents.

The above methods can likewise be used where gene expression is determined by nucleic acid amplification and detection methods. Preferably, such methods include the polymerase chain reaction (PCR) of RNA extracted from the sample. The PCR can be reverse transcription polymerase chain reaction (RT-PCR). The RT-PCR can further contain one or more internal control reagents.

The above methods can be used where gene expression is detected by measuring or detecting a protein encoded by the gene. Any method known in the art can be used including detection by an antibody specific to the protein and measuring a characteristic of the gene. Suitable characteristics include, without limitation, DNA amplification, methylation, mutation and allelic variation.

A method of the invention encompasses generating a bone relapse probability score to enable prediction of relapse to bone. This method can be conducted by obtaining gene expression data from a statistically significant number of patient biological samples and applying the PAM analysis as described below. In another embodiment of the invention, the bone relapse probability score can be obtained by application of the Cox regression formula using standardized Cox regression coefficients.

The inventive method of generating a breast cancer prognostic patient report (for relapse to bone) is conducted by obtaining a biological sample from the patient, measuring gene expression of the sample; applying a bone relapse probability score to the results and using the results obtained to generate the report. The report may contain an assessment of patient outcome and/or probability of risk relative to the patient population.

The inventive compositions include at least one probe set of Markers. The composition can further contain reagents for conducting a microarray, amplification or probe-based analysis, and a medium through which the nucleic acid sequences, their complements, or portions thereof are assayed.

The inventive kit for conducting an assay to determine breast cancer prognosis in a biological sample include materials for detecting Markers. The kit can further contain reagents for conducting a microarray, amplification or probe-based analysis, and a medium through which the nucleic acid sequences, their complements, or portions thereof are assayed.

The inventive articles for assessing breast cancer status include materials for detecting Markers. The articles can further contain reagents for conducting a microarray, amplification or probe-based analysis, and a medium through which said nucleic acid sequences, their complements, or portions thereof are assayed.

The microarrays useful in the inventive methods, articles, and kits can contain Markers where the combination is sufficient to characterize breast cancer status or risk of relapse in bone.

The preferred kits, articles, and microarrays include substrates to which probes are fixed or bind and to which target Markers bind or associate so they can be detected. It is most preferred that these substrates are suitable only for conducting the assay or described in this specification or that are suitable for conducting a discrete number of related assays (i.e., contain a small number of panels).

The invention encompasses a diagnostic/prognostic portfolio of Markers where the combination is sufficient to characterize breast cancer status or risk of relapse to bone in a biological sample.

Preferred methods for establishing gene expression profiles include determining the amount of RNA that is produced by a gene that can code for a protein or peptide. This is accomplished by reverse transcriptase PCR (RT-PCR), competitive RT-PCR, real time RT-PCR, differential display RT-PCR, Northern Blot analysis and other related tests. While it is possible to conduct these techniques using individual PCR reactions, it is best to amplify complementary DNA (cDNA) or complementary RNA (cRNA) produced from mRNA and analyze it via microarray. A number of different array configurations and methods for their production are known to those of skill in the art and are described in U.S. patents such as: U.S. Pat. Nos. 5,445,934; 5,532,128; 5,556,752; 5,242,974; 5,384,261; 5,405,783; 5,412,087; 5,424,186; 5,429,807; 5,436,327; 5,472,672; 5,527,681; 5,529,756; 5,545,531; 5,554,501; 5,561,071; 5,571,639; 5,593,839; 5,599,695; 5,624,711; 5,658,734; and 5,700,637.

Microarray technology allows for the measurement of the steady-state mRNA level of thousands of genes simultaneously thereby presenting a powerful tool for identifying effects such as the onset, arrest, or modulation of uncontrolled cell proliferation. Two microarray technologies are currently in wide use. The first are cDNA arrays and the second are oligonucleotide arrays. Although differences exist in the construction of these chips, essentially all downstream data analysis and output are the same. The product of these analyses are typically measurements of the intensity of the signal received from a labeled probe used to detect a cDNA sequence from the sample that hybridizes to a nucleic acid sequence at a known location on the microarray. Typically, the intensity of the signal is proportional to the quantity of cDNA, and thus mRNA, expressed in the sample cells. A large number of such techniques are available and useful. Preferred methods for determining gene expression can be found in U.S. Pat. Nos. 6,271,002; 6,218,122; 6,218,114; and 6,004,755.

Analysis of expression levels is conducted by comparing signal intensities and subjecting these measurements to statistical algorithms. Generating a ratio matrix of the expression intensities of genes in a test sample versus those in a control sample is one such method. For instance, the gene expression intensities from a test tissue can be compared with the expression intensities generated from tissue of the same type from a patient with the condition of interest (e.g., tumor tissue from a patient who relapsed to bone vs. one who did not). A ratio of these expression intensities indicates the fold-change in gene expression between the test and control samples.

Gene expression profiles can also be displayed in a number of ways. The most common method is to arrange raw fluorescence intensities or ratio matrix into a graphical dendogram where columns indicate test samples and rows indicate genes. The data are arranged so genes that have similar expression profiles are proximal to each other. The expression ratio for each gene is visualized as a color. For example, a ratio less than one (indicating down-regulation) may appear in the blue portion of the spectrum while a ratio greater than one (indicating up-regulation) may appear as a color in the red portion of the spectrum. Commercially available computer software programs are available to display such data including GeneSpring from Agilent Technologies and Partek Discover™ and Partek Infer™ software from Partek®.

Modulated genes used in the methods of the invention are described in the Examples. Differentially expressed genes are either up- or down-regulated in patients with a relapse to bone of breast cancer relative to those without such a relapse. Up regulation and down regulation are relative terms meaning that a detectable difference (beyond the contribution of noise in the system used to measure it) is found in the amount of expression of the genes relative to some baseline. In this case, the baseline is the measured gene expression of a non-relapsing patient. The genes of interest in the diseased cells (from the relapsing patients) are then either up- or down-regulated relative to the baseline level (from the non-bone relapsing patients) using the same measurement method. A patient with a gene expression pattern consistent with that of the condition of interest (likelihood of relapse to bone) is assessed as having such condition and treated accordingly. In therapy monitoring, clinical judgments are made regarding the effect of a given course of therapy by comparing the expression of genes over time to determine whether the gene expression profiles have changed or are changing to patterns more consistent with tissue of non-relapsing patients.

Statistical values can be used to confidently distinguish modulated from non-modulated genes and noise and establish expression profiles. One such statistical test that finds the genes most significantly different between diverse groups of samples is based on a Student's T-test. P-values are obtained relating to the inclusion of particular genes to a class of genes. The lower the p-value, the more compelling the evidence that the gene is showing a difference between the different groups. Since microarrays measure more than one gene at a time, tens of thousands of statistical tests may be performed at one time so one is unlikely to see small p-values just by chance. Adjustments for using a Sidak correction as well as a randomization/permutation experiment can be made. A p-value less than 0.05 by the T-test is evidence that the gene is significantly different. More compelling evidence is a p-value less then 0.05 after the Sidak correction is factored in. For a large number of samples in each group, a p-value less than 0.05 after the randomization/permutation test is the most compelling evidence of a significant difference.

Another parameter that can be used to select genes that generate a signal that is greater than that of the non-modulated gene or noise is the use of a measurement of absolute signal difference. Preferably, the signal generated by the modulated gene expression is at least 20% different than those of the non-modulated gene or the genes of the non-relapsing patient (on an absolute basis). It is even more preferred that such genes produce expression patterns that are at least 30% different than those of non-modulated genes.

The genes that are grouped so that information obtained about the set of genes in the group provides a sound basis for making a clinically relevant judgment such as a diagnosis, prognosis, or treatment choice make up the portfolios of the invention. In this case, the judgments supported by the portfolios involve breast cancer and its chance of relapse to bone.

Preferably, portfolios are established such that the combination of genes in the portfolio exhibit improved sensitivity and specificity relative to individual genes or randomly selected combinations of genes. In the context of the instant invention, the sensitivity of the portfolio can be reflected in the fold differences exhibited by a gene's expression in the state of a patient that relapses to bone relative to those without such relapse. Specificity can be reflected in statistical measurements of the correlation of the signaling of gene expression with the condition of interest. For example, standard deviation can be a used as such a measurement. In considering a group of genes for inclusion in a portfolio, a small standard deviation in expression measurements correlates with greater specificity. Other measurements of variation such as correlation coefficients can also be used.

The portfolios of genes of this invention were determined through SAM analysis. Gene expression patterns are analyzed using PAM analysis. SAM (Significance Analysis of Microarrays) is a statistical approach to identify genes whose expression patterns are significantly associated with specific characteristics of sample sets. This method is embodied in software developed at Stanford University and it is publicly available. SAM identifies genes with statistically significant changes in expression by assimilating a set of gene specific T-tests. The method is described in US Patent Application 20020019704 to Tusher et. al., filed Mar. 19, 2001 and incorporated in its entirety in this specification. It is also described in Significance Analysis of Microarrays Applied to the Ionizing Radiation Response; Tusher, Tibshirani, and Chu, 5116-5121_PNAS_Apr. 24, 2001_vol. 98_no. 9.

In a SAM analysis, each gene assayed is assigned a score on the basis of its change in gene expression relative to the standard deviation of repeated measurements for that gene. Genes with scores greater than a threshold are deemed potentially significant. The percentage of such genes identified by chance is the false discovery rate (FDR). To estimate the FDR, nonsense genes are identified by analyzing permutations of the measurements. The threshold can be adjusted to identify smaller or larger sets of genes, and FDRs are calculated for each set.

A value referred to as the “relative difference” or d(i) in gene expression is based on the ratio of change in gene expression to standard deviation in the data for that gene. The “gene-specific scatter” s(i) is the standard deviation of repeated expression measurements. The coefficient of variation of d(i) is computed as a function of s(i). To find significant changes in gene expression, genes are ranked by magnitude of their d(i)value s, so that d(1) is the largest relative difference, d(2) is the second largest relative difference, and d(i) is the ith largest relative difference. For each of the permutations, relative differences dp(i) are also calculated, and the genes are again ranked such that dp(i) is the ith largest relative difference for permutation p. The expected relative difference, dE(i), is defined as the average over the balanced permutations.

To identify potentially significant changes in expression, a scatter plot of the observed relative difference d(i) versus the expected relative difference dE(i) can be used. For the vast majority of genes, d(i) is approximately equal to dE(i), but some genes are represented by points displaced from the d(i)=dE(i) line by a distance greater than a threshold. To determine the number of falsely significant genes generated by SAM, horizontal cutoffs are defined as the smallest d(i) among the genes called significantly induced and the least negative d(i) among the genes called significantly repressed. The number of falsely significant genes corresponding to each permutation is computed by counting the number of genes that exceed the horizontal cutoffs for induced and repressed genes. The estimated number of falsely significant genes is the average of the number of genes called significant from all permutations. This method for setting thresholds provides asymmetric cutoffs for induced and repressed genes. An alternative is the standard t test, which imposes a symmetric horizontal cutoff, with d(i) greater than c for induced genes and d(i) less than c for repressed genes. However, the asymmetric cutoff is preferred because it allows for the possibility that d(i) for induced and repressed genes may behave differently in some biological experiments.

PAM (Predictive Analysis of Microarrays) analysis is a modified version of the nearest-centroid method. The method was developed at Stanford University Labs and is typically carried out using the Statistical package R. It provides a list of significant genes whose expression characterizes each diagnostic class and estimates prediction error via cross-validation. The method is a nearest shrunken centroid methodology. It is described in Diagnosis of Multiple Cancer Types by Shrunken Centroids of Gene Expression; Narashiman and Chu, PNAS 2002 99:6567-6572 (May 14, 2002).

In this method, a standardized centroid is computed for each class. This is the average gene expression for each gene in each class divided by the within-class standard deviation for that gene. Nearest centroid classification takes the gene expression profile of a new sample, and compares it to each of these class centroids. The class whose centroid that it is closest to, in squared distance, is the predicted class for that new sample. Nearest shrunken centroid classification “shrinks” each of the class centroids toward the overall centroid for all classes by an amount called the threshold. This shrinkage consists of moving the centroid towards zero by threshold, setting it equal to zero if it hits zero. For example if threshold was 2.0, a centroid of 3.2 would be shrunk to 1.2, a centroid of −3.4 would be shrunk to −1.4, and a centroid of 1.2 would be shrunk to zero. After shrinking the centroids, the new sample is classified by the usual nearest centroid rule, but using the shrunken class centroids. This shrinkage can make the classifier more accurate by reducing the effect of noisy genes and provides an automatic gene selection. In particular, if a gene is shrunk to zero for all classes, then it is eliminated from the prediction rule. Alternatively, it may be set to zero for all classes except one, and it can be learned that the high or low expression for that gene characterizes that class. The user decides on the value to use for threshold. Typically one examines a number of different choices. To guide in this choice, PAM does K-fold cross-validation for a range of threshold values. The samples are divided up at random into K roughly equally sized parts. For each part in turn, the classifier is built on the other K−1 parts then tested on the remaining part. This is done for a range of threshold values, and the cross-validated misclassification error rate is reported for each threshold value. Typically, the user would choose the threshold value giving the minimum cross-validated misclassification error rate.

Alternatively, gene expression portfolios can be established through the use of optimization algorithms such as the mean variance algorithm widely used in establishing stock portfolios. This method is described in detail in US patent publication number 20030194734. Essentially, the method calls for the establishment of a set of inputs (stocks in financial applications, expression as measured by intensity here) that will optimize the return (e.g., signal that is generated) one receives for using it while minimizing the variability of the return. Many commercial software programs are available to conduct such operations. “Wagner Associates Mean-Variance Optimization Application,” referred to as “Wagner Software” throughout this specification, is preferred. This software uses functions from the “Wagner Associates Mean-Variance Optimization Library” to determine an efficient frontier and optimal portfolios in the Markowitz sense is preferred. Use of this type of software requires that microarray data be transformed so that it can be treated as an input in the way stock return and risk measurements are used when the software is used for its intended financial analysis purposes.

The process of selecting a portfolio can also include the application of heuristic rules. Preferably, such rules are formulated based on biology and an understanding of the technology used to produce clinical results. More preferably, they are applied to output from the optimization method. For example, the mean variance method of portfolio selection can be applied to microarray data for a number of genes differentially expressed in subjects with breast cancer. Output from the method would be an optimized set of genes that could include some genes that are expressed in peripheral blood as well as in diseased tissue. If samples used in the testing method are obtained from peripheral blood and certain genes differentially expressed in instances of breast cancer are differentially expressed in peripheral blood, then a heuristic rule can be applied in which a portfolio is selected from the efficient frontier excluding those that are differentially expressed in peripheral blood. Of course, the rule can be applied prior to the formation of the efficient frontier by, for example, applying the rule during data pre-selection.

Other heuristic rules can be applied that are not necessarily related to the biology in question. For example, one can apply a rule that only a prescribed percentage of the portfolio can be represented by a particular gene or group of genes. Commercially available software such as the Wagner Software readily accommodates these types of heuristics. This can be useful, for example, when factors other than accuracy and precision (e.g., anticipated licensing fees) have an impact on the desirability of including one or more genes.

One method of the invention involves comparing gene expression profiles for various genes (or portfolios) to ascribe prognoses. The gene expression profiles of each of the genes comprising the portfolio are fixed in a medium such as a computer readable medium. This can take a number of forms. For example, a table can be established into which the range of signals (e.g., intensity measurements) indicative of the condition of interest (e.g., high probability of relapse to bone) is input. Actual patient data can then be compared to the values in the table to determine the likelihood of relapse to bone from the patient samples. In a more sophisticated embodiment, patterns of the expression signals (e.g., fluorescent intensity) are recorded digitally or graphically.

The gene expression patterns from the gene portfolios used in conjunction with patient samples are then compared to the expression patterns. Pattern comparison software can then be used to determine whether the patient samples have a pattern indicative of relapse to bone. Of course, these comparisons can also be used to determine whether the patient is not likely to experience relapse to bone. The expression profiles of the samples are then compared to the portfolio of a control cell. If the sample expression patterns are consistent with the expression pattern for relapse to bone of a breast cancer then (in the absence of countervailing medical considerations) the patient is treated as one would treat such a relapsing patient. If the sample expression patterns are consistent with the expression pattern from the normal/control cell then the patient is diagnosed negative for breast cancer.

The gene expression pattern of a patient can be used to determine prognosis of breast cancer (with respect to its relapse in bone) through the use of a Cox's hazard analysis program. Such analyses are preferably conducted using S-Plus software (commercially available from Insightful Corporation). Using such methods, a gene expression profile is compared to that of a profile that confidently represents bone relapse (i.e., expression levels for the combination of genes in the profile is indicative of bone relapse). The Cox's hazard model with the established threshold is used to compare the similarity of the two profiles (known relapse to bone versus patient) and then determines whether the patient profile exceeds the threshold. If it does, then the patient is classified as one who will relapse to bone and is accorded treatment such as adjuvant therapy, bisphosphonate therapy, or other appropriate therapy. If the patient profile does not exceed the threshold then they are classified as a patient without bone relapse. Other analytical tools can also be used to answer the same question such as, linear discriminate analysis, logistic regression and neural network approaches.

Numerous other well-known methods of pattern recognition are available. The following references provide some examples:

Weighted Voting: Golub et al. (1999).

Support Vector Machines: Su et al. (2001); and Ramaswamy et al. (2001).

K-nearest Neighbors: Ramaswamy (2001).

Correlation Coefficients: van't Veer et al. (2002).

The gene expression profiles of this invention can also be used in conjunction with other non-genetic diagnostic methods useful in cancer diagnosis, prognosis, or treatment monitoring. For example, in some circumstances it is beneficial to combine the diagnostic power of the gene expression based methods described above with data from conventional markers such as serum protein markers (e.g., Cancer Antigen 27.29 (“CA 27.29”)). A range of such markers exists including such analytes as CA 27.29. In one such method, blood is periodically taken from a treated patient and then subjected to an enzyme immunoassay for one of the serum markers described above. When the concentration of the marker suggests the return of tumors or failure of therapy, a sample source amenable to gene expression analysis is taken. Where a suspicious mass exists, a fine needle aspirate (FNA) is taken and gene expression profiles of cells taken from the mass are then analyzed as described above. Alternatively, tissue samples may be taken from areas adjacent to the tissue from which a tumor was previously removed. This approach can be particularly useful when other testing produces ambiguous results.

Articles of this invention include representations of the gene expression profiles useful for treating, diagnosing, prognosticating, and otherwise assessing whether it is likely that a breast cancer patient will experience relapse in bone. These profile representations are reduced to a medium that can be automatically read by a machine such as computer readable media (magnetic, optical, and the like). The articles can also include instructions for assessing the gene expression profiles in such media. For example, the articles may comprise a CD ROM having computer instructions for comparing gene expression profiles of the portfolios of genes described above. The articles may also have gene expression profiles digitally recorded therein so that they may be compared with gene expression data from patient samples. Alternatively, the profiles can be recorded in different representational format. A graphical recordation is one such format. Clustering algorithms such as those incorporated in Partek Discover™ and Partek Infer™ software from Partek® mentioned above can best assist in the visualization of such data.

Different types of articles of manufacture according to the invention are media or formatted assays used to reveal gene expression profiles. These can comprise, for example, microarrays in which sequence complements or probes are affixed to a matrix to which the sequences indicative of the genes of interest combine creating a readable determinant of their presence. Alternatively, articles according to the invention can be fashioned into reagent kits for conducting hybridization, amplification, and signal generation indicative of the level of expression of the genes of interest for detecting breast cancer.

Kits made according to the invention include formatted assays for determining the gene expression profiles. These can include all or some of the materials needed to conduct the assays such as reagents and instructions.

The invention is further illustrated by the following non-limiting examples. All references cited herein are hereby incorporated by reference herein.

### EXAMPLES

Genes analyzed according to this invention are typically related to full-length nucleic acid sequences that code for the production of a protein or peptide. One skilled in the art will recognize that identification of full-length sequences is not necessary from an analytical point of view. That is, portions of the sequences or ESTs can be selected according to well-known principles for which probes can be designed to assess gene expression for the corresponding gene.

### Example 1

Sample Handling and Microarray Work for Previously Established Distant Relapse Profile

This example describes the establishment of a portfolio of genes for the identification of breast cancer patients at high risk of a relapse generally (i.e., not restricted to bone relapse).

Frozen tumor specimens from lymph node negative patients treated during 1980-1995, but untreated with systemic neoadjuvant therapy, were selected from the tumor bank at the Erasmus Medical Center (Rotterdam, Netherlands). All tumor samples were submitted to a reference laboratory from 25 regional hospitals for steroid hormone receptor measurements. The guidelines for primary treatment were similar for all hospitals. Tumors were selected in a manner to avoid bias. On the assumption of a 25-30% in 5 years, and a substantial loss of tumors because of quality control reasons, 436 invasive tumor samples were processed. Patients with a poor, intermediate, and good clinical outcome were included. Samples were rejected based on insufficient tumor content (53), poor RNA quality (77) or poor chip quality (20) leaving 286 samples eligible for further analysis.

Median age of patients at the time of surgery (breast conserving surgery: 219 patients; modified radical mastectomy: 67 patients) was 52 years (range, 26-83 years). Radiotherapy was given to 248 patients (87%) according to institutional protocol. Patients were included regardless of radiotherapy status, as this study was not aimed to investigate the potential effects of a specific type of surgery or adjuvant radiotherapy. Furthermore, studies have shown that radiotherapy has no clear effect on distant disease relapse. Early Breast Cancer Trialists (1995). Lymph node negativity was based on pathological examination by regional pathologists. Foekens et al. (1989a).

Prior to inclusion, all 286 tumor samples were confirmed to have sufficient (>70%) tumor and uniform involvement of tumor in H&E stained 5 μm frozen sections. ER (and PgR) levels were measured by ligand binding assay or enzyme immunoassay (EIA) (Foekens et al. (1989b)) or by immunohistochemistry (in 9 tumors). The cutoff values used to classify patients as positive or negative for ER and PR was 10 fmol/mg protein or 10% positive tumor cells. Postoperative follow-up involved examination every 3 months during the first 2 years, every 6 months for years 3 to 5, and every 12 months from year 5. The date of diagnosis of metastasis was defined as the date of confirmation of metastasis after symptoms reported by the patient, detection of clinical signs, or at regular follow-up. The median follow-up period of surviving patients (n=198) was 101 months (range, 20-171). Of the 286 patients included, 93 (33%) showed evidence of distant metastasis within 5 years and were counted as failures in the analysis of distant metastasis-free survival (DMFS). Five patients (2%) died without evidence of disease and were censored at last follow-up. Eighty-three patients (29%) died after a previous relapse. Therefore, a total of 88 patients (31%) were failures in the analysis of overall survival (OS).

### Example 2

Gene Expression Analysis of Data Obtained in Example 1

Total RNA was isolated from 20 to 40 cryostat sections of 30 μm thickness (50-100 mg) with RNAzol B (Campro Scientific, Veenendaal, Netherlands). Biotinylated targets were prepared using published methods (Affymetrix, CA, Lipshutz et al. (1999)) and hybridized to the Affymetrix oligonucleotide microarray U133a GeneChip. Arrays were scanned using standard Affymetrix protocols. Each probe set was treated as a separate gene. Expression values were calculated using Affymetrix GeneChip analysis software MAS 5.0. Chips were rejected if average intensity was <40 or if the background signal>100. To normalize the chip signals, probe sets were scaled to a target intensity of 600, and scale mask files were not selected.

### Example 3

Statistical Analysis of Genes Identified in Example 2

Gene expression data was filtered to include genes called “present” in two or more samples. 17,819 genes passed this filter and were used for hierarchical clustering. Before clustering, the expression level of each gene was divided by its median expression level in the patients. This standardization step limited the effect of the magnitude of expression of genes, and grouped together genes with similar patterns of expression in the clustering analysis. To identify patient subgroups, we carried out average linkage hierarchical clustering on both the genes and the samples using GeneSpring 6-0.

To identify genes that discriminate patients who developed distant metastases from those who remained metastasis-free for 5 years, two supervised class prediction approaches were used. In the first approach, 286 patients were randomly assigned to training and testing sets of 80 and 206 patients, respectively. Kaplan-Meier survival curves (Kaplan et al. (1958)) for the two sets were examined to ensure that there was no significant difference and no bias was introduced by the random selection of the training and testing sets. In the second approach, the patients were allocated to one of two subgroups stratified by ER status.

Each patient subgroup was analyzed separately in order to select markers. The patients in the ER-positive subgroup were randomly allocated into training and testing sets of 80 and 129 patients, respectively. The patients in the ER-negative subgroup were randomly divided into training and testing sets of 35 and 42 patients, respectively. The markers selected from each subgroup training set were combined to form a single signature to predict tumor metastasis for both ER-positive and ER-negative patients in a subsequent independent validation.

The sample size of the training set was determined by a resampling method to ensure its statistical confidence level. Briefly, the number of patients in the training set started at 15 patients and was increased by steps of 5. For a given sample size, 10 training sets with randomly selected patients were made. A gene signature was constructed from each of training sets and then tested in a designated testing set of patients by analysis of receiver operating characteristic (ROC) curve with distant metastasis within 5 years as the defining point. The mean and the coefficient of variation (CV) of the area under the curve (AUC) for a given sample size were calculated. A minimum number of patients required for the training set were chosen at the point that the average AUC reached a plateau and the CV of the 10 AUC was below 5%.

Genes were selected as follows. First, univariate Cox's proportional hazards regression was used to identify genes for which expression (on log₂ scale) was correlated with the length of DMFS. To reduce the effect of multiple testing and to test the robustness of the selected genes, the Cox's model was constructed with bootstrapping of the patients in the training set. Efron et al. (1981). Briefly, 400 bootstrap samples of the training set were constructed, each with 80 patients randomly chosen with replacement. A Cox's model was run on each of the bootstrap samples. A bootstrap score was created for each gene by removing the top and bottom 5% p-values and then averaging the inverses of the remaining bootstrap p-values. This score was used to rank the genes. To construct a multiple gene signature, combinations of gene markers were tested by adding one gene at a time according to the rank order. ROC analysis using distant metastasis within 5 years as the defining point was performed to calculate the area under AUC for each signature with increasing number of genes until a maximum AUC value was reached.

The Relapse Score (RS) was used to calculate each patient's risk of distant metastasis. The score was defined as the linear combination of weighted expression signals with the standardized Cox's regression coefficient as the weight.

$$Relapse Score = A \cdot I + {\sum }_{i = 1}^{60} I \cdot {w}_{i} {x}_{i} + B \cdot ( 1 - I ) + {\sum }_{j = 1}^{16} ( 1 - I ) \cdot {w}_{j} {x}_{j}$$

$$where$$

$$I = \{ \begin{matrix}1 & if ER level > 10 fmol per mg protien \\ 0 & if ER level \leq 10 fmol per mg protien\end{matrix}$$

A and B are constants
wᵢ is the standardized Cox's regression coefficient for ER+marker
xᵢ is the expression value of ER+marker on a log₂ scale
wⱼ is the standardized Cox's regression coefficient for ER−marker
xⱼ is the expression value of ER−marker on a log2 scale

The threshold was determined from the ROC curve of the training set to ensure 100% sensitivity and the highest specificity. The values of constants A of 313.5 and B of 280 were chosen to center the threshold of RS to zero for both ER-positive and ER-negative patients. Patients with positive RS scores were classified into the poor prognosis group and patients with negative RS scores were classified into the good prognosis group. The gene signature and the cutoff were validated in the testing set. Kaplan-Meier survival plots and log-rank tests were used to assess the differences in time to distant metastasis of the predicted high and low risk groups. Odds ratios (OR) were calculated as the ratio of the odds of distant metastasis between the patients predicted to relapse and those predicted to remain relapse-free.

Univariate and multivariable analyses with Cox's proportional hazards regression were done on the individual clinical variables with and without the gene signature. The HR and its 95% confidence interval (CI) were derived from these results. All statistical analyses were performed using S-Plus 6.1 software (Insightful, VA).

### Example 4

Pathway Analysis of Genes Identified in Example 3

A functional class was assigned to each of the genes in the prognostic signature gene described in Examples 1-3 (non-bone specific relapse). Pathway analysis was done with Ingenuity 1.0 software (Ingenuity Systems, CA). Affymetrix probes were used as input to search for biological networks built by the software. Biological networks identified by the program were assessed in the context of general functional classes by GO ontology classification. Pathways with two or more genes in the prognostic signature were selected and evaluated.

### Example 5

Results for Examples 1-4

Patient and Tumor Characteristics

Clinical and pathological features of the 286 patients of examples 1-3 are summarized in Table 1.

TABLE 1
Clinical and Pathological Characteristics of Patients and Their Tumors

| | | | ER- | | ER- | | | |
|---|---|---|---|---|---|---|---|---|
| | All | | positive | | negative | | | |
| | patients | | training | | training | | Validation | |
| Characteristics | (%) | | set (%) | | set (%) | | set (%) | |
| Number | 286 | | 80 | | 35 | | 171 | |
| Age (mean ± SD) | 54 ± 12 | | 54 ± 13 | | 54 ± 13 | | 54 ± 12 | |
| ≦40 yr | 36 | (13) | 12 | (15) | 3 | (9) | 21 | (12) |
| 41-55 yr | 129 | (45) | 30 | (38) | 17 | (49) | 82 | (48) |
| 56-70 yr | 89 | (31) | 28 | (35) | 11 | (31) | 50 | (29) |
| >70 yr | 32 | (11) | 10 | (13) | 4 | (11) | 18 | (11) |
| Menopausal status | | | | | | | | |
| Premenopausal | 139 | (49) | 39 | (49) | 16 | (46) | 84 | (49) |
| Postmenopausal | 147 | (51) | 41 | (51) | 19 | (54) | 87 | (51) |
| T stage | | | | | | | | |
| T1 | 146 | (51) | 38 | (48) | 14 | (40) | 94 | (55) |
| T2 | 132 | (46) | 41 | (51) | 19 | (54) | 72 | (42) |
| T¾ | 8 | (3) | 1 | (1) | 2 | (6) | 5 | (3) |
| Grade | | | | | | | | |
| Poor | 148 | (52) | 37 | (46) | 24 | (69) | 87 | (51) |
| Moderate | 42 | (15) | 12 | (15) | 3 | (9) | 27 | (16) |
| Good | 7 | (2) | 2 | (3) | 2 | (6) | 3 | (2) |
| Unknown | 89 | (31) | 29 | (36) | 6 | (17) | 54 | (32) |
| ER* | | | | | | | | |
| Positive | 209 | (73) | 80 | (100) | 0 | (0) | 129 | (75) |
| Negative | 77 | (27) | 0 | (0) | 35 | (100) | 42 | (25) |
| PgR* | | | | | | | | |
| Positive | 165 | (58) | 59 | (74) | 5 | (14) | 101 | (59) |
| Negative | 111 | (39) | 19 | (24) | 29 | (83) | 63 | (37) |
| Unknown | 10 | (3) | 2 | (2) | 1 | (3) | 7 | (4) |
| Metastasis <5 years | | | | | | | | |
| Yes | 93 | (33) | 24 | (30) | 13 | (37) | 56 | (33) |
| No | 183 | (64) | 51 | (64) | 17 | (49) | 115 | (67) |
| Censored if <5 yr | 10 | (3) | 5 | (6) | 5 | (14) | 0 | (0) |

*ER-positive and PgR positive: >10 fmol/mg protein or >10% positive tumor cells.

There were no differences in age or menopausal status. The ER-negative training group had a slightly higher proportion of larger tumors and, as expected, more poor grade tumors than the ER-positive training group. The validation group of 171 patients (129 ER-positive, 42 ER-negative) did not differ from the total group of 286 patients with respect to any of the patients or tumor characteristics.

Two approaches were used to identify markers predictive of disease relapse. First, the data was divided randomly so that all the 286 patients (ER-positive and ER-negative combined) were put into a training set and a testing set. Thirty-five genes were selected from 80 patients in the training set and a Cox's model to predict the occurrence of distant metastasis was built. A moderate prognostic value was observed. Table 2. Unsupervised clustering analysis showed two distinct subgroups highly correlated with the tumor ER status (chi square test p<0.0001).

TABLE 2

| SEQ ID NO: | Cox's coefficient | p-value |
|---|---|---|
| 1 | 4.008 | 0.00006 |
| 2 | −3.649 | 0.00026 |
| 3 | 4.005 | 0.00006 |
| 4 | −3.885 | 0.00010 |
| 5 | −3.508 | 0.00045 |
| 6 | −3.176 | 0.00150 |
| 7 | 3.781 | 0.00016 |
| 8 | 3.727 | 0.00019 |
| 9 | −3.570 | 0.00036 |
| 10 | −3.477 | 0.00051 |
| 11 | 3.555 | 0.00038 |
| 12 | −3.238 | 0.00120 |
| 13 | −3.238 | 0.00120 |
| 14 | 3.405 | 0.00066 |
| 15 | 3.590 | 0.00033 |
| 16 | −3.157 | 0.00160 |
| 17 | −3.622 | 0.00029 |
| 18 | −3.698 | 0.00022 |
| 19 | 3.323 | 0.00089 |
| 20 | −3.556 | 0.00038 |
| 21 | −3.317 | 0.00091 |
| 22 | −2.903 | 0.00370 |
| 23 | −3.338 | 0.00085 |
| 24 | −3.339 | 0.00084 |
| 25 | −3.355 | 0.00079 |
| 26 | 3.713 | 0.00021 |
| 27 | −3.325 | 0.00088 |
| 28 | −2.984 | 0.00284 |
| 29 | 3.527 | 0.00042 |
| 30 | −3.249 | 0.00116 |
| 31 | −2.912 | 0.00360 |
| 32 | 3.118 | 0.00182 |
| 33 | 3.435 | 0.00059 |
| 34 | −2.971 | 0.00297 |
| 35 | 3.282 | 0.00103 |

Each subgroup was analyzed in order to select markers. Seventy-six genes were selected from patients in the training sets (60 for the ER-positive group, 16 for the ER-negative group). With the selected genes and ER status taken together, a Cox's model to predict relapse of cancer (not specific to bone) was built. Validation of the 76-gene predictor in the 171 patient testing set produced an ROC with an AUC value of 0.694, sensitivity of 93% (52/56), and specificity of 48% (55/115). Patients with a relapse score above the threshold of the prognostic signature have an 11·9-fold OR (95% CI: 4.04-35.1; p<0.0001) to develop distant metastasis within 5 years. As a control, randomly selected 76-gene sets were generated. These produced ROC with an average AUC value of 0.515, sensitivity of 91%, and specificity of 12% in the testing group. Patients stratified by such a gene set would have an odds ratio of 1.3 (0.50-3.90; p=0.8) for development of metastases, indicating a random classification. In addition, the Kaplan-Meier analyses for distant metastasis free survival (DMFS) and overall survival (OS) as a function of the 76-gene signature showed highly significant differences in time to metastasis between the groups predicted to have good and poor prognosis. At 60 and 80 months, the respective absolute differences in DMFS between the groups with predicted good and poor prognosis were 40% (93% vs. 53%) and 39% (88% vs. 49%) and those in OS were 27% (97% vs. 70%) and 32% (95% vs. 63%), respectively.

The 76-gene profile also represented a strong prognostic factor for the development of distant metastasis in the subgroups of 84 premenopausal patients (HR: 9.60), 87 postmenopausal patients (HR: 4.04) and 79 patients with tumor sizes of 10 to 20 mm (HR: 14.1).

Univariate and multivariable Cox's regression analyses are summarized in Table 3.

TABLE 3
Uni- and multivariable analyses for DMFS in the testing set of 171 relapse patients

| | Univariate analysis | | | Multivariable analysis* | | |
|---|---|---|---|---|---|---|
| | HR† | (95% CI)† | p-value | HR† | (95% CI)† | p-value |
| Age‡ | | | | | | |
| Age2 vs. Age1 | 1.16 | (0.51-2.65) | 0.7180 | 1.14 | (0.45-2.91) | 0.7809 |
| Age3 vs. Age1 | 1.32 | (0.56-3.10) | 0.5280 | 0.87 | (0.26-2.93) | 0.8232 |
| Age4 vs. Age1 | 0.95 | (0.32-2.82) | 0.9225 | 0.61 | (0.15-2.60) | 0.5072 |
| Menopausal | 1.24 | (0.76-2.03) | 0.3909 | 1.53 | (0.68-3.44) | 0.3056 |
| status§ | | | | | | |
| Stage\|\| | 1.08 | (0.66-1.77) | 0.7619 | 2.57 | (0.23-29.4) | 0.4468 |
| Differentiation¶ | 0.38 | (0.16-0.90) | 0.0281 | 0.60 | (0.24-1.46) | 0.2590 |
| Tumor size** | 1.06 | (0.65-1.74) | 0.8158 | 0.34 | (0.03-3.90) | 0.3849 |
| ER†† | 1.09 | (0.61-1.98) | 0.7649 | 1.05 | (0.54-2.04) | 0.8935 |
| PR†† | 0.83 | (0.51-1.38) | 0.4777 | 0.85 | (0.47-1.53) | 0.5882 |
| 76-gene | 5.67 | (2.59-12.4) | 1.5 × 10⁻⁵ | 5.55 | (2.46-12.5) | 3.6 × 10⁻⁵ |
| signature | | | | | | |

*The multivariable model included 162 patients, due to missing values in 9 patients
†Hazard ratio and 95% confidence interval
‡Age1 is ≦40 yr, Age2 is 41 to 55 yr, Age3 is 56 to 70 yr, Age4 is >70 yr
§Post-menopausal vs. pre-menopausal
||Stage: II & III vs. I
¶Grade: moderate/good vs. poor, unknown grade was included as a separate group
**Tumor size: >20 mm vs. ≦20 mm
††Positive vs. negative

Other than the 76-gene signature, only grade was significant in univariate analysis and moderate/good differentiation was associated with favorable DMFS. Multivariable regression estimation of HR for the occurrence of tumor metastasis within 5 years was 5.55 (p<0.0001), indicating that the 76-gene set represents an independent prognostic signature strongly associated with a higher risk of tumor metastasis. Univariate and multivariable analyses were also done separately for ER-positive and ER-negative patients the 76-gene signature was also an independent prognostic variable in the subgroups stratified by ER status.

The function of the 76 genes (Table 4) in the non-bone specific prognostic signature was analyzed to relate the genes to biological pathways.

TABLE 4

| ER Status | SEQ ID NO. | Std. Cox's coefficient | Cox's p-value |
|---|---|---|---|
| + | 36 | −3.83 | 0.00005 |
| + | 37 | −3.865 | 0.00001 |
| + | 38 | 3.63 | 0.00002 |
| + | 39 | −3.471 | 0.00016 |
| + | 40 | 3.506 | 0.00008 |
| + | 41 | −3.476 | 0.00001 |
| + | 42 | 3.392 | 0.00006 |
| + | 43 | −3.353 | 0.00080 |
| + | 44 | −3.301 | 0.00038 |
| + | 45 | 3.101 | 0.00033 |
| + | 46 | −3.174 | 0.00128 |
| + | 47 | 3.083 | 0.00020 |
| + | 48 | 3.336 | 0.00005 |
| + | 49 | −3.054 | 0.00063 |
| + | 50 | −3.025 | 0.00332 |
| + | 51 | 3.095 | 0.00044 |
| + | 52 | −3.175 | 0.00031 |
| + | 53 | −3.082 | 0.00086 |
| + | 54 | 3.058 | 0.00016 |
| + | 55 | 3.085 | 0.00009 |
| + | 56 | −2.992 | 0.00040 |
| + | 57 | −2.791 | 0.00020 |
| + | 58 | −2.948 | 0.00039 |
| + | 59 | 2.931 | 0.00020 |
| + | 60 | −2.896 | 0.00052 |
| + | 61 | 2.924 | 0.00050 |
| + | 62 | 2.915 | 0.00055 |
| + | 63 | −2.968 | 0.00099 |
| + | 64 | 2.824 | 0.00086 |
| + | 65 | −2.777 | 0.00398 |
| + | 66 | −2.635 | 0.00160 |
| + | 67 | −2.854 | 0.00053 |
| + | 68 | 2.842 | 0.00051 |
| + | 69 | −2.835 | 0.00033 |
| + | 70 | 2.777 | 0.00164 |
| + | 71 | −2.759 | 0.00222 |
| + | 72 | −2.745 | 0.00086 |
| + | 73 | 2.79 | 0.00049 |
| + | 74 | 2.883 | 0.00031 |
| + | 75 | −2.794 | 0.00139 |
| + | 76 | −2.743 | 0.00088 |
| + | 77 | −2.761 | 0.00164 |
| + | 78 | −2.831 | 0.00535 |
| + | 79 | 2.659 | 0.00073 |
| + | 80 | −2.715 | 0.00376 |
| + | 81 | 2.836 | 0.00029 |
| + | 82 | −2.687 | 0.00438 |
| + | 83 | −2.631 | 0.00226 |
| + | 84 | −2.716 | 0.00089 |
| + | 85 | 2.703 | 0.00232 |
| + | 86 | −2.641 | 0.00537 |
| + | 87 | −2.686 | 0.00479 |
| + | 88 | −2.654 | 0.00363 |
| + | 89 | 2.695 | 0.00095 |
| + | 90 | −2.758 | 0.00222 |
| + | 91 | 2.702 | 0.00084 |
| + | 92 | −2.694 | 0.00518 |
| + | 93 | 2.711 | 0.00049 |
| + | 94 | −2.771 | 0.00156 |
| + | 95 | 2.604 | 0.00285 |
| − | 96 | −3.495 | 0.00011 |
| − | 97 | 3.224 | 0.00036 |
| − | 98 | −3.225 | 0.00041 |
| − | 99 | −3.145 | 0.00057 |
| − | 100 | −3.055 | 0.00075 |
| − | 101 | −3.037 | 0.00091 |
| − | 102 | −3.066 | 0.00072 |
| − | 103 | 3.06 | 0.00077 |
| − | 104 | −2.985 | 0.00081 |
| − | 105 | −2.983 | 0.00104 |
| − | 106 | −3.022 | 0.00095 |
| − | 107 | −3.054 | 0.00082 |
| − | 108 | −3.006 | 0.00098 |
| − | 109 | −2.917 | 0.00134 |
| − | 110 | −2.924 | 0.00149 |
| − | 111 | −2.882 | 0.0017 |

Although 18 of the 76 genes have unknown function, several pathways or biochemical activities were identified that were well represented such as cell death, cell cycle and proliferation, DNA replication and repair and immune response (Table 5).

TABLE 5
Pathway analysis of the 76 genes from the prognostic signature

| Functional Class | 76-gene signature |
|---|---|
| Cell death | TNFSF10, TNFSF13, MAP4, CD44, IL18, GAS2, NEFL, EEF1A2, |
| | BCLG, C3 |
| Cell cycle | CCNE2, CD44, MAP4, SMC4L1, TNFSF10, AP2A2, FEN1, |
| | KPNA2, ORC3L, PLK1 |
| Proliferation | CD44, IL18, TNFSF10, TNFSF13, PPP1CC, CAPN2, PLK1, SAT |
| DNA replication, | TNFSF10, SMC4L1, FEN1, ORC3L, KPNA2, SUPT16H, POLQ, |
| recombination/repair | ADPRTL1 |
| Immune response | TNFSF10, CD44, IL18, TNFSF13, ARHGDIB, C3 |
| Growth | PPP1CC, CD44, IL18, TNFSF10, SAT, HDGFRP3 |
| Cellular assembly and | MAP4, NEFL, TNFSF10, PLK1, AP2A2, SMC4L1 |
| organization | |
| Transcription | KPNA2, DUSP4, SUPT16H, DKFZP434E2220, PHF11, ETV2 |
| Cell-to-cell signaling | CD44, IL18, TNFSF10, TNFSF13, C3 |
| and interaction | |
| Survival | TNFSF10, TNFSF13, CD44, NEFL |
| Development | IL18, TNFSF10, COL2A1 |
| Cell morphology | CAPN2, CD44, TACC2 |
| Protein synthesis | IL18, TNFSF10, EEF1A2 |
| ATP binding | PRO2000, URKL1, ACACB |
| DNA binding | HIST1H4H, DKFZP434E2220, PHF11 |
| Colony formation | CD44, TNFSF10 |
| Adhesion | CD44, TMEM8 |
| Neurogenesis | CLN8, NEURL |
| Golgi apparatus | GOLPH2, BICD1 |
| Kinase activity | CNK1, URKL1 |
| Transferase activity | FUT3, ADPRTL1 |

Genes implicated in disease progression were found including calpain2, origin recognition protein, dual specificity phosphatases, Rho-GDP dissociation inhibitor, TNF superfamily protein, complement component 3, microtubule-associated protein, protein phosphatase 1 and apoptosis regulator BCL-G. Furthermore, previously characterized prognostic genes such as cyclin E2 (Keyomarsi et al. (2002)) and CD44 (Herrera-Gayol et al. (1999)) were in the gene signature.

The patients providing the samples had not received adjuvant systemic therapy, so the multigene assessment of prognosis was not subject to potentially confounding contributions by predictive factors related to systemic treatment. From this analysis a 76-gene signature that accurately predicts distant tumor relapse that is not specifically prognostic of bone relapse. This signature is applicable to all relapsing breast cancer patients independently of age, tumor size and grade and ER status. In Cox's multivariable analysis for DMFS the 76-gene signature was the only significant variable, superseding the clinical variables, including grade. After 5 years, absolute differences in DMFS and OS between the patients with the good and poor 76-gene signatures were 40% and 27%, respectively. Of the patients with a good prognosis signature, 7% developed distant metastases and 3% died within 5 years. If further validated, this prognostic signature will yield a positive predictive value of 37% and a negative predictive value of 95%, on the assumption of a 25% rate of disease relapse in breast cancer patients. In particular, this signature can be valuable for defining the risk of relapse for the increasing proportion of T1 tumors (<2 cm). Comparison with the St Gallen and NIH guidelines was instructive. Although ensuring the same number of the high-risk patients would receive the necessary treatment, the 76-gene signature would recommend systemic adjuvant chemotherapy to only 52% of the low-risk patients, as compared to 90% and 89% by the St. Gallen and NIH guidelines, respectively (Table 6).

TABLE 6
Comparison of the 76-gene signature and the current conventional
consensus on treatment of breast cancer

Patients guided to receive
adjuvant chemotherapy
in the testing set

| | Metastatic disease | Metastatic disease |
|---|---|---|
| Method | at 5 years (%) | free at 5 years (%) |
| St Gallen | 52/55 (95) | 104/115 (90) |
| NIH | 52/55 (95) | 101/114 (89) |
| 76-gene signature | 52/56 (93) | 60/115 (52) |

The conventional consensus criteria. St. Gallen: tumor ≧2cm, ER-negative, grade 2-3, patient <35 yr (either one of these criteria); NIH: tumor >1cm.

The conventional consensus criteria. St. Gallen: tumor>2 cm, ER-negative, grade 2-3, patient <35 yr (either one of these criteria); NIH: tumor>1 cm.

The 76-gene signature can thus result in a reduction of the number of low-risk relapse patients who would be recommended to have unnecessary adjuvant systemic therapy.

The 76-genes in the prognostic signature belong to many functional classes, suggesting that different paths could lead to disease progression. The signature included well-characterized genes and 18 unknown genes. This finding could explain the superior performance of this signature as compared to other prognostic factors. Although genes involved in cell death, cell proliferation, and transcriptional regulation were found in both patient groups stratified by ER status, the 60 genes selected for the ER-positive group and the 16 genes selected for the ER-negative group had no overlap. This result supports the idea that the extent of heterogeneity and the underlying mechanisms for disease progression could differ for the two ER-based subgroups of breast cancer patients.

Comparison of these results with those of the study by van de Vijver et al. (2002) is difficult because of differences in patients, techniques and materials used. van de Vijver et al. included both node-negative and node-positive patients, who had or had not received adjuvant systemic therapy, and only women younger than 53 years. Furthermore, the microarray platforms used in the studies are different, Affymetrix vs. Agilent. Of the 70 genes of the van't Veer (2002) study, only 48 are present on the Affymetrix U133a array, while of the 76 genes of this profile only 38 are present on the Agilent array. There is a 3-gene overlap between the two signatures (cyclin E2, origin recognition complex, and TNF superfamily protein). Despite the apparent difference, both signatures included genes that identified several common pathways that might be involved in tumor relapse. This finding supports the idea that while there might be redundancy in gene members, effective signatures could be required to include representation of specific pathways.

The strengths of the study described above compared with the study of van de Vijver et al. (2002) are the larger number of untreated relapse patients (286 vs. 141), and the independence of the 76-gene signature with respect to age, menopausal status, and tumor size. The validation set of patients in this approach is completely without overlap with the training set in contrast to 90% of other reports. Ransohoff (2004).

In conclusion, as only approximately 30-40% of the untreated patients develop tumor relapse, the prognostic signature could provide a powerful tool to identify those patients at low risk preventing over treatment in substantial numbers of patients. The recommendation of adjuvant systemic therapy in patients with primary breast cancer could be guided in the future by this prognostic signature. The preferred profiles described in Examples 1-5 (for risk of relapse generally) are the 35-gene portfolio made up of the genes of SEQ ID NOs: 1-35, the 60-gene portfolio made up of the genes of SEQ ID NOs: 36-95 which is best used to prognosticate ER-positive patients, and the 16-gene portfolio made up of genes of SEQ ID NOs: 96-111 which is best used to prognosticate ER-negative patients.

### Example 6

Comparison of Breast Tumor Gene Profile Generated from Laser Capture Microdissection and Bulk Tissue in Stage I/II Breast Cancer

Gene-expression profiling has been shown to be a powerful diagnostic and prognostic tool for a variety of cancer types. Almost exclusively in all cases bulk tumor RNA was used for hybridization on the chip. Estrogens play important roles in the development and growth of hormone-dependent tumors.

About 75% of breast cancers express estrogen receptor (ER), which is an indicator for (adjuvant) tamoxifen treatment and is associated with patient outcomes.

To gain insights into the mechanisms trigged by estrogen in breast epithelia cells and their association with tumorigenesis, laser capture microdissection (LCM) was used to procure histologically homogenous population of tumor cells from 29 early stage primary breast tumors, in combination with GeneChip expression analysis. Of these 29 patients, 11 were ER-negative and 17 were ER-positive based on quantitative ligand binding or enzyme immunoassays on tumor cytosols. For comparison, gene expression profiling was also obtained using bulk tissue RNA isolated from the same group of 29 patients.

Fresh frozen tissue samples were collected from 29 lymph-node-negative breast cancer patients who had been surgically treated for a breast tumor and had not received neoadjuvant systemic therapy. For each patient tissue sample, an H&E slide was first used to evaluate the cell morphology. RNA was isolated from both tumor cells obtained by LCM (PALM) performed on cryostat sections and from whole cryostat sections, i.e., bulk tissue of the same tumor. RNA sample quality was analyzed by an Agilent BioAnalyzer. The RNA samples were hybridized to Affymetrix human U133A chip that contains approximately 22,000 probe sets. The fluorescence was quantified and the intensities were normalized. Clustering Analysis and Principal Component Analysis were used to group patients with similar gene expression profiles. Genes that are differentially expressed between ER-positive and ER-negative samples were selected.

Total RNA isolated from LCM procured breast cancer cells was subjected to two-round T7 based amplification in target preparation, versus one round amplification with bulk tissue RNA. Expression levels of 21 control genes (Table 7) were compared between LCM data set and bulk tissue set to demonstrate the fidelity of linear amplification.

TABLE 7
Control gene list

| SEQ ID | |
|---|---|
| NO: | Name |
| 112 | protein phosphatase 2, regulatory subunit B (B56), delta isoform |
| 113 | CCCTC-binding factor (zinc finger protein) |
| 114 | solute carrier family 4 (anion exchanger), member 1, |
| | adaptor protein |
| 115 | ribonuclease P |
| 116 | hypothetical protein FLJ20188 |
| 117 | KIAA0323 protein |
| 118 | cDNA FLJ12469 |
| 119 | translation initiation factor eIF-2b delta subunit |
| 120 | heterogeneous nuclear ribonucleoprotein K |
| 121 | hydroxymethylbilane synthase |
| 122 | cDNA DKFZp586O0222 |
| 123 | chromosome 20 open reading frame 4 |
| 124 | thyroid hormone receptor interactor 4 |
| 125 | hypoxanthine phosphoribosyltransferase 1 (Lesch-Nyhan |
| | syndrome) |
| 126 | DnaJ (Hsp40) homolog, subfamily C, member 8 |
| 127 | dual specificity phosphatase 11 (RNA/RNP complex |
| | 1-interacting) |
| 128 | calcium binding atopy-related autoantigen 1 |
| 129 | stromal cell-derived factor 2 |
| 130 | Ewing sarcoma breakpoint region 1 |
| 131 | CCR4-NOT transcription complex, subunit 2 |
| 132 | F-box only protein 7 |

The results obtained are depicted in Table 8.

TABLE 8
Clinical characteristics of patients

| Characteristic | No. of patients (%) |
|---|---|
| Age in years | |
| <40 | 1 (3) |
| 40-44 | 5 (17) |
| 45-49 | 8 (28) |
| ≧50 | 15 (52) |
| Tumor diameter in mm | |
| ≦20 | 11 (40) |
| >20 | 17 (59) |
| Histologic grade | |
| II (intermediate) | 5 (17) |
| III (poor) | 12 (41) |
| Estrogen-receptor status | |
| Negative | 11 (40) |
| Positive | 17 (59) |
| Surgery | |
| Breast-conserving therapy | 26 (90) |
| Mastectomy | 3 (10) |
| Chemotherapy | |
| No | 29 (100) |
| Hormonal therapy | |
| No | 29 (100) |
| Disease-free survival in months | |
| ≦48 | 13 (45) |
| >48 | 16 (55) |

A hierarchical clustering based on 5121 genes showed that LCM and bulk tissue samples are completely separated based on global RNA expression profiles. The expression levels of 21 control genes in RNA isolates from LCM samples and bulk tissues subjected to an additional round of linear amplification used for RNA obtained by LCM did not cause differential expression of the control genes. Differentially expressed genes between ER-positive and ER-negative sub-clusters in both LCM and bulk tissue samples were defined by Student T-test pathway analysis by Gene Ontology for genes exclusively associated with ER in LCM samples, exclusively in bulk tissues, and for those that are common in both LCM and bulk tissue were conducted.

The results obtained show several important conclusions. First, genes related to cell proliferation and energy metabolism were seen differentially expressed in ER−/ER+patients both in bulk tissue data set and LCM data set. Second, due to the enrichment of breast cancer cells via LCM, genes involved in cell surface receptor linked signal transduction, RAS signal transduction, JAK-STAT signal transduction and apoptosis were found associate to ER status. These genes were not identified in bulk data set. Third, microdissection provides a sensitive approach to studying epithelial tumor cells and an insight into signaling pathway associated with estrogen receptors. Therefore, it is clear that the application of the gene expression profile described herein to LCM isolated tumor cells is commensurate with results obtained in heterogeneous bulk tissue.

### Example 7

Validation and Pathway Analysis of the 76-Gene Prognostic Signature in Breast Cancer

This Example reports the results of a validation study in which the 76-gene signature was used to predict outcomes of 132 patients obtained from 4 independent sources.

In addition, in order to evaluate the robustness of this gene signature, this Example further provides identification of substitutable components of the signature and describes how the substitutions lead to the identification of key pathways in an effective signature.

Fresh frozen tissue samples were collected from 132 patients who had been surgically treated for a breast tumor and had not received adjuvant systemic therapy. The patient samples used were collected between 1980 and 1996. For each patient tissue sample, an H&E slide was used to evaluate the cell morphology. Then total RNA samples were prepared and the sample quality was analyzed by Agilent BioAnalyzer. The RNA samples were analyzed by microarray analysis. The fluorescence was quantified and the intensities were normalized. A relapse hazard score was calculated for each patient based on the expression levels of the 76-gene signature. The patients were classified into good and poor outcome groups.

In order to evaluate the robustness of this gene signature, two statistical analyses were designed and used. First, gene selection and signature construction procedures that were used to discover the 76-gene signature were repeated. As shown in Table 8, ten training sets of 115 patients each were randomly selected from the total of 286 patients. The remaining patients were served as the testing set.

Second, the number of patients in a training set was increased to 80% of the 286 patients and used the remaining 20% of the patients as the testing set. This selection procedure was also repeated 10 times. In both procedures, Kaplan-Meier survival curves were used to ensure no significant difference in disease free survival between the training and the testing pair. Genes were selected and a signature was built from each of the training sets using Cox's proportional-hazards regression. Each signature was validated in the corresponding testing set. Furthermore, the 76-gene prognostic signature was assigned into functional groups using GO ontology classification. Pathways that cover significant numbers of genes in the signature were selected (p-value <0.05 and >2 hits). The selected pathways were also evaluated in all the prognostic signatures derived from different training sets.

Table 9A:
Results from 10 signatures using training sets of 115 patients,
Table 9B:
Results from 10 signatures using training sets of 80% of the patients.

| A | | | B | | |
|---|---|---|---|---|---|
| AUC of ROC | 0.62 | (0.55-0.70) | AUC of | 0.62 | (0.53-0.72) |
| | | | ROC | | |
| Sensitivity | 86% | (0.84-0.88) | Sensitivity | 83% | (0.81-0.85) |
| Specificity | 34% | (0.21-0.56) | Specificity | 46% | (0.28-0.62) |
| Freq. of | 33% | | Freq. of | 33% | |
| Relapse | | | Relapse | | |
| PPV | 40% | (0.35-0.49) | PPV | 47% | (0.32-0.58) |
| NPV | 81% | (0.75-0.89) | NPV | 82% | (0.78-0.89) |
| Odds Ratio | 3.5 | (1.7-7.9) | Odds Ratio | 5.6 | (1.7-15) |

The results obtained in this Example show that:

The 76-gene signature is successfully validated in 132 independent patients, giving an AUC value of 0.757 in the 132 relapse breast cancer patients from 4 independent sources. The signature shows 88% sensitivity and 41% specificity.

The average AUC for the substitute signatures is 0.64 (95% CI: 0.53-0.72). This result is consistent with that of the 76-gene predictor (AUC of 0.69). Twenty-one pathways over-represented in the 76-gene signature were also found in all the other prognostic signatures, suggesting that common biological pathways are involved in tumor relapse.

These results suggest that gene expression profiles provide a powerful approach to perform risk assessment of patient outcome. The data highlight the feasibility of a molecular prognostic assay that provides patients with a quantitative measurement of tumor relapse.

### Example 8

Bone Relapse Signatures

From the sample set used to establish the 76-gene profile for predicting distant relapse, 107 samples were selected to further study bone relapse. These samples were all selected because the site of relapse was known and the samples could be grouped into bone and non-bone distant relapse sets. Those classified as bone relapse samples included those that had bone relapse and also possibly relapsed in other parts of the body. The remaining relapse patient samples were labeled non-bone.

The information relating to the samples used in these analyses are shown in Table 10.

Two different analyses were performed. First, Significance Analysis of Microarrays (SAM) analysis was used to identify differentially expressed genes in the case of relapse in bone relative to relapse elsewhere (i.e., non-bone). In the second analysis a bone relapse predictor was established to determine the likelihood of a patient for relapsing in bone. This signature is referred to as a Prediction Analysis of Microarrays (PAM).

In the case of the SAM analysis, 300 permutations of the data were used to calculate a false discovery rate (FDR). Genes were considered significant when the FDR was below 5% and when a minimum 1.7 fold difference in expression level was observed. To construct a diagnostic profile that would be useful in distinguishing those who (from among those likely to relapse) would be likely to relapse in bone, samples were divided into a training set (n=72, 46 with a relapse in bone and 26 with a non-bone relapse) and a testing set (n=35, 23 bone and 12 non-bone relapses) stratified by site of relapse, ER protein level and metastasis-free interval. A gene selection step using an optimal cut-off procedure was performed in the samples of the training set. All measured expression levels of a gene were used as the cut point to assign the gene being “high” or “low” in a particular sample, keeping a minimum of 20 samples in one of the groups. Knowing the site of relapse of these samples, the frequencies for the categories high/Bone, low/Bone, high/Non-bone and low/Non-bone were counted for each cut-off. The optimal cut-off was determined by using the χ² distribution. Genes were included if the maximal χ² score was 10.827 or higher (p<0.001) for analysis in a Prediction Analysis of Microarrays (PAM).

TFF1 was the most significant gene in the gene profiles established through this procedure (from a statistical point of view). Further experiments to determine TFF1 mRNA levels by quantitative RT-PCR were performed using the following primer pairs (TGGAGCAGAGAGGAGGCAAT and ACGAACGGTGTCGTCGAAAC). The samples selected for the RT-PCR study were matched for patient and tumor characteristics listed in Table 10. Gene expression levels were expressed relative to a panel of housekeeper genes and were ²log transformed. The difference in gene expression levels of TFF1 was correlated to the two relapse groups and p-values were calculated using Kruskal-Wallis anova, χ² approximates and corrected for ties. Statistical analyses were performed using Analyse-it software (Analyse-it Software Ltd, Leeds, United Kingdom).

Results

SAM Analysis

The samples described above were classified according to the site of relapse, 69 samples were labeled as bone and 38 as non-bone. Using SAM, 73 probe-sets representing 69 unique genes were seen as significantly differentially expressed between the bone and non-bone samples. The 5 highest ranking genes were TFF1, TFF3, AGR2, NAT1, and CRIP1 all of which are higher expressed in the bone relapse samples. The highest ranked gene, TFF1, was studied in 122 independent breast tumors by quantitative RT-PCR. TFF1 expression was significantly associated with the site of relapse (p=0.0015) with relative median expression level and 95% C1 for TFF1 of 3.02 (1.41 to 4.66) and −1.63 (−5.44 to 2.49) for the bone and non-bone relapse group, respectively. Genes corresponding to SEQ ID No.s 112-147 were higher expressed in bone relapse samples. The remainder were lower expressed.

PAM Analysis

The samples were divided into a training set (n=72) and a testing set (n=35) stratified by site of relapse, ER protein level and metastasis-free interval. Using the optimal cut-off procedure, 588 informative genes were selected for input in the PAM analysis. A 31-gene predictor was selected after 10-fold cross-validation of the training set that could identify the bone relapse samples in the testing set with 100% sensitivity and 50% specificity. The predictor showed a 79.3% positive predictive value and misclassified 17% of the samples. 17 genes in the profile, including TFF1, were also present in the SAM gene list (all 31 genes are referenced in the “PAM”-column in Table 11).

To ascertain the validity of the gene set, 50 sets of 100 randomly chosen genes were also analyzed. These random gene sets were used for input in a PAM analysis using the same training and testing set. The mean percentage of misclassified samples was 28.5% (SD 4.3%). This indicates that the 17% misclassified samples found by the actual PAM gene list is significantly lower (z-value 2.67, two-tailed p=0.008) than the random data sets.

TABLE 10
Clinical and tumor characteristics of patients for SAM and PAM
analyses.

| Characteristics | All patients | Bone relapse | Non-bone relapse |
|---|---|---|---|
| Number | 107 | 69 | 38 |
| Age (mean ± SD) | 53 ± 12 | 52 ± 12 | 54 ± 11 |
| ≦40 yr | 16 (15%) | 12 (17%) | 4 (11%) |
| 41-55 yr | 49 (46%) | 32 (46%) | 17 (45%) |
| 56-70 yr | 34 (32%) | 20 (29%) | 14 (37%) |
| >70 yr | 8 (7%) | 5 (7%) | 3 (8%) |
| Menopausal status | | | |
| Premenopausal | 51 (48%) | 33 (48%) | 18 (47%) |
| Postmenopausal | 56 (52%) | 36 (52%) | 20 (53%) |
| T stage | | | |
| T1 | 54 (50%) | 38 (55%) | 16 (42%) |
| T2 | 50 (47%) | 31 (45%) | 19 (50%) |
| T¾ | 3 (3%) | 0 (0%) | 3 (8%) |
| Grade | | | |
| Poor | 61 (57%) | 39 (57%) | 22 (58%) |
| Good-Moderate | 10 (9%) | 9 (13%) | 1 (3%) |
| Unknown | 36 (34%) | 21 (30%) | 15 (39%) |
| ER*^{†} | | | |
| Positive | 80 (75%) | 57 (83%) | 23 (61%) |
| Negative | 27 (25%) | 12 (17%) | 15 (39%) |
| PgR* | | | |
| Positive | 56 (52%) | 38 (55%) | 18 (47%) |
| Negative | 48 (45%) | 28 (41%) | 20 (52%) |
| Unknown | 3 (3%) | 3 (4%) | 0 (0%) |

*ER and PgR are defined positive when tumors contain >10 fmol/mg protein or >10% positive tumor cells.
^{†}Patient characteristics are equally distributed between the bone or non-bone relapses, except for ER status (p-value = 0.02), calculated using the χ² distribution.

TABLE 11
Genes involved in bone matastasis of breast cancer.

| SEQ | | | | | | | |
|---|---|---|---|---|---|---|---|
| ID | | | SAM | Fold | | | |
| NO | Probe-id | Gene Symbol | Score | Change | FDR (%) | PAM^{†} | Gene Title |
| 112 | 205009_at | TFF1 | −4.92 | 3.1 | 1.9 | yes | trefoil factor 1 |
| 113 | 204623_at | TFF3 | −4.23 | 2.6 | 1.9 | yes | trefoil factor 3 (intestinal) |
| 114 | 209173_at | AGR2 | −4.06 | 1.9 | 1.9 | | anterior gradient 2 homolog |
| 115 | 214440_at | NAT1 | −4.04 | 2.5 | 1.9 | yes | N-acetyltransferase 1 |
| 116 | 205081_at | CRIP1 | −3.80 | 1.9 | 1.9 | yes | cysteine-rich protein 1 (intestinal) |
| 117 | 214774_x_at | TNRC9 | −3.72 | 1.9 | 1.9 | yes | trinucleotide repeat containing 9 |
| 118 | 214858_at | — | −3.60 | 2.0 | 1.9 | yes | Pp14571 |
| 119 | 219197_s_at | SCUBE2 | −3.59 | 2.1 | 1.9 | | signal peptide, CUB domain, EGF-like 2 |
| 120 | 215108_x_at | TNRC9 | −3.57 | 1.9 | 1.9 | yes | trinucleotide repeat containing 9 |
| 121 | 206754_s_at | CYP2B6 | −3.57 | 2.1 | 1.9 | | cytochrome P450, family 2, subfamily B, polypeptide 6 |
| 122 | 210056_at | RND1 | −3.48 | 1.7 | 1.9 | yes | Rho family GTPase 1 |
| 123 | 205186_at | DNALI1 | −3.45 | 2.0 | 1.9 | | dynein, axonemal, light intermediate polypeptide 1 |
| 124 | 203130_s_at | KIF5C | −3.42 | 2.0 | 1.9 | | kinesin family member 5C |
| 125 | 216623_x_at | TNRC9 | −3.32 | 1.9 | 1.9 | yes | trinucleotide repeat containing 9 |
| 126 | 222256_s_at | PLA2G4B | −3.31 | 1.8 | 1.9 | | phospholipase A2, group IVB (cytosolic) |
| 127 | 210021_s_at | UNG2 | −3.29 | 1.7 | 1.9 | yes | uracil-DNA glycosylase 2 |
| 128 | 204607_at | HMGCS2 | −3.22 | 2.3 | 1.9 | yes | 3-hydroxy-3-methylglutaryl-Coenzyme A synthase 2 |
| 129 | 213664_at | SLC1A1 | −3.17 | 2.7 | 1.9 | yes | solute carrier family 1 member 1 |
| 130 | 211657_at | CEACAM6 | −3.16 | 2.3 | 1.9 | | carcinoembryonic antigen-related cell adhesion molecule 6 |
| 131 | 222348_at | — | −3.01 | 1.7 | 1.9 | | — |
| 132 | 209114_at | TSPAN-1 | −2.93 | 1.8 | 1.9 | | tetraspan 1 |
| 133 | 205645_at | REPS2 | −2.84 | 1.8 | 1.9 | | RALBP1 associated Eps domain containing 2 |
| 134 | 39763_at | HPX | −2.84 | 1.7 | 1.9 | | hemopexin |
| 135 | 214099_s_at | PDE4DIP | −2.83 | 2.2 | 1.9 | | phosphodiesterase 4D interacting protein |
| 136 | 203757_s_at | CEACAM6 | −2.77 | 2.8 | 1.9 | | carcinoembryonic antigen-related cell adhesion molecule 6 |
| 137 | 204485_s_at | TOM1L1 | −2.76 | 1.8 | 1.9 | | target of myb1-like 1 |
| 138 | 206378_at | SCGB2A2 | −2.76 | 1.9 | 1.9 | | secretoglobin, family 2A, member 2 |
| 139 | 211712_s_at | ANXA9 | −2.76 | 1.7 | 1.9 | | annexin A9 |
| 140 | 204378_at | BCAS1 | −2.73 | 1.8 | 1.9 | | breast carcinoma amplified sequence 1 |
| 141 | 206243_at | TIMP4 | −2.67 | 1.8 | 3.3 | | tissue inhibitor of metalloproteinase 4 |
| 142 | 210272_at | CYP2B6 | −2.58 | 1.8 | 4.4 | | cytochrome P450, family 2, subfamily B, polypeptide 6 |
| 143 | 205597_at | C6orf29 | −2.56 | 1.7 | 4.4 | | chromosome 6 open reading frame 29 |
| 144 | 221946_at | C9orf116 | −2.50 | 1.9 | 4.4 | | chromosome 9 open reading frame 116 |
| 145 | 210297_s_at | MSMB | −2.49 | 3.0 | 4.4 | | microseminoprotein, beta- |
| 146 | 204014_at | DUSP4 | −2.48 | 1.9 | 4.4 | | dual specificity phosphatase 4 |
| 147 | 204379_s_at | FGFR3 | −2.47 | 1.8 | 4.4 | | fibroblast growth factor receptor 3 |
| 148 | 205014_at | FGFBP1 | 3.66 | 1.9 | 1.9 | | fibroblast growth factor binding protein 1 |
| 149 | 209406_at | BAG2 | 3.65 | 1.8 | 1.9 | | BCL2-associated athanogene 2 |
| 150 | 210655_s_at | FOXO3A | 3.56 | 2.6 | 1.9 | yes | forkhead box O3A |
| 151 | 220559_at | EN1 | 3.53 | 2.5 | 1.9 | | engrailed homolog 1 |
| 152 | 209800_at | KRT16 | 3.46 | 12.7 | 1.9 | | keratin 16 |
| 153 | 209373_at | BENE | 3.39 | 1.9 | 1.9 | | BENE protein |
| 154 | 214595_at | KCNG1 | 3.33 | 3.4 | 1.9 | | potassium voltage-gated channel, subfamily G, member 1 |
| 155 | 216365_x_at | IGLC2 /// IGLJ3 | 3.28 | 2.7 | 1.9 | | Immunoglobulin lambda constant 2 /// Immunoglobulin |
| | | | | | | | lambda joining 3 |
| 156 | 206125_s_at | KLK8 | 3.22 | 1.8 | 1.9 | | kallikrein 8 |
| 157 | 211637_x_at | — | 3.22 | 2.4 | 1.9 | | Immunoglobulin heavy chain V region (Humha448) /// Similar to |
| | | | | | | | Ig heavy chain V-I region HG3 precursor |
| 158 | 209126_x_at | KRT6B | 3.20 | 2.5 | 1.9 | yes | keratin 6B |
| 159 | 219480_at | SNAI1 | 3.15 | 1.8 | 3.3 | | snail homolog 1 |
| 160 | 205347_s_at | TMSNB | 3.10 | 2.3 | 3.3 | | thymosin, beta, identified in neuroblastoma cells |
| 161 | 210683_at | NRTN | 3.02 | 2.7 | 3.3 | yes | neurturin |
| 162 | 1438_at | EPHB3 | 2.99 | 2.0 | 3.3 | yes | EPH receptor B3 |
| 163 | 217294_s_at | ENO1 | 2.87 | 2.3 | 4.4 | yes | enolase 1 |
| 164 | 215223_s_at | SOD2 | 2.86 | 1.9 | 4.4 | | superoxide dismutase 2, mitochondrial |
| 165 | 211908_x_at | IGHG1 | 2.85 | 1.9 | 4.4 | | immunoglobulin heavy constant gamma 1 (G1m marker) |
| 166 | 222242_s_at | KLK5 | 2.80 | 2.2 | 4.4 | | kallikrein 5 |
| 167 | 206391_at | RARRES1 | 2.79 | 2.2 | 4.4 | | retinoic acid receptor responder 1 |
| 168 | 219415_at | TTYH1 | 2.78 | 5.0 | 4.4 | | tweety homolog 1 |
| 169 | 209772_s_at | CD24 | 2.78 | 2.1 | 4.4 | | CD24 antigen |
| 170 | 217281_x_at | MGC27165 /// | 2.77 | 2.1 | 4.4 | | hypothetical protein MGC27165 /// immunoglobulin heavy |
| | | IGHG1 | | | | | constant gamma 1 (G1m maker) |
| 171 | 204855_at | SERPINB5 | 2.75 | 2.5 | 4.4 | | serine (or cysteine) proteinase inhibitor, clade B member 5 |
| 172 | 201387_s_at | UCHL1 | 2.73 | 2.6 | 4.4 | | ubiquitin carboxyl-terminal esterase L1 |
| 173 | 220425_x_at | ROPN1 | 2.73 | 2.6 | 4.4 | | ropporin, rhophilin associated protein 1 |
| 174 | 218484_at | LOC56901 | 2.72 | 2.4 | 4.4 | | NADH: ubiquinone oxidoreductase MLRQ subunit homolog |
| 175 | 215177_s_at | ITGA6 | 2.70 | 1.9 | 4.4 | | integrin, alpha 6 |
| 176 | 209372_x_at | TUBB /// MGC8685 | 2.70 | 2.1 | 4.4 | | tubulin, beta polypeptide |
| 177 | 202316_x_at | UBE4B | 2.67 | 1.9 | 4.4 | | ubiquitination factor E4B |
| 178 | 211641_x_at | IGHM | 2.67 | 2.0 | 4.4 | | Immunoglobulin heavy chain VH3 (H11) |
| 179 | 217404_s_at | COL2A1 | 2.65 | 4.4 | 4.4 | | collagen, type II, alpha 1 |
| 180 | 203126_at | IMPA2 | 2.65 | 1.9 | 4.4 | | inositol(myo)-1(or 4)-monophosphatase 2 |
| 181 | 221986_s_at | DRE1 | 2.64 | 1.8 | 4.4 | | DRE1 protein |
| 182 | 205778_at | KLK7 | 2.64 | 4.0 | 4.4 | | kallikrein 7 |
| 183 | 202134_s_at | TAZ | 2.60 | 2.0 | 4.4 | | transcriptional co-activator with PDZ-binding motif (TAZ) |
| 184 | 212065_s_at | USP34 | 2.60 | 1.8 | 4.4 | | ubiquitin specific protease 34 |
| 185 | 201952_at | — | | | | yes | |
| 186 | 202987_at | C6ORF4 | | | | yes | Chromosome 6 open reading frame 4 |
| 187 | 218489_s_at | ALAD | | | | yes | aminolevulinate, delta-, dehydratase |
| 188 | 213606_s_at | ARHGDIA | | | | yes | Rho GDP dissociation inhibitor (GDI) alpha |
| 189 | 201679_at | ARS2 | | | | yes | arsenate resistance protein |
| 190 | 217528_at | CLCA2 | | | | yes | chloride channel, calcium activated, family member 2 |
| 191 | 205830_at | CLGN | | | | yes | calmegin |
| 192 | 220363_s_at | ELMO2 | | | | yes | engulfment and cell motility 2 (ced-12 homolog, C. elegans) |
| 193 | 220622_at | LRRC31 | | | | yes | leucine rich repeat containing 31 |
| 194 | 202601_s_at | HTATSF1 | | | | yes | HIV TAT specific factor 1 |
| 195 | 206638_at | HTR2B | | | | yes | 5-hydroxytryptamine (serotonin) receptor 2B |
| 196 | 218211_s_at | MLPH | | | | yes | melanophilin |
| 197 | 218985_at | SLC2A8 | | | | yes | solute carrier family 2, member 8 |
| 198 | 209278_s_at | TFPI2 | | | | yes | tissue factor pathway inhibitor 2 |

^{†}Genes identified by the PAM analysis; the genes from this analysis that were not identified by SAM are appended after the SAM-identified genes.

Pathway Analysis for Bone Relapse Signature

Differentially expressed genes were compared to those found in the Gene Ontology and KEGG databases. As there were only 8 genes from the SAM list annotated in the KEGG database, that list was merged with a recently published bone metastasis profile. In that study, Kang et al. generated gene expression profiles from sub clones of the ER-negative breast cancer cell line MDA-MB-231 that when injected into mice, poorly or efficiently relapsed to bone. Differentially expressed genes between those two subtypes were considered as the bone relapse signature. Since Kang et al. used the same microarrays as those used in the examples above, it was convenient to merge their 127 probe-set list (122 unique genes) with the SAM gene list (n=69). Although the two profiles share only one gene (BENE), it is likely they address common pathways. To this end, both lists were mapped on the KEGG database. The in total 20 KEGG-annotated genes revealed that 5 of the 20 genes (FGF5, SOS1 and DUSP1 (Kang list) and FGFR3 and DUSP4 (SAM)) were located in the FGFR-p42/44 MAP-kinase pathway; this number of genes is statistically different from a random dataset (p<0.0001). All 5 genes were up-regulated in the bone metastasizing cells/tumors. The 142 genes from the combined list that were annotated in Gene Ontology database were studied. Determinations were made as to whether the Gene Ontology descriptions were over-represented in the merged SAM/Kang list compared with all genes printed on the U133a chip. Over-represented annotations point to biological processes, which are possibly linked to the site of relapse. For example, the description “extracellular” was linked to 21 of the 142 (14.8%) genes from the bone marker list, whereas 1350 out of 16367 genes (8.2%) of the U133a chip were annotated to this description. This means “extracellular” is 1.8 times over-represented (p=0.006, χ²-distribution) in the bone relapse list. Other examples are “cell adhesion” (17 genes, p=0.0007) and “cell organization and biogenesis” (22 genes, p=2.3 10⁻⁵) found 2.2 and 2.4 times over-represented, respectively. Additional, “immune response” was significant (p=8.7 10⁻⁵), but in contrast to the above-mentioned descriptions the genes linked to “immune response” originated predominantly from the Kang list.

Table 12 identifies the sequences referred to in this specification.

TABLE 12
Sequence identification

| SEQ | | | | |
|---|---|---|---|---|
| ID | | | | |
| NO: | psid | Gene Name | Accession # | Gene description |
| 1 | 213165_at | CDABP0086 | AI041204 | |
| 2 | 217432_s_at | | AF179281 | iduronate 2-sulfatase (Hunter syndrome) |
| 3 | 221500_s_at | | BE782754 | syntaxin 16/ |
| 4 | 208452_x_at | MYO9B | NM_004145 | myosin IXB |
| 5 | 220234_at | CA8 | NM_004056 | carbonic anhydrase VIII |
| 6 | 207865_s_at | BMP8 | NM_001720 | bone morphogenetic protein 8 (osteogenic |
| | | | | protein 2) |
| 7 | 201769_at | KIAA0171 | NM_014666 | KIAA0171 gene product |
| 8 | 218940_at | FLJ13920 | NM_024558 | hypothetical protein FLJ13920 |
| 9 | 209018_s_at | BRPK | BF432478 | protein kinase BRPK |
| 10 | 216647_at | DKFZp586L1824 | AL117663 | from clone DKFZp586L1824 |
| 11 | 213405_at | DKFZp564E122 | N95443 | from clone DKFZp564E122 |
| 12 | 202921_s_at | ANK2 | NM_001148 | ankyrin 2, neuronal, transcript variant 1 |
| 13 | 208401_s_at | | U01157 | glucagon-like peptide-1 receptor with CA |
| | | | | dinucleotide repeat |
| 14 | 218090_s_at | WDR11 | NM_018117 | WD40 repeat domain 11 protein |
| 15 | 218139_s_at | FLJ10813 | NM_018229 | hypothetical protein FLJ10813 |
| 16 | 202485_s_at | MBD2 | NM_003927 | methyl-CpG binding domain protein 2, |
| | | | | transcript variant 1 |
| 17 | 201357_s_at | SF3A1 | NM_005877 | splicing factor 3a, subunit 1, 120 kD |
| 18 | 214616_at | H3FD | NM_003532 | H3 histone family, member D |
| 19 | 207719_x_at | KIAA0470 | NM_014812 | KIAA0470 gene product |
| 20 | 202734_at | TRIP10 | NM_004240 | thyroid hormone receptor interactor 10 |
| 21 | 202175_at | FLJ22678 | NM_024536 | hypothetical protein FLJ22678 |
| 22 | 213870_at | | AL031228 | clone 1033B10 on chromosome 6p21.2-21.31 |
| 23 | 208967_s_at | adk2 | U39945 | adenylate kinase 2 |
| 24 | 204312_x_at | | AI655737 | cAMP responsive element binding protein 1 |
| 25 | 203815_at | GSTT1 | NM_000853 | glutathione S-transferase ζ 1 |
| 26 | 207996_s_at | C18ORF1 | NM_004338 | chromosome 18 open reading frame 1 |
| 27 | 221435_x_at | HT036 | NM_031207 | hypothetical protein HT036 |
| 28 | 219987_at | FLJ12684 | NM_024534 | hypothetical protein FLJ12684 |
| 29 | 221559_s_at | MGC: 2488 | BC000229 | clone MGC: 2488 |
| 30 | 207007_at | NR1I3 | NM_005122 | nuclear receptor subfamily 1, group I, mem 3 |
| 31 | 219265_at | FLJ13204 | NM_024761 | hypothetical protein FLJ13204 |
| 32 | 40420_at | | AB015718 | lok mRNA for protein kinase |
| 33 | 202266_at | AD022 | NM_016614 | TRAF and TNF receptor-associated protein |
| 34 | 219522_at | FJX1 | NM_014344 | putative secreted ligand homologous to fjx1 |
| 35 | 212334_at | AKAP350C | BE880245 | AKAP350C, alternatively spliced |
| 36 | 219340_s_at | CLN8 | AF123759 | Putative transmembrane protein |
| 37 | 217771_at | GP73 | NM_016548 | Golgi membrane protein (LOC51280) |
| 38 | 202418_at | Yif1p | NM_020470 | Putative transmembrane protein; homolog of |
| | | | | yeast Golgi membrane protein |
| 39 | 206295_at | IL-18 | NM_001562 | Interleukin 18 |
| 40 | 201091_s_at | | BE748755 | Heterochromatin-like protein |
| 41 | 204015_s_at | DUSP4 | BC002671 | Dual specificity phosphatase 4 |
| 42 | 200726_at | PPP1CC | NM_002710 | Protein phosphatase 1, catalytic subunit, γ |
| | | | | isoform |
| 43 | 200965_s_at | ABLIM-s | NM_006720 | Actin binding LIM protein 1, transcript variant |
| 44 | 210314_x_at | TRDL-1 | AF114013 | Tumor necrosis factor-related death ligand 1 γ |
| 45 | 221882_s_at | M83 | AI636233 | Five-span transmembrane protein |
| 46 | 217767_at | C3 | NM_000064 | Complement component 3 |
| 47 | 219588_s_at | FLJ20311 | NM_017760 | hypothetical protein |
| 48 | 204073_s_at | C11ORF9 | NM_013279 | chromosome 11 open reading frame 9 |
| 49 | 212567_s_at | | AL523310 | Putative translation initiation factor |
| 50 | 211382_s_at | TACC2 | AF220152 | |
| 51 | 201663_s_at | CAP-C | NM_005496 | chromosome-associated polypeptide C |
| 52 | 221344_at | OR12D2 | NM_013936 | Olfactory receptor, family 12, subfamily D, |
| | | | | member 2 |
| 53 | 210028_s_at | ORC3 | AF125507 | Origin recognition complex subunit 3 |
| 54 | 218782_s_at | PRO2000 | NM_014109 | PRO2000 protein |
| 55 | 201664_at | SMC4 | AL136877 | (Structural maintenance of chromosome 4, |
| | | | | yeast)-like |
| 56 | 219724_s_at | KIAA0748 | NM_014796 | KIAA0748 gene product |
| 57 | 204014_at | DUSP4 | NM_001394 | Dual specificity phosphatase 4 |
| 58 | 212014_x_at | CD44 | AI493245 | CD44 |
| 59 | 202240_at | PLK1 | NM_005030 | Polo (Drosophila)-like kinase 1 |
| 60 | 204740_at | CNK1 | NM_006314 | connector enhancer of KSR-like (Drosophila |
| | | | | kinase suppressor of ras) |
| 61 | 208180_s_at | H4FH | NM_003543 | H4 histone family, member H |
| 62 | 204768_s_at | FEN1 | NM_004111 | Flap structure-specific endonuclease |
| 63 | 203391_at | FKBP2 | NM_004470 | FK506-binding protein 2 |
| 64 | 211762_s_at | KPNA2 | BC005978 | Karyopherin α 2 (RAG cohort 1, importin α 1) |
| 65 | 218914_at | CGI-41 | NM_015997 | CGI-41 protein |
| 66 | 221028_s_at | MGC11335 | NM_030819 | hypothetical protein MGC11335 |
| 67 | 211779_x_at | MGC13188 | BC006155 | Clone MGC: 13188 |
| 68 | 218883_s_at | FLJ23468 | NM_024629 | hypothetical protein FLJ23468 |
| 69 | 204888_s_at | | AA772093 | Neuralized (Drosophila)-like |
| 70 | 217815_at | FACTP140 | NM_007192 | Chromatin-specific transcription elongation |
| | | | | factor, 140 kD subunit |
| 71 | 201368_at | Tis11d | U07802 | |
| 72 | 201288_at | ARHGDIB | NM_001175 | Rho GDP dissociation inhibitor (GDI) β |
| 73 | 201068_s_at | PSMC2 | NM_002803 | Proteasome (prosome, macropain) 26S |
| | | | | subunit, ATPase, 2 |
| 74 | 218478_s_at | DKFZP434E2220 | NM_017612 | hypothetical protein DKFZP434E2220 |
| 75 | 214919_s_at | KIAA1085 | R39094 | |
| 76 | 209835_x_at | | BC004372 | Similar to CD44 |
| 77 | 217471_at | | AL117652 | |
| 78 | 203306_s_at | SLC35A1 | NM_006416 | Solute carrier family 35 (CMP-sialic acid |
| | | | | transporter), member 1 |
| 79 | 205034_at | CCNE2 | NM_004702 | Cyclin E2 |
| 80 | 221816_s_at | | BF055474 | Putative zinc finger protein NY-REN-34 antigen |
| 81 | 219510_at | POLQ | NM_006596 | Polymerase (DNA directed) ζ |
| 82 | 217102_at | | AF041410 | Malignancy-associated protein |
| 83 | 208683_at | CANP | M23254 | Ca2-activated neutral protease large subunit |
| 84 | 215510_at | | AV693985 | ets variant gene 2 |
| 85 | 218533_s_at | FLJ20517 | NM_017859 | hypothetical protein FLJ20517 |
| 86 | 215633_x_at | LST-1N | AV713720 | mRNA for LST-1N protein |
| 87 | 221928_at | | AI057637 | Hs234898 ESTs, weakly similar to 2109260A |
| | | | | B-cell growth factor |
| 88 | 214806_at | BICD | U90030 | Bicaudal-D |
| 89 | 204540_at | EEF1A2 | NM_001958 | eukaryotic translation elongation factor 1 α 2 |
| 90 | 221916_at | | BF055311 | hypothetical protein |
| 91 | 216693_x_at | DKFZp434C1722 | AL133102 | |
| 92 | 209500_x_at | | AF114012 | tumor necrosis factor-related death ligand-1β |
| 93 | 209534_at | FLJ10418 | AK001280 | moderately similar to Hepatoma-derived |
| | | | | growth factor |
| 94 | 207118_s_at | MMP23A | NM_004659 | matrix metalloproteinase 23A |
| 95 | 211040_x_at | | BC006325 | G-2 and S-phase expressed 1 |
| 96 | 218430_s_at | FLJ12994 | NM_022841 | hypothetical protein FLJ12994 |
| 97 | 217404_s_at | | X16468 | α-1 type II collagen. |
| 98 | 205848_at | GAS2 | NM_005256 | growth arrest-specific 2 |
| 99 | 214915_at | FLJ11780 | AK021842 | clone HEMBA1005931, weakly similar to zinc |
| | | | | finger protein 83 |
| 100 | 216010_x_at | | D89324 | α (1, 31, 4) fucosyltransferase |
| 101 | 204631_at | MYH2 | NM_017534 | myosin heavy polypep 2 skeletal muscle adult |
| 102 | 202687_s_at | | U57059 | Apo-2 ligand mRNA |
| 103 | 221634_at | | BC000596 | Similar to ribosomal protein L23a, clone |
| | | | | MGC: 2597 |
| 104 | 220886_at | GABRQ | NM_018558 | γ-aminobutyric acid (GABA) receptor, ζ |
| 105 | 202237_at | ADPRTL1 | NM_006437 | ADP-ribosyltransferase (NAD+; poly (ADP- |
| | | | | ribose) polymerase)-like 1 |
| 106 | 204218_at | DKFZP564M082 | NM_014042 | protein DKFZP564M082 |
| 107 | 221241_s_at | BCLG | NM_030766 | apoptosis regulator BCL-G |
| 108 | 209862_s_at | | BC001233 | Similar to KIAA0092 gene product, clone |
| | | | | MGC: 4896 |
| 109 | 217019_at | RPS4X | AL137162 | Contains novel gene and 5 part of gene for |
| | | | | novel protein similar to X-linked ribosomal |
| | | | | protein 4 |
| 110 | 210593_at | | M55580 | spermidinespermine N1-acetyltransferase |
| 111 | 216103_at | KIAA0707 | AB014607 | KIAA0707 |
| 112 | 205009_at | TFF1 | NM_003225 | trefoil factor 1 |
| 113 | 204623_at | TFF3 | NM_003226 | trefoil factor 3 (intestinal) |
| 114 | 209173_at | AGR2 | AF088867 | anterior gradient 2 homolog |
| 115 | 214440_at | NAT1 | NM_000662 | N-acetyltransferase 1 |
| 116 | 205081_at | CRIP1 | NM_001311 | cysteine-rich protein 1 (intestinal) |
| 117 | 214774_x_at | TNRC9 | AK027006 | trinucleotide repeat containing 9 |
| 118 | 214858_at | — | AF070536 | Pp14571 |
| 119 | 219197_s_at | SCUBE2 | AI424243 | signal peptide, CUB domain, EGF-like 2 |
| 120 | 215108_x_at | TNRC9 | U80736 | trinucleotide repeat containing 9 |
| 121 | 206754_s_at | CYP2B6 | NM_000767 | cytochrome P450, family 2, subfamily B, |
| | | | | polypeptide 6 |
| 122 | 210056_at | RND1 | U69563 | Rho family GTPase 1 |
| 123 | 205186_at | DNALI1 | NM_003462 | dynein, axonemal, light intermediate polypeptide 1 |
| 124 | 203130_s_at | KIF5C | NM_004522 | kinesin family member 5C |
| 125 | 216623_x_at | TNRC9 | AK025084 | trinucleotide repeat containing 9 |
| 126 | 222256_s_at | PLA2G4B | AK000550 | phospholipase A2, group IVB (cytosolic) |
| 127 | 210021_s_at | UNG2 | BC004877 | uracil-DNA glycosylase 2 |
| 128 | 204607_at | HMGCS2 | NM_005518 | 3-hydroxy-3-methylglutaryl-Coenzyme A |
| | | | | synthase 2 |
| 129 | 213664_at | SLC1A1 | AW235061 | solute carrier family 1 member 1 |
| 130 | 211657_at | CEACAM6 | M18728 | carcinoembryonic antigen-related cell adhesion |
| | | | | molecule 6 |
| 131 | 222348_at | — | AW971134 | — |
| 132 | 209114_at | TSPAN-1 | AF133425 | tetraspan 1 |
| 133 | 205645_at | REPS2 | NM_004726 | RALBP1 associated Eps domain containing 2 |
| 134 | 39763_at | HPX | M36803 | hemopexin |
| 135 | 214099_s_at | PDE4DIP | AK001619 | phosphodiesterase 4D interacting protein |
| 136 | 203757_s_at | CEACAM6 | BC005008 | carcinoembryonic antigen-related cell adhesion |
| | | | | molecule 6 |
| 136 | 204485_s_at | TOM1L1 | NM_005486 | target of myb1-like 1 |
| 138 | 206378_at | SCGB2A2 | NM_002411 | secretoglobin, family 2A, member 2 |
| 139 | 211712_s_at | ANXA9 | BC005830 | annexin A9 |
| 140 | 204378_at | BCAS1 | NM_003657 | breast carcinoma amplified sequence 1 |
| 141 | 206243_at | TIMP4 | NM_003256 | tissue inhibitor of metalloproteinase 4 |
| 142 | 210272_at | CYP2B6 | M29873 | cytochrome P450, family 2, subfamily B, |
| | | | | polypeptide 6 |
| 143 | 205597_at | C6orf29 | NM_025257 | chromosome 6 open reading frame 29 |
| 144 | 221946_at | C9orf116 | AU160041 | chromosome 9 open reading frame 116 |
| 145 | 210297_s_at | MSMB | U22178 | microseminoprotein, beta- |
| 146 | 204014_at | DUSP4 | NM_001394 | dual specificity phosphatase 4 |
| 147 | 204379_s_at | FGFR3 | NM_000142 | fibroblast growth factor receptor 3 |
| 148 | 205014_at | FGFBP1 | NM_005130 | fibroblast growth factor binding protein 1 |
| 149 | 209406_at | BAG2 | AF095192 | BCL2-associated athanogene 2 |
| 150 | 210655_s_at | FOXO3A | AF041336 | forkhead box O3A |
| 151 | 220559_at | EN1 | NM_001426 | engrailed homolog 1 |
| 152 | 209800_at | KRT16 | AF061812 | keratin 16 |
| 153 | 209373_at | BENE | BC003179 | BENE protein |
| 154 | 214595_at | KCNG1 | AI332979 | potassium voltage-gated channel, subfamily G, |
| | | | | member 1 |
| 155 | 216365_x_at | IGLC2 /// IGLJ3 | AF047245 | Immunoglobulin lambda constant 2 /// |
| | | | | Immunoglobulin lambda joining 3 |
| 156 | 206125_s_at | KLK8 | NM_007196 | kallikrein 8 |
| 157 | 211637_x_at | — | L23516 | Immunoglobulin heavy chain V region |
| | | | | (Humha448) /// Similar to Ig heavy chain V-I |
| | | | | region HG3 precursor |
| 158 | 209126_x_at | KRT6B | L42612 | keratin 6B |
| 159 | 219480_at | SNAI1 | NM_005985 | snail homolog 1 |
| 160 | 205347_s_at | TMSNB | NM_021992 | thymosin, beta, identified in neuroblastoma cells |
| 161 | 210683_at | NRTN | AL161995 | neurturin |
| 162 | 1438_at | EPHB3 | X75208 | EPH receptor B3 |
| 163 | 217294_s_at | ENO1 | U88968 | enolase 1 |
| 164 | 215223_s_at | SOD2 | W46388 | superoxide dismutase 2, mitochondrial |
| 165 | 211908_x_at | IGHG1 | M87268 | immunoglobulin heavy constant gamma 1 (G1m |
| | | | | marker) |
| 166 | 222242_s_at | KLK5 | AF243527 | kallikrein 5 |
| 167 | 206391_at | RARRES1 | NM_002888 | retinoic acid receptor responder 1 |
| 168 | 219415_at | TTYH1 | NM_020659 | tweety homolog 1 |
| 169 | 209772_s_at | CD24 | X69397 | CD24 antigen |
| 170 | 217281_x_at | MGC27165 /// | AJ239383 | hypothetical protein MGC27165 /// |
| | | IGHG1 | | immunoglobulin heavy constant gamma 1 (G1m |
| | | | | marker) |
| 171 | 204855_at | SERPINB5 | NM_002639 | serine (or cysteine) proteinase inhibitor, clade B |
| | | | | member 5 |
| 172 | 201387_s_at | UCHL1 | NM_004181 | ubiquitin carboxyl-terminal esterase L1 |
| 173 | 220425_x_at | ROPN1 | NM_017578 | ropporin, rhophilin associated protein 1 |
| 174 | 218484_at | LOC56901 | NM_020142 | NADH: ubiquinone oxidoreductase MLRQ subunit |
| | | | | homolog |
| 175 | 215177_s_at | ITGA6 | AV733308 | integrin, alpha 6 |
| 176 | 209372_x_at | TUBB /// | BF971587 | tubulin, beta polypeptide |
| | | MGC8685 | | |
| 177 | 202316_x_at | UBE4B | AW241715 | ubiquitination factor E4B |
| 178 | 211641_x_at | IGHM | L06101 | Immunoglobulin heavy chain VH3 (H11) |
| 179 | 217404_s_at | COL2A1 | X16468 | collagen, type II, alpha 1 |
| 180 | 203126_at | IMPA2 | NM_014214 | inositol(myo)-1(or 4)-monophosphatase 2 |
| 181 | 221986_s_at | DRE1 | AW006750 | DRE1 protein |
| 182 | 205778_at | KLK7 | NM_005046 | kallikrein 7 |
| 183 | 202134_s_at | TAZ | NM_015472 | transcriptional co-activator with PDZ-binding |
| | | | | motif (TAZ) |
| 184 | 212065_s_at | USP34 | AB018272 | ubiquitin specific protease 34 |
| 185 | 201952_at | — | NM_001627 | |
| 186 | 202987_at | C6ORF4 | AW296296 | Chromosome 6 open reading frame 4 |
| 187 | 218489_s_at | ALAD | NM_000031 | aminolevulinate, delta-, dehydratase |
| 188 | 213606_s_at | ARHGDIA | AI571798 | Rho GDP dissociation inhibitor (GDI) alpha |
| 189 | 201679_at | ARS2 | NM_015908 | arsenate resistance protein |
| 190 | 217528_at | CLCA2 | BF003134 | chloride channel, calcium activated, family |
| | | | | member 2 |
| 191 | 205830_at | CLGN | NM_004362 | calmegin |
| 192 | 220363_s_at | ELMO2 | NM_022086 | engulfment and cell motility 2 (ced-12 homolog, |
| | | | | C. elegans) |
| 193 | 220622_at | LRRC31 | NM_024727 | leucine rich repeat containing 31 |
| 194 | 202601_s_at | HTATSF1 | AI373539 | HIV TAT specific factor 1 |
| 195 | 206638_at | HTR2B | NM_000867 | 5-hydroxytryptamine (serotonin) receptor 2B |
| 196 | 218211_s_at | MLPH | NM_024101 | melanophilin |
| 197 | 218985_at | SLC2A8 | NM_014580 | solute carrier family 2, member 8 |
| 198 | 209278_s_at | TFPI2 | L27624 | tissue factor pathway inhibitor 2 |

Ahr et al. (2002) “Identification of high risk breast-cancer patients by gene-expression profiling” Lancet 359:131-132
Chang et al. (2003) “Gene expression profiling for the prediction of therapeutic response to docetaxel in patients with breast cancer” Lancet 362:362-9
Early Breast Cancer Trialists' Collaborative Group (1995) “Effects of radiotherapy and surgery in early breast cancer. An overview of the randomized trials” N Engl J Med 333:1444-1455
Early Breast Cancer Trialists' Collaborative Group (1998a) “Polychemotherapy for early breast cancer: an overview of the randomized trials” Lancet 352:930-942
Early Breast Cancer Trialists' Collaborative Group (1998b) “Tamoxifen for early breast cancer: an overview of randomized trials” Lancet 351:1451-1467
Efron (1981) “Censored data and the bootstrap” J Am Stat Assoc 76:312-319
Eifel et al. (2001) “National Institutes of Health Consensus Development Conference Statement: adjuvant therapy for breast cancer, Nov. 1-3, 2000” J Natl Cancer Inst 93:979-989
Foekens et al. (1989b) “Prognostic value of estrogen and progesterone receptors measured by enzyme immunoassays in human breast tumor cytosols” Cancer Res 49:5823-5828
Foekens et al. (1989a) “Prognostic value of receptors for insulin-like growth factor 1, somatostatin, and epidermal growth factor in human breast cancer” Cancer Res 49:7002-7009
Goldhirsch et al. (2003) “Meeting highlights: Updated International Expert Consensus on the Primary Therapy of Early Breast Cancer” J Clin Oncol 21:3357-3365
Golub et al. (1999) “Molecular classification of cancer: class discovery and class prediction by gene expression monitoring” Science 286:531-537
Gruvberger et al. (2001) “Estrogen receptor status in breast cancer is associated with remarkably distinct gene expression patterns” Cancer Res 61:5979-5984
Hedenfalk et al. (2001) “Gene-expression profiles in hereditary breast cancer” N Engl J Med 344:539-548
Herrera-Gayol et al. (1999) “Adhesion proteins in the biology of breast cancer: contribution of CD44” Exp Mol Pathol 66:149-156
Huang et al. (2003) “Gene expression predictors of breast cancer outcomes” Lancet 361:1590-1596
Kaplan et al. (1958) “Non-parametric estimation of incomplete observations” J Am Stat Assoc 53:457-481
Keyomarsi et al. (2002) “Cyclin E and survival in patients with breast cancer” N Engl J Med 347:1566-1575
Lipshutz et al. (1999) “High density synthetic oligonucleotide arrays” Nat Genet 21:20-24
Ma et al. (2003) “Gene expression profiles of human breast cancer progression” Proc Natl Acad Sci USA 100:5974-5979
Ntzani et al. (2003) “Predictive ability of DNA microarrays for cancer outcomes and correlates: an empirical assessment” Lancet 362:1439-1444
Perou et al. (2000) “Molecular portraits of human breast tumors” Nature 406:747-752
Ramaswamy et al. (2001) “Multiclass cancer diagnosis using tumor gene expression signatures” Proc Natl Acad Sci USA 98:15149-15154
Ramaswamy et al. (2003) “A molecular signature of metastasis in primary solid tumors” Nat Genet 33:1-6
Ransohoff (2004) “Rules of evidence for cancer molecular-marker discovery and validation” Nat Rev Cancer 4:309-314
Sørlie et al. (2001) “Gene expression patterns of breast carcinomas distinguish tumor subclasses with clinical implications” Proc Natl Acad Sci USA 98:10869-10874
Sørlie et al. (2003) “Repeated observation of breast tumor subtypes in independent gene expression data sets” Proc Natl Acad Sci USA 100:8418-8423
Sotiriou et al. (2003) “Gene expression profiles derived from fine needle aspiration correlate with response to systemic chemotherapy in breast cancer” Breast Cancer Res 4:R3
Sotiriou et al. (2003) “Breast cancer classification and prognosis based on gene expression profiles from a population-based study” Proc Natl Acad Sci USA 100:10393-10398
Su et al. (2001) “Molecular classification of human carcinomas by use of gene expression signatures” Cancer Res 61:7388-7393
van de Vijver et al. (2002) “A gene expression signature as a predictor of survival in breast cancer” N Engl J Med 347:1999-2009
van't Veer et al. (2002) “Gene expression profiling predicts clinical outcome of breast cancer” Nature 415:530-536
Wang et al. (2004) “Gene expression profiles and molecular markers to predict relapse of Dukes' B colon cancer” J Clin Oncol 22:1564-1571
Woelfle et al. (2003) “Molecular signature associated with bone marrow micrometastasis in human breast cancer” Cancer Res 63:5679-5684

1. A method of assessing breast cancer status comprising the steps of
a. obtaining a biological sample from a breast cancer patient; and
b. measuring the expression levels in the sample of genes via a Marker wherein the gene expression levels above or below pre-determined cut-off levels are indicative of the likelihood of relapse in bone.

2. A method of staging breast cancer patients comprising the steps of
a. obtaining a biological sample from a breast cancer patient; and
b. measuring the expression levels in the sample of genes via a Marker wherein the gene expression levels above or below pre-determined cut-off levels are indicative of the breast cancer stage.

3. The method of claim 2 wherein the stage corresponds to classification by the TNM system.

4. The method of claim 2 wherein the stage corresponds to patients with similar gene expression profiles.

5. A method of determining breast cancer patient treatment protocol comprising the steps of
a. obtaining a biological sample from a breast cancer patient; and
b. measuring the expression levels in the sample of genes via a Marker wherein the gene expression levels above or below pre-determined cut-off levels are sufficiently indicative of risk of relapse in bone to enable a physician to determine the degree and type of therapy recommended to prevent or treat relapse in bone.

6. A method of treating a breast cancer patient comprising the steps of:
a. obtaining a biological sample from a breast cancer patient; and
b. measuring the expression levels in the sample of genes via a Marker wherein the gene expression levels above or below pre-determined cut-off levels are indicate a high risk of relapse in bone and;
c. treating the patient with adjuvant therapy, bisphosphonate therapy, or other relevant therapy if they are a high risk patient.

7. The method of claim 1 wherein the bulk tissue preparation is obtained from a biopsy or a surgical specimen.

8. The method of claim 1 wherein the Markers include all of those corresponding to SEQ ID NOs: 112-198.

9. The method of claim 1, 2, 5 or 6 further comprising measuring the expression level of at least one gene constitutively expressed in the sample.

10. The method of claim 1, 2, 5 or 6 further comprising determining the estrogen receptor (ER) status of the sample.

11. The method of claim 10 wherein the ER status is determined by measuring the expression level of at least one gene indicative of ER status.

12. The method of claim 11 wherein the ER status is determined by measuring the presence of ER in the sample.

13. The method of claim 12 wherein the presence of ER is measured immunohistochemically.

14. The method of claim 1, 2, 5 or 6 wherein the sample is obtained from a primary tumor.

15. The method of claim 1, 2, 5 or 6 wherein the specificity is at least about 40%.

16. The method of claim 1, 2, 5 or 6 wherein the sensitivity is at least at least about 90%.

17. The method of claim 1, 2, 5 or 6 wherein the expression pattern of the genes is compared to an expression pattern indicative of a breast cancer patient that relapses to bone.

18. The method of claim 17 wherein the comparison of expression patterns is conducted with pattern recognition methods.

19. The method of claim 18 wherein the pattern recognition methods include the use of a bone relapse predictor score.

20. The method of claim 1, 2, 5 or 6 wherein the pre-determined cut-off levels are at least 1.7-fold over- or under-expression in the sample relative to cells or tissue from non-bone relapsing patients.

21. The method of claim 1, 2, 5 or 6 wherein the pre-determined cut-off levels have at least a statistically significant p-value over-expression in the sample having metastatic cells relative to cells or tissue from non-bone relapsing patients.

22. The method of claim 21 wherein the p-value is less than 0.05.

23. The method of claim 1, 2, 5 or 6 wherein gene expression is measured on a microarray or gene chip.

24. The method of claim 23 wherein the microarray is a cDNA array or an oligonucleotide array.

25. The method of claim 23 wherein the microarray or gene chip further comprises one or more internal control reagents.

26. The method of claim 1, 2, 5 or 6 wherein gene expression is determined by nucleic acid amplification conducted by polymerase chain reaction (PCR) of RNA extracted from the sample.

27. The method of claim 26 wherein said PCR is reverse transcription polymerase chain reaction (RT-PCR).

28. The method of claim 27, wherein the RT-PCR further comprises one or more internal control reagents.

29. The method of claim 1, 2, 5 or 6 wherein gene expression is detected by measuring or detecting a protein encoded by the gene.

30. The method of claim 29 wherein the protein is detected by an antibody specific to the protein.

31. The method of claim 1, 2, 5 or 6 wherein gene expression is detected by measuring a characteristic of the gene.

32. The method of claim 31 wherein the characteristic measured is selected from the group consisting of DNA amplification, methylation, mutation and allelic variation.

33. A method of assessing breast cancer status comprising the steps of
a. obtaining a biological sample from a breast cancer patient; and
b. measuring the expression levels in the sample of genes via a Marker wherein the gene expression levels above or below pre-determined cut-off levels are indicative of the likelihood of relapse in bone.

34. A kit for conducting an assay to determine breast cancer prognosis in a biological sample comprising a Marker.

35. The kit of claim 34 wherein the Marker corresponds to any of SEQ ID NO 112-116.

36. The kit of claim 34 wherein the Marker corresponds to all of SEQ ID NO 112-116.

37. The kit of claim 35 including Markers corresponding to one or more of SEQ ID NOs. 117-198.

38. The kit of claim 34 including Markers corresponding to all of SEQ ID NOs. 112-198.

39. The kit of claim 34 further comprising reagents for conducting a microarray analysis.

40. The kit of claim 34 further comprising a medium through which said nucleic acid sequences, their complements, or portions thereof are assayed.

41. Articles for assessing breast cancer status comprising Markers.

42. The articles of claim 41 wherein the Marker corresponds to any of SEQ ID NO 112-116.

43. The articles of claim 41 wherein the Marker corresponds to all of SEQ ID NO 112-116.

44. The articles of claim 41 including Markers corresponding to one or more of SEQ ID NOs. 117-198.

45. The articles of claim 41 further comprising reagents for conducting a microarray analysis.

46. The articles of claim 41 further comprising a medium through which said nucleic acid sequences, their complements, or portions thereof are assayed.

47. A microarray or gene chip for performing the method of claim 1, 2, 5, or 6.

48. The microarray of claim 47 comprising a Maker sufficient to characterize breast cancer status or risk of relapse in bone from a biological sample.

49. The microarray of claim 47 wherein the measurement or characterization is at least 1.7-fold over- or under-expression.

50. The microarray of claim 47 wherein the measurement provides a statistically significant p-value over- or under-expression.

51. The microarray of claim 47 wherein the p-value is less than 0.05.

52. The microarray of claim 47 comprising a cDNA array or an oligonucleotide array.

53. The microarray of claim 47 further comprising or more internal control reagents.

54. A diagnostic/prognostic portfolio comprising a Marker sufficient to characterize breast cancer status or risk of relapse in bone in a biological sample.

55. The portfolio of claim 54 wherein the measurement or characterization is at least 1.7-fold over- or under-expression.

56. The portfolio of claim 54 wherein the measurement provides a statistically significant p-value over- or under-expression.

57. The portfolio of claim 54 wherein the p-value is less than 0.05.


SEQUENCE LISTING (54 sequences)

SEQ ID NO: 1 - DNA, 1044 bp, human
gccaggccccctcctgaaagaggctccttgaagataattctaacatctttgtcatcagtgttgacatctcttgattgtatcctgtcattccatttgagatcttcctggttctggctggcatcctctgacatcactccagcagaagaaagggaaggaacacgctgcattactgcgtgggatatcttctaaccatactgatgattctccgtatagctggtcttttccaccttatcatccttcctctgagaagtatgaaaacactaagacatgtcgagggccagatggagaactccatgccaacctgctttgccctgtggatgtgctggatgttcccgagggcaccttgcctgacaaacagagcactgagcaagccatacagttgttggaaaagatgaaaacgtcagccagtcctttcttcctggccgttgggtatcataagccacacatccccttcagataccccaaggaatttcagaagttgtatcccttggagaacatcaccctggcccccgatcccgaggtccctgatggcctaccccctgtggcctacaacccctggatggacatcaggcaacgggaagacgtccaagccttaaacatcagtgtgccgtatggtccaattcctgtggactttcagcggaaaatccgccagagctactttgcctctgtgtcatatttggatacacaggtcggccgcctcttgagtgctttggacgatcttcagctggccaacagcaccatcattgcatttacctcggatcatgggtgggctctaggtgaacatggagaatgggccaaatacagcaattttgatgttgctacccatgttcccctgatattctatgttcctggaaggacggcttcacttccggaggcaggcgagaagcttttcccttacctcgacccttttgattccgcctcacagttgatggagccaggcaggcaatccatggaccttgtggaacttgtgtctctttttcccacgctggctggacttgcaggactgcaggttccacctcgctgccccgttccttcatttcacgt

SEQ ID NO: 2 - DNA, 1152 bp, human
tctttttggcttgagcctgaggccttgtcgagaagcttccgtgaaagggtgggccagccgggccacgagaaagaaagtgaataaatcaggaatataagtgggcggggggcccctgagaggggggtcgcaaagggtgagacatggccaccaggcgtttaaccgacgctttcttgttgttgcggaataattccatccaaaaccggcagctgttagccgagcaagtgagtagtcacatcacctccagccctctgcattcacgtagcattgctgcggagctggacgagcttgctgatgaccgtatggcactggtgtcaggcatcagcttagatccagaagcagcgattggtgtgacaaaacggccacctcctaagtgggtggatggagtggatgaaattcagtatgatgttggccggattaagcagaagatgaaagaatcggccagccttcatgacaagcatttaaacagacccaccctggatgacagcagcgaagaggaacatgccattgagataactacccaagagatcactcagctcttccacaggtgccagcgtgccgtgcagccctgccgagccgggcccgggcctgctccgagcaggaggggcggctgcttgggaacgtggtgcctcgtggcgcaggccctgcaggaactctccaccagcttccggcacgcacaatcaggctacctcaaacgcatgaagaatcgagaggaaagatcccagcattttttcgacacatcagtaccactaatggatgatggagacgataacactctttaccatcggggttttacagaggaccagttagttctggtggagcagaacacactgatggtggaagagcgggaacgagagattcgccagatggtacagtccatttctgacctgaatgaaatattcagggacttaggggcgatgattgtagaacagggtacagtccttgacagaattgactataacgttgaacagtcctgtatcaaaactgaagatggtttgaaacagcttcacaaggcagaacagtatcaaaagaagaatcggaagatgcttgtgattttaatattatttgtcatcatcattgtgctcattgttgtcctcgttggcgtgaagtctcgataagtggcattgggttttcgtgtgtgccgcgcgtgtggat

SEQ ID NO: 3 - DNA, 7488 bp, human
atgagtgtgaaagaggcaggcagctcgggccgccgggagcaggcggcctaccacctgcacatctacccccagctgtccaccaccgagagccaggcctcgtgccgcgtgactgccaccaaggacagcaccacctcggacgtcatcaaggacgccattgccagcctgcggctggacggcaccaaatgttatgtgctggtggaggtcaaagagtcgggaggcgaggaatgggtgctggacgccaacgactcgcctgtgcaccgggtgctgctatggccccggcgggcacaggacgagcaccctcaggaggatggctactacttcctgctgcaggagcgcaacgcagatggaaccatcaagtacgtgcatatgcagctggtggcgcaggccacagccacccggcgcctagtggagcgtggcctcctgccacggcagcaggcggactttgatgacctgtgtaacctccccgagctaaccgagggcaacctcctgaagaacctcaagcaccgcttcctgcaacaaaagatctacacgtacgcggggagcatcctggtggccatcaacccctttaagttcctgcccatctacaaccccaagtacgtgaagatgtatgagaaccagcagctgggcaagctggagccacacgtcttcgcgctggccgacgtggcctactacaccatgctcaggaagcgcgtgaaccagtgcatcgtgatctcgggtgagagcggctccggcaagacccagagcaccaacttcctcatccactgcctcaccgccctcagccagaagggctacgccagcggcgtcgagaggaccatcctgggtgctggccctgtgctggaggcttttggaaatgccaagacagcccacaacaacaactccagccggtttgggaaattcatccaagtcagctacctagagagtggcatcgtgagaggagctgtcgtcgagaaatatctgcttgaaaagtctcgcctggtgtctcaggagaaggatgagaggaactaccatgtgttttattatttgttacttggggtcagcgaggaagagcgccaagaatttcagctcaagcagcctgaagattatttctacctcaaccagcataacttgaagattgaagatggggaggacctgaagcatgactttgagaggctcaagcaggccatggagatggtgggcttcctccccgccaccaagaagcagatttttgccgtcctctcggccatcctgtacctgggcaacgtcacttataagaagagagctacaggccgagaggaagggttggaggtcgggccacccgaggtgctggacaccctgtcgcagcttctgaaggtgaagcgagaaatcttggtggaggttctgaccaaaagaaaaacggtgaccgtcaacgacaagcttatccttccctacagcctcagcgaggccatcactgcccgcgactccatggccaagtctctgtacagcgccctgttcgactggattgtgctgcggatcaaccacgcactcctcaacaagaaggacgtggaagaggcagtctcgtgcctgtccattggggtcctggacatcttcgggtttgaagacttcgagaggaacagctttgagcagttctgcatcaactacgccaatgagcagctgcagtattacttcaaccagcacatcttcaagctggagcaggaggaatatcagggcgaggggatcacgtggcacaacatcggctacacagacaatgtcggctgcatccatctcatcagcaagaaacccacgggcctcttctacctgctggacgaggagagcaacttcccccacgccacgagccagaccctgctggccaagttcaaacagcaacatgaggacaataagtacttcctgggcaccccggtcatggagccagctttcatcatccagcacttcgcagggaaggtgaaatatcagatcaaggacttccgggagaagaacatggactacatgcggccagacatcgtggccctgctgcggggcagtgacagctcctacgtgcgggagctcatcggcatggaccccgtggccgtgttccgctgggccgtgctccgggctgctatccgggccatggcagtgcttcgggaggccggacgcctgcgggccgagagggccgaaaaggctgcaggtatgagcagccctggtgcccaaagtcacccagaagagctgccaagaggagccagcaccccttcggaaaaactttaccgcgatttgcataaccaaatgatcaagagcatcaaaggattgccctggcagggcgaggacccccgtagccttctccagtccctcagtcggctccagaaaccccgcgccttcatcctgaaaagtaaaggtatcaaacaaaagcagatcattccaaagaacctactggactccaagtccctgaaactcatcatcagcatgactctgcacgaccgcaccaccaagtccctactgcacctgcacaagaagaaaaagccaccaagcatcagcgcccagttccagacatcccttaacaagctcttggaggcactggggaaggcggagcccttctttatccgctgcatccgttccaatgctgaaaagaaagagctgtgctttgacgacgagctggtcctgcagcagctgcgctacaccggcatgctggagaccgtgcgcatccggaggtcagggtacagcgccaagtacacgttccaggatttcaccgagcagttccaggtgctcctgcccaaggatgcccagccctgcagggaggtcatctccaccctcctggagaaaatgaagatagacaagaggaactaccagatcgggaagaccaaggtcttcctgaaggagacggagcggcaagccctgcaggagacgctgcaccgggaggtggtgcggaaaatcctgctgctgcagagctggttccggatggtgctggagcgtcggcacttcctgcagatgaagcgggccgccgtcaccatccaggcctgctggcggtcctaccgggtccggagggcgctggagaggacgcaggctgccgtgtacctccaggcctcatggaggggctactggcagcggaagctctaccggcaccagaaacagagcatcatccgcctgcagagcctgtgtcgggggcacctgcagcgcaagagcttcagccagatgatctcggagaagcagaaggcagaagagaaggagagggaagccctggaagccgcaagagcaggtgctgaggagggcggacagggtcaggcggctggagggcagcaggtagctgagcaggggccggagccagcggaggatggcgggcacctggcatcggagcctgaggtgcagccaagtgacaggtcccccctagagcactcctcacctgagaaggaggccccaagcccagagaagactctcccaccccagaaaaccgtggcggctgaaagtcacgagaaagtccccagcagccgggagaagcgtgagtcgcgtcggcaaagagggctggagcacgtcaagttccagaacaaacacatccagtcctgcaaggaggagagtgccctcagagaaccttccagaagggtcacccaggagcaaggggtgagtctcctggaagacaaaaaggagagcagagaagatgaaacccttctagtcgtagagacggaggctgagaacacatctcaaaagcagcccacagagcaaccccaggccatggcagttggcaaggtctctgaagaaactgagaagacgctgcccagtgggagccccaggcctggccagttggagcggccgaccagcctggccctggacagcagggtcagcccaccggcccctggcagcgcccccgagacccccgaggacaagagcaaaccatgtggcagcccaagggttcaggaaaagcccgacagccccggaggctccacgcagatccagcggtacctggacgccgagcggctggccagcgccgtggaactgtggcggggcaagaagctggtggccgccgccagccctagtgccatgctcagccagtccctggacctcagcgacagacaccgggccacaggggccgccctcacgcccacagaggagaggcgcacctccttctccacgagcgacgtctccaagctcctcccgtccctggccaaggctcagcctgcagcagaaaccacggacggagagcgaagtgcgaaaaagccagctgtccagaagaagaagccaggcgacgcatcctccctcccagacgcagggctgtccccgggctctcaggtcgactctaaatccacgtttaagaggctttttctgcataaaaccaaggataaaaaatacagcctggagggcgcagaggagctggagaatgcagtgtccgggcacgtggtgctggaagccaccaccatgaagaagggcctggaagccccctccggacagcagcatcgccacgctgcaggtgagaagcgcaccaaggaaccaggaggcaaagggaagaagaaccgaaatgtcaagattgggaagatcacagtgtcagagaagtggcgggaatcggtgttccgccagatcaccaacgccaatgagctcaagtacctggacgagttcctgctcaacaagataaatgacctccgttcccagaagacgcccattgagagcttgtttatcgaagccaccgagaagttcaggagcaacatcaaaacgatgtactctgtcccgaacgggaagatccacgtgggctacaaggatctgatggagaactaccagatcgtcgtcagcaacctggccactgagcgtggccagaaggacaccaacctggtcctcaacctcttccagtcactgctagatgagttcacccgtggctacaccaagaacgacttcgagccagtgaagcagagcaaagctcagaagaagaagcggaagcaggagcgtgctgtccaggagcacaacgggcacgtgttcgccagctaccaggttagcatcccgcagtcgtgcgagcagtgcctctcctatatctggctcatggacaaggccctgctctgcagcgtgtgcaagatgacctgccacaagaagtgcgtgcacaagattcagagccactgctcctacacctacgggaggaagggcgagccaggcgttgagcctggccacttcggcgtgtgcgtagacagcctgaccagcgacaaggcctcggtgcccatcgtgctggagaagctcctggaacacgtggagatgcacggcctgtacaccgagggcctctaccgcaagtcgggtgctgccaaccgcactcgggagctccggcaggcgctgcagacagaccccgcagcagtcaagctggagaacttccccatccacgccatcacaggggtgctgaagcagtggctgcgggagctgcccgagcccctcatgaccttcgcacagtacggcgacttcctccgagccgtcgagctgccggagaagcaggagcagctggctgccatctatgccgtcctggagcaccttccagaagccaaccacaactccctggagagactcatcttccaccttgtcaaggtggccctgctcgaggatgtcaaccgcatgtcacctggggcgctggccattatcttcgcaccctgcctcctgcgctgccctgacaactcggacccgctgaccagcatgaaggacgtcctcaagatcaccacgtgcgtggagatgctgatcaaggagcagatgaggaaatacaaagtgaagatggaggagatcagccaactggaggctgcagagagtatcgccttccgcaggctttcgctcctgcgacaaaatgctccatggcctctcaaactggggttttcgtctccctatgagggggtcctgaacaagagccccaagacccgggacatccaggaggaggagctggaggtgctgctggaggaggaggcagccggcggcgatgaggaccgggaaaaggagattctcattgaacggatccagtccatcaaggaggagaaggaggacatcacctaccggctgccggagctggacccaaggggctcggacgaggagaacctggactcggagacgtcggccagcaccgagagcctgctggaggagcgggccgggcggggggcctcggaagggccccctgcgcctgctctcccttgccccggcgcgcccaccccgagccccctccccaccgtggccgcccctccacgacgaaggccgtcgtccttcgtaacggtcagagtgaagaccccccggcggacccccatcatgcccacggccaacatcaagctcccaccaggcctgccctcccacctgcctcgctgggcaccgggtgcccgggaggcggctgccccagtgcggcgccgggagccacctgcccgccgcccggaccagatacattccgtgtacatcacgcccggggcagacctgccagtgcagggcgccctggagcccctagaagaggatggccagccacctggggccaagcggaggtactcggatcccccaacgtactgcctgccccccgcctcgggccagaccaatggctgagagccacagctgacaaagtctgcatgtccgaggacggcccctgcactggagctgggcgccagagctgcagagctagtgttcggccctcagagaaggatccagaatcaaaagctcaagagtgacgtgaggtgggcaccggccccaagtgcagagtcaaggcagggagaggccggctggagccaggccccctcgcacgcagcccccaaatcatggacgcacctgtggggagcaccacatctccacctgcggcctcacatctccccactcccctttttgtacgtttaactgtttctttgtacgtggtttacgtaactttaaactgtaacagccttaatggaagaccaaatggttttttatatgtgtatgtacaaagttttctattaacgctgcccgtctcccttataacctggacgtgagctgtcagagcagaagccactaggccactgcgcgtctgaggctcagaccctgctgtggttggcttggggtggccaatgggctgggaccctccatgagagttttggacacttgggtcacctgacccgtgcctctctgacacatgtctccggggggcagccacctggccaatgtgcatttttgcacatgctggaaccttccatgggggtctgggctattggctggagccaggacatgagtcaggggcaccatggacctcacgtgccagggagacttgaatgtggctgtcactcttccggacgccaagggctgcaggaggctgcttttggcactacccaccccgtgtgacagaataggagccagcgactcaggactgctcacgggtcaggagggcaacgcctgaagtcagacctccctataggtcaacagggacaacctggggatctctggagcagggccctcctctctcaggcttggcccactcccccagacacctggacacgtggccacaaatctgggacaaggggcccccgcacagcatgaaataaaaagtgcctgagaagtgtgtgcaaaaaaaaaaaaaaaaaaaaaaaaaaaaaa

SEQ ID NO: 4 - DNA, 2172 bp, human
acagccgctccctcgctctgctggggcctccggacgcgcttcccacgcgggtctctggaacactcggtccgaacgcacgcctgcttgcactcacactgcggttcacacccgcaggcgctctcgcacccacactgccgctcacgcgcgctcacactcccccacgcgcgctccgctccggctccagccccgcgccccgcgaaggcgcaggcactgctgccgagagcgccgaggggccccgcggccttcccatggcggacctgagcttcatcgaagataccgtcgccttccccgagaaggaagaggatgaggaggaagaagaggagggtgtggagtggggctacgaggaaggtgttgagtggggtctggtgtttcctgatgctaatggggaataccagtctcctattaacctaaactcaagagaggctaggtatgacccctcgctgttggatgtccgcctctccccaaattatgtggtgtgccgagactgtgaagtcaccaatgatggacataccattcaggttatcctgaagtcaaaatcagttctttcgggaggaccattgcctcaagggcatgagtttgaactgtacgaagtgagatttcactggggaagagaaaaccagcgtggttctgagcacacggttaatttcaaagcttttcccatggagctccatctgatccactggaactccactctgtttggcagcattgatgaggctgtggggaagccgcacggaatcgccatcattgctctgtttgttcagataggaaaggaacatgttggcttgaaggctgtgactgaaatcctccaagatattcagtataaggggaagtccaaaacaataccttgctttaatcctaacactttattaccagaccctctgctgcgggattactgggtgtatgaaggctctctcaccatcccaccttgcagtgaaggtgtcacctggatattattccgataccctttaactatatcccagctacagatagaagaatttcgaaggctgaggacacatgttaagggggcagaacttgtggaaggctgtgatgggattttgggagacaactttcggcccactcagcctcttagtgacagagtcattagagctgcatttcagtagccaaagaggacaggaacaagtctgtcttcatgagggaggaagacaatggtcctataatgcccttggataagaaaaggaaacttttgagctgcaccttcagtttatcctcaaagcctgcgttgtttgtcttcatctaatccagctttgatggacatctgtgatggttgcctgtacacttgctgaaatgaaatattagaaatggctgtatattccaaagaaaccctatattatatatccacattactgctgctaggattcatagttgcacatactgtttattgcttatgtgtagaaggaatgaaactagtttccagagttgttattaatatgaatatatatcatgtgttaatattgagaaaggaaaaatacattcccggtgttagtagttcttcatttcctgtctccaacagaaaattcactcattttagaactagtgtaattcttgataataaaataagagttttgattaagaacagcatagagcttcaaaatgcaaagtgaatgattagtaaaattatgtctcattttattttttcagcacccataccacaattaatattaggctggattgccatgggaaacattttttggcattaatgcagcaacataatactcactttaggtattactacatagttgaaggatttaactgaatgtatggatcaaatttatttatttgacatattcgaagctgtggtttaataggaatttgagaaaggtgtaagaaataggataaaaagaaggtcagcaccatgtaccaggaatagctttactttccatacatagaaatataaatttagtggtatcctatattactttagtgtcgtacgctttgtaagacttaaatattttattctattgattccactactttggtatgttaagacatttctttaaagatgaccaacaatatccttattttaggtgccactagcagatgtaagcgtatacttagttgccgttagatgtgacagaatgagataatttatgtaaagcagtagagtacctggcacaaagcaaacaataaatattattgttattgttgttataa

SEQ ID NO: 5 - DNA, 3536 bp, human
ccgcccgtcccgccccgccccgccgcccgccgcccgccgagcccagcctccttgccgtcggggcgtccccaggccctgggtcggccgcggagccgatgcgcgcccgctgagcgccccagctgagcgcccccggcctgccatgaccgcgctccccggcccgctctggctcctgggcctggcgctatgcgcgctgggcgggggcggccccggcctgcgacccccgcccggctgtccccagcgacgtctgggcgcgcgcgagcgccgggacgtgcagcgcgagatcctggcggtgctcgggctgcctgggcggccccggccccgcgcgccacccgccgcctcccggctgcccgcgtccgcgccgctcttcatgctggacctgtaccacgccatggccggcgacgacgacgaggacggcgcgcccgcggagcggcgcctgggccgcgccgacctggtcatgagcttcgttaacatggtggagcgagaccgtgccctgggccaccaggagccccattggaaggagttccgctttgacctgacccagatcccggctggggaggcggtcacagctgcggagttccggatttacaaggtgcccagcatccacctgctcaacaggaccctccacgtcagcatgttccaggtggtccaggagcagtccaacagggagtctgacttgttctttttggatcttcagacgctccgagctggagacgagggctggctggtgctggatgtcacagcagccagtgactgctggttgctgaagcgtcacaaggacctgggactccgcctctatgtggagactgaggacgggcacagcgtggatcctggcctggccggcctgctgggtcaacgggccccacgctcccaacagcctttcgtggtcactttcttcagggccagtccgagtcccatccgcacccctcgggcagtgaggccactgaggaggaggcagccgaagaaaagcaacgagctgccgcaggccaaccgactcccagggatctttgatgacgtccacggctcccacggccggcaggtctgccgtcggcacgagctctacgtcagcttccaggacctcggctggctggactgggtcatcgctccccaaggctactcggcctattactgtgagggggagtgctccttcccactggactcctgcatgaatgccaccaaccacgccatcctgcagtccctggtgcacctgatgatgccagacgcagtccccaaggcgtgctgtgcacccaccaagctgagcgccacctctgtgctctactatgacagcagcaacaatgtcatcctgcgcaagcaccgcaacatggtggtcaaggcctgcggctgccactgagtccacccgcccggcccagctgcagccacccttctcatctggatcgggcccctcagaagcaggaaaccctcaaacccagccagaccccaggccggggcattgccagggaggaccctcacaaccacgtacatgaccctttctccttcatgccaggctcctatgctccccttgccctgccaggcatttgtgtgactgtcctgtttccagcccaggtggtctcaatcatcaggcagtgttctacccaaatgcaaacgcctctcccggaggcatgtcctggctggttctttggggttggcacagaagtcctgtctgaggtcctatccatgccccttactggctcaggtcgtgagatagatgtggaatgacctgagaggcacctggagcccactgttggccaccttgagctcttcaccatccatcacagggtgtggtgtgtgtagtcagggtctggttggctccccattgcctgcccgaggtgcaaggtggggtataaaactggataacccctgaagtattgtatattcatggatctgaagcactgatccactggtcacaggtagacatgtggagtcaactcaagaaaaagctgagtgaacagcatgatttagggctaaagccaatggcatttatcttcccttgtcttcctgctttgcatttgcctctgccatctaggaaagacatgtaagagcatggacattttactttggagaaacagaaaaatcttggggcttccaattgacccatctatctgccaccatgttgccccaccaggagctcagctctgtggagttttccctttgctgagcaagcatgtggttgcattgggtggcccaggatgacaatgcacagcacagatgccatcatttccctttcccctctgaatggcagacatcagtaatcaatctggaatgtttttcttccaaatctgagtggaattttcaaatgatcagcacagccactgccaacagatatgatgtaaagtgaaacctggttgccatcttctgccatgctgaggagcagtccatccctgcccgagcatgtatcggcaacatgggcagcctgtgaccgggtctggggcgaggccaggggccatcaaaaacaggctgatcaccaaagtcagtgtcaccctggatgcccagcagccctgtcctgtgtcttgggcctgtgagtcaaagaaaaggtccttttcagggagtgacaagtagtaattaggctgagttgggtggagaggtttgtctcagcctctgctgttctcggaaactgctgttctccttggagcagccactgggagttggagtgtttatttgatttctgacttgctaagcctgtaatttacctgctggaatagacagagtccagctgcccaaaccgtgtcattaaaagcagatcctgcgcccgccccatccacaggcacagcccggcagagtggttccacctccccatgggcccaaggatgcgcctctctggagttcacgtgctgcacccccagggaggggcctggggaaagctggtccagcagcaggggtggaggctggggccacactgcgggacagcagcccctccacctggaccagggagggcctccatgtgcaagcgcagaggaagagaccctcccatgtacgcaaagggcagccccaggctgtctggaagttggagaattccctatcagcacagggatctcagctctggcctggaggtgaagagacctgccttgtaggtggcttccttatctgcgcctccattttctatctgcactttttgatctccaaacaaccttcagccaaagaatctgtctaccaactcctcatagtgagccagaagcagcctcataaccctgaatgtggggctctggtggctgtcacgaagcagagttggcacataacatggaacctggccaggcatggtggctcacacctataaccccagcactttgggaggccaaggcaggcagatcacctgaagtcaggagttcaagaccatcctggccaacacagtgaaaccccatctgtactaaaaatacaagattacctgggcatggtggtgcatgcctataatcccagctactcaggaggctgaggcagaattgcttgaacctgggaggtggaggttgcagtgagcagagatcacaacattgcacttcagcctggtgacatgagcaaaactgttgtctcaacaaaatgaaattatg

SEQ ID NO: 6 - DNA, 3425 bp, human
agcgcctgtgctgccgcgtctcgacgtgtcaccggcggcgccgctgctgtggcaaaggaaggagctgactgggggagttcgaggcggcgggcggcggtgaccccggcctggaactgccccggtacggaagtgttccggggtccgtggggagcaggagagggaggcggcggaccgtcccgcgcggggcacgatgttgaacatgtggaaggtgcgcgagctggtggacaaagccaccaatgttgttatgaattattcagagatcgagtctaaggttcgagaggcaacgaacgatgatccttggggaccttctgggcaactcatgggagagattgccaaggctacatttatgtatgaacaatttccagaacttatgaacatgctttggtcacgaatgttaaaagacaacaaaaagaattggagaagagtttataagtcgttgctgctcctagcttacctcataaggaatggatcagagcgtgttgttacaagtgccagagaacacatttatgatttacgatccctggaaaattaccactttgtagatgagcatggtaaggatcaaggtataaatattcgacagaaggtgaaggaattggttgaatttgcccaggatgacgacaggcttcgtgaagagcgaaagaaagcaaagaagaacaaagacaagtatgttggggtttcctcagacagtgttggaggattcagatacagtgaaagatatgatcctgagcccaaatcaaaatgggatgaggagtgggataaaaacaagagtgcttttccattcagtgataaattaggtgagctgagtgataaaattggaagcacaattgatgacaccatcagcaagttccggaggaaagatagagaagactctccagaaagatgcagcgacagcgatgaggaaaagaaagcgagaagaggcagatctcccaaaggtgaattcaaagatgaagaggagactgtgacgacaaagcatattcatatcacacaggccacagagaccaccacaaccagacacaagcgcacagcaaatccttccaaaaccattgatcttggagcagcagcacattacacaggggacaaagcaagtccagatcagaatgcttcaacccacacacctcagtcttcagttaagacttcagtgcctagcagcaagtcatctggtgaccttgttgatctgtttgatggcaccagccagtcaacaggaggatcagctgatttattcggaggatttgctgactttggctcagctgctgcatcaggcagtttcccttcccaagtaacagcaacaagtgggaatggagactttggtgactggagtgccttcaaccaagccccatcaggccctgttgcttccagtggcgagttctttggcagtgcctcacagccagcggtagaacttgttagtggctcacaatcagctctaggcccacctcctgctgcctcaaattcttcagacctgtttgatcttatgggctcgtcccaggcaaccatgacatcttcccagagtatgaatttctctatgatgagcactaacactgtgggacttggtttgcctatgtcaagatcacagaatacagatatggtccagaaatcagtcagcaaaaccttgccctctacttggtctgaccccagtgtaaacatcagcctagacaacttactacctggtatgcagccttccaaaccccagcagccatcactgaatacaatgattcagcaacagaatatgcagcagcctatgaatgtgatgactcaaagttttggagctgtgaacctcagttctccatcgaacatgcttcctgtccggccccaaactaatgctttgatagggggacccatgcctatgagcatgcccaatgtgatgactggcaccatgggaatggcccctcttggaaatactccgatgatgaaccagagcatgatgggcatgaacatgaacatagggatgtccgctgctgggatgggcttgacaggcacaatgggaatgggcatgcccaacatagccatgacttctggaactgtgcaacccaagcaagatgcctttgcaaatttcgccaattttagcaaataagagattgtaaaagaagcagattgaatgaagaatttttagctgtgcagataggtgatgttgggatggaaaatgctaatcaactaccctttcttttatcaagtaattaaaataaatctacataaagaaccaaaaaggctgttttataaaagtgaaatatccagtatttcagagggccaggcaagagcacttcagatgaggcagtcaaaatcatttttttccagtgaggatagaccacaagtgggtggtgagaccattgaaagcctttatcaactgaagagtccatttaacagcataatttgtgggaagactggaatagggctgaataaatgtgtttgaatctctaattttatactttcttttcctgaggaacttgatttttctgtccctggatcgccttgtcataattgggtctgttccttttactaccactcttgagtccatatatgaaatcattaaagttggatgatcagttttttataaaaatatatatttttgtccaagaaaaaaaaaagcatacatatgtgattatggctaaatcaaaggtaactggaatgtatatacttttgctaatgttccagcaacactgctattatactatccaaatttttattgtaacaaaacctctttaagcaattggtgattgccatgggacttttcccatgtcttctgctgtaattatcctgtgcagaactaggaagaaatttttttcaggactgctctatggtttcctttaaaagaaaaaaacttctgtttgtttttagcagtcattatttacaatttgcagtgattaacttggcaaggcttccttccgtgtttatccctgtagccatcatttaagtcaggaacagtcagaaaaatatttattttattttttttttgggtgtctgcaaaggtaaaaatccattaaaaccttaagttaaatataaatgttacaactcaatgtttgcttttagattttatacagtatttgttttgttttggttttgagtgtatataatgcagcattagcaatatggttccaatagaggagttaaatatatattgttaaaggagacctgtagcagtcaaagattttattgatttaatgacaaaggaaattaatgaaaatgtttttgtttttctgctgtaattctgcattaagctcacatgaaaatcatgattctagagtttggaatgcaaaattaattgttttaccctcaagctgggaatatttttcaaaataaatactataatatagatatcaaattattacctccccatgttatgttgaaaatttttttattaaattgataaaactttatttccattatattcataatgttctgttatacataacattaaaatgttcattaaaatc

SEQ ID NO: 7 - DNA, 1767 bp, human
gcgcgccgctttctgttgccgggcgcaatggcggatacgctggagtcctcgctggaggacccactgcggagctttgtgcgagttttggagaagcgggatggtacagtgctacgactacagcagtatagctccggtggcgtgggttgcgttgtgtgggacgctgccattgtcctttctaaatacctggaaacgcccgagttttctggcgacggggcccacgcgctgagccggcggtcggtgctggagctgggttcgggcaccggggccgtggggctcatggctgctaccctcggggctgatgttgtagtcaccgatcttgaggaattgcaagacttgctgaagatgaatattaatatgaacaagcatcttgtcactggttctgttcaagccaaggggggaagaaatagaaggctttccttctccacccgacttcatactgatggccgactgcatatactatgaagagtctttggagccattgctgaaaactctaaaagatatcagcggatttgaaacttgtattatatgttgttatgaacaacgaacaatggggaaaaattcagaaattgagaaaaaatattttgagctccttcagctagattttgactttgaaaaaattcctttggaaaaacgtgatgaagagtatcgaagtgaagatattcatattatatacatcagaaagaaaaaatcgaaatttccatcgtgaagcctttaatcatcttacccaaggctctaacaacctgggtaaactaaagatgtgaatagagcatgtgaatacagcatgggaagattgtgttcacagatttttttttccggcacgtccttagagatccgatgtatagatgatgaccaccaggggctgtctgcaatacgaaaaattcctgcttgcctgcctgcctgccaatggccctgaatccagcttaggttacttagttcagcatcaagttcttcttaaatgttgggaatcagttattcaaataaaaattggtattgaggcgaggctgaaatctgccaaaaacagccaacatgatttgactggaccttttacaggaagctagagataaatttctgaagagaactatctgctattatataataatgttttaaattcaaactagtaaattttagtttgtcttcagagtttaaaaggttttcattttgtacataattatacaatatttatcatttgtattttccacttcatttcttttaaaatatctttaatactgaaatgttcttttaattttaaaaagaactgagatattgtcttgtatacttatattggccaaagttttcttttcctccaccatacatgtctatgtgatttaatcagtaaattgtactgtaatgagttgctaagaagacaaatcagaagattggaggaacaaaagatatctttacagatttttcatttgagcatggagtgagaatagaaagaccagtttcaggcttatgcacttacgtggctcatgcactttatgtatatgttccaggaaaatctggcttaaaaatactggtattgtttacatgaagcagtgaaaggtttttgaataactacaaatgtagttctatatgtatataccaaatgaatttctgttctgtgtctctctgttttatgttatgaagccattcgctcatatacaataatctgtcaaggactttaatcatacttgttccaaagagtagtaaccggatggaattctggtatttacaggcattggtgctagatggtacattttatgtgttaaaataaacattgtttttgag

SEQ ID NO: 8 - DNA, 2008 bp, human
cagcaactccgtggccaagctgaggaggcccagggcccgctcctgcttctccacgggtgtgctctgtggcccccgatggaagggggaaggatccaagcaggccctccctgcccagggcagaagattctggagtcctgggcgttgcctgtgagtgttctgagagataagggccccgcccgccccgccccgggaccaaggtcctcgtcccgtcccttgctgcgccgggaactcctgccccacgcaggcttctggtctccacactgggcttgttcctgggtgatgagattcggctccaccctcacgggctgccagcctcctgcccgatgggtgagcctctgctgctgcttcagcaaagggcggccccacttcccacactggcaggtggggacagggagaccctgggcctccccagagctcatgggttagcttccacaccctcatgctgtcatgagcctttctcacagggtcagaagagtctgcatctgacacagagaccccagtccacatagtcagggctgggccagagggagggacttggggctgatggggaggaggctcggcgtgagagtgaggtctttgcccaaggagggtatgaaagctggggctttagtgggtgggtaggagttggccagcagagagccgacggaagagttaatttaagtttatggacctgtaagtgctccgagtcccagggtgtctgggtgttagctgctgactggtgtcctcatgagtcgggctgggtgaggctgggttaggaagggttccacaagccccccgggtccactgaggtttgcccctgagatgatgaaccgaagcttttagagctgggcaggcgtgagggtgacccataactctctctgtcctgcctgcaggtcttgaggaccggcccagctcaggctcctggggcagcggcgaccagagcagctcctcctttgaccccagccgggtaggagtccccgtgaggcctgagaccctgagtttcctgtccccagagttgggggcgaggggtgtggggaaactgaggctgagagaaggggccagccttccaggggtttggactgggggacatgggtttgagtcctgcctctccagctgtaatgcggggtggagggggggcctgtgaagccccagcacctctcatccccaccccatctcccctagaccttcagcgagggcacccacttcactgagtcgcacagcagcctctcttcatccacattcctgggaccgggactcggaggtgagtgcctggcctggtgcggtccctctgctgtgcacagatgtgcagacgtgggccttggcatggtcagtgcacgcacagcgccaggcaagtccccagcgttcacccagagggtgtggacagccgaagcctctgcccgtcctgggagcagctgcggggaggtgcaggggacgtggcccggactccctggcacggcactctgcaactaggcttccccgcctgagcgtagaggcagggaggccgttccagaccccacttcctccctgggatttctgcagggctcctcctgcttcccggggaggggtcagccgggctggcctgtgcccgctgggaaggaggcgtaggagttctggagactcgggagggcggccagagtgtgcggtggtcagagcccatgcccttccgcagaccgcccgcccgccctctttccctggggggccctcctgttaaccgagagaatcgtgactcaggttgggctcggtggctcacgcctgtaataccagcactttgggagagcgaggtgggtggatcacctgaggtcaggagtttgagaccagcctggccaacatggtgaaaccccgcttctaccaaaaacacaaaaattagccggtcatggtgttggatgcttatagtgccaactactcaggaggctgaggcaggagaattgcttgaacccaggaggtggaggctgcagtgagccgagatcacgccattgccctccagcctgggtgatggagcgagattccatcttaaaaaaaaaaaaaaaaa

SEQ ID NO: 9 - DNA, 12533 bp, human
tccaaactgttcaaaatgatgaacgaagatgcagctcagaaaagcgacagtggagagaagttcaacggcagtagtcagaggagaaaaagacccaagaagtctgacagcaatgcaagcttcctccgtgctgccagagcaggcaacctggacaaagttgtggaatatctgaaggggggcatagacatcaatacctgcaatcagaatggactcaacgctctccatctggctgccaaggaaggccacgtggggctggtgcaggagctgctgggaagagggtcctctgtggattctgccactaagaagggaaataccgctcttcacattgcatctttggctggacaagcagaagttgtcaaagttcttgttaaggaaggagccaatattaatgcacagtctcagaatggctttactcctttatacatggctgcccaagagaatcacattgatgttgtaaaatatttgctggaaaatggagctaatcagagcactgctacagaggatggctttactcctctagctgtggcactccagcaaggacacaaccaggcggtggccatcctcttggagaatgacaccaaagggaaagtgaggctgccagctctgcatattgccgctaggaaagacgacaccaaatctgccgcacttctgcttcagaatgaccacaatgctgacgtacaatccaagatgatggtgaataggacaactgagagtggttttacccctttgcacatagctgcacattacggaaatgtcaacgtggcaactcttcttctaaaccggggagctgctgtggacttcacagccaggaatggaatcactcctctgcatgtggcttccaaaagaggaaatacaaacatggtgaagctcttactggatcgaggcggtcagatcgatgccaaaactagggatgggttgacaccacttcactgtgctgcacgaagtgggcatgaccaagtggtggaacttctgttggaacggggtgcccccttgctggcaaggactaagaatgggctgtctccactacacatggctgcccagggagaccacgtggaatgtgtgaagcacctgttacagcacaaggcacctgttgatgatgtcaccctagactacctgacagccctccacgttgctgcgcactgtggccactaccgtgtaaccaaactccttttagacaagagagccaatccgaacgccagagccctgaatggttttactccactgcacattgcctgcaagaaaaaccgcatcaaagtcatggaactgctggtgaaatatggggcttcaatccaagctataacagagtctggcctcacaccaatacatgtggctgccttcatgggccacttgaacattgtcctccttctgctgcagaacggagcctctccagatgtcactaacattcgtggtgagacggcactacacatggcagcccgagccgggcaggtggaagtggtccgatgcctcctgagaaatggtgcccttgttgatgccagagccagggaggaacagacacctttacatattgcctcccgcctgggtaagacagaaattgtccagctgcttctacaacatatggctcatccagatgcggccactacaaatgggtacacaccactgcacatctctgcccgggagggccaggtggatgtggcatcagtcctattggaagcaggagcagcccactccttagctaccaagaagggttttactcccctgcatgtagcagccaagtatggaagcctggatgtggcaaaacttctcttgcaacgccgtgctgccgcagattctgcagggaagaacggccttaccccgctccatgttgctgctcattatgacaaccagaaggtggcgctgctgttactggagaagggtgcttcccctcatgccactgccaagaatggctatactccgttacatattgctgccaagaagaatcaaatgcagatagcttccacactcctgaactatggagcagagacaaacattgtgacaaagcaaggagtaactccactccatctggcctcgcaggaggggcacacagatatggttaccttgcttctggataagggagccaatatccacatgtcaactaagagtggactcacatccttacaccttgcagcccaggaagataaagtgaatgttgctgatattctcaccaagcatggagctgatcaggatgctcatacaaagcttggttacacacctttaattgtggcctgtcactatggaaatgtgaaaatggtcaactttcttctgaagcagggagcaaatgttaacgcaaaaaccaagaacggctacacgcctttgcaccaggccgctcagcagggtcacacgcacatcatcaacgtcctgctccagcatggggccaagcccaacgccaccactgcgaatggcaacactgccttggcgattgctaagcgtctgggctacatctccgtggtcgacaccctgaaggttgtgactgaggaggtcaccaccaccaccacaactattacagaaaaacacaaactaaatgtacctgagacgatgactgaggttcttgatgtttctgatgaagagggtgatgacacaatgactggtgatgggggagaataccttaggcctgaggacctaaaagaactgggtgatgactcactacccagcagtcagttcctggatggtatgaattacctgcgatacagcttggagggaggacgatctgacagccttcgatccttcagttccgacaggtctcacactctgagccatgcctcctacctgagggacagtgccgtgatggatgactcagttgtgattcccagtcaccaggtgtcaactctagccaaggaggcagaaaggaattcttatcgcctaagctggggcactgagaacttagacaacgtggctctttcttctagtcctattcattcaggtttcctggttagttttatggtggatgcccgaggtggtgctatgcgaggatgcagacacaatgggctccgaatcattattccacctcggaaatgtactgctccaacgcgagtcacctgccgactggtcaagcgccacagactggcaacaatgcctccaatggtggaaggagaaggcctggccagtcgcctgatcgaagttggaccttctggtgctcagttccttggtaaacttcacctgccaacggctcctcccccacttaatgagggagaaagtttggtcagccgcattcttcagctggggcctcctggaaccaaattccttgggcctgtgatcgtggagatccctcactttgcggcccttcgaggaaaggaaagggaactggtggtcctgcgcagtgagaatggggacagctggaaagagcatttctgtgactacactgaagatgaattgaatgaaattcttaacggcatggatgaagtactggatagcccagaagacctagaaaagaaacgaatctgccgcatcatcacccgagacttcccacagtactttgcagtggtgtctcgtatcaaacaggacagcaatctgattggcccagaaggaggtgtactgagcagcacagtggtgccccaggtgcaggccgtcttcccagagggggcactcaccaagcggatccgtgtaggcctgcaggctcaacctatgcacagtgagctggttaagaagatcctaggcaacaaagctaccttcagccctatagtcactttggaacctagaagaagaaaattccacaaaccaattaccatgaccattcctgtccccaaagcttcaagtgatgtcatgttgaatggttttgggggagatgcaccaaccttaagattactatgcagcataacaggtggaaccacccctgcccagtgggaagatattacaggaactacgccattaacatttgtcaatgaatgtgtttcctttacaacaaacgtgtctgccaggttctggctgatagattgtcgacagatccaggaatccgttacttttgcatcacaagtatacagagaaattatctgcgtaccttatatggccaaatttgtagtgtttgccaaatcacatgaccccattgaagccaggttgaggtgtttctgcatgactgatgataaagtggataagacccttgaacaacaagaaaattttgctgaggtggccagaagcagggatgtggaggtgttagaaggaaaacccatctacgttgattgtttcggcaacttggtaccattaactaaaagtggccagcatcatatattcagtttttttgccttcaaagaaaatagacttcctctatttgtcaaggtacgcgatacgactcaggaaccttgcggacgactatcatttatgaaggagccaaaatccacgagaggcctggtgcatcaagctatttgcaacttaaacatcactttgccgatttatacaaaggaatcagagtcagatcaagaacaggaggaagagatcgatatgacatcagaaaaaaatgatgagacagaatctacagaaacatctgtcctgaaaagtcacctggttaatgaagttcctgtcctagcaagtccggacttgctctctgaagtttctgagatgaaacaagatttgatcaaaatgaccgccatcttgaccacagatgtgtctgataaggcaggttctattaaagtgaaggagctggtgaaggctgctgaggaagagccaggagagccttttgaaatcgttgaaagagttaaagaggacttagagaaagtgaatgaaatcctgagaagtggaacctgcacaagagatgaaagcagtgtgcagagctctcggtctgagagaggattagttgaagaggaatgggttattgtcagtgatgaggaaatagaagaggctaggcaaaaagcacctttagaaatcactgaatatccatgtgtagaagttagaatagataaagagatcaaaggaaaagtagagaaagactcaactgggctagtgaactaccttactgatgatctgaatacctgtgtgcctcttcccaaagagcagctgcagacagttcaagataaggcagggaagaaatgtgaggctctggctgttggcaggagctctgaaaaggaagggaaagacatacccccagatgagacacagagtacacagaaacagcacaaaccaagcttgggaataaagaagccagtaagaaggaaattaaaagaaaagcagaaacaaaaagaggaaggtttacaagctagtgcagagaaagctgaacttaaaaaaggtagttcagaagagtcattaggtgaagacccaggtttagcccctgaaccccttcccactgtcaaggccacatctcctttgatagaagaaactcccattggttccataaaggacaaagtaaaggcccttcagaagcgagtggaagatgaacagaaaggtcgaagcaagttgcccatcagagtcaaaggcaaggaggacgtgccaaaaaagaccacccacaggccacatccagctgcgtcaccctctctgaagtcagagagacatgcgccagggtctccctcccctaaaacagaaagacactctactctttcctcttccgcaaaaactgaaaggcaccctccagtatcaccatcaagtaaaactgagaaacactcacctgtgtcaccctctgcaaaaacggaaagacattcacctgcgtcatcatcgagtaaaactgagaaacactcacctgtatcaccctcgacaaaaactgaaaggcactctcctgtgtcatctacaaaaacagaaagacacccacctgtttcgccttcaggcaaaacagacaaacgtccacctgtatcgccctccgggaggacagaaaaacacccgccagtatcgcctgggagaacagaaaaacgcttgcctgtttcaccctccggaagaacggacaagcaccaacctgtatcaacagctgggaaaactgagaagcacctgcctgtgtcaccttctggcaaaacagaaaagcaaccacctgtatcccccacttcaaaaacagagaggattgaggaaaccatgtctgttcgggagctgatgaaggctttccagtcaggtcaggacccttctaaacataaaactggactctttgagcacaaatcagcaaaacaaaagcagccacaagagaaaggtaaagttcgggtagaaaaagaaaaggggccgatactaacccagagagaagctcagaaaacagagaatcagacaatcaaacgaggccagagactcccggtaacgggcacagcagaatccaaaagaggagttcgtgtttcctccataggagttaagaaagaagatgcagctggaggaaaggagaaagttctcagccacaaaatacctgaacctgttcagtcagtgcctgaagaagaaagccacagagagagcgaagtgcccaaagaaaagatggctgatgagcagggagacatggatctacagatcagcccagataggaaaacctccactgacttctctgaggtcattaagcaagagttggaagacaatgacaaataccaacaattccgcctgagtgaggagacagaaaaggcacagcttcacttagaccaagtactcactagtcctttcaacacaacatttccactcgactacatgaaagatgagttccttccagctctgtctttacaaagcggtgctttagatggcagttctgaaagcctaaagaatgagggggtagccggctctccgtgtggcagcctgatggaggggacccctcagattagttcagaagaaagctataagcatgaaggcctagcagagacccctgagacgagcccagaaagcctttctttctcaccaaagaaaagtgaggagcaaactggggaaacaaaggaaagcaccaagacagaaaccaccacagaaattcgttcagaaaaagagcatcccacgaccaaagacattactggtggctctgaagagcgaggtgccacagtcactgaggactcagagacctctactgagagttttcagaaagaggccactctaggctctcccaaagacacaagccctaaaagacaagatgattgcacaggcagctgtagtgtagcattagctaaagagacacctacaggactgactgaggaggcagcctgtgatgaaggtcaacgtacctttggtagttcagcccacaagacacaaactgatagtgaggctcaggaatccacagccacctcagacgagacaaaggccttgccgctgcctgaggcttctgtaaagacagatacaggaactgaatcaaaacctcagggagtcattagaagtccccaagggttagaacttgcactccctagccgagatagcgaagtcctcagcgctgtggctgatgactcattagcagtgagccacaaagactctctggaagccagccctgtgctagaagataactcttcacacaaaacccctgattctctggagccaagtcctctgaaagaatccccttgccgtgactctctggaaagcagccctgttgaaccaaagatgaaggctggaatttttccaagtcactttcctcttcctgcagctgttgccaaaacagaactcttgacggaagtggcctctgtgcggtcccggctactccgagaccctgatggcagtgctgaggatgacagtcttgagcagacatcgctcatggagagctcagggaagagccccctttctcctgacacccccagctctgaagaagtcagctatgaggttacacccaaaaccacagatgtaagtacaccaaaaccagctgtgattcatgaatgtgcagaggaggatgattcagaaaacggggagaaaaagaggttcacacctgaagaggagatgtttaaaatggtaaccaaaatcaaaatgtttgatgaacttgaacaagaagcaaagcagaaaagggactacaaaaaagaacccaaacaagaagaatcttcttcatcttctgacccagatgctgactgttcagtagatgtggatgaaccaaaacatacaggcagtggggaggatgaaagtggtgtccctgtgttagtaacttcggagagcaggaaggtgtcttcctcctcagaaagtgaacctgagttggcacagcttaaaaaaggtgctgactcaggccttttaccagaaccagtgattcgagtacaacctccttctccacttccatcaagcatggactccaattccagtccagaagaagtacaattccagcctgtcgtttccaaacaatatactttcaagatgaatgaagatactcaggaagagccaggcaaatcagaagaagaaaaagattctgaatcccatttagctgaagaccgtcatgctgtttccactgaggctgaagacaggtcttatgataagctaaacagagacactgatcagccaaaaatctgtgatggccatggatgtgaggccatgagtcctagcagctcagctcgtcctgtctcttcaggtctacagagtccgactggtgatgatgttgatgaacagccagtcatctataaagaatcattagctctccaaggcactcatgaaaaagacacagagggagaagagcttgatgtttctagagcagaatctccacaagcagattgccccagtgaaagcttttcatcttcatcctctttgcctcattgtttggtatctgaaggaaaagaattagatgaagacatatctgccacatcttctattcaaaaaacagaggtcacaaaaactgatgaaacatttgagaacttaccaaaggactgcccctctcaagactcatccattactactcaaacagatagattttccatggatgttcccgtgtctgacctagctgagaatgatgaaatctatgatccacaaatcactagcccttatgaaaatgtcccttcccaatcttttttctctagtgaagaaagcaaaacccaaacagatgcaaatcacaccacaagttttcactcttctgaagtgtattctgttaccatcacatcccctgttgaagacgttgtagtggcaagctcctctagtggaactgttttaagcaaagaatctaattttgagggccaggacataaaaatggaatcccaactggaaagtaccttgtgggaaatgcaatcagacagtgtctcttcatctttcgagcctactatgtccgctacaacaacagttgttggtgaacaaataagcaaagtcatcatcacaaaaactgatgtggattctgattcttggagtgaaattcgggaagacgatgaagcctttgaggctcgtgtgaaagaggaagaacaaaagatatttggtttgatggtagacagacaatcacagggtaccacccctgacaccactcctgctaggaccccaactgaagaggggaccccaacaagtgagcaaaacccatttctgtttcaggaaggaaaattgtttgaaatgacccgaagtggtgccattgatatgaccaaaaggtcctatgcagatgaaagttttcactttttccaaattggtcaagaatccagggaagagactctctctgaagatgtgaaagaaggggctactggggctgatcccctaccgctggagacatcagctgaatcactagcactttcagaatcaaaagaaacagtggatgatgaggcagacttacttccagatagcgtgagtgaggaagtagaggaaatacctgcttcggatgctcaacttaactcccaaatggggatttcagcctccactgaaacacctacaaaagaggctgttagtgtagggaccaaggacctccccaccgtgcaaacgggtgatatacctcctctctctggtgtaaagcagatatcctgccccgactcttctgaaccagctgtacaagtccagttagatttttccacactcaccaggtctgtttattcagataggggtgatgattctcccgattcttccccagaagaacagaaatcagtaatcgagattcctactgcacccatggagaatgtgccttttactgaaagcaaatccaaaattcctgtaaggactatgcccacttccaccccagcacctccatctgcagagtatgagagttcagtttctgaagattttctatccagtgtagatgaggaaaataaggcggatgaagcaaaaccaaagtccaaactccctgtcaaagtacccctccaaagagttgaacagcagctctcagatctagacacctctgtccagaagacagtggctcctcagggacaggacatggcaagcatcgcaccagataatagaagcaaatctgaatctgatgctagttctttggattcaaagaccaaatgcccagtaaaaacccgaagttacactgagacagaaacagagagcagagagagggccgaggaacttgagttagaatcagaagaaggggccacaagaccaaagatacttacatcccgattgccagttaagagcagaagcactacatcttcctgcagggggggcacgagccccacaaaagaaagtaaggagcatttctttgacctttacagaaattccatagaattctttgaggagattagtgatgaggcttccaaattagtggataggctgacacagtcagagagggagcaggaaatagtttcagacgatgaaagtagtagtgccctggaagtatcagtaattgaaaatctgccacctgttgagaccgagcactcagttcctgaggacatctttgacacaaggcccatttgggatgagtctattgagactctgattgaacgcatccctgatgaaaatggccatgaccatgctgaagacccacaggatgagcaggaacggatcgaggaaaggctggcttatattgctgatcaccttggcttcagctggacagaattagcaagagaactggatttcactgaggagcaaattcatcaaattcgaattgaaaatcccaactctcttcaagaccagagtcatgcactgttgaagtactggctagagagggatgggaaacatgctacagataccaacctcgttgaatgtctcaccaagatcaaccgaatggatattgttcatctcatggagaccaacacagaacctctccaggagcgcatcagtcatagttatgcagaaattgaacagaccattacactggatcatagtgaagggttctcggtacttcaagaggagttatgcactgcacagcacaagcagaaagaggagcaagctgtttctaaagaaagtgagacctgcgatcaccctcctatcgtctcagaggaagacatttctgttggttattccacttttcaggatggcgtccccaaaactgagggggacagctcatcaacagcactctttccccaaactcacaaggagcaagttcaacaggatttctcagggaaaatgcaagacctgcctgaagagtcatctctggaatatcagcaggaatattttgtgacaactccaggaacagaaacatcagagactcagaaggctatgatagtacccagctctcccagcaagacacctgaggaagttagcacccctgcagaggaggagaagctgtacctccagaccccaacatccagcgagcggggaggctctcccatcatacaagaacccgaagagccctcagagcacagagaggagagctctccgcggaaaaccagcctcgtaatagtggagtctgccgataaccagcctgagacctgtgaaagactcgatgaagatgcagcttttgaaaagggagacgatatgcctgaaatacccccagaaacagtcacagaagaagaatacattgatgagcatggacacaccgtggtaaagaaggttactaggaaaatcattaggcggtatgtatcctctgaaggcacagagaaagaagagattatggtgcagggaatgccacaggaacctgtcaacatcgaggaaggggatggctattccaaagttataaagcgtgttgtattgaagagtgacaccgagcagtcagaggacaacaatgagtaaagccatcacacagaagagggctgtggtgaaggaccagcatggaaaacgcattgacttggagcacctggaggatgtaccagaagcactagaccaggacgacctccagcgcgatctccagcagctccttcggcatttctgcaaggaggacttgaagcaagaggccaagtgagggactgcccagttctcacaccagaaaccacacattcactcaatatgcagcttcctgtttcagtaggggagtgacctaactggcctaattaatgggataccccgacatttccactgttagcaaatatacggcattttgctttagttttcccccatcctctttaactataaagctaatttgtgaccaaagatggcatccttcatactggatgctgtatccaatactttgttgtgtctgtgctaacctgggaactggccacctccattgttctttgcttctgcacaagatccatgaaaatccattgatcagaagaacttcacctgcagacctcttcaagtgacactatgtaggaatccttccaaggaatatctatgtacaatgtatatagctgaaatgctcagatgaacaacatattaaaattaaaaccactgcctattgtaactacactgggcatcagaataaaaggcctctaaaa

SEQ ID NO: 10 - DNA, 3071 bp, human
atggccggcgcccccggcccgctgcgccttgcgctgctgctgctcgggatggtgggcagggccggcccccgcccccagggtgccactgtgtccctctgggagacggtgcagaaatggcgagaataccgacgccagtgccagcgctccctgactgaggatccacctcctgccacagacttgttctgcaaccggaccttcgatgaatacgcctgctggccagatggggagccaggctcgttcgtgaatgtcagctgcccctggtacctgccctgggccagcagtgtgccgcagggccacgtgtaccggttctgcacagctgaaggcctctggctgcagaaggacaactccagcctgccctggagggacttgtcggagtgcgaggagtccaagcgaggggagagaagctccccggaggagcagctcctgttcctctacatcatctacacggtgggctacgcactctccttctctgctctggttatcgcctctgcgatcctcctcggcttcagacacctgcactgcaccaggaactacatccacctgaacctgtttgcatccttcatcctgcgagcattgtccgtcttcatcaaggacgcagccctgaagtggatgtatagcacagccgcccagcagcaccagtgggatgggctcctctcctaccaggactctctgagctgccgcctggtgtttctgctcatgcagtactgtgtggcggccaattactactggctcttggtggagggcgtgtacctgtacacactgctggccttctcggtcttctctgagcaatggatcttcaggctctacgtgagcataggctggggtgttcccctgctgtttgttgtcccctggggcattgtcaagtacctctatgaggacgagggctgctggaccaggaactccaacatgaactactggctcattatccggctgcccattctctttggcattggggtgaacttcctcatctttgttcgggtcatctgcatcgtggtatccaaactgaaggccaatctcatgtgcaagacagacatcaaatgcagacttgccaagtccacgctgacactcatccccctgctggggactcatgaggtcatctttgcctttgtgatggacgagcacgcccgggggaccctgcgcttcatcaagctgtttacagagctctccttcacctccttccaggggctgatggtggccatcttatactgctttgtcaacaatgaggtccagctggaatttcggaagagctgggagcgctggcggcttgagcacttgcacatccagagggacagcagcatgaagcccctcaagtgtcccaccagcagcctgagcagtggagccacggcgggcagcagcatgtacacagccacttgccaggcctcctgcagctgagactccagcgcctgccctccctggggtccttgctgcaggccgggtggccaatccaggtgggagagacactcccagggacaagggaaggaagggacacacacacacacacacacacacacacacacacatacatcctgctttccctccccaaacccatcagacaggtaaatgggcagtgcctcctgggaccatggacacattttctcctaggagaagcagcctcctaatttgatcacagtggcgagaggagaggaaaaacgatcgctgtgaaaatgaggaggattgcttcttgtgaaaccacaggcccttggggttcccccagacagagccgcaaatcaaccccagactcaaactcaaggtcaacggcttattagtgaaactggggcttgcaagaggaggtggttctgaaagtggctcttctaacctcagccaaacacagagcgggagtgacgggagcctcctctgcttgcatcacttggggtcaccaccctcccctgtcttctctcaaagggaagctgtttgtgtgtctgggttgcttatttccctcatcttgccccctcatctcactgcccagtttctttttgaggggctttgtttgggccactgccagcagctgtttctggaaatggctgtaggtggtgttgagaaagaatgagcattgagacggtgctcgcttctcctccaggtatttgagttgttttggtgcctgcctctgccatgcccagagaatcagggcaggcttgccaccggggaacccagccctggggtatgagctgccaagtctattttaaagacgctcaagaatcctctggggttcatctagggacacgttaggaatgtccagactgtgggtgtagattacctgccacttccaggagcccagagggccaagagagacattgcctccacctctccttggaaatactttatctgtgaccacacgctgtctcttgagaatttggatacactctctagctttaggggaccatgaagagactctcttagggaaaccaatagtccccatcagcaccatggaggcaggctccccctgcctttgaaattcccccacttgggagcttgtatatacttcactcacttttctttattgctgtgaatagtctgtgtgcacaatgggcaattctgacttctcccatctagtggaaatgagcgaaatcatggttgtagtgatgttgtttgggagagtgcagtagtaattgatttgacccactcacacttggagctaattaaggtttgccctgcctgcagcctcccccacaaataatgaacagcagaaagactggacggggaaacctatcaatcctgcccccagccatggtgaggaagccccaagccatggtgacacacagcagcactgcagatagccagacacatggctatcctagagaggctggcaaggagttcgtggctgcaaaagaagtttctggagcaagagagagctcgctcttgggagtcaggacctccggggagagcagagggttccgacggattcctttatgagtcagtctctctctcccttttaaatggtgggaaccctccccaaaacctttccccagacacattctcctgtgcccctcagagaggcatgtgatgtgcaaggaaaataataggatgtaaaacacatcaagtagaaaatttcttatacttccaaaaaaaaaagc

SEQ ID NO: 11 - DNA, 3088 bp, human
cgcgccagggaggttgtcgggaggggccggcgaataaaacgagcggcgaaagaaccgaaaaaaggctcgacgctaccgtgtatgaggaactttgatccttgcgggccaccattccggaagtagaatttagaggaagaaaataccggagttgcagggtataggtaaatttctcaaggttataggttggggttcttagaactttttgtggtgtgtgttggcctagagcgactcagaagcgttagtgacttcacctaaaaaagctaacctctctgctgagcgcgaccggtatgcggcgcaggatgagcctcagggcttctgttaagagtctgtctgagaaagccggtctgcgctgttcctcggtggcgaccttaattatgagatgagctaatgctttactgacttaaccatggcgcagcgggcagtgtggctcataagccacgaaccgggaactccactttgtggcaccgtgagattctccagacggtatccaactgttgaaaaacgagccagagtcttcaatggagcaagttatgtgcctgttcctgaagatggtccctttcttaaagcactgctctttgaacttagattattggatgatgataaagacttcgttgagagtcgtgatagctgttcacgcatcaataaaacatccatttatggactcctgataggaggtgaagaactctggccagttgttgcttttctgaagaatgacatgatatatgcttgtgttccactagttgaacaaactctgtcccctcgtccgccactaattagtgtcagtggagtttcacaaggctttgaatttctttttgggatacaggattttctttattcaggtcaaaaaaatgactctgagctgaatacaaaattgagccagttgcctgacttgcttctgcaggcttgtccatttggtactttattagatgccaacttacagaattcattagataataccaattttgcatctgtgactcagccacagaaacagccagcttggaaaactgggacgtacaaaggaaaaccacaagtttctatttctatcactgaaaaggtaaaatccatgcaatatgataaacagggtatagcagatacatggcaagttgttggaacagtgacttgcaagtgtgatttggaaggaatcatgccaaatgttaccatcagcttgagtctccccaccaatggatctccacttcaggatattctagttcacccttgtgtaacttctcttgactctgcaattctgacttctagtagtattgatgcaatggatgactctgcatttagtgggccttacaaatttccattcactccacctttagagtcattcaacttatgcttctacacttcccaggtccctgccccaccaattttgggtttttatcaaatgaaggaggaagaagtacaactaagaataaccattaatttaaaacttcatgaaagtgtgaaaaataattttgaattctgtgaagcccatatacctttttacaatagaggtccaattacacatttggaatacaaaactagttttggccagcttgaagtatttcgagagaaaagcttattgatctggattattggccagaagttcccaaaatcaatggaaattagtctttctggaactgtaacttttggagccaagagccatgagaagcagccatttgacccaatttgtactggagaaacagcatatttaaagcttcattttaggatcttagattacacacttactggatgttatgcagatcagcattcagttcaagtttttgcatcaggaaaaccaaaaataagtgcacaccggaaactaatttcttctgattattacatctggaattctaaagcccctgctccagtaacatatggatcattattattgtaatagtctcatgtttaaatgggattatataatgataacagtttaaagaaaatcataatcttatatttttaatgtggatgcatataacctgtgagtgaaaaatcactgaatgatttaattgtaaaagtagtcttatgtggtgtttgtagtctgatagagcttgaaaggacattttaaaagctaatgtctccaattttgtcaaccttcgattttatgccagtataattcagaacatagaaaagtaatgattcacttgggctcattttagactggtcctgggtcaccctgccacacttgtttcctagtgtttctgtggcagacattgctaatcaattacagcccttttctgtactgagccttggataaagggtcaggctcctttttagttcagagattcaggcagccactcccagtgggttgtagataatgtgcaagataaaaactattttctcttccaaatctaagtactaagctcctagtataaggtgttgttacagaataccagagaccatgttagagacaactacatctcttcaaaaaacagccaacagagacaaaggaaaagtgtttaaatagtaagctgttcttcttaatcagaactatcctattgactaataaataatctgcataattctacttaaggtgtgtaatctctgttctagagttagtttttaagtaagcttgttaatctgccactttgacattttgcttaggatgtcagtagccatattaagatgtgtagaataccttcagaagatgatcatagtgttttgtaatcatttaatgtctgcagccaaatttttaaaggtaatttagacctaatactgctcttgctgtgtcttattaagttaaaattaatgaatgaattctggtaaaaattcaaaaggcactctgtgagtagagagtatcatttaagcttattttagtcacatgtagtatatatctccttaaagctgtcactctcactttcttaccattctcttgatttcttcagaaaccatctagtcatcatctttatactctacctgcttctgcaattatatatcatattatgttttcagagcagttcattgtcaagttggactttaagtgaccattcaagaaaagatgaaatctcacgaacctcaaaacttcattcatgtctttttacaaatgagaaaaaaaaatgcattaaagattaatactcaatttgaaaaaaaaaaaaaaaaaaaa

SEQ ID NO: 12 - DNA, 2584 bp, human
gggggcgtggccccgagaaggcggagacaagatggccgcccatagcgcttggaggacctaagaggcggtggccggggccacgccccgggcaggagggccgctctgtgcgcgcccgctctatgatgcttgcgcgcgtcccccgcgcgccgcgctgcgggcggggcgggtctccgggattccaagggctcggttacggaagaagcgcagcgccggctggggagggggctggatgcgcgcgcacccggggggaggccgctgctgcccggagcaggaggagggggagagtgcggcgggcggcagcggcgctggcggcgactccgccatagagcaggggggccagggcagcgcgctcgccccgtccccggtgagcggcgtgcgcagggaaggcgctcggggcggcggccgtggccgggggcggtggaagcaggcgggccggggcggcggcgtctgtggccgtggccggggccggggccgtggccggggacggggacggggccggggccggggccgcggccgtcccccgagtggcggcagcggccttggcggcgacggcggcggctgcggcggcggcggcagcggtggcggcggcgccccccggcgggagccggtccctttcccgtcggggagcgcggggccggggcccaggggaccccgggccacggagagcgggaagaggatggattgcccggccctcccccccggatggaagaaggaggaagtgatccgaaaatctgggctaagtgctggcaagagcgatgtctactacttcagtccaagtggtaagaagttcagaagcaagcctcagttggcaaggtacctgggaaatactgttgatctcagcagttttgacttcagaactggaaagatgatgcctagtaaattacagaagaacaaacagagactgcgaaacgatcctctcaatcaaaataagggtaaaccagacttgaatacaacattgccaattagacaaacagcatcaattttcaaacaaccggtaaccaaagtcacaaatcatcctagtaataaagtgaaatcagacccacaacgaatgaatgaacagccacgtcagcttttctgggagaagaggctacaaggacttagtgcatcagatgtaacagaacaaattataaaaaccatggaactacccaaaggtcttcaaggagttggtccaggtagcaatgatgagacccttttatctgctgttgccagtgctttgcacacaagctctgcgccaatcacagggcaagtctccgctgctgtggaaaagaaccctgctgtttggcttaacacatctcaacccctctgcaaagcttttattgtcacagatgaagacatcaggaaacaggaagagcgagtacagcaagtacgcaagaaattggaagaagcactgatggcagacatcttgtcgcgagctgctgatacagaagagatggatattgaaatggacagtggagatgaagcctaagaatatgatcaggtaactttcgaccgactttccccaagagaaaattcctagaaattgaacaaaaatgtttccactggcttttgcctgtaagaaaaaaaatgtacccgagcacatagagctttttaatagcactaaccaatgcctttttagatgtatttttgatgtatatatctattattcaaaaaatcatgtttattttgagtcctaggacttaaaattagtcttttgtaatatcaagcaggaccctaagatgaagctgagcttttgatgccaggtgcaatctactggaaatgtagcacttacgtaaaacatttgtttcccccacagttttaataagaacagatcaggaattctaaataaatttcccagttaaagattattgtgacttcactgtatataaacatatttttatactttattgaaaggggacacctgtacattcttccatcatcactgtaaagacaaataaatgattatattcacagactgattggaattctttctgttgaaaagcacacacaataaagaacccctcgttagccttcctctgatttacattcaactctgatccctgggccttaggtttgacatggaggtggaggaagatagcgcatatatttgcagtatgaactattgcctctggacgttgtgagaattgtgctttcaccagaatttctaagaatttctgctaaatatcacctagcatgtgtaattttttttccttgcctgtgacttggacttttgatagttctataagaataaggctttttcttcccttgggcatgagtcagatacacaaggacccttcaggtgttactagaaggcgtccatgtttattgttttttaaagaatgtttggcactctctaacgtccactagcttactgagttatcaggtgcaggtcagactcttggctacagtgagaggcagcttctaggcagagttgcttaatgaaagggtttgtaatactttacaaaccattacctgtacctggcctggcctccaaaatattaacattctttttctgttgaaactcgcgagtgtaactttcataccacttgaatttattgatatttaattatgaaaactagcattacattattaaacgatttctaaaatc

SEQ ID NO: 13 - DNA, 2935 bp, human
ccgcaactcgctcggccgccgccatcttgcgagctcgtcgtactgaccgagcggggaggctgtcttgaggcggcaccgctcaccgacaccgaggcggactggcagccctgagcgtcgcagtcatgccggccggacccgtgcaggcggtgcccccgccgccgcccgtgcccacggagcccaaacagcccacagaagaagaagcatcttcaaaggaggattctgcaccttctaagccagttgtggggattatttaccctcctccagaggtcagaaatattgttgacaagactgccagctttgtggccagaaacgggcctgaatttgaagctaggatccgacagaacgagatcaacaaccccaagttcaactttctgaaccccaatgacccttaccatgcctactaccgccacaaggtcagcgagttcaaggaagggaaggctcaggagccgtccgccgccatccccaaggtcatgcagcagcagcagcagaccacccagcagcagctgccccagaaggtccaagcccaagtaatccaagagaccatcgtgcccaaagagcctcctcctgagtttgagttcattgctgatcctccctctatctcagccttcgacttggatgtggtgaagctgacggctcagtttgtggccaggaatgggcgccagtttctgacccagctgatgcagaaagagcagcgcaactaccagtttgactttctccgcccacagcacagcctcttcaactacttcacgaagctagtggaacagtacaccaagatcttgattccacccaaaggtttattttcaaagctcaagaaagaggctgaaaacccccgagaagttttggatcaggtgtgttaccgagtggaatgggccaaattccaggaacgtgagaggaagaaggaagaagaggagaaggagaaggagcgggtggcctatgctcagatcgactggcatgattttgtggtggtggaaacagtggacttccaacccaatgagcaagggaacttccctccccccaccacgccagaggagctgggggcccgaatcctcattcaggagcgctatgaaaagtttggggagagtgaggaagttgagatggaggtcgagtctgatgaggaggatgacaaacaggagaaggcggaggagcctccttcccagctggaccaggacacccaagtacaagatatggatgagggttcagatgatgaagaagaagggcagaaagtgcccccacccccagagacacccatgcctccacctctgcccccaactccagaccaagtcattgtccgcaaggattatgatcccaaagcctccaagcccttgcctccagcccctgctccagatgagtatcttgtgtcccccattactggggagaagatccccgccagcaaaatgcaggaacacatgcgcattggacttcttgaccctcgctggctggagcagcgggatcgctccatccgtgagaagcagagcgatgatgaggtgtacgcaccaggtctggatattgagagcagcttgaagcagttggctgagcggcgtactgacatcttcggtgtagaggaaacagccattggtaagaagatcggtgaggaggagatccagaagccagaggaaaaggtgacctgggatggccactcaggcagcatggcccggacccagcaggctgcccaggccaacatcaccctccaggagcagattgaggccattcacaaggccaaaggcctggtgccagaggatgacactaaagagaagattggccccagcaagcccaatgaaatccctcaacagccaccgccaccatcttcagccaccaacatccccagctcggctccacccatcacttcagtgccccgaccacccacaatgccacctccagttcgtactacagttgtctccgcagtacccgtcatgccccggcccccaatggcatctgtggtccggctgcccccaggctcagtgatcgcccccatgccgcccatcatccacgcgcccagaatcaacgtggtgcccatgcctccctcggcccctcctattatggccccccgcccaccccccatgattgtgccaacagcctttgtgcctgctccacctgtggcacctgtcccagctccagccccaatgccccctgtgcatcccccacctcccatggaagatgagcccacctccaaaaaactgaagacagaggacagcctcatgccagaggaggagttcctgcgcagaaacaagggtccagtgtccatcaaagtccaggtgcccaacatgcaggataagacggaatggaaactgaatgggcaggtgctggtcttcaccctcccactcacggaccaggtctctgtcattaaggtgaagattcatgaagccacaggcatgcctgcagggaaacagaagctacagtatgagggtatcttcatcaaagattccaactcactggcttactacaacatggccaatggcgcagtcatccacctggccctcaaggagagaggcgggaggaagaagtagacaagaggaacctgctgtcaagtccctgccattttgcctctcctgtctcccaccccctgccccagacccaggagcccccctgaggctttgccttgcctgcatatttgtttcgctcttactcagtttgggaattcaaattgtcctgcagaggttcattcccctgaccctttccccacattggtaagagtagctgggttttctaagccactctctggaatctctttgtgttagggtctcgatttgaggacattcatttcttcagcagcccattagcaactgagagcccagggatgtcctacaggatagtttcatagtgacaggtggcacttggctaatagaatatggctgatattgtcattaatcattttgtaccttgacatgggttgtctaataaaactcggacccttcttgtgaaaaaaaaaaaaaaaaaa

SEQ ID NO: 14 - DNA, 462 bp, human
atggcgcgtactaagcagacggctcgtaaatccacaggcggtaaagcaccgcgcaaacagctggccactaaggcagctcgcaagagcgctccggccacgggcggcgtgaagaagccccatcgctaccgccctggcaccgtggctctgcgcgagatccgtcgctaccagaagtctaccgagcttctaatccggaagctgccgtttcagcgcctggtgcgagaaatagctcaggacttcaagaccgacctgcgcttccagagttccgcggtgatggcgctgcaggaggcctgcgaggcctacttggtggggcttttcgaggacaccaacctgtgcgctattcatgccaaacgcgtgaccatcatgcctaaagacatccagcttgcccgccgcattcgtggggagagggcgtgaattgttttgagtacaaaccttaaatccaaaggctcttctcagagccaacca

SEQ ID NO: 15 - DNA, 6830 bp, human
ccacggtaaggggatgacgtagctttgccaaagacttagaagctaagcagaaaatgagcttaacatcctggtttttggtgagcagtggaggcactcgccacaggctgccacgagaaatgatttttgttggaagagatgactgtgagctcatgttgcagtctcgtagtgtggataagcaacacgctgtcatcaactatgatgcgtctacggatgagcatttagtgaaggatttgggcagcctcaatgggacttttgtgaatgatgtaaggattccggaacagacttatatcaccttgaaacttgaagataagctgagatttggatatgatacaaatcttttcactgtagtacaaggagaaatgagggtccctgaagaagctcttaagcatgagaagtttaccattcagcttcagttgtcccaaaaatcttcagaatcagaattatccaaatctgcaagtgccaaaagcatagattcaaaggtagcagacgctgctactgaagtgcagcacaaaactactgaagcactgaaatccgaggaaaaagccatggatatttctgctatgccccgtggtactccattatatgggcagccgtcatggtggggggatgatgaggtggatgaaaaaagagctttcaagacaaatggcaaacctgaagaaaaaaaccatgaagctggaacatcagggtgcagcatagatgccaagcaagttgaggaacaatctgcagctgcaaatgaagaagtactttttcctttctgtagggaaccaagttattttgaaatccctacaaaagaattccagcaaccatcacaaataacagaaagcactattcatgaaatcccaacaaaagacacgccaagttcccatataacaggtgcagggcatgcttcatttaccattgaatttgatgacagtaccccagggaaggtaactattagagaccatgtgacaaagtttacttctgatcagcgccacaagtccaagaagtcttctcctggaactcaagacttgctggggattcaaacaggaatgatggcacccgaaaacaaagttgctgactggctagcacaaaacaaccctcctcaaatgctatgggaaagaacagaagaggattctaaaagcattaaaagtgatgttccagtgtacttgaaaaggttgaaaggaaataaacatgatgatggtacgcaaagtgattcagagaacgctggggctcacaggcgctgtagcaaacgtgcaactcttgaggaacacttaagacgccaccattcagaacacaaaaagctacagaaggtccaggctactgaaaagcatcaagaccaagctgttactagctctgcgcatcacagaggggggcatggtgttccacatgggaaattgttaaaacagaaatcagaggagccatcggtgtcaatacccttcctacaaactgcattattaagaagttcagggagtcttgggcacagaccaagccaggagatggataaaatgttaaaaaatcaagcaacttctgctacttctgaaaaggataatgatgatgaccaaagtgacaagggtacttataccattgagttagagaatcccaacagtgaggaagtggaagcaagaaaaatgattgacaaggtgtttggagtagatgacaatcaggattataataggcctgttatcaacgaaaaacataaagatctaataaaagattgggctctcagttctgctgcagcagtaatggaagaaagaaaaccactgactacatctggatttcaccactcagaggaaggcacatcttcatctggaagcaaacgttgggtttcacagtgggctagtttggctgccaatcatacaaggcatgatcaagaagaaaggataatggaattttctgcacctcttcctttagagaatgagacagagatcagtgagtctggcatgacagtgagaagtactggctctgcaacttccttggctagccagggagagagaaggagacgaactcttccccagcttccaaatgaagaaaagtctcttgagagccacagagcaaaggttgtaacacagaggtcagagataggagaaaaacaagacacagaacttcaggagaaagaaacacctacacaggtataccagaaagataaacaagatgctgacagacccttgagtaaaatgaacagggcagtaaatggagagactctcaaaactggtggagataataaaaccctacttcacttaggcagctctgctcctggaaaagagaaaagtgaaactgataaggaaacttctttggtaaagcaaacattagcaaaacttcaacaacaagaacaaagggaggaggctcagtggacacctactaaattgtcttccaaaaatgtttcaggtcagacagataaatgtagggaggaaacttttaaacaagaatcacaacctccagaaaaaaattcaggacattctacaagcaaaggagacagagtggcacaaagtgagagcaagagaagaaaagctgaggaaattctgaaaagtcagactccaaagggaggagacaagaaggaatcctccaagtcattagtgcgacaagggagcttcactatagaaaaacccagcccaaacatacccatagaacttattccccatataaataaacagacttcctctactccttcttctttagcattaacatctgcaagtagaatacgagaaagaagtgagtctttggatcctgattctagtatggacacaacccttattctaaaagacacagaagcagtaatggcttttctagaagctaaactacgtgaagataataaaactgatgaaggaccagatactcccagttataatagagacaattctatttcaccagaatctgatgtagatacagctagtacaatcagtctggttactggagaaactgaaagaaagtcaacccaaaagcgaaagagtttcactagcctctataaagataggtgttccacaggttctccttccaaagatgttacaaaatcatcatcttcaggtgctagggaaaaaatggaaaagaaaacaaaaagtcgttccacagatgtgggttcaagagcagatggtcgtaaatttgttcagtccagtgggagaataagacagccctcagtagacttaacagatgatgaccaaacctctagtgtacctcattctgccatctctgatattatgtcatctgatcaagaaacttactcttgtaaacctcatggacggactccacttacctcagctgatgagcatgtacattccaaactggaaggaagtaaagtaacgaaatctaagacttctccggtggtatctggttcatctagtaaatcaaccacccttccaaggccacgacctaccaggacttccctcttgcgcagagcacgacttggtgaagcttcagacagtgaacttgctgatgctgacaaagcatctgttgcttctgaagtatccacaacaagttctacatcaaaacctcccacaggaaggcgtaacatctctcggattgatttattggctcagcctcgtagaacacgacttggctcactgtcagctcgtagtgactctgaagcaacaatttctagaagtagtgcctcttcgaggaccgcagaagccatcattagaagtggagccagactagtaccatcagataaattttctcctagaattagagctaacagtatctctcgactctcagactccaaggtcaaaagtatgacctcagctcatggctctgcttcagtaaattcaagatggaggcgctttcctactgattatgcttccacctcagaagatgaatttggatcaaaccgtaattcccctaaacatacccgtctacgtacttctccagccctgaaaaccactcgcttgcagagcgctggatcagcaatgcctactagttcttcattcaaacaccggattaaagagcaggaagactacatccgagattggactgctcatcgagaagagatagccaggatcagccaagatcttgctctcattgctcgggagatcaacgatgtagcaggagagatagattcagtgacttcatcaggcactgcccctagtaccacagtaagcactgctgccaccacccctggctctgccatagacactagagaagagttggttgatcgtgtttttgatgaaagcctcaacttccgaaagattcctccattagttcattccaaaacaccagaaggaaacaacggtcgatctggtgatccaagacctcaagcagcagagcctcccgatcacttaacaattacaaggcggagaacctggagcagggatgaagtcatgggagataatctgctgctgtcatccgtctttcagttctctaagaagataagacaatctatagataagacagctggaaagatcagaatattatttaaagacaaagatcggaattgggatgacatagaaagcaaattaagagccgaaagtgaagtccctattgtgaaaacctcaagcatggagatttcttctatcttacaggaactgaaaagagtagaaaagcagctgcaagcaatcaatgctatgattgatcctgatggaactttggaggctctgaacaacatgggatttcccagtgctatgttgccatctccaccgaaacagaagtccagccctgtgaataaccaccacagcccgggtcagacaccaacacttggccaaccagaagctagggctcttcatcctgctgctgtttcagccgcagctgaatttgagaatgctgaatctgaggctgatttcagtatacatttcaatagattcaaccccgatggggaagaggaagatgttacagtacaagaatgactttctcttgattgttgaaaaatcattacctgtggaatggctaggaatattggaagcagcatagtgttgatgtacgcaaaacaagacagcttggtcagctacaatcttggaatccctgtcttcttaattttatttatttatttttgacgtataatgtagtatatcaatcctttcaaactatttagataaccacttgatgcacaaataggaaaaagcagattgtggcagtgtcgccttttgtggttttatgattttcaaattgaatttaatgattacaccctttcccttcatagatcttttttcttttttttaagccatgctgtgacctacaagcaaactaaatagccaacatttctgaacccctaagtctcctgtgccaagctgctccctgaaatggacttcttcatctgtacagatttgttaaaccattctatttgcttcttaataataggatttatattagtactcattaccattggacacaatgacataagtactctccacagtaaagcagacctttcacaacagtcactctgtgtcctaaaattttccaacatagatgtgatttatataactttgttgatacgtaaattgtcttggggtttacggaaattaactattatgtttgcactaagatttgctgggagtggtaggtggacatatctatatatcaataaggactaaccgtcttttttgtacataggggattgataatactgtatttgttttaagcccacagtgtttttactccactttcaaaaagtcaatttgggcactttttttcatttttttttaatgggaataagatttggtctctcattttaggttaaatgataactagaaagattaaactagacagatagtttaggtggagtatatttttaaaactcagaacatgtatattggtcctgtgttaccaagtttatatgtgacagttgaaaaagaaattcccttgaaatgatcatgaggttaaaattttcttcattaggggacttggagaaccagtagtcgtaagattagttgatagtttcactcccaagcaatgaattgcttctgtgtgtttccctgtaggactcaatagtaaatgctgtctgtcttacacatttataaggaccctgcaagacgacgacaaaggcctttggcctgtgctactaaacaagaagcctatgaaaaatttcttctttaaacttgttttttctctttccagtaagttcacatttggataattttaaaaagaaaagtaattacctttgtgtttccagaacactataattggggtgtatcttaattcagttaaatattattagtagacctggattttcccccttgaccccatcagtctataaaggttaaactgcaacttttatgaaatggtctttaatatttccacaataatcctgtgctatatttgttttaagaaacaaagtaactctatacacttcaagactttacaggattttttaaatcctgtattgttggatcaattaataaagatgcaaaaaaactttatagagatgtaaaaacaaaactataatggatctcctatttttctttaaatacaaaaaaaaaaaggtaaatgaactattctccttgtaaagctaaattccccattctgtctaataaaggaagactgaaaaaaggttttaaagaacataaatggaaagatacaaatgctttgaaggaataaacgaaatgttaaaacagggtcaatccatttgaagaaaaagttggacaaaataatcagcattgctccctcttttatttaatatttgggtactgattatatccacatggaagtagaaggtaaggagtttaggaaaatactagaatctactctgcttacattcttgtttaagtgtttacacagtttggttcaattataaacatttggccttttactatgttatttttattttgtatgaatacattatcctccttatttatttttgttacatttattacttatgttattttgttatgcatttacaactccatttttaaataaagtgtttctggaacttt

SEQ ID NO: 16 - DNA, 2001 bp, human
ggccgcggtggtggctgcggcggcggcggcgggagcagcatggattggggcactgagctgtgggatcagttcgaggtgctcgagcgccacacgcagtgggggctggacctgttggacagatatgtaaagttcgtgaaagaacgcaccgaagtggaacaggcttacgccaaacaactgcggagcctggtgaaaaaatatctgcccaagagacctgccaaggatgatcctgagtccaaattcagccagcaacagtccttcgtacagattctccaggaggtgaatgactttgcaggccagcgggagctggtggctgagaacctcagtgtccgtgtatgtcttgagctgaccaagtactcacaagagatgaaacaggagaggaagatgcacttccaagaagggcggcgggcccagcagcagctggaaaatggctttaaacagctggagaatagtaagcgtaaatttgagcgggactgccgggaggcagagaaggcagcccagactgctgaacggctagaccaggatatcaacgccaccaaggctgatgtggagaaggccaagcagcaagcccaccttcggagtcacatggccgaagaaagcaaaaacgaatatgcggctcaactgcagcgcttcaaccgagaccaagcccacttctatttttcacagatgccccagatattcgataagctccaagacatggatgaacgcagggccacccgcctgggtgccgggtatgggctcctgtcggaggccgagctggaggtggtgcccataatagccaagtgcttggagggcatgaaggtggctgcaaatgctgtggatcccaagaacgactcccacgtccttatagagctgcacaagtcaggttttgcccgcccgggcgacgtggaattcgaggacttcagccagcccatgaaccgtgcaccctccgacagcagtctgggcaccccctcggatggacggcctgaactccgaggcccgggtcgcagccgcaccaagcgctggccttttggcaagaagaacaagacagtggtgaccgaggattttagccacttgcccccagagcagcagcgaaaacggcttcaacagcagttggaagaacgcagtcgtgaacttcagaaggaggttgaccagagggaagccctaaagaaaatgaaggatgtctatgagaagacacctcagatgggggaccccgccagcttggagccccagatcgctgaaaccctgagcaacattgaacggctgaaattggaagtgcagaagtatgaggcgtggctggcagaagctgaaagtcgagtccttagcaaccggggagacagcctgagccggcacgcccggcctcccgacccccccgctagcgccccgccagacagcagcagcaacagcgcatcacaggacaccaaggagagctctgaagagcctccctcagaagagagccaggacacccccatttacacggagtttgatgaggatttcgaggaggaacccacatcccccataggtcactgtgtggccatctaccactttgaagggtccagcgagggcactatctctatggccgagggtgaagacctcagtcttatggaagaagacaaaggggacggctggacccgggtcaggcggaaagagggaggcgagggctacgtgcccacctcctacctccgagtcacgctcaattgaaccctgccagagacgggaagaggggggctgtcggctgctgcttctgggccacggggagccccaggacctatgcactttatttctgaccccgtggcttcggctgagacctgtgtaacctgctgccccctccacccccaacccagtcctacctgtcacaccggacggacccgctgtgccttctaccatcgttccaccattgatgtacatactcatgttttacatcttttctttctgcgctcggctccggccattttgttttatacaaaaatgggttttttttttttctttaatatatttcaagagatttttttttttttttttttt

SEQ ID NO: 17 - DNA, 2956 bp, human
cgaggcgcggctccggggattcggctcgggccgctggctctgctctgcggggagggagcgggcccgcccgcggggcccgagccctccggatccgccccctccccggtcccgccccctcggagactcctctggctgctctgggggttcgccggggccggggacccgcggtccgggcgccatgcgggcatcgctgctgctgtcggtgctgcggcccgcagggcccgtggccgtgggcatctccctgggcttcaccctgagcctgctcagcgtcacctgggtggaggagccgtgcggcccaggcccgccccaacctggagactctgagctgccgccgcgcggcaacaccaacgcggcgcgccggcccaactcggtgcagcccggagcggagcgcgagaagcccggggccggcgaaggcgccggggagaattgggagccgcgcgtcttgccctaccaccctgcacagcccggccaggccgccaaaaaggccgtcaggacccgctacatcagcacggagctgggcatcaggcagaggctgctggtggcggtgctgacctctcagaccacgctgcccacgctgggcgtggccgtgaaccgcacgctggggcaccggctggagcgtgtggtgttcctgacgggcgcacggggccgccgggccccacctggcatggcagtggtgacgctgggcgaggagcgacccattggacacctgcacctggcgctgcgccacctgctggagcagcacggcgacgactttgactggttcttcctggtgcctgacaccacctacaccgaggcgcacggcctggcacgcctaactggccacctcagtctggcctccgccgcccacctgtacctgggccggccccaggacttcatcggcggagagcccacccccggccgctactgccacggaggctttggggtgctgctgtcgcgcatgctgctgcaacaactgcgcccccacctggaaggctgccgcaacgacatcgtcagtgcgcgccctgacgagtggctgggtcgctgcattctcgatgccaccggggtgggctgcactggtgaccacgagggggtgcactatagccatctggagctgagccctggggagccagtgcaggagggggaccctcatttccgaagtgccctgacagcccaccctgtgcgtgaccctgtgcacatgtaccagctgcacaaagctttcgcccgagctgaactggaacgcacgtaccaggagatccaggagttacagtgggagatccagaataccagccatctggccgttgatggggaccgggcagctgcttggcccgtgggtattccagcaccatcccgcccggcctcccgctttgaggtgctgcgctgggactacttcacggagcagcacgctttctcctgcgccgatggctcaccccgctgcccactgcgtggggctgaccgggctgatgtggccgatgttctggggacagctctagaggagctgaaccgccgctaccacccggccttgcggctccagaagcagcagctggtgaatggctaccgacgctttgatccggcccggggtatggaatacacgctggacttgcagctggaggcactgaccccccagggaggccgccggcccctcactcgccgagtgcagctgctccggccgctgagccgcgtggagatcttgcctgtgccctatgtcactgaggcctcacgtctcactgtgctgctgcctctagctgcggctgagcgtgacctggcccctggcttcttggaggcctttgccactgcagcactggagcctggtgatgctgcggcagccctgaccctgctgctactgtatgagccgcgccaggcccagcgcgtggcccatgcagatgtcttcgcacctgtcaaggcccacgtggcagagctggagcggcgtttccccggtgcccgggtgccatggctcagtgtgcagacagccgcaccctcaccactgcgcctcatggatctactctccaagaagcacccgctggacacactgttcctgctggccgggccagacacggtgctcacgcctgacttcctgaaccgctgccgcatgcatgccatctccggctggcaggccttctttcccatgcatttccaagccttccacccagctgtggccccaccacaagggcctgggcccccagagctgggccgtgacactggccgctttgatcgccaggcagccagcgaggcctgcttctacaactccgactacgtggcagcccgtgggcgcctggcggcagcctcagaacaagaagaggagctgctggagagcctggatgtgtacgagctgttcctccacttctccagtctgcatgtgctgcgggcggtggagccggcgctgctgcagcgctaccgggcccagacgtgcagcgcgaggctcagtgaggacctgtaccaccgctgcctccagagcgtgcttgagggcctcggctcccgaacccagctggccatgctactctttgaacaggagcagggcaacagcacctgaccccaccctgtccccgtgggccgtggcatggccacaccccaccccacttctcccccaaaaccagagccacctgccagcctcgctgggcagggctggccgtagccagaccccaagctggcccactggtcccctctctggctctgtgggtccctgggctctggacaagcactgggggacgtgcccccagagccacccacttctcatcccaaacccagtttccctgccccctgacgctgctgattcgggctgtggcctccacgtatttatgcagtacagtctgcctgacgccagccctgcctctgggccctgggggctgggctgtagaagagttgttggggaaggagggagctgaggagggggcatctcccaacttctcccttttggaccctgccgaagctccctgcctttaataaactggccaagtgtgaaaaaaaaaaaaaaaaaaaaaaa

SEQ ID NO: 18 - DNA, 889 bp, human
gcgaactggtggcagtgagagacttcggcggacatggctcccagcgtgccagcggcagaacccgagtatcctaaaggcatccgggccgtgctgctggggcctcccggggccggtaaagggacccaggcacccagattggctgaaaacttctgtgtctgccatttagctactggggacatgctgagggccatggtggcttctggctcagagctaggaaaaaagctgaaggcaactatggatgctgggaaactggtgagtgatgaaatggtagtggagctcattgagaagaatttggagacccccttgtgcaaaaatggttttcttctggatggcttccctcggactgtgaggcaggcagaaatgctcgatgacctcatggagaagaggaaagagaagcttgattctgtgattgaattcagcatcccagactctctgctgatccgaagaatcacaggaaggctgattcaccccaagagtggccgttcctaccacgaggagttcaaccctccaaaagagcccatgaaagatgacatcaccggggaacccttgatccgtcgatcagatgataatgaaaaggccttgaaaatccgcctgcaagcctaccacactcaaaccaccccactcatagagtactacaggaaacgggggatccactccgccatcgatgcatcccagacccccgatgtcgtgttcgcaagcatcctagcagccttctccaaagccacatgtaaagacttggttatgtttatctaatgttgggtccaagaaggaatttctttccatccctgtgaggcaatgggtgggaatgataggacaggcaaagagaagcttcctcaggctagcaaaaatatcatttgatgtattgattaaaaaagcacttgcttgatgt

SEQ ID NO: 19 - DNA, 2964 bp, human
agtcggcggcggctgctgctgcctgtggcccgggcggctgggagaagcggagtgttggtgagtgacgcggcggaggtgtagtttgacgcggtgtgttacgtgggggagagaataaaactccagcgagatccgggccgtgaacgaaagcagtgacggaggagcttgtaccaccggtaactaaatgaccatggaatctggagccgagaaccagcagagtggagatgcagctgtaacagaagctgaaaaccaacaaatgacagttcaagcccagccacagattgccacattagcccaggtatctatgccagcagctcatgcaacatcatctgctcccaccgtaactctagtacagctgcccaatgggcagacagttcaagtccatggagtcattcaggcggcccagccatcagttattcagtctccacaagtccaaacagttcagatttcaactattgcagaaagtgaagattcacaggagtcagtggatagtgtaactgattcccaaaagcgaagggaaattctttcaaggaggccttcctacaggaaaattttgaatgacttatcttctgatgcaccaggagtgccaaggattgaagaagagaagtctgaagaggagacttcagcacctgccatcaccactgtaacggtgccaactccaatttaccaaactagcagtggacagtatattgccattacccagggaggagcaatacagctggctaacaatggtaccgatggggtacagggcctgcaaacattaaccatgaccaatgcagcagccactcagccgggtactaccattctacagtatgcacagaccactgatggacagcagatcttagtgcccagcaaccaagttgttgttcaagctgcctctggagacgtacaaacataccagattcgcacagcacccactagcactattgcccctggagttgttatggcatcctccccagcacttcctacacagcctgctgaagaagcagcacgaaagagagaggtccgtctaatgaagaacagggaagcagctcgagagtgtcgtagaaagaagaaagaatatgtgaaatgtttagaaaacagagtggcagtgcttgaaaatcaaaacaagacattgattgaggagctaaaagcacttaaggacctttactgccacaaatcagattaatttgggatttaaattttcacctgttaaggtggaaaatggactggcttggccacaacctgaaagacaaaataaacattttattttctaaacatttctttttttctatgcgcaaaactgcctgaaagcaactacagaatttcattcatttgtgcttttgcattaaactgtgaatgttccaacacctgcctccacttctcccctcaagaaattttcaacgccaggaatcatgaagagacttctgcttttcaacccccaccctcctcaagaagtaataatttgtttacttgtaaattgatgggagaaatgaggaaaagaaaatctttttaaaaatgatttcaaggtttgtgctgagctccttgattgccttagggacagaattaccccagcctcttgagctgaagtaatgtgtgggccgcatgcataaagtaagtaaggtgcaatgaagaagtgttgattgccaaattgacatgttgtcacattctcattgtgaattatgtaaagttgttaagagacataccctctaaaaaagaactttagcatggtattgaaggaattagaaatgaatttggagtgctttttatgtatgttgtcttcttcaatactgaaaatttgtccttggttcttaaaagcattctgtactaatacagctcttccatagggcagttgttgcttcttaattcagttctgtatgtgttcaacatttttgaatacattaaaagaagtaaccaactgaacgacaaagcatggtatttgaattttaaattaaagcaaagtaaataaaagtacaaagcatattttagttagtactaaattcttagtaaaatgctgatcagtaaaccaatcccttgagttatataacaagatttttaaataaatgttattgtcctcaccttcaaaaatatttatattgtcactcatttacgtaaaaagatatttctaatttactgttgcccattgcacttacataccaccaccaagaaagccttcaagatgtcaaataaagcaaagtgatatatatttgtttatgaaatgttacatgtagaaaaatactgattttaaatattttccatattaacaatttaacagagaatctctagtgaattttttaaatgaaagaagttgtaaggatataaaaagtacagtgttagatgtgcacaaggaaagttattttcagacatatttgaatgactgctgtactgcaatatttggattgtcattcttacaaaacatttttttgttctcttgtaaaaagagtagttattagttctgctttagctttccaatatgctgtatagcctttgtcattttataattttaattcctgattaaaacagtctgtatttgtgtatatcatacattgttttcaataccacttttaattgttactcattttattcactaagctcgataaatctaacagttactcttaaaaaaaaaaaaaagactaaggtggattttaaaaattggaaactgacataatgttaggttataatttctcatttggagccgggcgcagtggctcacgcctgtaatcccagcactttgggaggccaaggtgggtggatcacctgtggtcaagagttcaagaccagcctggccatcatggtgaaaccccatctctactaaaaatacaaaaattagccaggcgtggtggctggcgcctgtaatcccagctactcaggaggttgaggcagcagaattgcttgaacccaggaggcagagggttgcagtgagccgagatagcaccattgcactccagcctgggcgactccatctcaaaaaataaaaaaaaaaaaaaa

SEQ ID NO: 20 - DNA, 1004 bp, human
atgggtctggagctctacctggacctgctgtcccagccctgccgcgctgtttacatctttgccaagaagaacgacattcccttcgagctgcgcatcgtggatctgattaaaggtcagcacttaagcgatgcctttgcccaggtgaaccccctcaagaaggtgccggccttgaaggacggggacttcaccttgacggagagtgtggccatcctgctctacctgacgcgcaaatataaggtccctgactactggtaccctcaggacctgcaggcccgtgcccgtgtggatgagtacctggcatggcagcacacgactctgcggagaagctgcctccgggccttgtggcataaggtgatgttccctgtgttcctgggtgggccagtatctccccagacactggcagccaccctggcagagttggatgtgaccctgcagttgctcgaggacaagttcctccagaacaaggccttccttactggtcctcacatctccttagctgacctcgtagccatcacggagctgatgcatcccgtgggtgctggctgccaagtcttcgaaggccgacccaagctggccacatggcggcagcgcgtggaggcagcagtgggggaggacctcttccaggaggcccatgaggtcattctgaaggccaaggacttcccacctgcagaccccaccataaagcagaagctgatgccctgggtgctggccatgatccggtgagctgggaaacctcacccttgcaccgtcctcagcagtccacaaagcattttcatttctaatggcccatgggagccaggcccagaaagcaggaatggcttgcttaagacttgcccaagtcccagagcacctcacctcccgaagccaccatccccaccctgtcttccacagccgcctgaaagccacaatgagaatgatgcacactgaggccttgtgtcctttaatcactgcatttcattttgattttggataataaacctggctcagcctgagcctctgcttct

SEQ ID NO: 21 - DNA, 8093 bp, human
ctcagctccctgtggtgggggatggcagaggcttcccgctccccgcagctggtttgcacagcctcccgactgtgccgtggtgtctccttccctaaagtgggacagtcgtttctgtgtcctgctggcctctgaggaacagacgtgtgtgagaggccttcagggcctgatggctggggtggtgacagtgcctggagaatgggtggggactggaggggccaggtggctaaccactctcctcttccatggcagcggagctggagttcgcccaaatcatcatcatcgtcgtggtggtcacggtgatggtggtggtcatcgtctgcctgctgaaccactacaaagtctccacgcggtccttcatcaaccgcccgaaccagagccggaggcgggaggacgggctgccgcaggaagggtgcctgtggccttcagacagcgccgcaccgcggctgggcgcctcggagatcatgcatgccccgcggtccagggacaggttcacagcgccgtccttcatccagagggatcgcttcagccgcttccagcccacctacccctatgtgcagcacgagattgatcttcctcccaccatctccctgtccgacggtgaagagccacctccttaccaggggccctgcaccctgcagctccgggaccctgaacagcagatggaactcaaccgagagtccgtgagggccccacccaaccgaaccatatttgacagtgatttaatagacattgctatgtatagcgggggtccatgcccacccagcagcaactcgggcatcagtgcaagcacctgcagcagtaacgggaggatggaggggccaccccccacatacagcgaggtgatgggccaccacccaggcgcctctttcctccatcaccagcgcagcaacgcacacaggggcagcagactgcagtttcagcagaacaatgcagagagcacaatagtacccatcaaaggcaaagataggaagcctgggaacctggtctgattccttccaacgtgcacttcagctggagaaagaaaccaagaagggaagcggccgctgggcccctcctgcgcacagtgttgttcagtttcacatggtacaaataagtaaaaccaaatgagcaaacacggtctttgtttctgattccttttaggggaattgcatgcaaactagactgaaatgatacaaacttccatctggtctgaccgcaaacagtgtttatttggggacaggggttgggatgggggtgtgggcaggggaaaacagagaacgggatgctttgaagataccatgaaataaaacccacagaggtatttgatgtatttaattgtgaaaggagactttgcagataaatgaggccagaatggcatgttttataattaactgaataaagaaggaagcattattatatattattgtggggaagaaccagccagttcgctttttctcctaaggtgtggacttttattttgttttaaaaatatgaatcaaaattcctgtgttgtgtgccaaggtataaagtggagaagttagatgagtgcaaggagctcctttgtgttgtgatgatgtgttttaaaagttgcactatcttaatgttgaaaatatttacaagggaactgttttacgtgaagttctgtatgttgtcttttcacctgtggattgtaatcaggcccaaggaatatcctggagtggtccccagaagcatccaagaaaagatatttggggacgtagcctaacattttaccaacttacgtaaatcaaaaaagtcattattgttgcaggagtttgcatcaaatagcagtgcatcgctgaagcttttggagacttttggatggaagataagatagggaagattaagttccagcatttctgacttgttattttgagttactctgctactcttaggctgcatagtttatgagaaaatgaacacatgcatttatggatccagtatcatgcagtgctgccctcatcctccagcagtgcaatttcttcagtaatttagatttttttcactatagcatgaaatatattcaaatacataccttattttatgcaataaattgtttaaaatgcaaggtggttattctgcatactgttgaaatatgtgactcctcagtatattcccattgcctctccccctttcctcgacagcttagttcagttctgcagggctgctcagttcacaggaggctcccagcagccaccccacatccagcctacacagaactttcgtgtgggagtggtgtgggtggtggttttcttatgctttggaagcccctagaaataatgacggaagaatgccatgttgctgatcgtggtaataagccattgtgggttattgtatgtcactagtattagcatagcattcttaaaggaatgcagtgttcaaaacctacccaaattccccgcaggattttaccaaacccttccccaggccagttttgtactgaaggcaagaactggacagtcagagaacagtggagggggcaagtgactgaagagcaccgggtaaaaagcacaacatgcagttaaaatgcaaactagaaaactaattttaaatattgttagttttaatatttcctgatatttacaaatattcattcttatatacaatgaaaaaaataactttcttctgcagatgtaagcactggcttttataagagcagcagccaacacgtttagcagacactgcgcgtggagaagggcttatctgcagtacactctgccatgtggagggtgggcctctgtggcctcttcacataacaagatgagctggaatgatgattccatgactcccacctatgcagccttaaagccaaatccgcgtgtgtgtgtttgtgtctgtctgtgggtctcgaaggtgatccgtcggtgcggtggctctgtgctgtaactggagagactgttccaaaccccaagagttgtctgatcctagtctgttcccttctgcttcttacctctgtagataggtcactggtttttgtttgtttgttttgaggattggaatttccattacattcatcctttgcacacagtaacatccacagaactagtccaactcttaaaaggagagaggaaaaacacaggcaccagttgtcagctcatgcttacaacctgtgtggaagtatatacagttgagagtcacagtggaggttctgagactggattcagtcttgttccagtgacagttggaaggcctctgctggagagacaccagctctcagggcagagattggcttggggccagaaggaccctccccaaccctggagacaccctgaaggttcactggctctccagattagcctctcttcctctgtcaggcaaagatgaggagcccgtgttcccatcgggccctgctggcagggacttgcagtggattcttggtcaggtgtgcccacagatgcggaggcgaggtgagtgattccatcatttcagttctcacctgcagttttggtgaagcaggagatgcaccccacagctctagctctcaaatggcttcacagtccttacttctctacctgcctcaagaaggggctcagagcagagacttgtgaattccttagtaactgtgagtatatgaatgtgttgcacatgtccacagtattggcgagataattacataattcagatacctttaatcatctttcaagaaagaggctcctcccattcaaccaccctagagaactgcctttgttaaatagttatttaaagactcatacatatcaaaccatgactttgaaaggtcttcgaggctggggctctgtaatgaattagtttaaaagccaaggtcataacatgaattgatggtcaatttcccttcagcagaaggaaaaggtgatttagatcagtagctcttttgaaggttgtggctgacctgttcataccgtgtcgcctcatggctagtgtggcgttgaaagagtagcgactgggaagatacaacttacacagtggggcctattgttctttcaagaaccctttttttagcttatagaacccatgggtccagtttagtaacgagtgatttaggcaatcaatgataggtttataatcttagattattccagcaaagtgtggattgcattgttaggaagaacatttggtgggaatgaacactcctgggcataccgctgacttttgtcccttgttcccggtgtaggagacccaaggcatcttgaatcccatctataagaacacaatcttccagcatacgtttgctttttcagaaactctagcattctctttaaatactgacgcaatccttaatggaaaagagatttcatgaagcaaattatgtatttcaatagttcttctatttttagtgtccaaaatttactaatacagaagcttgacaagcatgtcctcaccctccccaccacataaacacatggacacacacccaagccacaagaaatcccaagagagcagaagcgaatttttaaaagatttatcgtgaggactgcatttccattcactaattttggctcaaacttatgaggcaggaaataggggccaacagtaaatgggggaggcctcctgacaccagcagaggaattttgtacccaggcgaggacttcttgaacttctgcgtatctccgtttgatctctttcacctttatttcatcttcataagaatgagaaaggctcaaaaggaagcacttttagaaatcttctctgacctagaagaatccatccaaatccctgccttcctctctgaaccaacagttcccttctctgacagggggccatcctctatcttccatccagcggctcttccttttaggaaggctctggtgcagagcacttcaaatatgtcctcaggccagatactgattgctagtagagagacacccggcacccagtccgaagccctccctcaaaggaccggcttatggcgttggtcactggcaggctcagagacattctactgtgggcgcagggagcccggccccccatgcagccatgactggatgcgcccccatctcgggggcttgctgcactgcttgtttattgaattttgctacttagaatggcaacattaactttgtgtaccattcattttttaaaaattttccaaagctcggcagtgtatgaaagaaaaaactgggaaagatacttggtttctgttaacttttgtgttgcttgcttaagtgattaaagccagtgcttggagccaagccttcatgccacgaacatgctccacagcctgccctttgctctcctgctcacactgaccaagaatgccgcgtgcttggcctactgaggtgaaaggacaattgaatgacaggtgggcaaagggagaacttccccttcttggtgcgaggaaagtcacaaatttaaaaatgttgcttccagcccagatcctaaatgctagttctcagcagctgcgtggcttaccgttcgccatttccaccaccgccagctgccagcaccgctacagatcacagagatgtgaacagacaatggaaagcactcttagccttgcagtggtctacattttttaggaaccaatatttcagcattctttattacccggcacgctgtgtcctttgcagagttcaagtttatgttactgccagggtcagacagtcatttgctgctgctgctgctgctgctgctgcttctcgaactggatgcattaggaagctgctgtctgagtgtaggaatgtcttgctaagaaagcaatgtcttccttcatccttttctttcttccctctgcgtgtccttgtttttgtgtaatgcgggagagggttagagctatagagattatatatacactatccgtgcacattatatatatgtagatatacccctatcatgtcagagatctgcatgtcagtttttcagcaactaaggtgcctcatgttctgagttcagcagatataggaaccaagccgccccctcctgcacttgatgctcccacctttgttgtgcctcacttaaaatggtgcttttttcagttgtctgtcttttcttatgtttttatttgtaaggtgctgtatataagttgaatatattatgcacatatcctacccaatgggtagaacaaaaagttgttaatactgtaatataatgtatagatgataccaattttaacagaaatggcatagaatttgtgaatgcctatgtgctttgtcctcttttgtaaggaaatttgcaaatggatgcatacagattaaagtctatgtagtttattttcctattaaatatcaatattataacacaagagaaagaagtgtgaacaaacaagcaacagtttatgaccagcgtatatatagcaatggaaagttgcatctttgctgtgaaaacactttaaagaaaatactttttaaaaaatcccacagctttttggttgccactagacgcttcttattttaatcattttagtaatgctcagctggaccagtgttagttatatttgagtcagaaaaatgttgtttttcaacttgctttataatctcctgcatctatctcctgctgtagcatcaygaaggtgtcaggcaacagtgaaaagtgcacatttttgttgttgcagaaactgtgtcagaggaataagtaaatcagcctgcagcagaagactttgttcagctccagaggcatctgtgaccgtctgtgtccaagtctctctgtgcctttttcttttacaaactgaagctgtggagccaatgaagtaacagtagagattgtagggaaagaatacctcaggaaaaacaaatacacttacaagaagaccctgttcttagaaaatgtgtttagttatgggttagcactagaagagacttggctgtcagccagccaagtgaaggacctctcatccattcccattcatgtcccatcataatacggacmcaaaaagcaaactcggttttgccatcagttagaaattacgttttggattgtatattgttacatctctcttccagcttagtttttagtgtctgattgtgacctctgcatttatcttcaaataccctaattttaaaacaaaagaacaagaaaagtttataacaccatgttcactaaaaccacggttgaatcttgggtgtgggcatcctttcgagtgttgtccataagagcagttcgtggaattttgcccatctgacccatattatcagcttattctgccaccagagtagagtctaataaattccaaagtttttatttgctccatggtgtatgttctgactttgaaaatgtcagattctataatcatacccctaacatccaggagacaaatgacagattatctttaaactgaaattgactctacaatgcaacccttaatgctgaatggattaaaaaagtcagcccttttagtatctgtttgaaagggccgtaaaaagttgacacttttgttgttgtggatcctgcgtgtctagacccacgtgttgtttccatcgtatactgtagggtgcaccccttgggattcatcattaagaactgaggctcactgttgtcagaaacaaagctcccaccccccaggttcaaccttgtgggagaactgttgagcatgagaatgttctagactcagaggtactaaaatttgttaccacatcattgcttcctttctacaggacgaattgaggcttaaactttactgttaatgatactggttcattttaatgtgcttgttggtatgttgctatttttcatttcatagctttcaaaaatcatgctaattgtatacttgtctagtttaaggctattttaaaatatgtacaatactattcacagcatttagttcgtttaatttttattataaagcaatctactaaaaaagtacaactgtatttgaacttttcaatagttgtttgtgagctatgataatcaaaagtcattaaagtcttttttaacaaacattcgtgcttacttttcaacataattcccagttatatacagaaaaagatttccacctgtcacgtatctgcctcttttacctgagcaatggtgtagttcttagacctaaggtctgtaattgcaatacttttaaagaaagatgttgctctaagtgctgtttgttagttatgaaatcagatttttctgcttgttcttaatgctgtggtcaaaccatagcacaaaatcattaaaaataatcagcggcatac

SEQ ID NO: 22 - DNA, 999 bp, human
tttttttggcggtataaaagtgcttctttatttggtcatacagctttcaaatgttgcttaacgaacagatagcatttttataatcagaggaaagactcagtattaaagtgcgcgtcccatcattgcaaccgggccctcgggcagccagctctctcttgcttcgcgtccggctctcaggccctggagctcggcctccctaccttctccaggcgcgggtcctgctccttttccggccgcccttcctcagcccccggctcggacactctgcaggagaccaagagaagggggaaatggggctgggggccgtccccgggagacaggcggccttccgagagggactggagcaggccgtgcggtatgccaaagccctgggctgtcccaggatccacctgatggctggccgagtaccccagggagctgatcgaatagcagtcaaggctgagatggaggccgtttttctggagaacctgaggcatgcagctggggttttggctcaggaggacctcgtgggactgctggagcccatcaacacccgcatcactgacccccagtacttcctggacacgccccagcaggcggcagccatcttacagaaggtaggaagacccaacctccaattacaaatggacatattccactggcagatcatggatgggaacctgacaggaaacatccgggagttcctgcccattgttgggcatgtgcaggtggcacaggtcccaggccgaggggagcccagcagccccggagagctgaatttcccctatctgtttcaactgctggaagatgaaggctacaaaggcttcgtgggctgtgagtatcagcctcgaggagacacagtagagggcttgagttggctacgttcatactgggataggcggggccacccagaggctggccagtgagggcccgcacaccacccacgtgcctccagacagcgagtgacatcccatctcctcctctgcattaaagatgacctgctgaaaaaaaaaaaaaaaa

SEQ ID NO: 23 - DNA, 2316 bp, human
tcaaaataaaattcctgaatttgtacaagccacaggaagctagattgagatcattatatgacaactggaaggccaaggctatgggttacctcaaattgaggaattttggcacctactcacaggctccatgagcagatgaagtagacagctttactcagtatctcagaccaagaacttcatctccatctccaactagctgaaacatcttccctcctcaacctggaaaattctctgacttagaaatttaaacaaaaccctcccctttcattgaatctccattgtctggagtttgcttgttttaatctagcctgttcctccactatgggctccctttcaaactatgccctgcttcaactaacccttactgcttttttgacaattctagtacaacctcagcacctgcttgctccagttttccggacactatctatcttgactaatcagtctaattgctggttatgtgaacatctagataatgcagaacaacccgaactagtttttgttcctgccagtgcaagcacctggtggacctattctggacaatggatgtatgaaagggtgtggtatccacaagcagaagtacagaatcactctacttcctcctatcgtaaagtgacttggcactgggaagcctccatggaagctcaaggtctatcctttgctcaagtaaggttattggagggaaatttttctctttgcgtagaaaataaaaatggcagtggacccttcctaggtaatatacctaaacaatactgtaatcaaatactatggtttgattctacagatggcaccttcatgccctctatagatgttacaaatgaatccaggaacgatgatgatgatacaagtgtttgcctaggcactagacaatgttcctggtttgcaggttgcacaaaccggacctggaacagctcagctgttcccttgattggtctgcccaatacccaagactacaaatgggtagatcgaaattctggattgacctggtcaggtaatgacacctgtctctatagctgccaaaaccaaaccaaaggccttctgtaccagctatttcgcaacctattttgctcttatggcctgacagaggcacatgggaaatggagatgtgcagatgccagcataactaatgacaaaggtcatgatggacaccggacccccacctggtggctcacaggttccaatctgaccttgtctgtgaacaactctggcctcttttttttgtgcggcaatggggtgtacaaagggtttccacctaaatggtctgggcgatgtggacttgggtatcttgtaccttccctcaccagatacctcaccttaaatgctagccaaattacaaacctgagatccttcattcataaagtaacaccgcatagatgcacccaaggagacacagacaatccacctctgtattgcaaccccaaggacaattcaacaataagggccctttttccaagtttgggaacttatgatttagaaaaggcaattctaaacatttccaaagcaatggaacaggaattcagtgccactaagcagaccttggaagcacaccaatcaaaagttagcagtttagcctctgcatcccgaaaggatcatgtcttggatataccgaccacccaacgacaaacggcttgtggaactgttggcaaacagtgttgcctctatataaattattcggaagaaataaagtctaatatacagcgtctccacgaagcatccgagaacctgaagaatgtaccgttacttgattggcaaggcatatttgcaaaagtgggagactggttcagatcatggggctatgtgcttttaattgttcttttctgcttattcatctttgttttaatctatgttcgtgtctttcgcaaatctcgcagatcccttaactcccaacctctgaacctagccttatctccacagcaatcagcacagctccttgtcagtgaaacttcatgtcaagtttcaaatagggcaatgaagggactaacaacccatcaatatgatacaagtctactttgagaatatctgaacaaacagcagctgcagacaaaaagccttagctaaactttgatgagtaaagcaggtcttaccgagaattcagctgccaaaaccctcctctgagtgttcctcttataagggcacttagcactaggacctcccaaggtattgtaaataagccttatcagaactttttgtagtttcattctgaagccttaagacacacaccataaagctgatctgtaaacccttaccccttgctgttcagagagctactctttgtagtgttcttgcatgcatatataataaatgttttttctattg

SEQ ID NO: 24 - DNA, 2133 bp, human
caaccgcgccgtctgtccctggcaagccagcggcggtttaaaggaggtggcgggaagcctgtgtgtgcttcaaatcgtcaccctcatggtcgctccggtttttcacgactgaaaataacatagcaaaataagccaagatgtctgtggatccaatgacctacgaggcccagttctttggcttcacgccacaaacgtgcatgcttcggatctacattgcatttcaagactacctatttgaagtgatgcaggccgttgaacaggttattctgaagaagctggatggcatcccagactgtgacattagcccagtgcagattcgcaaatgcacagagaagtttctttgcttcatgaaaggacattttgataacctttttagcaaaatggagcaactgtttttgcagctgattttacgtattccctcaaacatcttgcttcctgaagataaatgtaaggagacaccttatagtgaggaagattttcagcatctccagaaagaaattgaacagttacaggagaagtacaagactgaattatgtactaagcaggcccttcttgcagaattagaagagcaaaaaattgttcaggccaaactcaaacagacgttgactttctttgatgagcttcataatgttggcagagatcatgggactagtgattttagggagagtttagtatccctggttcagaactccagaaaactacagaacattagagacaatgtggaaaaggaatcgaaacgactgaaaatatcttaattgctcagtagtcaaaaggaggagcctgtcaaaaagtagaatcataaggactgttcaaaccataaggactgttcaaatcataccagtgactgttcaaaccaaccatactttttattagatttgctttgtcaactctttcttgtattctgtgttttcctcttttttggtccactttgctgaggtatgaagtgtactactttgaactaggctgaagcatctgagtcttctaataagtgggaagggatccaacaaagaagccatgaccagttaaagatatttgcagagttacaccttggtcataagtcctttgtgaccttgattattttggcttactctttggatgagaccagacaagaaaaggattaaacgggtggctcctttaatattattattattgtttttgagacaaggtccctttctgtcacccaggttagagtagatttcagtggcacaatcttggctcactgcaacctctgtgtcctgggctcaagtgatcctcctgcctcagcctcccaagtagctaggaccacaggtgcgtgtcaccatgcttggctaatttttttgcagaaacgaggcctcactatattgtccaggctgagtggctcttttattaaccagtcattacactgcggaacagccaacatagagtacttgctctcgccctgtgaattttctttcatgagggagtcaatatgtagtggaaagaagcatgtagcaaaaaagacaaccttgatctttaataaaaaagaagttggtttatttccaaaataaatcccctgacaaaaaacctggtgatgttaagcaattgactgtcttagagtccagcagaagaccttagacaaaaaaagcagaacccactggagtagaaaaggaagcatgtagcatatactcagtagtgaaatttaattttactgactgttaggtatctatgccaatttgttttcatacttcagttggttttggaatctgccttatacctaatatttaattatttattcacactcataagcatcaaatatttaatgccctcagtgggaaaattgtgtttaaactcaatggaatctaatatttctttatgtcgttagtccctgtaaaatgttaggtcacccaaggaaaggggagaaatagcaatggttgttcctaaggtattgcttgccctccatgtcttcctaaagagcagaacttggagtttctcctttatgtagagaagaagtaacttagggtgtatttgcaatgaaatattcatagatattgaaagcttgtgtttacatgaaatatgtttattatcaagaattcctttttccaattctgtacattaaatatatgtgttttaaaggaaaaaaaaaaaaaaaaaaaaaaaaaaaaaaaaaaaaaaaaa

SEQ ID NO: 25 - DNA, 1336 bp, human
cctaactccaatcactggcaactcctgagatcagaggaaaaccagcaacagcgtgggagtttggggagaggcattccataccagattctgtggcctgcaggtgacatgctgcctaagagaagcaggagtctgtgacagccaccccaacacgtgacgtcatggccagtagggaagatgagctgaggaactgtgtggtatgtggggaccaagccacaggctaccactttaatgcgctgacttgtgagggctgcaagggtttcttcaggagaacagtcagcaaaagcattggtcccacctgcccctttgctggaagctgtgaagtcagcaagactcagaggcgccactgcccagcctgcaggttgcagaagtgcttagatgctggcatgaggaaagacatgatactgtcggcagaagccctggcattgcggcgagcaaagcaggcccagcggcgggcacagcaaacacctgtgcaactgagtaaggagcaagaagagctgatccggacactcctgggggcccacacccgccacatgggcaccatgtttgaacagtttgtgcagtttaggcctccagctcatctgttcatccatcaccagcccttgcccaccctggcccctgtgctgcctctggtcacacacttcgcagacatcaacactttcatggtactgcaagtcatcaagtttactaaggacctgcccgtcttccgttccctgcccattgaagaccagatctcccttctcaagggagcagctgtggaaatctgtcacatcgtactcaataccactttctgtctccaaacacaaaacttcctctgcgggcctcttcgctacacaattgaagatggagcccgtgtggggttccaggtagagtttttggagttgctctttcacttccatggaacactacgaaaactgcagctccaagagcctgagtatgtgctcttggctgccatggccctcttctctcctgaccgacctggagttacccagagagatgagattgatcagctgcaagaggagatggcactgactctgcaaagctacatcaagggccagcagcgaaggccccgggatcggtttctgtatgcgaagttgctaggcctgctggctgagctccggagcattaatgaggcctacgggtaccaaatccagcacatccagggcctgtctgccatgatgccgctgctccaggagatctgcagctgaggccatgctcacttccttccccagctcacctggaacaccctggatacactggagtgggaaaatgctgggaccaaagattgggccgggttcaaagggagcccagtggttgcaatgaaagactaaagcaaaac

SEQ ID NO: 26 - DNA, 5638 bp, human
gcagctggagtcaggcgctcgcccgtccgccggttggctcgccgggacctcgcgcaccggcggcagagtcccttgcgtggattggcaagcgacgccccacctgccccgagctcaccattttctttcgcgctggctgcagctgacccggcgaagggagccgaccgggccctgggctggaggtaaaaccccacggaaagaacatgaggttcccttggaaatcattcaagaggaagatggaaggggctgtttagaagagcttaaaagctacaggctgtaagactggggcctgagtgctggcagtggaaaacactgttgggctgtgatctctccctgaaaagttccaggtgcctttttgcttcctgcaaaagaaaggaagcgaaagagaagaccatgtccatagccctgaagcaggtattcaacaaggacaagaccttccgacccaagaggaaatttgaacctggcacacagaggtttgagctgcacaaacgggctcaggcatccctcaactcgggtgtggacctgaaggcggctgtgcagttgcccagtggggaggaccagaatgactgggtggcagtacatgtggtggacctcttcaatcggatcaacctcatctatggcaccatctgtgagttctgcaccgagcggacctgtcctgtgatgtcagggggccccaaatatgagtatcggtggcaggatgatctcaagtataagaagccaacagcgctgccagctccccagtacatgaaccttcttatggattggattgaggttcagatcaacaacgaggaaatatttccaacatgcgtgggtgttcccttcccaaagaacttccttcagatctgcaagaagatcctgtgccgccttttccgggtctttgtccacgtctatatccaccacttcgaccgggtcattgtgatgggtgcagaggcccatgtcaacacctgctacaaacacttctattactttgtcacagagatgaacctcatagaccgcaaggagctagagcctttgaaagaaatgacgagcaggatgtgtcactaatgctccacctcaccctttggaagaaaggaaagctgtttcctcctggtgccctgagcgggcaggaggtggaccaccctggctgaaatgacacacctactcccaggaacagcagaggtggaggcaagcagtgactcctgagagacattccccactcactttgtgtgctcttaaccttctgagtgctgctagcccagacctgtggacgaggcagaccacaacgtgaaagaaggaccagccccttgaccgttctggctggggaattgtccacgaggaagcctctgcacttccacacatggcacagttctgcctgtgacctgccgcctaagctttactggaattcagggttttgagactgagatgcgtgtcgtatttttcacttatctgtcttgtcagctggccgacttctctgtgattggttttttaagtgccgagtgaattttggacctctggatgtgcagcaagtttttatgcaataagccttcctttcaggtctctaaaagctcctgctctgatctgtggtttaacactgtgcagggctgtggagctctgagagacctgaacccctacccatcccctgcacctccctactctccctgccgaggcgtccatagcatttccctaataaatagttttatcagggacactccatggggtggcttgtctctgccccaaacagggtcttcacgttctaactgcagggaagagactggttactagcgtaaagctgacaagttaccagttcacctgatcagaatgtattttttataaccaccatcattccttgtctcttcagtggaagatattctttcctgtttcccagaagagagaaataaaagtcctaaataactaagaatgatcagcgggagcgttgggcatgattactgcagtgtttgttctttattaaagacagggagtcgtggctgtctctacataatactaacatttcagtgtaaaaagtacaattggttatttggtacgtgtagatttaacacccagacggctgacttgtaaccctccccactagccttctgagcatttaaacccagccgtcattctctccccactccacgctccccactcctctgccacgttatgttacggcacaaaattaatttctgcccctgtgattgggccacggttgccaaggtaacagtggagctggagctaacttcctcctttcatcctttccaattttctattccagaagacagtcagaataatgcatctgtactacatcctgccttttgaacctaaatgagtttcgttgatgaaatgtgcctctctgattcatcacaaactgtggtgttctgaccacctgtgacgagggggtcataatacttccagtgatccttttaatttagcaaaatatttgtcggtggagggaagtagataagaatgtattagtgtattttaaagtaataatgattaaagaataactaaatgactctgattttgttttatcaatcataatgtgttaaagccctctgtggctgccacaaaagactagaatcatgaaatctttgtccaagctaagaaagggctgtctctgtgggtacagtggcattctctgttgacctaaccaagcagcctgaaccttgctttgtgtctcgtaaaggtcatctcacggaatccaattgctgctacctcaccatgttgctgtgccctggtcaacaataaacatacttttttcccccttcctaagtaactttgagaggacaagataggcaaagttttttccctacatctcttttggccaaaagaatatctgaccatcacagagatccagagaacagaaacaagagcccctaagtcccagcctctgcaatacacagttaccagatgggaatgcagcacagtggcacagagaggtaactacggagcccatgccctggaggaactctgtagctgtgcatcatggaacgccacaggatttttacattaaaaagttgaatatgttcctgcatttcagcagtgcataatcgagcaggaggctggaatctggggacctttgttattcattctttttaaggttattttttattaactgtgcatatgaaatttccactcaatttaaaaatccaaatctttttaagcttcttggaagaatttcctcctgcttatattgttaattgcactaatgaactcttctagggtaaattctggcaaactaccctttttttttttttaatcacagaatattcctgttcccacagaactttggcacattctgaagaaaatctctgcagtcaatttttatctcctctgcttgactgcatctaactgttcatctctgttataaatggagcttcagccctctgggtcagagactgacaaccctccttcttcttcctaacctgtcctactctccttctttttctgctttcattttaattttgggaaattttaaagtaaaagtttcaaagttgaaagattatagtgagtacccttatgtcctttagtgcatcagttgttaatgttttgccttgttttcttttcttcctttttttttttaagagaaggggtctctccctattttacccaagctggccttgaaccccttgcctcaagtgagcttcaaccttagcctcctgagtagccaggactatggcatgtaccaccgcacctgcctcacattttctttctcactgtaaatacacattcttattcttgaaccttctgaaaataagttgcagacatcatgaaaatgtaaaatacttcatcatattctttcttttaataagagcattgtcctataaatctgcaatattttcacgcccatgaaactgaacactgatactgcataaatctaatatatgatccatattcaaaattctccagttgtccccaaaatatcctttatagctgtttatttttaaatccaggatccaatcaagaattatacgttatatgtagttgtcattctcttttgtttcctttaattgattagagttgcccttttgtgtgtgtctgagtggtgggtggtggggtggtgtttgtgtatgatgtgcagtgtgtgtgtgtgtggtgtgtctttcatgacactgacattattaaagagttcaggcaagtttgtggaatgtcccacaatctagacttttttactgattgtttcctcatcacgagatacaggttacattttttttcccaagactgctacatgtgtgatgttgtttccttactactgcctcacctcaggaagcacataatgtctacttgtctcttttctgatattaggactgatcaggtgttgtctgcctgatccaatcattatcaagttccataattcattattaagctctgaaattcaggacacatacatttatagagtgtgaggctgtgcatacgtacattcatcccttaatgttctcaaggaggcccaggatccccaaaatattataagcattgcagtcagatgatatagcactagcaaagcattcatccctttccatataacaggagggaggggaagaaggaagtttccatgtgcaggcaatacgtgagagcatgggcaagtgaggatttgtatgctccctgtagccagaggtgaaatattcaacgcttttattttttctcccgtcttcaatgacctcccatttggacccatagtttatttatagagagcacacactccctaattgtcagatggtttttttgggaaaactcttagatagtacagtgagttgtgaccctccaaaggatcacaagtacataaacagatacaaagtatttatgtggtattaaaaattcatggtgatggtgcaagggagagatgattaggaagaaaatgtcttaaacggctgtaattgagaaatactgatctagagtcatttcctgcttcttaaattgttcgttttctcttccctgggtattttctcatttccaaagacccagagtctcagagccagtttaagtggaagaaactttgagaggtttcttaatcaattccttctatctctcaaaccatgccagtaacttccttggtgaaaaaatctatactcctctcacagttcttcgggcatggaaattctatagcattctttggtataaccaacccactgaaccctccacaacatcttgaattccattcaactcgagctgaagtattctttttgagtttcagtatatcttttctagtgtcccacttttagaaaggtccaaagatgatatataataagtgaaaatacaattaatcagagctttatttgcatatccattaaatatctgaaggctattaccagttgtgcttcagcctcctttcccttgcaattctcccagggtggcatcctctacggagtcttgttgctccattgttgaccactgatgtaactcaacgtgaaaccttcccaaagctctccacatcactctctgttatggctgtgaccagcagtgaacatgaaaagaacttatttcatgccttctccaaactatattgtttctttgctaagtccagacctgctttttctacatttataggttttctattttaatcgccatgcaaaattctttatcatactcaatcattttggtttatcttcctgacttcttacctttaacatccctgttaaagattacactgttgttgtttcagaaaaaaaaa

SEQ ID NO: 27 - DNA, 4221 bp, human
cagcccgagcccgagcccgagcccgagccggcgccaccgcgcccccggccatggcttttgccaatttccgccgcatcctgcgcctgtctaccttcgagaagagaaagtcccgcgaatatgagcacgtccgccgcgacctggaccccaacgaggtgtgggagatcgtgggcgagctgggcgacggcgccttcggcaaggtttacaaggccaagaataaggagacgggtgctttggctgcggccaaagtcattgaaaccaagagtgaggaggagctggaggactacatcgtggagattgagatcctggccacctgcgaccacccctacattgtgaagctcctgggagcctactatcacgacgggaagctgtggatcatgattgagttctgtccagggggagccgtggacgccatcatgctggagctggacagaggcctcacggagccccagatacaggtggtttgccgccagatgctagaagccctcaacttcctgcacagcaagaggatcatccaccgagatctgaaagctggcaacgtgctgatgaccctcgagggagacatcaggctggctgactttggtgtgtctgccaagaatctgaagactctacagaaacgagattccttcatcggcacgccttactggatggcccccgaggtggtcatgtgtgagaccatgaaagacacgccctacgactacaaagccgacatctggtccctgggcatcacgctgattgagatggcccagatcgagccgccacaccacgagctcaaccccatgcgggtcctgctaaagatcgccaagtcagaccctcccacgctgctcacgccctccaagtggtctgtagagttccgtgacttcctgaagatagccctggataagaacccagaaacccgacccagtgccgcgcagctgctggagcatcccttcgtcagcagcatcaccagtaacaaggctctgcgggagctggtggctgaggccaaggccgaggtgatggaagagatcgaagacggccgggatgagggggaagaggaggacgccgtggatgccgcctccaccctggagaaccatactcagaactcctctgaggtgagtccgccaagcctcaatgctgacaagcctctcgaggagtcaccttccaccccgctggcacccagccagtctcaggacagtgtgaatgagccctgcagccagccctctggggacagatccctccaaaccaccagtcccccagtcgtggcccctggaaatgagaacggcctggcagtgcctgtgcccctgcggaagtcccgacccgtgtcaatggatgccagaattcaggtagcccaggagaagcaagttgctgagcagggtggggacctcagcccagcagccaacagatctcaaaaggccagccagagccggcccaacagcagcgccctggagaccttgggtggggagaagctggccaatggcagcctggagccacctgcccaggcagctccagggccttccaagagggactcggactgcagcagcctctgcacctctgagagcatggactatggtaccaatctctccactgacctgtcgctgaacaaagagatgggctctctgtccatcaaggacccgaaactgtacaaaaaaaccctcaagcggacacgcaaatttgtggtggatggtgtggaggtgagcatcaccacctccaagatcatcagcgaagatgagaagaaggatgaggagatgagatttctcaggcgccaggaactccgagagcttcggctgctccagaaagaagagcatcggaaccagacccagctgagtaacaagcatgagctgcagctggagcaaatgcataaacgttttgaacaggaaatcaacgccaagaagaagttctttgacacggaattagagaacctggagcgtcagcaaaagcagcaagtggagaagatggagcaagaccatgccgtgcgccgccgggaggaggccaggcggatccgcctggagcaggatcgggactacaccaggttccaagagcagctcaaactgatgaagaaagaggtgaagaacgaggtggagaagctcccccgacagcagcggaaggaaagcatgaagcagaagatggaggagcacacgcagaaaaagcagcttcttgaccgggactttgtagccaagcagaaggaggacctggagctggccatgaagaggctcaccaccgacaacaggcgggagatctgtgacaaggagcgcgagtgcctcatgaagaagcaggagctccttcgagaccgggaagcagccctgtgggagatggaagagcaccagctgcaggagaggcaccagctggtgaagcagcagctcaaagaccagtacttcctccagcggcacgagctgctgcgcaagcatgagaaggagcgggagcagatgcagcgctacaaccagcgcatgatagagcagctgaaggtgcggcagcaacaggaaaaggcgcggctgcccaagatccagaggagtgagggcaagacgcgcatggccatgtacaagaagagcctccacatcaacggcgggggcagcgcagctgagcagcgtgagaagatcaagcagttctcccagcaggaggagaagaggcagaagtcggagcggctgcagcaacagcagaaacacgagaaccagatgcgggacatgctggcgcagtgtgagagcaacatgagcgagctgcagcagctgcagaatgaaaagtgccacctcctggtagagcacgaaacccagaaactgaaggccctggatgagagccataaccagaacctgaaggaatggcgggacaagcttcggccgcgcaagaaggctctggaagaggatctgaaccagaagaagcgggagcaggagatgttcttcaagctgagcgaggaggcggagtgcccaaacccctccaccccaagcaaggccgccaagttcttcccctacagttctgcggatgcttcttaacaaccgcccggggctgtggctggcagcttggtgggccccagggccttctccttcattctctgtgaacatgtaactcaggaccccttttccctcttgcgtctgtgccagctcaaatccagcccctcgccctgtgccaccccaactgtgcctgatagacctgccccagcgttcctgacttcttgctggcctgtggagggtgaggtgtaattatttgtcacctgaacctaatgtatattctccttgagccccagatcccttcaagctggaagggatggggctgttggtggggtcagggtccaagaggaatgggtgttctgtggcctcgagtcctctcctgtttgcgaaaataccagttttgctctctgtgggacaaagcactgctgatgaagtccccgtgggctcatccgggctggaattcttggtttttcagccattccctgcagagtcactcaatcatcaagtccctcacagccatttctgttcccagaggagccaggcctgcagctggctgctcaggagatggcctcattcctcctgttctcccagtttgctttccacttaagacaaagccttgtctatgtggggggcggggaccggggaaagagggaggctgaaatgtttattctgcttctcccgtgtttcatgccatctcgcgtcccccttcctgcacatgggtgtgaatgcacacacatacgcgtacacacaggacttggtctgctggcctggcctcttctgcccaggtgggttggaacacgtttgctgcctgagcctgtgccactgagcatgttaggtggagcagttggtgtggcacgtgcggggtgttggcaccggaggcatggaaaagcacaggctgtactgccaggctgcgatgcgtgctggcccccgcacaggctcctgtgtgcagggactgattcctcagcacacgaggcttccacaacccagtctgctccatagcactctggcccaccctgtctgcaggtgaaacaggagggctgtttgcctctgccccatccccccgactgtgttcaggagtcccaccttgcatttcagacctgggctggcagtctgttggacttctcttcaggaagaaaaagcatcagggggaaatggaatgcccctgccccaggaacatggcagaagcacaggttctgtacctcagatggactcctgctgggccttcggggtctcagttggcttcccccagattctgattctacagctgcagaatgtatataacacaataaaagcaaatgtttgaaccagt

SEQ ID NO: 28 - DNA, 1940 bp, human
ggggcggtgcagaggcggcaggaagatggagttggggagttgcctggagggcgggagggaggcggcggaggaagagggcgagcctgaggtgaaaaagcggcgacttctgtgtgtggagtttgcctcggtcgcaagctgcgatgccgcagtggctcagtgcttcctggccgagaacgactgggagatggaaagggctctgaactcctacttcgagcctccggtggaggagagcgccttggaacgccgacctgaaaccatctctgagcccaagacctatgttgacctaaccaatgaagaaacaactgattccaccacttctaaaatcagcccatctgaagatactcagcaagaaaatggcagcatgttctctctcattacctggaatattgatggattagatctaaacaatctgtcagagagggctcgaggggtgtgttcctacttagctttgtacagcccagatgtgatatttctacaggaagttattcccccatattatagctacctaaagaagagatcaagtaattatgagattattacaggtcatgaagaaggatatttcacagctataatgttgaagaaatcaagagtgaaattaaaaagccaagagattattccttttccaagtaccaaaatgatgagaaaccttttatgtgtgcatgtgaacgtgtcaggaaatgagctttgccttatgacatcccatttggagagcaccagagggcatgctgcggaacgaatgaatcagttaaaaatggttttaaagaaaatgcaagaggctccagagtcagctacagttatatttgcaggagatacaaatctaagggatcgagaggttaccagatgtggtggtttacccaacaacattgtggatgtctgggagtttttgggcaaacctaaacattgccagtatacatgggatacacaaatgaactctaatcttggaataactgctgcttgtaaacttcgttttgatcgaatatttttcagagcagcagcagaagagggacacattattccccgaagtttggaccttcttggattagaaaaactggactgtggtagatttcctagtgatcactggggtcttctgtgcaacttagatataatattgtaaaatgcttttcaagtgtgggttttgccctgattgttgcaaatacaatttccaccttctggaaaggtaggtttgctgtggaggaaataatgtactagatcattgtcacagaaaaaccaactatgatttatggttgtgttttcagaattcaacattaaagattaatgtttatttaaacgaacacattcctgcattcaggatgtgaggccatttaataaaaagggcacaaagcctgtcagagttttcaacggtgcttatagctgccagctggattccaaacaggtaccacattgtctctgagctaatgtttatatttttccattcaggcaccgaaatagttaatatttgaaataagtcttcaaaagaaaacataagagattattgagttcttgggactggatcctttatttcataagttcagatcatcttaaatgaaaatgccatgattatctgcagttaagtagatgacagctattctacatcagacttgatttttgtcagctaattacataattggtaagctataattgaaaccttatggcttaaaattccttaactcctttttgattcatgtttgtagtcatgttgtcaacagaggcaaagttaagcttgatgatggttaaaatcggtttgatagcaccatgggacatttttctaacaaaaataaatgcatgaagagacatagccttttagttttgctaattgtgaaatggaaatgctttacaggaagtaaatgcaaattacttttaagtgtgctttaaagaaaaatattttccccacaagagaaatttaaataaagaattttatttgtttaaaaaaaaaaaaaaaa

SEQ ID NO: 29 - DNA, 2367 bp, human
gcggccgcgatggggccgaagcgcccgaagccccggagcccacaaactgccgggcccgcctcgccgccgggacccgggtgcctgggctcggcttgaagcggcggcggcgcaccggcacagccgcgggagcatgggcaggaggatgcggggcgccgccgccaccgcggggctctggctgctggcgctgggctcgctgctggcgctgtggggagggctcctgccgccgcggaccgagctgcccgcctcccggccgcccgaagaccgactcccacggcgcccggcccggagcggcggccccgcgcccgcgcctcgcttccctctgcccccgcccctggcgtgggacgcccgcggcggctccctgaaaactttccgggcgctgctcaccctggcggccggcgcggacggcccgccccggcagtcccggagcgagcccaggtggcacgtgtcagccaggcagccccggccggaggagagcgccgcggtgcacgggggcgtcttctggagccgcggcctggaggagcaggtgcccccgggcttttcggaggcccaggcggcggcgtggctggaggcggctcgcggcgcccggatggtggccctggagcgcgggggttgcgggcgcagctccaaccgactggcccgttttgccgacggcacccgcgcctgcgtgcgctacggcatcaacccggagcagattcagggcgaggccctgtcttactatctggcgcgcctgctgggcctccagcgccacgtgccgccgctggcactggctcgggtggaggctcggggcgcgcagtgggcgcaggtgcaggaggagctgcgcgctgcgcactggaccgagggcagcgtggtgagcctgacacgctggctgcccaacctcacggacgtggtggtgcccgcgccctggcgctcggaggacggccgtctgcgccccctccgggatgccgggggtgagctggccaacctcagccaggcggagctggtggacctagtacaatggaccgacttaatccttttcgactacctgacggccaacttcgaccggctcgtaagcaacctcttcagcctgcagtgggacccgcgcgtcatgcagcgtgccaccagcaacctgcaccgcggtccgggcggggcgctggtctttctggacaatgaggcgggcttggtgcacggctaccgggtagcaggcatgtgggacaagtataacgagccgctgttgcagtcagtgtgcgtgttccgcgagcggaccgcgcggcgcgtcctggagctgcaccgcggacaggacgccgcggcccggctgctgcgcctctaccggcgccacgagcctcgcttccccgagctggccgcccttgcagacccccacgctcagctgctacagcgccgcctcgacttcctcgccaagcacattttgcactgtaaggccaagtacggccgccggtctgggacttagtgtcaccgggaggaaaagagagagatctggggctggggtatggatgatggggggaagggcggtcgcctctgccactgtcagggaccagccggccaacgcccacccgcaaaggtgtctaaaaacttcagcttttcacccacctgcccctttctttcaatcccacgctgtttcctttcaaagttctgggaggacgaactcaccgaggcgagaagtgtaacattctctccacccagcttataaaaggattctttactgtgccagcacggggattggatccgaagaaactggctactggggtttggcccccgagtggccgtccctgtgggagatgcaccccattcttgggcccccctcattccctttccgaaaaaggaaaacttgcgtttgagccgttgagctaattctgcaattttctaccaaacagagcgctggtggccccggagcagggctgtgacattggctggtggagccccttcctgtgttctccctttgttccagcgccgcgatggtgagatcactgttccaagcagggggacggctcgcgataggacaaagagagcaggacctccagactctggggagccctgcagaccttgacaatttgcctgactcattcctgacctcttgtcattttggcctgaaggctacaaattcagggtcagctgtatgcactaagtcaaataatgaatttcttcctccctctcgcaaccgaccaaaattttgacaacgatgatgttcaccagaaggaaaaaaaaatcagttttatgcactttattttgttttgattttcattttttattaagaaaaaattttattttacagaatttaccttctctgtatatatgtgcataaagtgtggtgtaaatatactaaacaaacttatatttcaataaaagggagtttaaaatttaaaaaaaaaaaaa

SEQ ID NO: 30 - DNA, 548 bp, human
tttaatttataaatcagtttatttgcagattttatctttcttaaagtatatttcagttaacaaatctatttacacaggtatacatgggaattcatcccagttattttacagatcgtacttacaaaccacacccttaagtgttcggtgcaaaattgcactggatgtgactattggctgaaaatggcctgatgaccaggctatttacacgcacacagtaaacgggtttattttttcatgtctgcacagtacagtacacacaaggaggctgaacctcccagagaatgctttgcaatgtgtttgccaagaaggctttgcaaagtccagaaggaaaagctattaaacataggttaattaaaatgacagtacctcctttacatcagtttacattttttcttcacatttttataatacaaagatattttattgaattgctgcacaaccagttgaagatgatttgtaaacatgtaaattcctacaatttgcattaaatgtcattgtagtttctataatttgctctttgaattaatgttttattcactagtattctt

SEQ ID NO: 31 - DNA, 654 bp, human
ggatgccaagttcttttagtaattcaccagctccatgcagggacatcacagggctgccctccatgagcagaggaggagggctgcctggcagagcgtttcacactccaggttagccagaaagagcatcttcatttttgtttccacacaacacttctctgtgagcctgttggccaacaaagtggcggccgattgttggaggagccacccaaccatcttggctaaccttaaattcttcagggctagaatatgttcaccccagaggcttagatgaagcacatttgcggctactcgggcagatggtctcttgctggcctctcgctggagcagtgccctcaccaactgtctcacgtctggaggcactgactcgggcagtgcaggtagctgagcctcttggtagctgcggctttcaaggtgggccttgccctggccgtagaagggattgacaagcccgaagatttcataggcgatggctcccactgcccaggcatcagccttgctgtagtcaatcactgccctggggccaggacgggccgtggacacctctggggccatcaaacagccgttttcgccccgatccacgtaccagctgctgaaaggcaactgcatgccgatgctctcatcagccagacagcaacccaaatctgcgatcaccag

SEQ ID NO: 32 - DNA, 610 bp, human
  misc_feature (448)..(448): n is a, c, g, or t
  misc_feature (479)..(479): n is a, c, g, or t
  misc_feature (528)..(528): n is a, c, g, or t
  misc_feature (548)..(548): n is a, c, g, or t
  misc_feature (556)..(556): n is a, c, g, or t
  misc_feature (592)..(592): n is a, c, g, or t
tttttatattcgtaaacatagcattttattaacaaaatgtttacacctttcttacaacaaaacagtgatttagcatgttatactctataaaagtgtattatagaatattcaagctaaaacaatattatttaaaagtatggcaaatcaactttcatttgcaaactatctactatgagcataggcatgaaaacaaaaatcttacaacaaaaaacaaaaggtctagttttcttttgtcagcatctataaacaatacagacatcatatggtttacatacacttaggtccagcccaaggttaagactgcaaaaataaagtcttcaaacatccacagaagcttggcaaaccaccactgcagcattccatgacaggtaaccaggccatgtgtcatgcagtaagtccaaggccataaatttgggctgtttataatagtgctccaaagcggccaaantgtaagcactgaattttcttccgtaatagantattataaaggcccaatacttattcttcacaggcttgagaagctgcatnttggagaagagagatctggnagcgcttnctttttttgaaagctaggcatgcaagtgttctcagnttaattggcttccgtaca

SEQ ID NO: 33 - DNA, 4515 bp, human
ggtcagcgcccaggtcctgggctggccgccgggatgttgccctacacagtgaacttcaaggtgtcggcgcgcaccctcacgggggccctcaacgcccacaacaaggcggcggtggactggggctggcaaggtttaattgcttatggatgtcattcacttgtggtagtgattgattccattactgcccaaactcttcaagttttagaaaagcataaagctgatgttgtaaaggttaaatgggccagggaaaactatcaccataacattggctcaccatattgcttacggttagcttctgctgatgtcaatgggaagatcatcgtctgggatgtagcagcaggagtagctcagtgtgagatccaagagcatgccaagcctatccaggatgttcagtggttgtggaatcaagatgcttcccgcgatttactgcttgctatccacccgccaaattacattgtgctctggaatgccgacactggcaccaaactatggaagaagagctatgcagataacattctttctttttcttttgacccttttgatccctcacatttaactttgcttaccagcgagggtattgttttcatctcagacttctccccatccaagcctccctcaggccctgggaaaaaagtttacatatccagcccacactctagcccagctcataacaagctggccacagccacaggtgccaagaaagctctaaataaagtaaaaattttaatcactcaagagaaacctagtgctgaattcataactctcaatgattgccttcagttggcatacctgccttcaaaaaggaatcacatgttgttgctctatcctcgagagattttaatccttgaccttgaggtgaatcagacggtgggtgtgattgcaatagaacgcacaggagttccatttttacaggtaataccctgctttcagcgtgatggtttattttgtctacatgaaaatggttgtataactttacgtgttcgaagatcttataataacatttttaccacttcaaatgaggaaccagatccagatccagttcaggagcttacctatgatttacgaagccagtgtgatgcaatcagggtgacaaaaaccgtccgtcccttcagtatggtgtgctgtcctgtcaatgagaatgcagccgccctcgtagtgagtgatggcagggtcatgatatgggaactcaagtctgcagtttgtaatcgaaattcacggaacagtagttctggtgtgtcacctttatattcaccagtgtctttctgtggaattcctgtaggagtgctacagaataaactcccagacctttccttagataacatgattgggcaaagtgcaattgctggggaagaacatcccagaggttcaattctgcgggaagtgcacctcaagttcctgctgacgggactgctttcaggactgcccgcaccacagtttgctattcgtatgtgtccaccgttgaccacaaaaaacatcaagatgtatcagccactgctggctgttggtacaagtaatggttctgtcctggtgtaccatctcaccagtggtctgctacacaaagagttaagcatccactcatgtgaagtcaagggtattgaatggacaagtttgactagttttctttcttttgctacctcaacaccaaacaatatgggattagtgagaaatgaacttcaactggttgatcttccaacaggtaggagcattgcttttcgtggtgaaagaggcaatgatgaatctgccatcgaaatgattaaagtatctcatttgaagcagtatttggcagtcgtattcagagataaacccctggagctatgggatgttaggacttgtacccttcttagagagatgtccaaaaacttccctacaataactgctttggagtggtcaccatctcacaacttgaagagcctgagaaagaagcaacttgctactcgagaggccatggcccgccagaccgtagtctcagacacagagctgagtattgttgaatcatctgtgatcagcttgctgcaggaggcagaaagtaaatctgaacttagtcagaacatctctgcccgggaacattttgtatttaccgatattgatggccaagtgtatcatctcactgttgaaggaaactcagtaaaagacagtgctcggattccaccagatggaagtatgggtagtattacctgcatcgcttggaaaggtgatacattagtgcttggagatatggatggaaatttaaatttctgggacttgaaaggcagagtatccagaggaatacccacacaccgaagttgggtgaggaagattcgttttgctcctggtaaaggaaatcaaaaattaatagcaatgtacaatgatggagctgaagtgtgggatactaaagaggttcagatggtgagcagtttaagaagtggcagaaatgtgacctttcgtatattggatgtggactggtgtacgtcagataaagtgatcttggcctcagatgatgggtgcatcagagtcctagagatgtctatgaagtctgcgtgctttagaatggatgaacaagagttaaccgagcctgtgtggtgcccctatctccttgttccaagggcctctcttgccttgaaagccttcttattacaccagccttggaatggacagtattctttggacatttctcatgttgactatccagaaaatgaagaaataaagaatctcctccaagaacagttgaattcattgtctaatgacataaagaaactgttgcttgatccagaattcactctcttgcagaggtgcctgcttgtttcaaggctctatggtgatgaatcggagctgcacttctggactgtcgctgcccactacctgcacagcttatcccaggaaaagtcagccagcacaacagctcctaaagaagctgctcctcgagacaaactgagcaacccactggatatatgctatgacgtgctctgtgaaaatgcctactttcagaaatttcagctagaaagggttaatctacaggaagtgaaacggtcaacttatgatcatacaaggaaatgtacagaccagctactgctcttgggtcaaacagacagagctgtgcagttgctgttggaaacaagtgcagataaccagcattattactgtgattcactgaaagcctgtttagtcactactgtcacctcgtcaggcccctctcagagcaccattaagttggtggcaacgaatatgattgccaatggcaaattggcagagggcgttcagttgctctgcctgatagataaggctgcagacgcctgccgctacctgcagacatacggcgagtggaatcgggctgcatggctggcaaaagtccgtttgaatcctgaggagtgtgccgatgttttaaggcggtgggttgaccacctttgttctccacaagtcaatcagaaatcaaaggctctcctggttctcctctctctgggctgcttttttagcgtggcagagacgcttcacagcatgagatactttgatagagcagccttatttgtggaagcttgcctcaagtatggagcatttgaagtcactgaggacacagagaaactcatcactgctatatatgcagattatgcccggagtttgaagaacctcggttttaagcagggagcagttctctttgcttcaaaagccggagcagctggcaaagacttattgaatgagcttgagtcccccaaggaagaacccattgaagagtgacagcttaataaatgccagggaatctgacctggaaggcagatgggagggggctggtctggctgtggccaccgtcacagtccaggatgaagaggagtacagggtcctgtgagctgtttgaccactgttctaagactatgtgtgcccaaaagcacataagcatctatgttgagagtaagtttgtatcctgcgttggtctcagaaagaacgtgaatgcttaagattttgaaagtacataatattttatactttgggagagagctttaagagtccctggaaatactttttaatttttttaacttaaaattcaagagactgaatcacttttctcattgattaaatgtaaagattattgagaaacctatagtaaatgaaatttgtaagatgttttctcaaatatatgctgtgcctgtacttatatacagtctttcaagagagatacaaacaaggcagaaacatttaaactagtattaaaggtagtttaccaaagcattttttgttttcttaccttgaaaacacagaaccgttaattccttggtttaagcagttgctaagttttttaattttaggctcagaggcccataggaggttttaagatttatgtttagtccgataggtgaggtctttgatattttgaattttaactccttttatgatacatcacagtaacctcatttttgaagtctttctttgtactttaatgttctctctgttctaatagttgaagtatgagatgtaactattataaactgttgctgaaaacataaatgtctgtaacttacaaacatgataaataaattaaaaattcca

SEQ ID NO: 34 - DNA, 175737 bp, human
gatctgcccgcctcagcctcccaaagtgctgggattataggcgtgagccactgcgcctgacctttttttttttaaatcttttgagagagacgtagtcttgctctgtctcccaggttggagtgcagtggcgtgatctcggctcactgcaacctccgcctcccagattcaagcgattctcctgcctcagtctcccaagtagctgggattacaggcacctgccatcatgcccagctaattttgtatttttttgtagagacggggttttactgtgttggccaggctggtcttgaactcctgacctcagatgatctgcccgcctcggcctcccaaagtgttgggattacaggcgtgagccactgcgcctggcccacacatttttaggttataaaattaaacgtaatatggccaggtgcggtggctcacgcctgtaatcccagcactttggaggccaaggcgggtggatcgcctgaggtcaggagtttgagaccagcctggccaacatggcgaaactctgtctctactaaaaattcacaaaattagccgggtgtcgtggcgggggcctgtaattccggcaacttgggaggctgaggcaggagaattgcttgaacctgggaggcaggggttgcagtgagccaagactgtgccattgcactccaacctgggcaacaagagcaaaactccgtctcaaaaacaaacaaacaaacaaaacataatatgagactggacacagtggctcatgcctgtaatcttaacagtttggtaggctgaggtgggcagatcacttgagcccagaagttcgaaacaagccatgtcacccatgacatggcaaaactctgtctctacagaagatagaaaaattagccgggtgtggtggtgcatgcctgtagtcccagctactcaggaggctgaggtgatcctcccacctcagcccaggaggttaaggctgcagtgagctgtgatcatgccactgcactctagcatgggcaacagagtgagacccggtctcagaaaaaaaaaataataataatcaaatatatttgtgtaatacagatctactaatgggaagaactgaatttctctttttgaggttaacattttgcctaattgatgtacaaagttagtgttccagatggtcaaatttgactgtagatattcatgttcatgctgatctgtagagattgcaagtatttcatctttgaaaacatcttttcacacaggtaatgataggtgatatgtgaggtgcttgaaatgctgtgaagcacttacgactgtgtcactgtgacttgtagtgtacaaagcagcagtgcaaatcaggatgctgtagtcgctgtcgtgaccactcggctgtgtgttgtagagcaaaagcagccacagtattaagtaaatagtgtggccccgttccaataaaactttatttgttggatattggaatttgaatttcatacggttttcacagtctcaaaatattcttttgattttttttcaaccacttaaaaatgtaaaaaccattcttggcttgtgggctatacagaactagatgatgggccaggtttggcccatgggcagtagtttaccaagccttggtttaaagccctcatataagctgttgtagacattaagatgagttaaggcatatagtttagcacagcgcttagcaaaatagggagcgctgtgttcatcattgtcattcaagatgatcgttctctccaggtctggctgatgtgaggggtggaggtggtgtctgctttggatttctgctgttcctgagggagtatctgcattttccacagcttttctgtctgatttgtattttcctctgattccttttgtcatcaggtattcactgggcacctgctgtgggcagggctctgcgctgaggttctggagacaaaaggatgaatcgttgagcctgccctggtgtggtgctcccgttatcctttaggtataaaaacttgtggctattttttttttttttttgagacagagtcttgatctgtcacccaggctggagtgcagtggcacaatctcagctcaccacaacctctgccccccggggttcaagcgattctcctgcctcagcctcctgagtagctgggattacaggcgcccacgaaccacgcccagttaattttttaatgtttagtagagatggggtttcaccatcttggccaggctgatcttgaactcctgacatcgtgatccacctgcttcggcctcccaaagtgcaagtgttgggattacaggcgtgagccactgcacccggccatggctatggtttttgagaatgattggccaggtgatgcatttatttattttattactatttttcgagacggagtcttgatctatcacccaggctggagtgcagtggcgcgatctcggttcattacaacctccgcctcctaggctcaagtgattgttctgcctcagcctccaagtagctgggagtacaagtgcatgccactgcatgcgctaatttttgtatttttagtagagatggggttttgccatgttggcttggctggtctcaaactcctgacctcaggtgatccacccacctcggcctcccaaagtgctgggattacaggcatgagccaccacgcctagctcaggtgatgccattagtttctacacagttatcctctgtcatcccagactcaatgtgtccatcactggataggtcaccgcctcctaaagtttctcttgacctgctctcatctcccagaattttcctgtcacccagaatttgactcaggtacacaccaccacacctggataatttttctattttttgtagagatggggtttcaccatgtagcccaggctggtctctgtcttgaactcctgggctcaagcggtcctcctacctcaacctcccaaagtactgggattacagagtaattactgtgagccaccacatccagcttggccaccagcttattcaaagtctccaggatgctgggaccaccccccccaccacccgctccctgtttttgcataattgtatcttttttttttgagacggagtctcactctgtcacccaggctggagtgcaatggtgtggtctcggctcactgcaacctccacctcctgggttcaagcgattctcctgcctcagcctcctgagaagctgggactacaggtgtgtgccaccacacccggctaatttttgtatttgtagtacagatggaatttcaccatgttggccaggctggtcttgaactcctgaccttgtgatccacccgcctgtcctcccaaagtgctgggattataggcgtgagccaccgtgtctggcctttgtaattgtatctttgtgaatgagtgatttggttctgccctttttactccatatttataccagcctggctctaggagagtcagaaggcctgcccaagggtttctgccctctctgggccactgggccaaagctgtagcttgccctccgtgggctacctgggtcagccactcctgtgcttagggcttaatcactagttcgctgaggcctgaagtttaatcaacaccttgaggctaaacagctctggtccttgtgatcttcagcccatccctgcttttctctggtcctcctcaggagcttgtcaggccacgggggctccagtgatgaggctgaccatctttcttaagaggtgttctggtagcttgtattatgattggattgcgttgacttctcaaagccgaactgctgcttactagtagtacagtagactcccatttggcactgggctggtctttacccaggggtcccatagatgggtgtgtggaggggagaagagccagcatgcatccttgagttgttagttcaccaaaggatgtgaccctgtgttccacgagcgctcggtaattcctggtttgcactgatggcctttctctttggtaaagtcagggcgctgattatcttgtcggaagtatccagctggtcctctcttgggctccatcactgtgcaagtctctgctcataagaactcttcagttcccttctcctgggttcacagccaaggcaaaaaagagactccttggcctcttggcaaacatacatcccctacctggctggtcccccttcagcttccctctctaaggaacaacttgaacatagaaactccaacctcacctgtaatcccagcactctgggaggccaaggtgggcaggtcatttgaggccaggagttcaagaccagcgtggtcaacatggcgaaaccctggctctactaaaaattagcggtggtggcgcatgcctgtaatcccagctacttgggaggctgaggcacgagaatcacttgaaccagggaggcagaggctgcagtgagccagggtcatgccactgcactccagcttgggcaacagagagaccctgtctcaaaaaaagaaaaggaactccaacttctgaaagtaaacaacttatgcagtacatgattcctggagaggatttggtcaccaaccttccctccttccttccagggaaagcacacagaggggcagggcaagggcctccagtgcattacttcaccacttccctgagtaggtcttctcaagtccttggagccttcactttgtgatttgtcactgccagagagcaatgttgggatcatgagctgtgggtcctcagtctgactttaaagaaacctcacaaattgtctgctcctgattttcctgaccatgtttcccaccaatcctaacaaacctctgcttccctctcagtctcaacactctggtgttaatgcatttgaacctgctgtgtatctacttcctgtaattttttgagaattatctagaatggctcaaatcccacctttcccatgaagcctttctgaccactgcacatgctccttccccttgctgggctgtgtgctgcatgtgtgtacagggggacatcacatttgggtgcagcctgtcgtgtgggcgttccttttgacgcccgtgtcttgagagggctgtgtggtgttagcaggttagaggcagatgctaaagagacaggcctgaccattcactgacctagctttcctttccctgagcttcagtttcctcacctcttaaagggaaataaaaatagtatgagttgtgtgcagcttaactgagaggatgcctgtggagtgttcagggtgatggcagtggttattactattaatctttttctccccagccagacttgatgcctggacttggccacagcttgcttggtaaacccatttactgacatattaattgatccctcttctctcaccaggtacatggccacctctggcctagaccaccagctgaagatctttgacttgcgagggacgtaccagcctctgagcactcggaccctgccccatggagcagggcacctggccttctcccagaggggactgctggtggcgggaatgggtgacgttgtcaacatctgggcagggcagggcaaggccagcccaccctcccttgaacagccctacctcacccaccggctctcaggccctgtgcatggccttcagttctgcccctttgaagatgtgctgggggtggggcacactgggggcatcaccagcatgctggtccctggtgagtgggccaggggatggggcttgaagtgaatgaactctgggtggaagtttgggccagagacatccaggaactgggggcttagttgggctggagctgttggactgaggttcttccttatggctccatgcttctccctccctcccttcaggggccggtgagcccaacttcgatggcctggagagtaatccatacagaagccggaagcagcgccaggagtgggaggtgaaggccctgctagagaaggtgaggctcccctgctggagagggtgagatcccccccattgctggagagggtcaggttcccccgtcctggagagggtgaggttcctccttcctggagaaggtcaggttcccccctgctggagaaggtcaggttccccactgctggagaaggtcaggttcccccctgctggagaaggtaaggtttcctcttcctggagagggtgagattcccctctcctggacggggtcaggtttccctctgctggagagggtgagattccccctgctggagagggtcaggtttccctctgctggagagggtgaaattccctctcctggagagggtcaggttcccccctgctggagagggtgagattgcccctgttggaggtgaggttcccctgctgcagagagtgaggcccctgctgctggggaaggtgtggtttggggttggaggtagagaaagctctcctgatgtctcatgctgggggtcttgggtagaggggttcagcagcctccctggcttctctttttggtgctaccctcctgtggctccttgctgagtcctgtccctctgtgacacccccaggtacctgcagagcttatttgtctggacccacgagccctggccgaggtggatgtcatctccctggagcagggaaagaaggagcagatagagaggctggtatggagcaggcccctgaatgcccaggcccgtcttctcccccacatcctctccccaccatggcttgtccccagaagtgcggccagcaggtgggtgctgccagttccctgactccgaggctgtaacagatatcactcctgtcccctccctcagggctatgacccgcaggctaaggctcccttccagccaaagccaaagcagaagggccgcagctccacggcaagcctggtgaagaggaagaggaaggtcatggatgaggaacacagggtaaatgagcattggcatgggccgggccttccacaggctgcaccctcctgcttgtgcctctgcccttgtcagcctgccacttctcactctgtgcctgtgtcctccccgtctccggtccccaggacaaggtccggcagagccttcagcagcagcatcataaggaggcgaaggccaagcccacgggggcccggccatctgccctggacagatttgtgcgctgagccagactccagggttgcctgggaacagtctctccccaagatcacctgtagggaaatgagtgttccctggaacaaggaggtgggggcagtgtggccccttccccaactgggggtggacagctgtctcctggggtgggttggtattaaagaggaaagcgattttttggataatgtgtctggatcatcttgtggacctgttcccctcccccttgccaagcatgaggccattctaggatcggaggggtcagtgtcctgcctctacccccaccccataccttgtggctgctgtggccttagctttgccttttaggggccttcctcctcagagaggctgtcctctctcccatgtccctggagatgtccttcccatgcagcagggtctggcagcctctgggctctccaggtttgtggtgttctgggggtcctggtgaagaccacactgcactcgaagaagtggagacaagtttattgaggagcttgacacccctcttctgccctagcttgagagaacaactgcagcatttttttctttttctcttcccgatgaccatcttttgggctggcgggccaggcccctgggtgtctcccatatcgctgtctttagtgagactgaggatctggtataaggaaacagatcagctgcagccagtgagggcccctgcgttgggcctgggagagagaaggtcctgttcctgcctttcctgtggccccaggcactctcagctctgaagccaggctattgctcgacaccgcaggatgcccaggactccctggaaccaggagcaaaagggcgcagtcctttccccgtcagagccacccaccagcttccaggcttcctgcatcttccaggggtccagcctgtgggacgtcagcaggaatttcccatagcagcaccggtctagcgggtagtgggtggcaccagccagcttgacacactgtgttggggcgaggcctcctcgtcgggcacttacccccacaaagacatcctctaatgggagaaggggtgcccggctggccaccttgagaatgagctgcacagcagacgctgacagcacataccccgtgcctgaggcatagggtggaaaggggccccaggtgtgaggccactgctcctctgatacgcggtgcctgccccccggtgtccgagaggggttcacgcgccagtgcacccggcccaagtacagaagaggcacttcctcgctgtgcaaaacctggcctccttcctgctcagcctctctctggggttccgtgcttctctcccattgcccccaacggccccctcgcaagaccagctctgataccagttcagggacgttgacatacacatcatcgtccgtcttgaggacgtatcgggccatggggcagtgtttctcagcccagttcagcccgctgagggtctttagggtgaggttgcggtaggagtcctggaaggcggcctgcaagatatccccctgggctgctgactccgaggccaggtcactcccctgggaaccccacacggggtgctgtgcgttcggctctcccagcaagaatagcgtctgtaccctgagcccccgggcctcgcgcagcccgccccacgaagcccgaatggcgtttctctggttcaggttctccggagccgtgcacaccaggatgagcaggaagggaggggccccgggaccactgcaagcttcctggttggggatcaagaggcggggcagggccaggggcggccccggtgaggcgggggctgggagcagggaggctagtgagaggctcagcagctcctcccccaaccccgaaggcccgaagagggtccagacgatcaccagcagcaaagcggcgagaaggaggcgccggaagagcctgagctgcatggtgcggcgtactccgaggggatccgtaagagcgaagggcggagactagctccccgggcctgggtcacggccggggacttaaccgaccacgcccgggatgcggaggtctgagcgcgcgaccgggaccagcgcaccctggggggcggggagaggcggtgcagctgcggaccgcggtcgaggcatgctgcgggggtggctaccttgtcgccgcggcctcgccgccagcaagctccgctccatggctggccgactgcggcactcacggcccctcccccgcctcgccccggcggccggtctgccgcccgcttccgcgttgcgcgctcccatccccgccggaagcggcgtcggagcagcgcggcagggcgcggaaacccgggcgggtggagtggcaccaaacggagtgagaccggtctgtccagccgctccccatcccaacccaccgacgtactccaccagggagaccacacccttcataaccaacgtccgcgtttttgcctttgttgcttcggtcacagcccgaaggattttcccttatgactagatgaccccttaaaagaaaacaaaaacaaaaacaaaaaaacccagagtgaccaagacctgcaaactcaccttaactgcaactggctaggctcagctaacccactccacaatgatgctgcacaccaaccccagggtgttggtctctaaactggtctgggtcagcccctcaaggctgaactgttttaccctcacatttctctttacagagagggacatcactagctacatatagggagactggggggtctgcagcatcaccactgctaaaactaatagcactgtgtgggcaactgagtagggcagctctaaatggaaaaaggacccagcactttaacattccctattagaatggacagaaaggacaggaagccataggtatataaactatttattaacagacaaggcctacagacttatttcttcttggacacacccacggtgcggccacggcggccagtggtcttggtgtgctggcctcggacacgaaggctgcaggtaaaaagagaagagtcacaggtcatgcacagcagaaacaaaaggttggggaaaaagaagagcgagtaggaggaatgctgagtcgaggtaggcagggagatgagaccccccactcacccccagaagtgacgcagccctctatgggcccgaatcttcttcagtcgctccaggtcttcacggagcttgttgtccagaccattggctaggacctggattcagaggagggggcaagggatgttttatcccctgacctcctatgcccaattctgttaggaccctccacccttcctctaatcctgccctcatttcagtacacacctggctgtattttccatcctttacatccttctgtctgttcaagaaccagtctgggatcttgtactggcgtggattctgcataatggtgatcacacgttccacctggcaggcagaccaaggtcagatctagatggacccctttctgaggctgaccccacccccagcccccttccttgtcctcacctcatcctcagtgagttctcccgccctcttggtgaggtcaatgtctgctttcctcaacaccacatgagcatatcttcggcccacaccctgaatgaagtgaggggtttcggaattagtgttgaatctgatttttaaattcctttatagcctcattcaaaaagctcacaaaggtaggatctgcaataatctattttgcaatcccctaggtcaccctttcctcacccacctttccagatataaaccgttcgacagaaggttaactttgggccggtcgcagtggctcacgcctgtaatcccagcactttgggaggcccaagtgggcagatgacattgagggcaggagttcaaaaccagcctggccaacatggtgaaaccctgtctctactaaaaatacaagaaaagttgctgagcatggtggcaggtgcctgtaatcccagctacttgggaggctgcagcagaatagcttgaacctaggaggtggaggttgcagcaagccaagatcacgcaaccacactctaacctgggcgacagtgagaccccgtctcaaaaaaagaaaaaagtaactttgttcaggggtatgactgaatcatgaacacagaatttagtattacagacaaccctttgaaagcaggcagtatcttttttttcccccattgctcagcatatgtagttgtcactaagtgccttctgattcttgcatttaggagaacaaggggcctgccactccaagctgatgcagtaaaccagggtcttatgagttacaggctttgattaaggtattgggctgtcagttcctcagaatctctaagggtcacaattatttaggcaaatcactgtatccccataatcaaagtattccaagatcacaaactcaattttgtcagtgcccagaactgactgcccaagttattataaaaagaaactaatgcttacattggtagcatcacatcaacaccagcactagcacccatgccatctgaactcatgaaccaaagtgatttggagagtgaaggaggaactgaatactgagccctgcccctcattttgtccaactagaacatcagaatgtggccctatttggaaataaggtcgttacggaaataattaggtaaaatgacgttatatttgattagggtgggctctagatccaatgacaaatcatgagagagacacgaagaggaccatgagatgacagcaacagattggagtaatgcagctacaagccaaggaacactaaggattactgacaaccaccagaagacaggaagaggcaaggtaggattcttaagtgcctccagcgccaagagtaattttctgcttagtttatatgaacttgttacagtaggctccaggaaactaaaacaaatggcagttcctatctgcaagaaagagatcaagggttggcttcaccgtggctcatgcctatatgcctgtaatcccagcactttaggaggctgaggctggaggatcactgagcccaggagttcaagaccagcctggacaacacagcaagatccagattagccagatgtggtggtgcacacctgtagccccaactacttggaaagttgaggtaggaggattgacttgagccctggaggtcgaggctgcagtgaagagtgattccgccactccagcctgggcgacagtctcaaaaaataaatgtggctgggtgcagtggctcacgcctataatcccagcactttgggaggctgaggtgggtggatcacctgaggtcagaagtttaagaccagtttgaccaacatggtgaaaccccatctcttctaaaaatacaaaaattagctgggcgtgtgcctgtaatctcagctacttgggaggctgaggttgcagtgagataagattgcaccattgcactccggcctgggtgacggggtgagactctgtctcgggataaataaataaacatgaccaagggtcaaattattaggttgattcagaagagagcagggtctttcaaatttattaattaaatgacgaggtttcaccatgttgaccaggctggtctcgaactcctgacctcaggggatccgcccacttcagcctccgagtgagccactgcactaagcctcaaatttaattatatgtttacaagtgaaaagatttacttcaaaatgggtgtgggaagagatatagatgaaataagatttctaatttttagttagatacatccatgaaggttattctctattacacttatgaatgctgcacctctacttaagcctcacagccggtctctgaaataagtacagtatttcacactgccttattagtttcacagagcacccaacagaaggaatttccctctacaaattcctcaacagagccaacagtatccctactactatccattccctaacgtcgcctaagtacctaccatgcaacagaagccaagaagacaggcctagccctgcacgcatggacacatactggtaggctatttccacaactaactacacattctgacttaagggaaaaaaaaagaacgttatgtatctagaaagccaagaattcattaggtttgtggagagaaatactcaagtgaaataatattcttgcattcattgattcgattcttaaaagccaaagaaagatggatgtactccttcacacttcgacaggaaaggagtgtcaattatggctcatctgagtaaatgtcccactcagacttcttccaccctccatctacaggcttcttcacactttttatctgttggtatttctacttatccctcaagatgcccaataatcctccaattctcagggaatataaatttcgcaggaagggttgctgttatgatggtgggatgggtaaagaaaccgaatctagaagaaatctcacaagctcgtggttcaggaaaatcttagcgaattcgggtgagcaaccatacctcctccccacttccccaggtgaaatgttttggcttgaccttagaccactatcttggctttaatcacttattttctgtggtaaatgctaaaggggtcagtctctccctaaaccactttcttttcttaatccagcagtcaaggtgatctggatgagactcaagtctccccagccttgcttcatttttgcttatgtcttcacaacccaattctcatttacattccctattccttaccctacttcacttaccttaatggcagtgatggcaaaggctattttccgccgcccatcgatgttggtgttgagtactcgcaaaatatgctggaacttttcagggatcactagagactagttgaaaaagtacgggggaagaaatcagacacagtaaactatcaaacagtaaggccgataaggcaaacgaacttcatacaaaaaggttaagcctgtcacacaagtggcaagacacccgccccccattccttaggttttcacaccacagagcaaactcctcagtcctgcagagtaggcacatgaagcgttcaagcttgggaaaatgcaacctctggagagaggaagcaggtctcctgcccattcccaaaagtgcgtggcaaaagccagagttccccatatacgcccaagccttagacagctttcgtgcgcccgagcagtgacccttcctcgggcccgctctccggaattggaaatctttggtgtccctttccagggacgcgatccaaggctccatacagaaagtcgcaggccctcagggcaggacgtttccagaaccctgacagccggctttccttgcccgagctgcgatgccgctggatcacggcacggattccagtcttaccatggctgcagcacaagcggcggcgtgtaggcctcctgtggaagagagagcggaagtgacgcgatatcattatatagcttccaggaggaagaggcggtgcgatctaggagaagtgcttccggaaactgtttcagcgcgacctggctggatttatgagagtatggcttgggtaccacaacttccggtgcgcctttctttacagttcgtaaggttcataggtaagagaacagcgaaggttccggggctagtttgtgtttcagacttccatatttccatttgcagttgggtgaagtggttagagctgaaaggggacagctggagtcaaacaacttaactcaaatatctgggaacgatttcgcgctgaggaagaccctttgggacttgtagctccactccggggaacggactcgccgggactgacagttgccggaagtgaggctgcggggaatggccgccgctgcgaccatggcggctgcggcccgggaactggtgttgcgggctgggacctcagatatggaggaggaagagggcccgctggtgagagcgcggagctgtttctcctccgtagttccggtgctcctagcttcagaaagacctgagctgggtttaggcacaagggtgggaacttggctgctacccgtgatattacctgtttggctccccgtacccttccctgtgatcagacccgaccctgagcgggaccctagtggctgaaactcatgttctggggcacaaaatttcaacttcactccacattttggggtagagtcacgaagtagtatagtgtaaaggttaagaacatggcctctgaagccagactgcctgagttctatctctgccacttacacgctgtgtgcccttgggcaagttaaccagcttctctgtgccatcatttctcacacctttaaggtgaggaagatagaacctattgtatacatcgtcgttaggattagatgaatatgtgtaagattgcacctggcatgcaggtaaacactatgtgttttgacatttctagaaagcctggggttccccagaggtgtgatagaacacccaagatggcaggagtctgagggtcttaaatttaagtgcaagttctgctctatcctctgcaattcctagctttcctcattcggtcatctccactgcacttttttttttttttttttttttttgagacggagtcttgctctgtcgcccaggctggagtgcagtggcgcgatcttggctcactgcaagctccaccttccgggttcacgccattctcctgcctcaacctcccaagtagctgggactacaggcgcccgccaccgcgcccggctaattttttgtattttttagtagagacggggtttcactgtgttagccaggatggtctcgatctcctgacctcgtgatcttcccacctcggcctcccaaagtgctgggattacaggcgtgagccactgcgtccggcctccactgcactgtcttgtcttttacctttttttgttttctttagacgggtttccctgtgttacccaggttgaccttgaactcctgatctcctcaaacaatcttcccgtctcagcttctaagtagttgggactacaaggagcccaccaccatgctgggctacctccactgtctcactggatcgtctcactccttgtcgtattccttacccatcagcacaaaataggagagtgggtacaacgtgagactcttagaatttttcaaagcagtacctgtggctcccagtaaaagcagctccttctcacttgaaggactggggatatccaattatattttttctttctgtctttcaccgtaccctactcccattaacttatccctttccaggcgggtggtcctgggctccaggaaccactgcaacttggggagttggatatcacttctgatgaattcatcctggatgaagtggatggtaagcgatggcatggggtgggtgcagtacctgtaatctgagaggcatgggggctggaaggaataaggtagggattggtgtgcagatactcaatgtggggagagggagctgtggagctggacccttatgataaacttctatttccagttcacattcaggcaaatctggaggatgagttagtaaaggaagctcttaaaacggtgaggctttcccctgtactaatctgcccttctatctattctaccccaaaaggaaaaacaacagtctcttgcttttgttgtatcagcctggtatctgaccctttagtccttcactgattctgttacagtgtccctttactgagacttccttctgacctgtcatctttatgttctcccagggtgtagatctccgtcactattcaaagcaagttgagctggagctacagcagattgaacagaaatccattcgggattgtatcctccagcagagggaaggggtggtgctattgacatggggattttgtggcgtgtggtacagatgtcccagctggggtcaacatctctgaggtttctatcacttcaaagagaaattggagaggtactacaaatctgaggatctcccatcttcaccatgaggccccttgactttttgtggatcagatattcaagagagtgagaatatagcatctctacacaaccagatcacagcctgtgatgctgtcctggaggttagtaatcatgacctgtgacccctaatagctactctgaaccagatcatggactctggtgccatttttaggtcatcagcctcaagatggggacaggttagggtcattttcaagatgacttcaggagccttttttgttattactgccatatttaatgttatttcctgataatctggggtgtctcatcatctgccaccctcaaggactgataaatactgaagacttcacaaagagtctttggtggcccacttatcccctacaaaccctatagcactccggcaacatctaccctccctacagcgaatggagcagatgttgggagcttttcagagtgacctcagctccatcagctctgagatccggacactgcaggaacagtcaggagccatgaacattcgacttcgaaatcgccaggcagttcgggggaaacttggggagcttgttgatggtctggtggtgccttctgctctggtcacgtgagttaccagtttgaagggttataatgtccatagcaggtggatgggctggggtgacccaccctatttaccatatgggtctcctggctccaggttaagtgctttttagaaattagtagccaggttaaccaccatttctttaggtatgcattaggcatcaaagattaagattaagaatgatgtaagatcaggtgtatagtggggaggtccaggaatagaaaactttgtacatgtgtcctgactatatggtacaatgcaaggtggtagagatgacaggggctggaggtaaggagtgtggtctgggtctggtttagggatgtctgggtctcccttgtaactcatcccacccctccctgctaaccagggcaattctggaggctccagtgacagagcccaggttcttggagcagctacaggagctggatgccaaggcagccgcagtcagagagcaggaagctagaggcacagcagcctgcgcagatgtcagaggcgtgctcgatcggctccgggtcaaggtgggaagtagggatggataccctggaggctactggaagcagccttcccaagatcttcactatggatttccttggcagagctcactgtagcctgtctccagggacttcaccctgccttttctgggcagccccattcttgtctctctgggtttccctgaaaatcttgagaaataccaaagagatattcaccacccttctgcctatgtttaccatttgttgcttcccccagttcctctcatacagcaaaagggttgcagcttcatgccctgtgtttggttccccacatctaggcagtgacgaagatccgagagtttatcctccagaagatttattccttcaggaaacccatgaccaactatcagatcccccagacggccctgctgaagtacaggtcacaaccccaaggaagtgcatgggatggcctttgagtttttgagcggcctagtgtgggcatctggtttcttgggagacaggtagactgacatgttcctgaccccctaggttcttctatcagtttctgctgggcaatgaacgagcaacagcaaaggagatcagggatgaatatgtggagacgctgagcaagatttacctgtcttactaccgctcttacctggggcggctcatgaaggtgcaggtaaggtcaggagggagaggtattcctgggcacatggactgggaggaggggacatccctgggcaaggaatattttattgtgttgtgggaagagacggttatcagttctcttttctctttccttagtatgaggaagtcgctgagaaagatgatctaatgggtgtggaagatacagcaaagaaagatatcctagtactggggaaccctcatgccaggccttgagagtgggaggactttcgactccagccaccaatgcactgctccttaccttttcctatgggactgaaaccttttagaatgtgatgaaagctatagcctccctccccagaaagatgcacacattattttgacacatagttttgcatatgattgcaagggagggagagacaccctgaagcccattcacagatgtcagcacgtggggccctagtactgggaaagagggctggctaggccacgtctgcctgtctggggtccccacagatagtgagtctttgggcccagttatggtctcattgttttccctgactctgacccatcttacgattcttctcaaagccatcgctccgcagcaggaacaccattttcaccctaggaacccgcggctctgtcatctcccccactgaacttgaggcccccatcctggtgcctcacacagcgcagcgcggagagcagagggtatgaggaggaacaagcttcagtcacgaaagaggctctgagcacggtggggagggaaggccagacctcagggcagagcttcccaacctgcaggccctgtcttaatccccttagacttgggagtggccttctccaatcactccagcctgctgtgatgtgaaaagagctgggaaccattgacttaagggcatgcagaggggagagactggatgaggggaatgacagtcagaggcaggtgttgaggtggatgcaggagtagcaacagcaggtgggtgtaggccctttttgtacccctatgttcccccaccttttcagtgcccattcagcttgctcttagacagcccggacctagatagatgcgaagggtctgcctagctcttattactcaggtttactgattttccagccatatccatgggaagtcagtgttggggtccatcccttctgtttttcttctttactcctcctctcccagtatccatttgaggccctcttccgcagccagcactacgccctcctagacaattcctgccgcgaataccttttcatctgtgaattttttgttgtgtctggcccagctgcacacgacctgttccatgctgtcatgggccgtacactcagcatgaccctggtaagactcctgtttcctgtaccattcaacacctttgtccccagtcttcatcagcagcatagtggggtcatgactctggtagaactgaaatcaggatcgcctcattctaaaatgtctaagccttggctgggcgtggtagtgcacacccataatcccagcactttgggagtccaaggcagtaggattgcctgagctcaggagtttgagaccagcctgggcaacaacctcatctctacaaaaaatgaaaaattagccgggtgtgtgttgatgtgtgcctgtggtcacagctactaaagatgctgaggtggaaggattgcttgagcccaggaggtcaaggctgcagtgagccatgcactccagagtgagacctgtctcaaaaaaaaaaaaaaaaaaaaatagccaggtgcagtggcttacgcctgtaatcccagcactttgggaggctgaggcgggaggatcacaaggtcaggagtttgagaccagcctgaccaacatgatgaaaccctgtctctactaacagtacaaaaattagccaggtgtgcgcggcctgtaatcccagctactcgggaggctgaggcaggaggattgcttgaacctgggaggaggagattgcagtgagccaagattgcgccattgcactccagcctgggtgacagagcgagactccatctcaaaaaaaaaacttgttacttagacttgtcacagtgttactcccccatgaaatcctcagtgcgtagggtccctgagactacccctactgctgacccacaatcaggcaatagcacgtgaaaactgagggtcccccaaatcttggtgctcaaacccctgacccaagctcagccctgaactctgctgtgataggcttgtgatcatagatcctggtgtggccaggtgcagtggcttatgcctgcaatcccagtactttgggagaccaaggcaggaaggttgcttgaggccaggagttcgagaccagcctgggcaatgcggcaagaccttgtctctacaaaaaatttaaaaataaacttaaccaggcatggtggtacacacccatggtcccagttgcttggcaggctgaggtgggaacatcacttgagcccagatgtttgaggctgcagtgaactatgattgtaccattacaatccagcctaggtgacaaaacaacctgtctctaaagccgaaaacaccgggtgcagtggctcacacctgtaatcccagcactttaggaggctgaggtgggcggatcgcctgaggtcaggagttcgagaccagcttgaccaacatgatgaaaccccatctctactaaaatacaaaaattagtcgggcgtggtggcaggcgcctataattccagctactcaggaggctgaggcaggagaattgcttgaacctgggaggcaggcggaggttgcagtgagccgagattgtgccactgcattccagcttgggtggcaagagtgaaactctatctcaaaaaacaaaaaaaaagccaaaaacatagatcctggacaggaaagcagggggcagttccattttacgtctctcccttctgtcctgtccagtgcatcatccccaaagaaacaaatgcgtttgttcatagtcactgaggacaacaaactgacattctcttttaatcctctgtttgttccccaatcacacagaaacacctggattcttatctagctgactgctacgatgccattgctgtttttctctgtatccacattgttctccggttccgtaacattgcagcaaagagggatgttcctgccctggacaggtcactgaattctggtccttgatgcccaacaggccacgaactctcttggtcttgcatgactctgccctgaccttagactcctactgacctggtatcatcaacctttattcccattcatttgctccggaggcagtagtgctgctttttgactcacctttctccaaccactgccccccaacccccacaactagctattgaccattaaactgaatgatgccccactccctcccatccccctccaacattccttgctggctgacctctgattctgattaagcccacaggtactgggaacaggtgcttgccttgctatggccacggtttgaactgatcctggagatgaatgttcagagcgtccgaagcactgacccccagcgcctaggggggttggatactcggccccactatgtgagggagggcaagggtaacaaagggttcttgaaaggcagggctctaagcatcattgtgggggttggccaaggtcacagttgtgtcatgggccacgctatgagcaggtgttctgggagggtggtgaaactcatggtgagtggtgcactgtgggtaggagggactgaaggtagagtcacatcacatggaggtgggttggctaaggaagttgggaggttgtgttggttgggccaggaggtgggtgtgattttccttcccactctctcctagatcacacgccgctatgcagagttctcctccgctcttgtcagtatcaaccagacaattcctaatgaacggaccatgcaattgctgggacagctgcaggtgagggtcggacaggagacacttcccagcagcagggctgcttccagtgggtggagtctgaggaggagccagatggggcccagatgaccttgctttctggtgtcacctcttcctctctgccttttcaggtggaggtggagaattttgtcctccgagtggcagctgagttctcctcaaggaaggagcagcttgtgtttctgatcaacaactatgacatgatgctgggtgtgctgatggtaataggtgctccttctccttgccttcctacccagaaaattcggtttcccccactgctgttagctctgtgagggcagagatgatgcctgtcttgttcagtgttttactcttggagtctggcacagtgtccagcttagtacaataagtgtggaatgaaggggacatcctgaagtgaaggactgctttccccaaactagacatttccctttgtcttcccttaggagcgggctgcagatgacagcaaagaggttgagagcttccagcagctgctcaatgctcggacacaggtaggggtgtggggaaaagggaagagacagccttctgtgctgttttcagctctctctctctctctctctgtgtgtgtgtgtgtgtgtgtgtgtgtgtgtagggagtggcataatggatctcaggtatgcggaattactgtaactcttgattcacaacatactgtcattattggtctattttattttattttgtctttggtttttttttttttttttttttgaggcaaagtgtcactctgtcacccaggctggagtgcagtggcatgaacatggctcactgcagcctcaacagcctgggcttaagtgatccttctaccttagccccctgggtagctgggaccacaggtgcgagccaccatgcctagctaattttttatttttagaatgaattttatttttttctttatctatcctctcaagcatttatcctttgtgttataaacaatccgattacactaagttattttaaaatgtacaattaagttataattgactatagtcaccctgttgtgctgtcaaatagtaggtcttatttattcttcctatttttgtttgtacccattaatcatccccaccttctccccagccttccactagccttcccagcctctggtagccatccttctactctcttcgtacatgagtacatgagttcaattgttttgatttttagattccacgaatcagtgagaacacatgacgtttgtctttctgtgcctggcttatttcacttaatataataatttccaattctatgcatgttgttgcaaatgactgagtctcattcttttttatggctgagtagtactccattgtgtatatatcccacattttctttacccattcatctgttgatggacacttaggttgcttccaaatcttagctattgtgaacaatactgcaacaaacatgggagtgcaaatatctcttcaatgtactgatttcctttcttttgggtatatacccagcagtgggattgctgaatcatatggtagctctacttttagttttttgagcaaccttcaaactgttctccaaagtgtttgtactaatctacattcccaccaacagtgtataagggttcccttttctccacatccttgccagcattgctattgcctgtcttttggataaaagccattctgactggagtgagatgagatctcattgtagttttgatttgtatttctctgataatagtgatggtgagccccttttcatatatgtctgtttgccatttgtattcttttgaaaaatatctattcaaatcttttgcccattttttaataggattattagactgtcatgtagagttgtttgagccccttatatattctggttattaatcccttggcagatgggatgtgcaaatattctttgtctcttcactttattgtttctttagctgtgcagaagctttttttttttttttttttttttttgagatggagttttgttcttgttgtccaggctggagtgcgatggtgcgatctcagctcaccgcaacctccgcctcccaggttcaagcaattttcctgcctcagcctcctgagtagctgtgattacaggcatgtgccaccatgcctggctaattttgtatttttagtagagatggggtttcaccatgttggtcaggctggtctcaaactcctgacctcaggtgatctgcccagctctgtctcccaaagtgctgggattacaggcgtgagccaccgtgcccagctgtgcagaagctttgtaacttgacgtgatcccatttggccatttttgctttggttgcctgtgcttgtggggtatggctcaagaaatttttgcccagacccgtgtcttggagattttccccagtgttttcttgtagtagtttcataatttggtcttagatttaagtctttaatccatttgatttggttttcatatgtggcgagagataggggtatagtttcattcttctgtgtaatggatagccagttttcccagcaccattattgaagagactatctctctttttttttttttcagacggagtcttgctctgtcacctaggctggagtgcagtggtgcagtattggctcactgcaacctctgcctccttggttcaagtgattctcctgcctcagcctcctgaatagttgggattactgatgcctgccaccacgccgggctagtttttgtgtttttagtagagacgggatttcaccatgttagccaggcttgtcttgaactcctgacttcaagtgatcctcccacctgggcctcccaagtgctgggattacaggcgtgagctaccgtgcctgaccaagagactatcttttccccagtgtatgttcttggcacttttaccaaaaatgagtttactgtaggtgtgtgccagctaattttttaattttgtaaagacagttgtctttacaaaagacaactgttgcctgggctggtcttgaattactgccctcaagtgaaaaatcttatgattcttttttttttttaaagacggagtctcactctgactcccacactggagtgcagcgatacgatgtcagctcactgcaacgtccgcctcccaggttaagtgattctcctgcctcagcctctgagtagctgggactgcaggcacgtgccaccatgtccagctaatttttgtatttttagtagagatggggtttcaccatgttggcccagctggtcttaaaactcctgacctcaggtgattcacccgcctcagcctcccaaagtgctgggattataggcatgagtcactgcacccggcctagattctgtttttaatgacactggaaaacaacgaatttttgttttggcatatatatagctttacttttaaaagcaagaacacacacaaaaaaagggggaaaaaagcaagaacacaataaatgcaacatttagatcaacgagtacctgtgcacgagaagcacagggatggtttcagtaggatttcatagtttgatggtgacttttgccaagctgctgctacttggattgtgtggtgtgtttgtgagtgtttatatcattaaaaaacaaaacagccactgcactgggttgttttattttttaatgatataaacactgatgagacaaggcaagaaaacagcaaggcaaaaaagaaaaaaaaaaaagccaggcatggtggtatgcacctgtagttctagctatttgggagacaaaggcgagaggaccactttgaaatccaggagttcgagattacagcgagccatgatcatgacattgtagtctacccttggcaacacagcaagaccctgtctcaaacagcaaaacaaaacaaaaaaccccagaatcacaggatttttttcatgcataatttttctaatattgtcatgttattgcatatggctatagctcattccttttcactgccatatatgacttgtgtgattaaacatattctataaatggattgtttccagtttcttgcttctgtgagcattgttttatgaataatcatgtcttttagcacacatagatattgattatcttcatcaaaagtttctcttgggtataaacctaggacagtagtcttgctgctaataagatatgcagtattagtctttatgagacaatgataaattgttttcctaagtagttccttctcaccagcaatatgtaagaattcctactgagccacattttctccaacatgtggtattatcaggtctttgccattttttttctttttttttttagagaccaaggatgctgctaaagatgctcgggatcagaaatgtttcagattttgatttttttcggattttggattccttgcattatttttactggttagcatctgtagtctgaaaatctgaaatctgaaatgctccaaagagcattacctttgagcatcatgttgatactcaaaaacttccggattttggagcattttggatttcagattattggattagggatactcaacctgtatttcagtgggttttatttgtaattttctgattactaataacattgagcatcatttcaaatgtttagtaactatatgtaggggttcagtcaggctggtgggaaaaatattagtgatgatagccacaaaccctcttggaaggcctaagagtttgcataacttcagtaatagatatggttgaaggcgacctgatctttacctttagttaaataaattagagtaataacaaaggaatgtggggaagttatctaggtagcttgtttactcatatggtcttaagactaatctttgatgtaccgcaggtgcttaactgctttctactcaggaagtccacaatgtcagttaccccgtagtggtgttgactcaagtctttgtcaattaatctttactgaataaatgcgagtctcactagctggtcagggccgccattgcaactgtttacagtactcttcagggagtctgtaaacagcctggacacactcagctggactggcaaagcagagtatctgtgtgtcagtgtacctcattcatccgttgccgggtcaggggtctgcaagggacagactccctgaagctggtgctctgtgtgaggagcgtcaccacaactatacaatttttttttttcctgtaaaatggctgtcatagcatttgaccttttaaaaatcggcttgtggctgggcagggtgtacacctgtaatcccagcactttgggaggctgaggcggatggatcagttgaggccagtaattcaagaccagcctggccagcatggcagaaccctgtctctactagaagtacaaaaattaaccaggcatagtggcgcatgcctgtagtcccagctcctcaggggcgctgaacctgggaggcggaggttgcagtgagccaagattatgccactgcactccagcctcagttacagagcgtgactctctcaaaaaataaagaaaaaaataagtctgctagtattttttatttataagagttctttgcatcatctacatactaatcttgttgattatacgtaggttacagattatcttttagtatgtatctttttacttttttttttttttttggtgagatgagggtctttctgtgttgcccaggctcctgggctcaagaaatccttgcacctttttttttttttttttgagatggagtttcactcatgttgccgaggctggagtgcaatggtgtgatctcagctcactgcacctctgcctcccgggttcaagcacttatcctttctcagcctcctgagtagctgggattacaggcgtgtgctaccacgcccggctaatttttgtatatttagtagagatgggttttgccatgttggccaggccagtcttgaacgcctgacctcaggtgatccatccgcctcagcctcccaaaatgctgggattacaggcatgagccaccgcgtccagcctttgtttttttaatgttgctttttgattaaagaaatttgtaattttaatatgattgaatttatctgtccttcatttttatgtctttgtttttagtaacagtcttgctctgtcacccaagctggaatgcagtagtgtgatcatggctcactgtagcctcaacctcctgggctcaggcaaccctcctccctcagcctcccaagtaactaggactacaggtgtgtgccatatgcccagctaatttttgttgttgttgttgttttgtagagacagagtctcactatgttgcccaggctggtctcaaactcctggcctcaagtgatcctcccgcatcagcctcccaaagtgctgggcttacagccaccatctaccacgcctgactatttttgtcctgtttaagaaattcctgggctgggtgtggtggctcacgcctgtaatcccagtactttgggaggctgaggcgggtggattacgaggtcacgggatcgagaccatcctggccaacatggtgaaacctcgtctctactaaaaatacaaaaaaattagctgggcgtggtgatgcgtacctgtagtcctagctactcatgaggctgaggcaggagaatcacttgaaccagggaggtggaggatgcagtgagccaagatcgtgccactgcactccagactggtgatagagtgagactccgtttaaaaaaaaaaaaaatgaaattccttattactccaaggccagaaaaatattatgctacattttcttcttaatattgtagaattttggccaggtgcagtggctaaagcctgtaatcccagcactttgggaggccaaggcaggaagattgcttgaagccaggagtttgagaccagcctaggcagtatagtgagacctcatctctacaaaaaattaaaaaaatatattagccaagcatagtggcacacacctgtagtctctgctactcaggtagctgaggtgggaggatcacttgagcctgggaggtcgaggctgcagtgagccctgtttgtgctgctgcactccagcctgggcaacagagtgagaccctgtctcaaaaaataataataaaattaaagaataaacaaatatatagagagaatttttttgttacctttaagtctcaaatttacttggaattaatttttgtgtgtgatgtgaggtagggatctatttcttttttttatgtggatggttggcccagaaccatttatagaaaatctttttcttttctttttttttcttttctttttttttttttttagacagagtctcactcttgtcacccaggctggagtgcagggccacaatctcagcgcactgcaacctctgcctcctgggttcaagtgattctcctgcctcagcctcctgagtagctaggattacaggcatgtgccaccacacccagctaatttttgtatttttagtagagatggggtttcaccgtgttggctaggctggtctcgaaaagttttcacattttaaaaagtctttaaggattttgattagaattacattgtctatagactaatttggaagaattgatactttatgatattgaaacttcctatccatgatcatgatcatgggctccctatccttttttttttttttttttttttttttttttttttttttttgagacaggttttgctctgtcactcaggttggagtgcagtggcacaatcatagctcactgcagccttgaattcctgggttcaagtgcttctcttgtctcagcctcccgagtagctaggactagaggcacacagcaccacacttggcttttttttttctgtagagaatacaaaatttaaaaatttaattttttgtagtgtctcactaaactgggattacagatgtgagccactccacctggcctcctcctccaatttttaagagagtcttttaatgtcttttgataacctttttaaattttcagcacaaagacactgtaagtcgtttgccatatttttaggtgggtataatacgtatgtttagttctactaggaataaataaatcttttctagttttttgtctttaacatcttatgtttttgatttcttattctactggctaacactcccaggatgatgtttagaagttgaacagaaaggtgatagtgagaacccttttcatattcctgattttgaaagaattgttgctggtgctgtggctcatgcctgtaatcccagcactttgggatgctgagatgggagaatctcttgagcccaggagtttgagagtttgaggggatctgcctgggcaacatagtgagactccatttcttttttttttttttttttttagacagagtctccctctgtctcccaggctggagtggtgcagtggtgcaatctcagctcactgcaagctccgcctcccgggttcatgccattcttcctgcctcagcctcccgagtagctgagactacaggtgcccagcaccatgcccggtattttttttgtattttgtatttttagtagagacggggggtttcaccatgttagccaggatggtcttgatctgacctcgtcatctgcccgccttggcctcccaaagtgctgggattacaggtgttagccaccatgcccggctaagaccccatttctttaaaaaaaaaaaaaaaaaaaaaaaaaagccaggcgtagtggcatgcatttgtagttctagctgcttgggaggctgaggcgggatcattgcaggagattaaggcacccacaaagatgcacttaaaccagttgtttctgctgagagcagctgggatttgtcactggatttgccaggtatagagactcgccagctgccatcaatgcttctggtcctttttggtcagctgctgcttcatactacgtaggaaagattgttaaagcccagtgcactgtttcctctggtcactcctctgctatcaaggtgttctcttttgcgttatagaaagcttaaagttcatctaagtccagaccagtactcctataggaatataatgcatgccacaaatgtgagccacttaaataattttaaattttctagaagctacattaaaaagtaaggagaaacagatgaaataattttttaaaccactacatccaaaatattattatttcaacatataatcaataaaaaatagtactgaggccagcacggtggctcacacctgtaatcccagcacaggccaaggcagggaggatcgcttgagcccaggagtttgagaccagcctggacaacatggtgaaaccccatctctacaaaaaataaaaaaaaaatgagtgggacatgttggcacatgcctgttagtcccagctactcaggaggctgaggtgggataatcactgagccaggaaagttgaggttgtagtgagctgtgattgtgccactgcactccagcgtgggtgatggagcaagaccctgtttccgaaaaaaaaaaagaaaaaaaagatatacttaaaatttattctaagatgaagtcttcaaaatcaggtgtatatatcatgcttacaacacattatataatttatggctcctgcctagcaggggtcatcactgttagttcaacttttctttggcaagtctactccctgtgccccaccgtacactgctacattattgtttgtatatgtgtgtgtgagactgcatttaggcaactgtaataggttgaatttggttatttaatctctgtccaaaatcacatcacaaagatgccttgattatagaaagatccttccttgcagcatattagttatttgacaccctcagtggatataagtgaatattttgagggaagaatagaaaagatacaatttttatttatttatttatttatttatttactgagacaagttgctgctctgtcgcccaggctggagtgcagtgacatgatcttggctcactacaacctctgcctcccggttcaagtgattcttctgcttcagtctccctgagtagctggggttacaggcatgcaccaccacacctggctaattttagtattaataatttatttttaaatggaataataatttcactttcttcttaccagatctttctgtctgggttgtttgcttttatgagtttttttgtttgaatctcccttttacattaaacaaaaattaacattttattatgggaaatttcactatgctattagaaaagcatagtgaacccccaggcatcatcagtgatattcggtgtatagcagttcacggacagttttgtttcatctataccaacacctattgcttatcctccttctgtgtcccccagttactttgaattaaatcttatacatatacagatgaatattatcacttcatctgtatcagaatgttatttttattaagaaaattatttttgagatagggccttgctgtagtgcccaggctggagtgtagtagcatgatataactccctgcagcctcgaactcctgggctcaagtgatcctcccacctcagcctcctaagtaactgggactacaggtgtgtgccaccacacctggtaacagatggttttaaaaactacaatacattatcacacttaaaaaaattatcttatctaaaataactattcagattttcccagttatttcataagtgatttttttttttttttttttgagacacagtttcactcttgttgcctccaggctggagtgcaatggcccgatcttggcacactccaacatctgcctcccgggttcaagcaattctcctgcctcagtctcctgagtagctgggattatagacacccaccaccacgcccagctaatttttgtatttttagtagagacaggatttcaccatgttggccaggctggtctcaaactcctgacctcaggtgatctacccgcctcagcctcccaaaatgctgggattacaggcatgagccactgtgcctggccttcataaatgatttttatggttcaaatcaggatccatataaagtccatatatgtgtctttttttagtctatagatttcccttccatctcctctgcaatttatttttggaagaaactgagttattgagtcatttgtcatctagaatgatgtacagtttagattttgctgactgcattccatggtgtggccaaacttcctctgtctggtgtatttctcataagtgtgtaattagatggaaaggcttggtcagatttaaattagtttttttttttctacagggatatttgagaggtagtggtatgtatttccatcaggaagcacataatgtcttcttggctctctttgtgatgttagtagccatcaatttcagtgcctagatacattaattagatttctgtatctcctactatctcaaaattttccatcttctgtctgtgctacattctgagtcctttcttcagttataaattctaattcattgtataattcttgttaatctgttgactttctaaaactggcttttcatatctaaaaaaatttccttttttcaagtctgttcattcctcatagttcttactaattggtcatttttacatcctttttttttttttttttttttgagacggagtcttgctctgtcatccaggctggagtgcagtggcatgatctctgctcactgcaacctccgcctcccaagttcaagtgattctcttgcctcagtctcccgagtagctgggattataggcatccaccatcatgcccagctaatttttgtatttttagtagagatggggtttcatcatgttggccaggctggtcttgaactcctgacctcaggtgatctgcccacctcagcctcccaaagtgctgggattacaggcgtgagccactgtgcccggcctatccttaactttagacattgcacacagtacaactgctatccgtatcggatagttcttaaggtcagcagctattgcttattgtgtctgctgactctcttggtagctgcccttcttttgtgtctagtgatctttgagttcattgtttgatcttaaccaggggaactgtatgggccaaaattaggattgaggatatttttctccatagaggattttccttaacttttgcagcagctgaaagatgccattcagatggatctacattagtcatgatgccagaattgggcaaatctgctgacaccaggacacctgtgtatatgtgtttgtgtctttccaggaattcattgaagagttgctgtctcccccttttgggggtttagtggcatttgtgaaggaggctgaggctttgattgagcgtggacaggctgagcgacttcgaggggaagaaggtatgaggaaaatatggtaatgatgggatcagtggtaagggaagtggaaaagaaaaatgaaaggatgcagattacatggtgggggaatagagtatgaaagactggattgagagaataccagaaaagagggtttgatgataaggatggctataccttggggagaacttagtggagttgaagatcagccagatccctctctgacactgtttcctcctgctattagcccgggtaactcagctgatccgtggctttggtagttcctggaaatcatcagtggaatctctgagtcaggatgtaatgcggagtttcaccaacttcagaaatggcaccagtatcattcaggtgacctgcaagtcccaggccccactcagatcccccatcattaattattttccccatctttttgctgggctcagcatcatctaagtgacccttggtcttcagttactagctgtgcccaaagttctgtatgagccctaacctgattcttttttttttgtttgttttgttttttttttgagacagagtctagctctgttgtccaggctggagtgcagtggcacagtctcggctcactgcaacctctgcctcccaggttcaagtgattttcctgcctcagcctcccaagtagctgggattacaggtgcctgccaccacaaccagctaattttcgtatttgtagtagagatggggtttcactgtgttggccaggctggtctcgaattcctgacctcgtgatctgcccgccatggcctcccacagtgctgggattacaagtgtgagccactgcgcctggccgcctaacctgattcttaatcatcactattagcaccattttacggtttgatccctcaatgactttctttgaccagggagcgctgacccagctgatccagctctatcatcgcttccaccgggtgctgtcccagccgcagctccgagccctccctgcccgggctgagctcatcaacattcaccaccttatggtggagctcaagaagcataagcccaacttctgatgtgccagaaaccgccctgagatctgccggtcatctccatggacttctgcaccccattccatacccttcttcacctggggtaccccttccagttttccccttgcttcccaggcccttgacatggcttacctgccttcactcccagcaccttgcccaacaggataagctggatccccttggccttctgaatatcccagtgtcttcaggtttcccaagaccacttccctgtgggcttccaaaatggcctttatcatttctccagtctgtcaccctcctttcctgctcccatacacccaaggcttgtttcttcccctgtaaaaaccactgcctcaatctctggttcactcaactagtcaccatgtcctgaggcatgaagcctcctcagctcttggaattgctggcaaggggtgactgcctctgagtcattgtgtttttcaaagtgatttcttttctgtagctttttgacctaagatctcagcaatttgaacactaacctctcccctcctggctcaagaattactccgaagtcagtctgcagaaaataaatatttagtatgacatgacacttatcccatttcctttctccttcctcctgaaggtttttcaggtggccccatctctagaatgacctttcctttctgactacttcctgggcttacatcccttctggtgggtgtaacctctaacctctagctactcccaggccattggaggaaggtggcccctggctaaaatagcagatgctgcaagggatgagaaccaggccgcgcagccccagacactgtcccaagggctcccttgggacaagcccaagctgactctggaatccctcctccgtagcagctccatagctcctctggttgtggctgtgacagccccttgctcgggggctgcagcacccaacccacacccacaccgttggggcagggctagaccaggcgaaattcgtaccatccgtttgtttgacttggagccttcctgctgtcctcgcctatacgcctcgtttgaacttaggctctagactggagagacacggggacccctttaaggcctaagaagagaggcgcagttaaggaaaaatgttacgtttcctgtgtcctccacccctacggcctaaacattccctccccaggtccctggagagtggtggaaagcggttcctccccgcttaggcccttcggatgcaggtctagcccgtcggcaacgggaggtgtcctgagtgggtctgtgactgccgagcacacccgccgcggagcggaggctgctgcttcctgaggctgagagtggatccggctccgggctctcctaattggcggacgctggggggcggcgtggtaaggcagaacggagcggcatctcagctctcgccttttcagggttccgccccatatccgagagccgctctctaattggcttgggaaaccgtatctcagcgctttggcctagcgactttgaacgtgtttgcgcctgagaccgaagtgcagaagagggcgagcgcaggaggagaggcttggtgaatcagcgattcctgattggccaggcgtgccttgagggcggggccagaactgcgtccttaactgacctcgcccttgcccagatcaccgcctccgcgttgctccgggtttaccccgcctgactcgctgcgctatgcgttccctcacgcctgccggatgccaggcggcgatgtgccaggctctgaggggccgcgagctcaccccagacgccggccccgggaatcctctgctcctccactttcccttcccgctactggtttcttgcccacccactcccaggtgtcactcttgggacatccagatgttctgacatttgacctgactccagcctcagcgcgaggacgggagaagggccaagggtatgagattttggacaggagaggcattggctactctgacaaagagcgtggattcccaaagaaagggtccccagataccccgcaggggagactgtcgagacaggcgactttagccaactaatgctcgcacgcgaggagggctgtgcaggcaggcaccacggttcttcacagccttccttctttccttcccctgtccgtcgcagaacccaaatcctcagagctgatcgagaagcgcgtgttgttgcagaagtcactgaggggcagacctggaatgtgagccgtggggcgaagggacagctctggagactcgtgattccggaacccggtgggcattcaagtgatactggagcacgcagttttcggggacattaagctcgccccaggggtccacaggaaccctctgacccaacggtctctctggacaggatgatccagaacagacggtgaagagacaaaacagcgtgaaagggagcggcgatacctaaactgacctccggagggcagcatgatcaagggaaggagctgttctcgctccgctcaaacccccgcgcagcctctgcagcttactcctgcttcggaaggcggcgaggttccacccccaccaccccggtccccgccgccctcttcgcgctgaagctgcggagggtctttttcttcagcccccaaatcctctgctctgtggcttaagattcccaggcttaaacccataactgctagggtcatcactcctcaaacttctccctcacttgttttgctgatgacggacacaggggcctcacagactcagaagcatctggagtcatttcgaaggacagaactgtggcaatatcctctttccccctcctaatctctgagaacagtgtctgtatatggcacgggggtccctagtgtcatatagaaggactcggagatgtcctcatgggctgtaacggcccccggtgctattcagggatctctgttctttaaagaaatatcatttccccttatttctctacctttgtgccctcacctcgccctgaattgtttcttaaactgggctttctgggaaacgtttacttcgtgttcaacagcaaagtctcggcataaggcgagggcggcagggggtggggggcgggcgtttgctcgccttggcattaatttcagagctgctgaacgtggacaaaagagagggaacatcctcctttcttccattcctgtaaatatggacccacccaccctccacttaagatgggtggcttttctgattttaagatatcaagcagcctggcgaggtggctgacacctgggatcccagaactttgggaagccgaggcgggaagatcccttaaggccaggagttctagaccagcctggccaatatggcgaaaacccatctctactaaaaatacaaaaattagcctggcgtggtggtgcctgcctataatcccagctactagggtggctgagtcaggagagtcgcttgaacctgggaggcggaggttgtaatgagccgagatggccactgcactccagcctgggtgacagtgagactctgtctcaaaaataataataataataataataaaaagatataaattggctgggctttaaaataaagtagtcagaggcccagggtggtgactcatgcctgtaatccttttggaaggcagaggccagaggatcttgagcacaggagtttgagactagcctgggcaacatagggagacccatctctaccaaaaaaaaaaaaaaaaaaaagcaaaaagccaagcatggtggcccatgcctgtggtcccagctagttgtgaggctgaggcaggaggattgcttgagaacaggagttagagattgcagtgagctatgattgcactactgcactccagcctgggtgacagagtgagaccctatctctttttaaaaaattaaaaaacaaagtagatttagtgtgaagattcaaggaaagaaagagaaagcaagacaaaactttaaggagaggagaatcgaaattggctgacttcaccctggacataatggacacagctattcatgttacgtactagcgttaacaaagtgttttcagaagcagttgtcattaaaccaacacatttattgaagctcttctctgtgttctgtgataggcactgcggattcagcagagactaaatcaaagtccttgatctcggagagcacatatttcatctaattgaattcacataccagccttatgaggaggtgttcttcctgtttcaggtaatgagcttgaatttcaaataatgagttcaaaaccatataggcactcagtggctgaaccttctgtgcccacattcagcttttgttctatgactccaggctgggagtcatggagctgaggaggcagccggttgtagagaagcaacagattaataagtaatagtaaattattgaaaaaatatttacagaactgaaggaaaagctgaacaggcggtctttggaaaggacagaagccaggatagttccagcatccaggcagcagacaccaatggtcagtgcttctactttggatcaaaaagagagaactccaataggcttagggtgggtcaggtgctcacactcatctgaggaaggggagggtcccttgaggaatagtaccatagactacccaatgttggaggataatggccccaacgcaaaaatgggatcttgccaccagacaaatgggatatagatgccaggctgcaaaatccaacaaatgtacacgtgcccaaatcccgactccatcactaatagccatgcaatcctctgactatcaattagattaattgacactcttttttctctcgctactgtggtacatgcagtagttacctttattcttcactttgttttctcataagactaacaagcagttttttaaaataaagaaataaaagcaaaattgcttattgtcttagtctattcattggcaaactagtaacattaaataaactccagacagagccactggagaagaatcaagtacaacttagtagaaataacaccgtatgtgtgaaggtgagtgtgtgtgtgtgtgtgtgtgtgtgtgtgtgtgtgtgtgtgatggaaaatactttgtcctgcccaaactgtaagtcatgaccatgcccctctcagcagaagtttatcttcatctcatttggaagtttataagaacacagcttaaaaaaaacctgtgattttggagcaaggtcaagcctttgctattgcttccacattttgttttgttttatttatttatttacttttcagttttttggagttttctttatttgtttgtgtttttgagacagggtctcgctctgttgcccaggctggagtgcagaggcaccatctcggctcactgcaacctctgcctcccgggttcaagcgattcccctggcttagcctactgagtagctgggattactggcacgcaccaccacatccagctaatttttgtattttcagtagacactgaaatacatgtttgccatgttggctgggctggtctcaaactcctggcctcatgtgatctgcctgcctcggcctcccaaagtgctgggattacaggcatgagccaccacgcctgcctagtttttagttttttgagacagagtcttgctcagttacccaggctggaatgcagtggcatgatcacagtttactgcagcctcgacctcatggatttaagcaatcctcccacctcagcctctgggatagctgggactacagtcatgtgccaccacacctggctaatttttgtattttttggtaaacgaggtttcaccatgttgcccaggctggtcttgaactcctgagctcaagtgatctcctacctcggcctcccaaagtgttggaattacaggcatgagccactgccccagctgttttattaatttagttaaaaatatatgaaaagaggccctgcgcggtggctcacgcctgtaatcccagcactttgggaggctgaggtgggcggatcacctgcggttttggagttcaagaccagcctgaccaacatggagaaaccccatctctactaaaaatataaaaaatcagctaggcatggtggcacatacctgtaatctcagctactcgagaggctgaggcaggagaattgccctaacccagaggaggttgtggtgagccaagattgcgccattgcactccagcctgggcaacaagagcgaaactctgtctcaaaaaaaaaagtatatatatatatatatatatatgaaaaggctgggtgcggtggctcacgcctgtaatcctaacactttgggaggctgaggcaggtggatcacctgaggtcaggaatttgagaccagcctggccaacatggcaaaaccgtgtctctactaaaaataaaaaaattagccaggcatggtggcaggcgcctgtaatcccaactacttgggaggctgaggcaggtggatcacctgaggtcaggaatttgagaccagcctggccaacatggcaaaaccctgtccctactaaaaatacaaaaattagccaggcatggtggcaggtgcctgtaatcccaactacttgggaggctgaggcaggagaattgcttgaactcagggggcagaagttggcagtgagccaagatcacaccatttcactccagtctgggcaaaagagcaagattctgtctcaaaaaaaaaaaaaaaaaaaaaaaaaaaaatatatatatatatatatatatatatatatatatatatatatatatatatatacatacatacacacacacatacacacatacaggcactagccaggcgtggtggctcatgcctgtaatcccagcactttgggaggccgaggcgggcagatcacaaaacaggcagggcatggtggtccacgcctgtaatcctagcactttgggaggctgaggtgggcagatcacttgagcacaggagttcgagactagcctgagcaacatggcgaaactctgtctctaccaaaaatacaaaaattagctgggtgtggtggtgcatgccagtaatcccagctactccggaggctgagaggcaggaggattgcttggggaggcagaggttgcagtgagccgagatggcaccactagactccagcccaggtgacagagggagaccctgtctcaaaaaaaaaaaaaaaaaagaaagaaaagaagaaaagaaaaacaaatcacctgccttatgaagaaacagtttgtaatagatataagacacagaatgtgcagcccctggtatggaataagtccttagaaaatgttagtttcctctctgtccctgtgtgacatgaagggaattccagggccagtgccacttcctggtggtttccctcacttaagtgctgtgtaccccattgcaaaagcatgtcttccaggttcctgataccacatcccatgtcctctgcctttggggctctcataggattaagtgtcattttccctatgaactagaaaagaatcttctgaacgtctgccttgggactgatgatatagctgagctgaaaggacaattagttttcctccccagaccaacccaaccttcaaggctggggactcatttgatgctcccattcaaacatttggagaaaattgtctctgagtaccaggtctttttagcatattttctccaaatgttacatccttaggggctttgaatagtagaagagccttcaacattggttcagatggaagcatgcaggcatgtggagagttgccactccctgatccctgaaacaaggcttaggatggaaaagagcctcctggggaggttggctctgaatgtaaagctgggagaggcacctcctgtcaacctaagggcttgttggtaggggcaacatgttgacctggcttccttcccactctgagacatcactggctctcatgcagagctattttccttctttctttctttctttctttctttcttctttctttctttctttctttctttctttctttctttctttctttctttctttctttcttttcttttcttttctttctttctttttttttttgagacggagtttcactcttgttgcccaggctagagtgcaacggcacgatctcagctcaccacaactgccacctccctgggccaagcaattctcgtgcctcagcctcccgagtagctgggattacaggcatgtgccaccatgcccagctaattttgtatttttagtagagatggggtttctccttgttggtcaggctcgtctcaaactcccaacctcaggtgatccacctgccttggcctcccaaagtgctgggattacaggagtaagctgccgtgcctggcctttcattcttttctttccttctttccttctttccttccttccttctttccttctttccttccttccttcctccctccttccctccctccctcctttcttctttcttctctttctttctccttccttccttctctctctcttttcttttctttcttctttctgggtttcgctctattgccgaggctggagtgcagtgagtggcacgatcacggctcactgtagcctcaacttcctaggctccagcgatcctcctacctcagcctccttagtagctgggattgcaggcatgagccactgtgcccagtgtaatgcagggctcttcatccaggatcccaaagggcaaacaaaaataggagaaaattacatttcattttcactaatctctaactgaaatttagcattttctccaatgataaatgtaggcaataaacctacaccggtatttgtagtacatgtgactgttgttaatagaatttacacatttgtgtcatcttccagtagttgcagaaatttcaaaatgtcatgtatgctcacggttactttgtagttagtggtagtggttagactcaccactagatcttcttcttacacattttgtttcattttgacaactgtacttcaatatgattgttttcctttgtaatcccatttattttactttatgcagttgaagattattctgacactccacagacttcaccagacttccaaaacctgtcctagacgtaaaaagttaagaaaccttaaatctctaatgatgctgtcatttcctgttttgtattatcttacaaccagggtaattaggaatgccttcttcttcttctttttttttttttttttttgagacggagtcttgccctgtcacccaggctggagtgcagtggcacaatcttggctcactgcaacctctgcctcccgggttcaagtgattctcctgcctcagcctcccaagtaggtgggattgcaggcatccaccaccatgcccagctatttttttgtatatttagtagagatggggtttcaccatgttggccaggctggtcttgaactcctgacatcaggtgatccacccacctcagcctcccaaagtgctgggattacaggcatgagccaccgtgcctggcttgctttcttcttgtttaaactaaaggtcttcttgtaaaattcctgcttatattttcttcattattactcaaattcaattattcatttatttgccaaatatttagaaaacacttctcttgtacaaaaccatgctgcataaaactgttattatgcgaaaaataatccttactcgccatgccctctaccccgaccccccaaccccatttacaatctggtagagctggactgtttacccacataggacacacaggctgcagcttaccatgggcagggagtggggatggttgttccaggtgcaggtaataagaaggtgtattgtctgtggagaatttaaaaataattctaaaaccaactttttttttttttttttttttgagatggagtctgactctttggcccaggctggagtgcagtggcaccatcttggctcactgcagcctctgcctcccgggttccagtgattctcctgcctcagcctcccaggtagctggcattacaggcacacaccaccacgccaggctaatttttgtgtttttagtagagacggggttttaccacgttggccaggctggtcttgaactcctgacctcaggtgatctgcccacctcaccctcccaaagtgctaggattacaggtgtgagccactgcgcccagcctgaagccaactttttattgatactgcgcaccagcaattataaaaaaatgttagtgacaaaataccccttgcagtgtagcctcctccccccactccaccctgcttggtatgccactgcacacaggggagtttaaccatttccagtttacacacacacacacagacacacacacacacacacacacacacacacacacaaacacacagagcctttgagacttccttttctcctcaactcccccagcccagttctcatgtcactctactcagggtaggatctgcagcactgctgggccagtatccccaaaggtgagccctgtaagctccttggcctctggatggtctcattaatagtccaggatgagccctgcaatcttggcatctgtaggcaggtctcagctcttcttcacacccctgggcttgaaccccttggcaactgcgtttgacagtacatgattcccattcttcatctccagattttccatccttcaagaaaacactggatttcctggtcaagccaaggcttaatgaactggcctggcagcttctctggtcctcaagtttccttttcttgccttactttctgaagacttctagtagttttacttggaagtgtcactgtctccatttttattcctgagttttctgcagttcttttttctgtggccccacggactcatccagagcctgcagggagctgacacctcatctgagctgctagaattgaggtgagaaaggaggttcctaagtgcagaaatgtgttttctcctctccagccaccactgaaggctttggatgtgattgagtcagacctctcctgcacagtctccctttggattacctcaagataaaccaatttgtgactttaattacatccgcgaagtcccttcccagctgcacctaagctaaagtgtgattgaatgaatgaaagagggtgtgctggcgatcagctagggaagtgattccaagggtccctagactccacattaccaactgcaaaatgtgacgttcttacgttctgacaagcgaactctcctcccaaattcattgcctgagttgatgcaaatgaattcagccacgccttttgggtttggcatctgagaagtgtctgtgacttgggaagagtggtcagaatggcgataatgggaagagaccacatttcagctggtggagagtgaaaggacagccaacatcttgccctttctgaagcatacacacattggagattgaaagtgtctgttggcctcacaggcactgaggaaagcctcccatccccttagattttcctcctttttcctgaggtcccctgaacctaatagtctgtaaccttattccatgtctgggtcaacaaaccaccttccaaggaggacttgaatataataatccgtttcctctgcattcctccagaacacagagtccacaccatccatgagtctgtcaaacaaaacattcctcattggtctcaatgttgagcactgaaagttctctgagctcatttagcaccaactcatcatccttaaaatccacaggatgggccgggtgcagtgacccatgcctgtgactacaacaccttgggaggccaaggggcagatctcttgagcccaggagttcaagaccagcctgggaaacatggtgagacaccttctctacaaaaaaaaatttttttttttgagacggaatctcactctgtcgcccaggctggagtgcagtggtgcaatctcggctcgctgcaacctccgcctccagggttcaagcgactctctgcctcagctcccgagtagctgggattgcaggcatccaccaccatgcccagctaatttttgtatttttagtagagacagggttttaccacgttggccaggctggtctcgaactcctgacctcgtgatctacccgcctcagcctcccaaagtgctgggattacaggtgtgagccaccatgcccggcctacaaaaatttttttaaaaaaattagccaggaatggtggtgtacacctgtggtcccagctacttgggaagctgagataggaggattccttgagcccaggaggttgaggctgcacagtgagctgtgatcacgccactgtactccagcctgagcaatagtgagaccttatctcagaaaacaaacaaaacaaccaaaaatatccataggatgaaacatatgtttgattatgacaacttgcttatggtaccttctggatggcccagctcccttgcagatccccagaccaacagcttttgttgtagtgacagcttcttcttgtaacctctttttttttttttttgagatggaatttcactcttgttgcccaggctggagtgcaatgacatgatctcggctcactgcagcctctgcctccagggttcaagcgattctgctgcctcagcctcctgagtagctggaactacaggtatgcaccaccatgcccggctaatgtttgtatttttaatagggacggtgttttgccatgttggccaggctggtctcgaactcctggcctcaggtgatccacctgcctcagcttcccaaagtgcttggattacaggcgtgagccactgcacctggcctacagcctctaatttcattttcatccaaaagctttaatggaaggcccacagattccctcttcaagtaacagcaaagcgcatctcctttaagatttctttgatcatcttcataaagcttgactttgaagtcttctgaggatttctcctggaggcaaaccagatatatagacatttgtatttctgtcttcttcaacatgaaattatcctcattccatttttcttttatctcccctgtttttaggatcagtgggtttggggggttttctttgcagaaattctgcattcctagcattgacattttgtacatttgcaggagagctagatgccccaccattagaaaagccacacttggctgggtatgtagcaatgaaaccttcagtggccttgggaaaccacgccttcttgtccagatcccactcattgtgggagtcagttggctgctgccctaaataacccgtttgtcactgagatcttcttgtctgttgcagtccttagtgtctccatataatttatgcattcacaactgctcatcaagtcatcattatcatccacttcatgtttcctacctagccaggggcagggttcaggtaatcaaagaggctgctctgctgggcctgcaaggctccaccaagtgtgtctgaggtgacagcccaagcactgttataaatatttgcgcactgttgtaaacatttgcattagtcccttctcgcactgctataaagaaatacctgaaactgggtaacttataaagaaaagaggttgccgggcgcggtggctcatgcctgtagtcccagcactttgggtggctgaggcaggtggatcacttgaggtcaggagtttgagaccagcctggccaatatggtgaaaccccgtttctactaagaatacaaaaattagccgggcatggtggccagcacctgtaatcccagctacttgggaggctgaagcaggagaatcacttgatccggggaggcagaggttgcagtgagccaagatcatgccactgcactccagcctgggtgacagagcaagactccgtctcggaaaaaaagaaaagaaaagatgtttgctgggcacagtggctcatgcttgtaatcctagcagcactttgggaggctgaggccggagggttaatcacttgagctcaagagtttgagaccagcctgggcaacatggcaaaaacccgtctctataaaaaattcaaaacttagctgggtatggtggcatgtgcttgtggttctagctacttggggagctgaggtgggagaattgcttcagaccagaaggtagaggctacagtgagctgtgattgcgccactgcctgggcgacagagcaagaccctatctcaaaaaaaaaaagaggtttaattggctcatggttctgtgggctctacaggaagcatgatgctggcatctgcttgccttctggggaggtctccagaaacttacaattatggcagaaggcaaaggaggagcgagccttctcacatggcaggagccgagagagagggaggaggtgcggcacacttttaaacaaccagagcttgccagaactctctatcatgagaacagcaccaggaggatggtgctaaaccattcgtgagaatccactcccataatccaaccacctctcaccaggctccacctccaacactgggactacatgagatttggtggggacacagatccaaaccatatcaacatttaacacattgcatctttgaattgtcacagcaaccctgtgaggtgggtactagtgttatccccattttacagagaggaaattgatgcccagagaccttgccaaggttatagacaggacagttagtgcatgacaaagctggaattggaatgcagccagttctgctttagaagccactttcttgcctgcctttctttctttatttctttatcttttctttctttctctctctctccttccttccttccttccttttctttcttttcttttcttttttttttttttttttgagacagggtctggctttgttgctcaggctggagtgtggtggcaccatcttggctcactgcaacctccaccttctgggctcaagtcatcctcctacctaagcttcccaagtagctgggattacaggtgcataccaccacacctggctaatgtttgtattttttttagtagagatggggtttcaccatgtttgctaggctggtcttgtactcctggcctcccaaagtgctgggattagaggcgtgagctattgcaccccgcctgaagctacattctttttttttttttttttttaatgagacgagtcttgctctgtcccccaggctggagtggtgcagtggcgtgatcttggctcactggaacctccgcctcccgggttcaagcaattctcctgcctcagcctctcgagtagctgggattacaggcacctgccaccactcctggctgttttttccatttttaatagagacggggcttcaccatattggccatgctggtctcaaactcctgacctcaagcgatccgcctgccttggccttccaaagtgctgggatacaggcataagccatcgtgcctggccaaggctacattcttaaacgccactttttacagcttccaagaaactaactgggtgaaatccagcctgcctcctgtccttgtttttatcatttcatagtgaaaacataaactcccaagcccacctactctattgcactgtgtctccatgacactctgtaccttctggctctgactcagccctccactttccccctccctaacctgcctgtgccctctaataaactgtagcccccctcatacccttagcttctccttggagcattccctccacttcctggcgttaagtgaaatttaactctcccttgagggcactacctcctttgcaggcttctgaaatgagagctgttgattttcttacagctgagggtgctctcctggcttcagattgctgcctccggactactcctcccccagcccccaactctagtttaaaataacccgcggccttccaggcactgcaattaggctaaactttcctcttcctttctcactgctactgtctgccagacccctccacctcactcaggggaacgctggcccagctgaccgtctgtctttgcattgagtcctccctctgggagacttcattgtccacctggaggatccattccctacttgacctctcaataacctggccacttaatccccaagaatcttctcctctccaactcggccacacattctcattgtcataccctggaccttatcacctgaaagagcccctccttgagaatcagtcaagcagcctgctcttggcccccacctcctaaccgtctgtccatcagtctgtctgccctcctcgcaaccaattttcagtctcctaggacctttggatcaatgatgccttccgtcttccagcttatgagcccctccctattcagctcaactccacaacccttcatttcaacacctctctggacaatgtcgccaactccccttcccctctgtattttcatcctttgtacccaagtggcaaaaccccaaccctggatgagcccagtgatctgttttcttcaggattttactctgatagccaagaactgttggtggtggtgggggaaatcacacaatcaggaaatggttcccactcctcactcatgatcatgaactctaaggaggctcccagaattgtttagtggcccaaccacatcactctggtcaatggctacgttaaacctccctgatccttgaacctctgaggctcttctgtctctcacctcatgctcacttgatgacctccctcttatgtcacagagaaaatgaaagtcatcagaggtgagctccctcaaccatctgtcaccagatccacacccctccaagaggaacacaatgcatgcagcgcatgtaccttgatttgatcctgatttgaaatatatgcataggctggatgcagtggctcacgcctgtaattccagcactttgcttgagctcaggagttcaaggcaggtggattacttaagctcaggagttcaagacccacctgggcaacagagaaaaaccctgtctctacaaaaaaaaaaaaatacaaaaattagtcaggtgtggtgggtcacacctaaatagtagttccagctacttaggaggctgaggtgggaggatcacttgagcctgggaggtggaggttgtagtgagctgtgattgcactgctgcactctagcctgggtgacagtgagacccagtcaaaaaagaaagaaggaaaggaaggaaggaaagggaggagagagagaaaggaaggaaggaaggaaggaaaaacagaagaaaggaaggaaagaaattaaaaagaaagatatatatatatataaatatatatataaaagacattttgaggctaactggggaatatttgagtatattctgctatcagatgaagtgaagaaatgatcgttattttctaattttgtttttttttttgagactgagtcttgctctgtcaccaggctggaggtcagtggcgcaatctcagctcactgcaacctgtccctactgggttcaagcaattctcctgcctcagcctcccaagtagctgggattataggtatgcaccaccacgcccggctaatttttgtatttttagtagagacggggtttcaccatattggccaggatggtctcaatctgttgaccttgtgatcctcctgcctcagcctcccaaaatgctgggattacaggtgtgagccaccgtgcctggccatgatcattattttcttagctgtgattacgtattgtggttatggaatctccttatttttgggagatgcatgctgaggtacttaggtgtgaagcacatgctttcctatattttcatatggtccaagaatgttttagtagtcactcaacaatttctatgtttaaaaaaaaaaaaaagggctgggcacagtggctcatgcccgtaatcaatccgagcactttgggaggctgaggcaggcggatcacctgaggtcggaagttcaagaccagcctgaccaacatggagaaaccccatctatagtaaaaatacaaaattagcttggcgtggtggcgcatgcctgtaatcacagctactcgggagtctgaggcaggagaattgcttgaacccagtaggcggaggttgcgtacagccgagatcgcgccattgcactccagcttgggcaacaagagcgaaattccatctcaaaacaaaaaactaaaaacaagcccacaaagacgtatataggccttggcactgtggctcatgtctgtaatcccagaactttgggaggccaaagcaagaggatcacctgagtccagtttgagaccagctgggcaacaaggcgataccttgtctctacaaaaaatttaaaaattagccgggcatggtgccatgcacctgtggccccagttacttgagaggctgaggtgggaggatcgcttgagcccaaaagtttgaagctgagatgagctatgatcaggtcactgcactccaccctgtgcgatgaagtgagaccctgtcttggaaaaaaaaaaaaagaaccatatatatatgtgtgtgtatatatatatgtttgtatatatataatatataaatatttgtatatatgtataaataacaattgttaaatctaagtaaaatgatcgacatacaaacaatattgtattatattttcaagttttctgtatgtttgaaatttttcataataaaaaaactgggcaaaattctctcacttccactcactgctcgaccctttcaggctggtgtttatctctaccttgctgctactcaaatgtttttttgcaatgaactttatgtcattaagtccattgtttcgttttcttcttacttgaactgccactagtagctgacgttgctggccagttctttcttgaaacactgtcgttttttggcctccaggatttggcctcttctggcttcctcccatccctccatcggctgctcttccacccctcagccggctcactccgcctttgactgaaatcacctggtcatattggagttcatcaagagaggtcggtcctggaccctcctcttcttatgctaccctttccttctaggggatcacacctgtcctgtggcttcagttcccatcagctggttactctcaaatttatctcttgcctcaaatctccagatttgaccaccttcctgcctggcatccctacttggatgtcccaaaggtattactaacttagcttttttttttttttttttgggagacagagtttcgctcttgttggccaggctggggtgcagtggcaagaccttggctcactgcaacctccgcctcctgggttcaagtgattctcctacctcagcctccggagtagctgggattacaggcatccgccacgacacccggcaaactttttgtattttcagtagagatggggtttcaccatgttggccaggctggtctcaaactcctgacctcaggtgatccacctgcctcagcttcccaaagtgctgggattacaggcatgagccacggtgcccagcttaacttagcatatttgaaacagaagtcatgatcttcccacagaagcagtatcatcctcagatctgaaccacaggggccctgccctggcccacatactttagggggattcacctatcacaaacaacttatcacacacatttattacatgacacctgggcacctctgtcactgatctgctgtcagtgctggaacagcacaacctgaaatcttacacatctgctcttgtaatcaacgccgttcaagccctggggaatggtgacttctgtaatctcttgccctgtatcctggaaacaaaagatgactatagccaattctgtgaaaatctgaggttgtgcctctgcctggattctgaagcatgtactgctttggagtgtagggaggatttaagcagtggaaacatgtaagtgaaatcactttaaccagcttgttaaatattacctctcaaatagcatgacaaagtatcatttcccaatagtgttgagtgtggtttttcttgtttctctttgttttagaggtatggatgtatgttgtgctgcatttacaacagttgcaaaggatagctgaggaacatgttgctgtattaaacaatccttattactgttaaatttatatgcatgtggtgggaggggtggtgagggataaaaaactacacatttacagtgtacactcctcctgtgatgggggcaccaaaatctcagaaatcaccactaaataacttattcatgtaactgaacaccacctgttccccaaaagcctattgaaaaaaaaattgaatacatgtaggtctagatataactcttgtgaagtatgatattgattctttatgttgtaatagatggacattgaatacgagattatgaatacaatcatcctcagtattcaccagggattgattccaaaacccccactgatgttcaaaccccttgtataacatggcatagtatttgcatataacctatgcacattcttccacatactttaaatcatccctaggttatctatggtgcctatcgcaatgtaaatgctgtataaatatatgttatactgtattttttatttgtattattttttctttctttccttccttccttcccttcccttccttccttccttccttccttccttccttcctttctttctttctgacagtcttgctgtgtcacccaggctggagtgctatggcaggatatcagctcactgcaaactctgcctcccaggttcacgccattctcctacctcagccttccaagtagctgggattacaggtgcccgccaccatgcccggctaatttttttgcatttttagtagagatggggtttcaccgcgttagccaggatggtcttgatctcctgactttgtgatccacccgccttgacctcccaaagtgctgggattacaggcgtgagccaccgcgcccggactggccatatatattttaaaaatatttttattcttcagttggttgaatccataggtgtggacctacagatacaaaatgccaactgtagtttaatttttataaaactatctgtcttagtccattcagactgctataacaaaataccttacactgagcaatttataaataataaacactgcttacagttctggaggctagaatgtccaagcaggtggtgccagcagatttggtagccagtgagggcacactttgcttcatagatggcgccttcttgctgcatcttcacgggtggaaggggcaaacaagctcctttgggcctcttttttttaagacagggtctcgctgtgtcacctaggctagagtgcagtggcgtgatctcggtttactgcaagctctgcctccagggttcaagtgattctcttgcctcagcctcctgaatagctgggattacaaatgcctgccaccatgactggctaatttttgtgtttttagtagagatggggtttctccatgttgaccaggcttgtcttgaactaccgacctcaaatgattagcccgcctgagcctcccaaagtgctaggattacaggcatgagccaccgcacccagcctgggcttcttttataacggcattaatcccattcatgaggacaggcccctcatgacctaatcaccttctaaaggccccatctcttaatatcaccacattggggattaggtttcaatttatgaattttaggggggcacaaacatttatgccatagcaatacataataaaaattaataaatatgtatatatttttttgagacagggtctcactctgtcaccaggctggagtgcagtggcacaatcatggctcactacagcctcaacctcccaggctcaggcaatccttctacctcagcctccagagtagctgaggccacatatgtgtgccaccatgcccggctaagttttctattttttgtagagatggcatttcaccatgttgcccaggggggtctcaaaacactgggctcaagcagtctgcccaccttgacctcccaaaacactgggatttcaggtgtgagctactgggcccagcccaaaatttcaaaaaaaagcaggcaaaaagtcagctttatctaaatgtgtttgatttaaaatattgatgtgtattcattatcatttatattttgtccagatgccgtgtctcatggctgagattccagcactttgggaggctgaggtgggaggatcactcgagctcagaaatttgagaccagcctggacaacatagtgagaccatctctctacaaaaactaaaaaaattagctgggtgcgatggctcatgcctgtaatcccagcactttgggaggccaaggcgggtggatcacttgaggtcaggagttcgagaccagcctggccaacatggttaaaccccacgtctactaaaaatacaaaaaattagcagaagaatgcttgaacccatatgtatacatgtatatatacatatgtgtatgtgtatgtttatatatatatgtatatgatacaaaattagccaggtgtggcagcacacacctgtagtcccagctatttgggaggctgagatgggaggatcacttggacctgggaggtcaaagctgcagtgaactgggatcatggcactgtactccagcctgggtgacaacgtgagatcctgccaaaaaaaaaaaggaaaaataaaaagagtgattttacagaaaaattgagattttcaatttatcacggcagtgccagctctgaattcctatgcggcaacgctggaccaggagctgctgcccctctaggaggggagggtgccctccagcctgttatagccttcccttggcacttggcacattacttgcctggcccttacaggaatttgaggtgcaaattgatctagatcattgaaggaagagtctgggtcttctcagttttggggcactttcgttgccaagtaaggttcttatgagatttttagaagttgagattaaaaaagagtttatatgcagctggaggtgatacctaacaacaaccataataacagcatcaacaacaaaaacagtagcctagagcagttactctaagccaggtaagtgttctaaagcacttcacacaacttatttactctcacgacaatgctattaggtagcactttgaatacacacattttgtaggtgaggaaattggggcatgtagctgttaagatttatatcagccgggcgtggtcactcatgcctgttcccagcactttgggaggccaaagcgggcggatcacgaggtcaggagtttgagaccagcctgaccaacatggtgaaaccccgtctctactaaaaatacaaaaattagctggacatggtggcggctgcctgtgatcccagctattcaggaggctgaggcaggagaatcgctttaatctgggaggcggaggttgcagtgagctgagatcatgccactgcgctccagcctgagtgacagtgagactctgtctcaaaaaaaaaaaaaaaaaaaagatttataccaatttataccgtccaataggaggcgtgagactcaacacagggtggaggagagaataggtcaggaaatcacatctctccacctgcacagccaccatcattgtacccttctaaagagactggcaacaactttcattcagcactgtcccattgattggctcatttcatgatcacagcaacccagttaagcaaatcctcacctttgcatcgcctgctttgtatcacttttctttaaaattaaaaataaaaaaaatggattacaaattcggttaacgcccatcccatttctctccttccttgtagtaattatcattctaaagttgttgggaattattcccatgcgtactttgcacttttaaaaaatgtttgctttcctaaacatataccactgttttgtgtattttaaaactcacacaaataatatcatacaacatccatttttgcaagtttcttactcattacattgctaaatatgtctataattcatttaactgctgtatagtatcccattataagaacaaaccatactttatttatccattttacttaacaacagttagcttatttccacttcttcttgacaattaactgatactgcaatgaccatttttatcctcgcctttttgtgcatgtgtagaaatgtttttctaaggaatagatctagaagcaagattgctgggtcatagggaatattcattttcagcattaactggtgccaaaattctcttcaaatttgttgtatctgtttacacttcaacacaaaatatatgcattcctattgtcccacatttttgtcaacagtgttagaataattttaattttggtgattttgtgggtgtaaaatggtaactcgcgctgtgatttgcatttctacattactggttaaattgagcatcttttcttatgtttattgaccattaggtttctcttttgtgactcacctattcacagcttttgctcatattctcttgcgttgtccttttcatatggatctgcaacttctttatgaatcctggatataatcctttgttgtttatgtagattgtgaatattctctaagtctgtggcttgtcttttactttacgtgatttcttttttttttttttttgagacggaatcctgctctgtcgcccaggctggagtgcagtggcacaatctcggctcgctgcaagctccgcctcatgggttcacgccattctcctacctaagcctcccgagtagctgggactacaggcacccactaccacgtccagctaatttttttgtatttttggtagagacggggtttcaccatgttggccaggatggtctcaatctcctgaccttgtgatccacccgccttggcctcccaaagtgctgggattacaggcttgagccatcgcgcccggcctatgtgatttcttataaaagcttaaaaatgttttaacgtggtcatatgtatcaatattttcctttattgcttgtgcatttatttatagtcgacaaataaaaattgtatctatggtgtaacaacatgatgtgtgtatatatatgtgtgtgtgtgtgtgtgtaatggctagagtaattactgtatctgttatctcacatactatcattttaaacgtgatatttgaaatgtactctcctagcaattatttttatttttattttttttttgagacaggatcttgctctgtcgcccaggctagagtgcagtggcaagattatagctcactgcagcctggacctcccaggctcaagtgattttctcttctcagcctcccaagtagctgggactactggcacgtgccaccacatcctgctaattttttcaattttttgtagagacagggtctcactatgttacctaggctagtcttgaattcttggggtcaagcaatcaagcaatcggcatcccgaaatgctaggattacaggcatgagccactgcgcccagctaatttttctattttttttagagatggggtctcaccatgttgcccaagctggtgtcgaactggtctcaagcgatccacccacctcagcctcccaaagtgctgggtttacagacataagccactgcagctggccagcaattttcaagtatacaatacattgttattaactctagtcatcatgttatacaatagatctcctgaatttattctacctaactgaaattttgtatcctttgactaaagctctccaatccctgcccacacccgccccccgccccctagcccctgtgcagccaccattctattctctgcttctgcaattttattttatttatttatttttttaaatttattttgagacagggtctcactctgttgtctgggttggagtccagaggtctgatcacagctcactgcagcctcaaactcttggattcaagagatcctcctgcctcagcctcccgagtagttgggactacaggcacgtgccaccatgcctggctgagtttgactctttaggttccacatgtaaatgaaatcatggtatttgtctttctgtgcctggtttatttcatttagcataatatcctctagttttatccatgttgtcacagatgacagaatttccttctttttaaagactgaatagtattccattgtgtatatgttccataatctctctatttattcattgatggacacttaggttgtttccatgtcttggctattgtgaataatggtgcagtgaacttgggaatacagatatctcttcaacatactgagttcatttctttcgaaatatacctgataatggaactactggatcacatggtagttttaattttttgaggaagctccattctgttttccaaaataggtgtacaaatttacattcctaccaacagtttgtgagggttcctttttctccacatccttgccaccacttgtaatctcttggtttatttttattttttttatcagagccatcttagcaggtatgaggtgatatctcactgtggtttaaatgttcatttccttgataattagcaatattgagcatttgtttttcatatacctgttggccatttatgtgtcttcttttgagaaatgttttttcagatcatttgcccattgtaaaatcagatttttggctggatgcagtggcttatgtttgtaattccagcactttgggaggccaaggcaggaggatcacttgagcctaggagttcaagataaatctgggcaacatagggaaaccctgtctctacaaaaaaattttaaaaattagcagggcacggtggggtgtgcctgtggtcccagctacttgggaagctgaggtgggaggattgcttgagcctgggagttgaaggctgcagtgagttattattgcggcactgtattccagcctgagcaacagggtgagaccccaacttaaagaaaaaaaatcagatttttggttttcaacaactcttgctattaagtttcttataaaaatattcaccccttatcagatgtatagtttacaaatattttgtccctttccgtaggttgtctgttgattgtttcctttgctgttcagaagctttcatttgatgcaatctcatttgtccaaaaaaattattgcccagaccaatgtcaagaagccaagatcatgccactgcactccagcccagcaacagagcgagactccatctcaaaaaaaagatctgaaattgtaaaaatactagaagaaaatataggagaaaagcaattctcctgcctcagcctcctgagtggctgggactacaggtgtgcaccaccacacccagctgatttttgtatttttagtagagatggggcttcaccatgttggccaggatggtctcaatctcttgatcttatgatccacctgccttggcctcccaaagtgctgggattataggtgtaagccaccgcaccccgccatttcagatcttatgtttaagtctttaattcattttgagtagtttttttttttttttttttttttggaggcagtctctctctgttcttcaggctggagtgcagtggtgtggtcttggcaacctctgcctcccagattcaagtaattcttgtgccttagcctcctgagtagctgggactataggtgtgtgccaccacccctggctaatttttgtgtttttggtaggtttcaccatgttggccaggctggtttcgaactcctggcctcaagtaacccattggccttagcctcccaaagtgctgggattacagatgtgagccactatgcctggcctgagttgatttttgtatatgaggtgagatatggtcaaattttattcttctacatgtggatatccagtttccccaggactgtttattgaggagactgtcctttccccattgtatgttcttggcatatttgtaaaggatcagttggccataaaatgtgtggatttatttttggcctctattctggtcttatatgtctgtttttgttccagaaccatgctgtttttattactatagctttgtggcagcttctgaaattaggtagtgtgatggctctagctttgttccttttgttcaagattgctttggccattttggtccttgtcttttgtggttccatatgaatcttaggattttttttctatttctgtgaaaatgtcattggaattttgatagaggttggatggaatctacagtttggggtagtgtggacattttaacaatattaatctttccaatccattaacatggaatatctttccatttatttgtgtctttaatttctttcttcaatgctttatggttttcagtgtacagatttctcacctctttgattaaatttattcctaagtatatatttttgatgctattataaatgtgattgctttcttgatttcttttttggttggtgcattattagtatacagtaatgctactgacttttgaatattgattttgtatcctgcaactttactgaatttattagttctaatagtttttttggtggtatctttagggatttctctatataagatcatgtcatctacaaagtgacaatttaacttctttctttccaatttggatgcctagggagcatcagtggctcaagccagctgtcctggtgcacagggaggcgaggctatgagttcgaggccaacctggtcaacattgaccaatttggatgtcttgtatttctttctcttgcctatctgttctgactagaacttctagtactatattgaatagaaatggtgagaatggttatccttgtcttgttcctgatcttagaggaaaatcttttaacttttcatgattatgtatgatgttagctgtgggtttgtcatatatgggctttattgtgttgagatacactttttctatacctaatttgctaagaatttgtatcatgaaaggatgttaaattttgtcaaacgcctttcccccatctattgatataatcatatagtgttcttcattttgttaactgcttgtacttttgtacttttaaaattttgtataagaaatccttctctattctgatatcatcaatatgtgatcttatattttctttgaatagttttcagttttgctttttactctagctacctgaaatttacttctgggtaggatgtaatgtagaatttattttttgtgtgtttgtttggatagccagttaactcaaaaccattgaactttcccactgatttatgtcacttccattatagccaagtctcatatctttgagggtctgtattagtccattcttgcactgctatagataaatacctgaaatcaggtaatttataaagtttaattggctcatagttccacaggctgtataggaagcatggctggggaggcctcaggaaacttacaatcatggtggaaggtgaaagggaagcaggcatgtcttacgtggctggagcaggaggaagagagggaagaggtgccacacgctttttttttttttttttgagacagagtcttgctctgttacccaggctggaatgcagtggcacaatctcagttcactgtaacctccaccctctgggctcaagcgatcctcccacctcagcctcctgagtagctgggaccacaggcatgcatcaccatgctcggctactatttttgtatttttgtagagatgatgtctcagcatgttgtccaggctggtcttgacctcctgagctcaagtgatcctcctgcctcagcctcccaaagtgctgggattataggcaagagccactgcacctggcctccacacacttttaaacaaccgggtctcatgataacttactatcatgagaactgcacccagtgggaaatccatttccatgatccaatcacctcccaccagaccccatctccaacaaaggggattacaattcgacatgagatttggagagggagggacacagatccaaaccctattaggatctgattcaaaaaagttttgttctgtactgtggtctatttatgtgtgcatataccaggactagactgttttaatcactctaagtttcaaccagtaaacttgtgacatatggttaggcaagggatctcatattcttctgcttcaagttttgttttttcttggccatttactcttccataagaattttaaggccgggtgtggtggctcatgcctataatcccagcacttgaggaggccgaggcgggcagatcacgaggtcaggaattcaagaccagcctgaccaacatggtgaaaccccgtctgtactaaaaatacaaaaaaattagccgggcgtggtggtgcgcacctgtagtcccagctactcaggaggctgagccaggagaatcacttgaacctgggaggtggagcttgcagtgagccgagatcgcgccattgcattccagcctcggtaacagagtgagactccgtctcaaaaaaaaaaagaattttaagaacagtctgtcaagttccataaaatatctgattggagttttgcttagaattgtactgaatttttagatttgagaaaaattatcagctttatatcaaacttccgtctttgtgagtaggggatctcttcctattcttttatatcttctttatgttctctaataaaactttataattttctccataaatatcatacacagactttgttatatttattcctaagtatcttatgatttttgttatgacaaatggtattttcttttctttttttttttttttgagacggagtctcgctctgtcgcccaggctggagtgcagtggcgcaatctcggctcactgcaagctccgcctactgagttcacgccattctccttcctcaacctccccagttgctgggactacaggtgcctgccaactcgcccagctaattttttgtatttttagtagagacggggtttcactgtattagccaggacggtctcgatctgctgacctcatgatccgcctgccttggcctccggaagagctgggattacaggcgtgagccacgatgcctggcctaaaatgaaatcttttatctgtttattgctgtggtgtaaggatattgatgtttgtgtattctccttgcccccggcaaggaaattgttatttctaatagttggtaggttcttgtgaattttctttgtatagtaagcaatctatgtatagtaattgtctatgtatagtgatggctttatttcttctttttcttcttgtgtcacttcagacttcttggcagaaaggtgaatagaaacagtaaaaactggtatccttgtcttgactttggaagttactttctattcctagttagctagattaaaaagattaaaaaagaaatagacaggttctcactatacagtggttattcacagcctcaatcatagagcactacagcctcaaactcccaggttcaacgaaccttaccaccttagcctcccaagtagctgggactacaggcacaagccaccacacctggctaatttttaaatattttgtagttcactacagcactattcacaatagcaaagacatggcgtcaacccaaatgcctatcaatgaaagactagataaagaaaatgtagtatgcagccaggcgtggtggctcacacttgtaatcccagcacttaggaagcctgaggcaggtggatcaggagttcgagaccagcctgaccgacatagtgaaaccctgtctctactaaaaatacaaaaaaattagccaggtgtggtggcaaatgcctgtaatcctagctactcaggaggctgaggcaggagaatttcttgaacccgggaggcagaggttgcagtgagctgagattgtgccaatgcgctccagcctgggtgacagagtgagactctatctcaaaaaaaaaaaaaaaaaaaaaaaaatgccgggcacgatggctcatacctgtaatcccagaactttgggaggccgaggtgggcagatcacaaggtcagaagttcacgaccagcctggccaacatggtgaaatcctgtctctactaaaaatacaaaaattaacagggtgtggtggcactcgcctatagtcccagctacttgggaggctgaggcagaagaatcgcttgaacccaggaggcagaggttgcagtgagccgagattgtgccactgctctccagcctgggtgacagagcaagacaccaccttaaaaaaaagaaaaaagaaaatgtagtatgcgtacaccatggaatactatgcagccataaaagggaatgagatcatgtcctttacagggatgtggatggaactggaagccactatcttcagcaaactaacacaggaacagaaaaccaaacaccacatgttctcacttataagtgggagctgaacgatgaaaacacctggacacatgtgggtggggggaacaataacccactgaagtctgtcagaaggggaagtggcaggagggagagcatcaggaagaatagctaatggataccgggtttaatacctaggtgatgggttgacaggtgcagcaaaccatcatagcacatgttcacctatgtaacaaacctgcacatcctgcacttgtactccggaacttaaaataaaagttgaaaaaatattttgtagagacgaagtctcactatgttgcccaggctggtcttgaacttctgagctcaagcaatcctcttgccttggcctcccaaaattctgggattttagcagtgagccactgcacctggcctccagttctggatcctgggtcttgcgacattattccatgctggcttctgaagtcaatgaggcaaaaccagaaaggggaactaaaaagttaaagagggagagggtctcacaggcaagatggaggaagcctgggatgaaggcagggctgtggacctgaatgatgccaggaattaaggaggaagtaacagtgttaggtaatagcacatgggattcttgggctgcagctacaggagggagaatcatggccttaattcatcacctctgaaatcctgaaatatctagcctcctgcccagatcctgccatgaatgcatctattcaaccactatttataaagtatccagtgttaggtccagggagtggcaagagtgagcatctgcagtcatagtccctgccctcagggagcttacattcaatggggcagacagacatcagtctgtacataatcatcaacaaatgtagaactccaacagggccaagtgcctctgaggaaaggtacaccttagtgtgagcgctcataacaggagaatttgaccctgaaaagaaggtcaggaaggcttccctgaggctattgagaaggtgaggcctgaagaatgaagaggagctaagaggagaggaacagaggaaggagccgttccacagagggtccagcctccctggagactctgtgcaggaggaagcacaaggtggacagcaactggagcacacgagtgagggggtgtggtgggagatgaggctggagaggaggggccaccccttgcagggccttagaggtcaggttaaggacttgggtctttaacctaagatcaatggaaaaccactgaataggtttaaggaggctgactgaaacaatcagatttgcatttttaaaggatccctcttaggtgtgtgtcatactgtagagaaggccattcccagcctcttcagctactgcaatgactcagagaaggccacatggtccacatggtggctagcccagggatgtggagggatgaaaacgagtggacagacttgagatatattagagcagataaaatggataggactaggtgctggattcaatattgagatgctcattctaaaccgcccctacatcaaatccttcgaaggcaggggctatgccacatgactatgatacccaggtcccagaacaactcctgacacatggtagactcaatacacatttcccgaaagggtggaaatgattagcagtctctcaagtaatcctttgaccactaagcgtatttaatttcttctagcatgtgtcagtaatcaacctccttggtacatcatagggagaaatattgccgagttccaaagagtatactctggggcaagttaatacaagaataaggtctcacttaccctataacatgaagagtaacagtaaactcgtaagaacaggacctggtgaagcaaggagtaggcacaggaagatggctcatcaaataggggcgccgggtgtagtgtgaacacctgaggcaggaagaacccagtagcctctgggggttgggtgggattagttgggggactaggatttgatggtagggggttgtcctggagatctctggaaattaagttatctattttttttctgtttttatttttataaaagaactcttaagggcatttttttctttgtttgttctgttttgagatagaggttcactctgtcgcccaggctggagtgcagtggcgtgatctcggccactgcaaactccgcctcccaggttcaagtgattctcatgccttaacctcctgagtagctgggattacaggcatgggccaccacgccttgctaatttttgtgttttttgtatagccagggttttgccaggtttggtcaggctggtcttgaactcctggcctcaagtaatccacctaccttggcttcccacagtgttggaattacagccatgagccatcatgcccagcctaagctatcactttaaaatgacccttcaagaaagtcccaaggagtctaatccatccacgacaacagcacgtctgagacagcctagtgaaatttctccgatggaggacaggagatggaaaagtgccccaggacctcttatttatataaaaaacaaaattttttaagaccctgagaagtatggattcttctccagagaaaagttcctggcttgggagctcccttaggacttggcactttggtctagagtgaagtggacctggccattccagctttgagtgggttcaggcgagacactcgagctaccccttatggttgctctgaaggtcaccctggctgggagtgggtggatctctgctctgttatgcctcccacaggtggaggaagtctgtctctgaggctgccctgccaccctcatagagcgctgcagattgggggcggtgatgcagccacaggggctctgattccctgcctccccactgtgttcatctcattcctctccacatatgaggcctggtgtcatggcaggtgggtgttccaagtgaaaccacagccacagaggcaagcagtagagcctgtccctgatgtggccctggtgatgctgcaccagcctggggctgtatcagaaaccatgcctgcagcctttgcttctgcagacttgctggtgctgcaccagccctgagcaatgtccatagcccttgcctcactggtcactgtgatggggacgcccccaggtatggttctgctgctggtgttgcaggttggagtgggggccagcaccctcatctaccacaagaaggcttaggtggcacagacgtaaggctgcttgcctgtgtgtagagtggcaggctgcaacgggctggagccttggggagaggcccttgccacactggtggcagtgattgggttcaggcaggtgtgtgccgcagtgctgggccaggtgtgagctgtggcaagagctgtggccacactttctgcaggagtagggcctatcacctgtgtgaaagtgttcgcacagggtcaggtgggccccttggctgaagtacttcctatattagggggaggagaagggccccttgcccaggctgtgctgagagaggtgagagcagtgggaagagactccacattccaggcaggaaatttgagtggatttgtgggtggagatgggggtcccgggtaggggagttggggaagggttggttgtgacagctggcaggtaaaggagtgccatagtaggtagagggctgggtctgtcctctcttctcctcctcctctccctttgctcccagcctgcagggagaggggagagaggtgagagcagcatgggggaggaggaagcagaggagacagaggtgggagaggagaggctaagccccacacccaactgaagaagtggttggcggtttcagcggcacctccctcctcgctgaggtggactgtgaaggtggagaagcccagagcacagcctcctgtgagcagcttcccagggtgttgagggctgccaggcaaggacagggccctcagagttaggccttgcctgggcaggggtctcctgggggaaccttccctcagggccctgaggctctgggctccagagctcagcttccgcgttcagatacatggtctgctctgctttggagactgagaagcctgagaggtaaaatcgtgctgggtggtaggtagggtaagggagagggagcttccccccatctccctcacccactgcagaaaggcttctctgtttccttccccagcccagtcccacctcctctgtgaggccgctcctcacctccctactggcctgcagtgatcctcctgcttgggagtcacagagcacccctacacgggccctttgccattctttatagtataaggcaaggccgtaacattttgtggggcatcttcttcagtgtcctcagccttgtgtgaacatcagaagcacttgggggcttgggaaaatgcaattgctggccccatctccagtttcttaaacagcagatctggggtggggcccaagaatttgcatttctaacaagttcccaggcgatgctgatgctgctagtctgggatccacactgggagagccactgatctgttaggcgtgtaactttacttgtattatctctcaattctcagggcaacctgttactctcccccattttgcaggtgaagaaacagagggaagacccagtgccttgtgcaaggtggctcagtttgtaagtggcagagccgatttgaacccacgtttgtctgactctaatactgtatcaaccctccttcctctaccctgttcctctgcattgtcaactattccacatgtatcttgttcctcaacaaaatagtaacgttttcttttgcatactttatatctctcaccttgcctgacccagtaacataggcttggttgggagggactgaaggacttggaaaggaaatgccccagtggagccaggatggaaatgggttcctctgtgtctcccacatggtgtcacctaaataaacagccatgtctgtgaggttcagtcatggggagaggagggcaagagagcactcgcaccaagcgatggggttagggggacacgaaaggaaatgtgataaatagagaggggggcgtgtggggcaaaggtgaggactggggacccgggagggggcaggctgtttcagagtgaaggcaggacgggcagcagagacagagaagaggctgccgggtggttagggctggggaggacaaggagggtgaggacagacgatcccggcagcccctggagccagaagagaagtcaggttaactccagacttttggcttccacctctgtatctcacccaactcctccccacctgcttctctaccctgaaactccaggaacaacgcccacctgcctcccactacaacttccccttataccgtacacgtcctggatgcttggggtccgggaggaggggagtcaggatcacgggttccagggaagtgagtgtagaaaggggctcctacgagggtagaagtgggtgttgtctgcttctgcaggaaaggggccagccagcccaactggtccgcagtacttgctggggctggaggatgcccacctgcatctacgggattgagggtaccggagaggaggcgaacaaacgagaaatcccaagggtggagaagggagccatgggatcggcgggcgggccgcgcagctgcggaactgggagggccaagcgggctttagggggaaacacggccggctcctctgccagtgagacagagaactgagcatgggggaggaatttggggccagaggggctgggggtacccagagcagaggaaacaggaagtgaggggcagagaggttacgggtggtggaaggaacagcagtcggttctggagaggcgattcctcttccccgaatacctgcactgccccggaccgggagtgcagtcgtgcgcccctgtccccccagcctcaggcttggtgcccagccccgctcgcggaactcgaggccagtaaggcagccgcggctttccttgatgcccccactcctctgctctcagccgctgcctggacgagcacagaactagtgttcccaggcgaccctccctccgctcagccccgctgcccggcgggcaaaggctgtgcactcacactccgagtctgcagcccagcctgcccttttttgggaggtggcccggggactgggatgtcgaacagggagcttctccgcagtcctctccacctccctccctctttccagctatagaaaccggctgctcgcagtgacttccaaacatcacggtcattcccacatctacacacatccctttctcttttgagccattttcctgctttccctttggtgttgaaaccctggatttcagcctgggcgactcaggttttaggggggaaagtttggggtctaacttggcacctctgttttttatcctctcttccagcttcatagatggttcctattttcatacatatttgctaatttggagtttccgacgacatatctcgaataacccgacattttctccccatttccaaggctgattttacattatttctaggcctccctccccaatctattttcctttctttttcttgtacacccctgataaacagacacacagaactgcaggtttcaaagaggaaaaaagcacgtttgttgttggtaggcgggcgtgaatgggagggttcttcaccttctccaacacctcgtatagatgggacgttgcagggcaatagaaaaggcagcagatgggggcgtgttttgccccactctgtacaatatagaagaatctcatatagatactttgtataaaagccacgtgtcctcatttgtgtcctcttattggctggctgggctgggggtactgggggacaaagaagctggggtgatagaccaggacacagggataccccatggtccccttcctctggctgtcccctggtggggtcactttggatccttggtgggagcatagcacagctccagtggccgggacaccttccagaatttctcattcaccagctccagggtcagcgagccattcaacgtctgaggagggggtaaaagggagagaggggaaaagagagaggaaagttggagaagatggaaaaggaaatgaatgagaggcaaaaggtgaccaagggaggaggaaagaaaaaaggaataataaaagcgataaggcaaaaaaggacatggatgatgggaaaggagaggggacatagaaagagatagaggatgtataggggaagagtgctgatgagttagaccaggctcatggttctatgtggcccagaccaccctccctcctctcccctcttctaccactgccctcagaagctctcaccgtgaacgccccgcctccaggtgcgatgtagatggtgtactgcttttcactgacgccctccaggctgggcaaagcaggctcctcaggaccgtcacctcctccggcacccccaccctccccgccacacccatcaggtcctccggtgtcagaggcgccccctccaggccctcctggctcccctgcttcctgctgttgcctccgctcgagggcaatgcgcagggccaagtacttggagaggtggtccactgtggcattcccagttgtcttcacatacctggaatgggagggaggacaggcttagagccagagcgcctcagatgaggactggaggtaggggctttccaaaatggattctgggttggggtggtattttgggtttgggcaaagcccacttctgatggaaatctaggtgatctttggtttctcagtggctggggaaagacagggctcctcacctcgtctggcagtattctcccttctccacgagcagggggtggggccggaacacgagctcaatttctccacctggctctggggggctgggggccccaggagggcttggggggcccagcgttccccctcccagagtccctccccggtcaccagagtcttccgaaccggcacccccacccaccccaccagtgccgcctccccctgtccctacactgctcccccctgcgccccctccacggggtcgcttgggagcagggcctggggcagagtcaggggcggagtctgagctcacatcttctccatccccttctccctccccgggctctccttcccccccactcatcgttgtggtctgatctgaccctggtatcggccgcctcacacgctgggccctgtagaagcaagtggttaaggttagaaggcatccaggataaagggcttagactttaaacttcagagaattaagagataagaaaaatcctaatgacgatacctaatatttattaaacacaatgtgctaagcactcagtatactaagtgttttgcaccaatccccttacctaatacaatgaatcctatgacacaggtaattatattacctctattttatggctgaggaaactgaggcttagagaggttaagtaacttgttagtgatcataggctgtcacctctcaaagtgcggtagcagcatacacatcccctgtgagcttgtgaaatgcagactctcgggcccaactctagacctactgaatcacagtttgcatctgaagatctacaggtaatctggatacacatgaaagactgagagatgctggcctaggaggtgatttaacttccagcagtatgaatccggggcttattctcaaaatcgctctactgttctaacttctagaaagtcatggggtaaactacatttttcctttttttttttttgtgacagggccttgttctgtcgcccagcaggctggagtgcagtgtgcagtggtgtgatcatggttcactgcaacatccactttcttcccaggctcaagtgatcctattacctcagcctcccaagaaggtaggactacaggcatgtgccaccatgcccagctaaatgtttaatttgttgtagagatgagatttccctatgttattcaaggtggtctcaaactcctgggctcaagtgatcctcccatcctcacctcccaaagtgctgggattacagatgtgagtcactgtgcccagccctatatttttcttaagaagagcagggccagttgtttactaagaaactgcaatttacacccatgatttccagccctaatgatcagtctgtggtcagctcctaggatgctgaggcttagaagctagcaggcaacaaggactgacccacggacccatcccatcagttctgcttctctcctgaccctcacacctgtgcatggcctgcatgcgtagcccctcctcaatgctggagctcaatgcctgctggttgtgcaggcggctcaggcggataagcactcggtcttgatgggcctcgtattcctcccggctaggatagatcttagagatcagggcatcaaagttggggtctggccgtagggatcgcttggacaccagcttctttcggcaggtaggacactccttgttcctggggtaggagaagggaagccctgagtggtcagtcaagggaactgaagcctaggctgaaccaagaccagtgagagggccctaactttgtaccccttcatctcatctcaaacatgtctctcctattacccgctccgtagggctgtgacaatgcagtcagagcagaatctgtggaggcactccttggtggtcatcgtattcttcagcatgtccaggcagatagggcacatgagttctgaatgcagtgaccgaggggaaacagcaatctctgtgccatccattatggcttcctgaaggcatcagtgagtagaaaggtggtgtgggttagagggaaaggggttttagaaacagcagcccataaaataagaattttgagaaatacagtgagaagacccatgttggtctctataggagacaagaattctgagaaagagaaggactgatgacttggagcaaatggagaaaagacagactttagtgtccattaactaagagtaggagctttggaataggaggatctgaggtggctaagtggcctaggagtgaagatggcacaaatctgtggttgggaaaaactattcaacaaaattttttaaagtcctggtgctacctgtattagaaccccatagggtgcctgttaaaaattcagattctgagaacccaattcactactgaatcagaatgttggggggcatgaagctctgcttttaataaactcccaaagtgagtcttatgtgcactgacttatgacactgaatgaggaacagggaaggtaatttttgtgtttgtgatctttcaaatgaagataataatacttgttccacctctctggcagggctgttttgaggattaaatgagctaaatgcgaaagtgctttggaaagattaaggtgattcaaaggtagggccaggcaaggcagaatagagcaaattacggaaaagaagctgtgtgatgcggtctgtggatataagggtttgggggagggggtaagcctgaaagggagaatgcctgtcacctgcggggtccggtgcagctcatacagactcagttcccacgttttgctggcattctgggcattcgccggcgtcgtcatggtgaccgggcgcagaccccccaccagggctccacagccgaggagaaggcgaagaaggctgggggtggggggaaggggaggagggcgtcaggggggccgcccctcgcgtagaccccgcccctccagaagcgcccctctccgcccccgctcccgcccccgcgccggcactgcccctcacccagctccagccgttcgcgctcccgccgccgccgcgcctctccctaggcccgggctcctgggccaagttcgctcggtccgcaaccgctgctcagacagcagctcccgccaccgccgccgccatggcccgggcgctggggccctacgtcacttccgcctgctcctccgctacacccgctccccccgccggcggagacgtcacccaccattcgcggcgcgggtgggacgggacgtggacgccgcggttctcccgcccacgggacgggctcgtcccgcgtgaggatcctgcgtcccgggagttctgcgccacttagggaaccacggtctccgcccccggcccagcaccgtcgcgcggccaacaaccttagcctcggttcccttccagaggcccccacgcggggcctcagctccggggggccggggcctggaatatctgggaccccaggaggggacagagggctcgtgaaaagggctacctcccccactctgcgtaccctggcgactctggggatcaacccccctcacttctccgtaacccccattccaggatcccgggggaaaaggctgcaaaagatctgccgctttagccttctagtcctgagaacacacctaggtcccgatgtgcccgggattccccttaccaccctggggactccctccaccctctgctttggctctccctagaccctccgacccgctctcccgctcgaggtttgggggcggcggctcgggacgtccagactcaactctcggccggagccatagactcgagaattgcgtttggacaatcaggagccgcggcccggggcggggaagggagggaggcgcacgggaggaaagggggaatggagacaccacgcggaaacagggacatacacaagatggagctcccattgggaagggggggtgcgcgtgtgggtggggtacacaggggcagaatgtctggagtgggaagtgggcagaagcctcttcgaggactctagagtaaacgaaggggatggggaggaaagagaagaggaaggggcagagcgatccccattgagtgtagggaatgagagggatacagggcgagggggtggggggcagttagagaccctgatgggcatcttgtccccgcccgagctgaggctcccggacttgggctgcggctgccagggggtggcctgtccctgggacaaggcagcagtgggtgccctcgccccccaccccagttcccccctacttccctgtcctctccttcagtccgtggccagtccttatgtgggcgccagagtcgattagcgcagacccccctccccttcctcctcctcttcccagccccctccctcctgctctctccagcccctttcatcggctggttcattcccctaatcctggccccctcccctaatcccccccatccgaccccaggccggctgcagctattgtaaggtggagagaaaggggagggggggactcagagaaaggagaggggtgggggagggccacctgttccaagaccccctttcaaggccagactggacaccaagatggggccatgaacaaatcacccttggggaccataagaacccagggagttggggggaggggactggtgctgcagaaccagtggaaaggggtgacgcacgaacccctcccttcaaaaagacccggagtgtcacgcatacacagtgacacatactctttcctctcacacccggcggcgggggttgccctgggagaccaggcagagaaagggaacaatccttcgggaaagggaaaggagggggaggtggggaagggtctgagggcttggacacaagaagagccggaggtggcagggaagaggacttggaatttattagggtcacaagcacccctgcttcccatattcagcattcctgaaccatacactgccaccccttttgtatcctgggaacttagagtcctcatcagcaggaggccaggcagagtggtgggggggtgagcagagtccagggtccttgaggcagttacatgaaaagacctcctgggaggggcagaaacatttggacagatgcatgggtggagacacaaatgggctagggggtacccacttctgtccctggccttgggaacctctgcccttcccagtggctccagactccccttacttgctttctcatatagtctgggttctacttctctgccctctccctccccatgctggcctcataccagtgacttccactgaggtccctgtgatgtatccactatcttcagatgccaagaatgcgaccacatctgccacatctatgagaatggtggggagagggcagaattctgctcactcccatagacccaaaacagccataatctcccaccaaagacatccctccccacaaacctctccacagacaccaaccaccagtcccctagaaaatccccaaactttggggatctcttcattcaggcccccttcccagggaccccactacattcagtgctcaccctcagggtcccccaagtgtcccatcgggatcatttcagtaatctgtaaggaaatactcatgtgggtgaaagcttcttttatggatttttttgaagactgagtctgtgacccttttttgtcttcatttctcatacattcattgaatatttaatgtttgccaggaactgtgcccagtattctctctctctctctctctctctctctgtgaagcccagagagattgagtctctgaatgattctgccctccacccacagcctcctaccttgtccaccactttctgtggcactttctgtgtcatgggtgttgcaatgaaccctgggaggacagagttacagcggatcccatgtctgtgggaaggagagtggctgccgattctggagagggctgaatgctggtacctccctcagaccccccaggtgctccccagcaccctcaagcatctgaccaaccgtccaagctcccgggctgcggtctgggtcagcccaatcactccagccttggatgctgcatagtttgtctgccccacgttccccacctataggtgagtcagagatgtggaatgagtcaggagtctcaaggaggggttctcctctctataccacttggctggctgacctcgtccaactcaacctgacctttcctacgatgctactgatgttgatgatggaaccacgacaaccattggacaccagggcttgtgctgcagcctgagtgactaggaaggtgccctggcgggaaagggtaacaagggagaaaagatcagctggggtgacagccctcctccaactccctccccaggctaagggggccaaacgtcgcaggttcagagatcgccaccttgaggttgacagctatgactttgtcccagtcatcctcagacatgtgcagcagaaactcatcctgggtgatgcccgcacaggacacaacgacagatggtgggcgagaaaagcaggcctatgggagggggaggttacgcatcaaaaaccccccacaaaaagccggggcagtggaggcaatatcagagctttagagggggaaagtggcctagcgttcacctgcacttgttccagcaggcacctggcggccctggcctcagacacgtcagcctggaaggcagcatggttccctcggggcggcccctccttgctccctggcccgcccagcagccgcaccgtctcctgtgccgctgcccggtccaggtcgcaggcagctacggtggccccctctccggccaggcgtacactgaccgctcggccgatgccgctccccgcacctgaggtaggacagaacacgccggaccgggcacggggaggagagggcagggatcagaggtcacagggcagaaagtaaaatgtgggtaaggggccagaggtcacagcggccgcgcactccaagggcacgcccctgacctcactccaataccccaaaccgcccggggaaagaaccccctcaacctgtgaccaaggccagtgcggagcggagtcggttctggagctgagacgccatggcgggctgtgggtgggtgggaatcccagcactaagccaatcccagcgcagaggggcgtggcaagggcaaagccggggccaatcaaaggcccgccagccttaggccatctgcggccaccagcgcaggccgacctcccaccgagggggcgtggcccgacactgctgcttaggggccccgggtgtggagaagacaaggggccccattccccccaaaaagacaaagactgcatgtggggcggaagatcggaggtctttatttccggttaccacccctccccctcgattcaaacacagtgatataaaaggaatagaaacaaaaaaaatcaggccatgagaataccaactaaccctctcccaccccatccccccaatcactcccaaatcaggacacacgattgatgtttgatgtttatccccgactcctcctgttaccctctctgtccaggactctggtcttccctgaagcctccaccaactggtgggagaaaggaaactggaccttggagaacttcctgtgtagcaacttggttctccgagaacaggtggggtaggggacaccaggtaggctgccatcaccccctgtcccttccagccttcttcatggccccatttagccttccaaatgcaacatggtcagacagtgcccaacactatggtccctctggcctctcattctcaccgctccagcctctgtaagactgccagcccttgaaggccctaatgccaaagtaaccattctcccaggctacagttccttcctttggcagatgtccctggccagggcccccaacctctacgcacccctgacctggagttaggggtagaggtttggggcaggggtagtttatccacccctcactcaaggtgggcaatcagcaccatcatgataactccccccagcagccccagcacctccagaagtgattgcaatggtgatgcctccctcagcagctcgggcaacacagacactgttgctacgtagataaagccacctgcagtaaatggcaggacccagccaggacctgcaccacctgcaatttcactgcccactgctcctccttcagtgagaagggcacaggctgtgcctgccagtgcccctactgctgtcagtagttgcagacgcatcgcctgatgggcagaaaagagaaaaaggcactcaccaatagctaaagattatcagtggtttagaagtggctggtggggtataggtataggggacatggatttggaaactaaacatcaaaataaaaagagaagagggagcagtctagaatagacaatttctgaaaaatcttaagagatcagaggtcctttccacagactttctgattgagtaggcgtgtgtgtgtaagactccactaaaccattcagatggtttatagaccagtttaagaaatactgagctaaaggacaaagtaaccagaacttcttccccctcatttctagaggacttgtcaatgacagtatgaattgtttggaactaataagaggctgtttgggtttagggtgagtgaggagagataaagggtttgaggcagctgtgtttggcagacatcaccaacctgctttttgctgcagccagactggaccaagatggcaaagtctccgacctcgtggggcacttcatgtagcaggacagtcattgtggtcaggatccctagtccccggccccctcgaaaggaagccccaatggccagaccatcagtgaagttgtgtgccaagtcagcagccagattcaggtaccccgacacacgcaggtcttgtacaggacaagaagagaaaatgatcagaggcttccatcaccacttgttccagtcttggtcagctattctcacacctcccctcagagaccactttacacccagtcctttagtgtttctcagatacctgccattacccacagcacatattccatgtgatttgtgatgatgtcccttgcccaaatttatcaccaactctggcccttacctaagcctcttttttcttcttcagcgttctgaggtctcactggcccatctttgggtactgtgctccctcctcgcctcttctgaacccctcttgtttccttttcttcttcctctgagctctgcttctccttggtagaacgctctgagggaagaaaagagttggtcagagtggagaacagatcttccaacatgtcctctccctcctgagggccataagatgaggagattctgaggcaggcaagtcagctttcaggaaagttgttcctgggctcaccttgtcttccatgtccatgacttccacgtgtatgactgtgagcgtgtccatgtccatgactgtgaccatgtcctcctttcacatgtctcacaaatttctccacgacaagaaaggcaacaattccactgagaacccacagtcccacagacagaatggggccctggcctgggaatggaggcattgagacattaagggagatgtgcattccagactcctcctcctcaagtgcgggaggtgaggaatagggagagacggaccttccccagcaccccaactcccatccccatctgtctcttcctcaccactgtgggagtgtccatgtccgggttgctccagagtgtggtgagaatgaggttctggaagaaggggggaaaaccagatgagggaatatctgacacaatgagtgtgtatttgggggaatatcgaaaggtcagggatcataaaggagaaagaacccttttcctccaaagaacgaaataggttagatttgaggtagaagtcagaggttacttaccaagagcatgaggaatgaggtgcaggaaagcatctcccaggagcccaccggaagcaaaactgagcaagatctgaagtagagagcgatgccggggagagttcgactccacggggataaggaagaggacaaaaaatggagctgctgagatcagcactgtggcccccagtgcctggtgagggagagtcaaggtcaagtactgtggagtttcccaacaatccagaaccagcccttctctctcccatcccctggagactcacataagcccagagagtgacagcatccaggtcctgcttgatgcctggagccccagactccccatagcctccatggctatgctcatggtcatgtccatgtcctctgtggtagaggctctcatgggagtagccatggctatggccatggtgtaaatcctcatgtgaatgtccatggtcgtgatcgtgggtatgtccatgccagatgctctcgtgagtgtggccatggccatgggcatggctgtggccatggtggaaatcttcatgtgagtgcctgtggctgtggccatggaagtcctcttgcagatcgtcgtgcaggtcgtcatgacccccgagtccagccaccagaagccccaaggtcgcccaggtcagcagtcccacggccacccagtggggggcccccaggcctctggccatcacgctgaccagagggacagaggcagtgactccacttgactcaactctatacctacacagggtccgctttactccaatcgcgccctccgtccacctctatgggccgctctcccattcccttaagttcccacctacattgttcccaagactagttctctttatcccgaaccacctcccggatgtagggccctgggacactaatctttcaccagccactttacagactctctgggccctcgtctctacgactcattaggtgaggaaaaggcctggaaaggatggtagagaaaaaacagagaattctcaccggctccgggatcctctcctttcccggtttgcttggacgtggcagtcccgcctttagatcccgcgagaacaggaagttccgttccaccttcgccctaatgcctttaccaatatggcggcactccggtagcgacgaatccatcctgcactgacacgcatgcgcgagtgtggaggtgatttgaggggagggtgggcgaggcgggctaacaggccggaggagaccgcctcctcggtttccaactagacgagggggcgggaactcgcgccgagacttcccgtctgtacaaagatggctgccacattggcgctgtcattttggtactgagcagagcgacgggcttaattcgacccaatccaggccagagtctttctctcaggggcttcctcgtgctcagctaatcctccgatcaatccttgggaatccctgggacctcttcggtatccctactctcagccagggatcatgtcttgggccgctcgcccgcccttcctccctcagcggcatgccgcagggcagtgtgggccggtgggggtgcgaaaagaaatgcattgtggggtcgcgtcccggtggcggcggcgacggccctggctggatcccgcagcggcggcggcggcggcggtggcaggcggagaacaacaaaccccggagccggagccaggggaggctggacgggacgggatgggcgacagcgggcggggtgagtgccccagcagaagggcaggcgagccagtgaccgctttatctgcgacccctcccccacaccctcccccggtgctcttttccttgcgctcccgcccccttgtttgtaaacacgcgtcccctccctcccgagggccttagcaccctcccccccggggcggggcggggaaggggagcttccgccctcctagctgtgaccgcttgaggccttgggcaccggaggtcgcgggcctgggaagggcacgattcctcttaaacctcaggactttgggcgtttacaggcagatcctgcgtcttaggaggggtctctcctgcctgtctctttcccttgcacgctctggctacctggctttacgctgggaatagaggggctcgatggtctaccccacgtgctgccccacccacgagacaggtgtggctttgggatgacctggtattcattcaaatggattggttggaaaattttcctcacctgaactatcctcctctccttgtcaactccctcctctcaccacaatgaggagttacgctgtttttgggtttttttagccagtcaaatatagcagtgggaggttgtataccaattttagtgacacaaatgttaataagttctgataacccactaccatcggaccagccggagttacactgttgtttgacagcagggtgtctctgaacttggggatgggtggagtgggctgggtccggcacggtggggtattagagaattaaagtctctagttggatgtttgaagactcattttctttttttgtctcttctgtctcttgtgtgtctgtgtgcctgtacatacccctccctcagactcccgaagcccagacagctcctccccaaatccccttccccagggagtccctcccccttctcctcctgggccacccctacccccttcaacagctccatcccttggaggctctggggccccacccccacccccgatgccaccacccccactgggctctccctttccagtcatcagttcttccatggggtcccctggtctgccccctccagctcccccaggattctccgggcctgtcagcagcccccaggtgagaggttgtgaccaacttactgcctggccactttcattttcttgttttccttgtaaccccagatccttgtgtgaacttccccatggcccattgaccttccaatcctctaaccttgtcaggcctgtggtctttatgagaccccttgcctttcctgaggtgactgatctttcagtgcatgcttcctcccctaaaagaactctaatattctttgtttttgtgtgtgtgtgtgtgtttttttttttttttttttttgagatggagtcttgctctgttgcccaggctggagtgcagtggcacaatctcagctcactgcaacctctgcctcctgggttcaagcaattctcctgcctcagcctcctgagtagctgggattacaggtgcctaccaccacgcccggctaattctgatattcttgatgtccttttcatctatctgattttctgcccttttgatcccatctgctttcccaagacacctgcccttcagggtcactgtgttgcacaatttcagggggggcctgtttattcactttggggttgtgctccagggatggcctttcacatagactgcagtgtaaatgacagcctctggaatgtgcattgcagggccttgcttagtggtagggaatgatttccatcacttctgtgacattctgcttcccaataagtcttcctgtgacttccctatttcccccatcccagattaactcaacagtgtcactccctgggggtgggtctggcccccctgaagatgtgaagccaccagtcttaggggtccggggcctgcactgtccaccccctccaggtggccctggggctggcaaacggctatgtgcaatctgcggggacagaagctcaggtatgtggctcagaggatgaacagagagggagagtctgggccatgtatcatcacctgtgggattcccagggcttatggagtttggtcagagcaagtgacctgggggaggcctgatgggagtaaagaagctgaagctgagatgtaggacgcgattggggggaaggtcagagggaaaaggaagcagcgtgtagggtttctgaacagtgaggagactgggactggatcatcactcagctcctggttccctatctctgtatttggcaaacagggaggactttaagttttgctagggggagattggaaacagactaaatggtgaaggtgtctccatgcaaccttcttattgctcctcccctttccctgtaggcaaacactacggggtttacagctgtgagggttgcaagggcttcttcaaacgcaccatccgcaaagaccttacatactcttgccgggacaacaaagactgcacagtggacaagcgccagcggaaccgctgtcagtactgccgctatcagaagtgcctggccactggcatgaagagggagggtaaggacccgccctgcccaggcgtcttagaccacatgcccttctcccttatcccactcagactgtgtacctctctggaagggtggactgctctctctcctctagagggcgatatttgatttctctctcccttcgcagctctcctccctgttaccctgttctggcccttgagtttccttcttaatggttaggcctctgtggcctgcacatatgacccctccctagactgatcctaggataggataacttttccttagccatgatgggctgtctccccatttctaacctgagtggttcctcctctgattccatggaccttctctggtctctgtctcctgttatctgtgatctttcctctaactcctgggactgggcactcttaagcttacagactttactgaagacctcagggcttcccttccttcttctgtgtctccatcttatcttcttaagtcagattcctaggattctgagagacactttagagcatatcttggtcaaaacacccactttagagaagatgagcctgaggcttggctttcccaaggtcatgcaacaaaggagtggcaggcaggtttgggatcagaacccacatcttcaaagacctaggccagtgctcctttcactgcctatttgttgagtcagaagagcacaagatttgcactcagacacacttagagtcagttgtatgacctgccatgtattatatgaatttaagcaaactaatctctttgaaccccgagttcttcatcttaaaaatgagaataaatgcttccagcatcattatgataattgagtgaatggtaaaatgtgcttagcatgtttcttggcatattttatttaaaaaaattttttttttcaatcagctttctcaggttgaatcttggcatgttttttttaaccatgtagtaggctctccatttaaattcctccctaattcttttacctacctcacccttcccgactgacttacctgcccttttataacccttccttcagggctgcccccagtccaccctttctagtgacttccccaattcctataaatatctcccaagggccatgagatcctgatgaggccgtaaggatatttgtggaaccccttcatggctggctgctgactttccattttttctctcccccttccccctgcagcggtacaggaggagcgtcagcggggaaaggacaaggatggggatggggagggggctgggggagcccccgaggagatgcctgtggacaggatcctggaggcagagcttgctgtggaacagaagagtgaccagggcgttgagggtcctgggggaaccgggggtagcggcagcagcgtgagtgttggggtcaatccactctccttcgtgatgggggttgggggaggcagtctaggtctgttctacatcccctccccctcctttcccctcataaccttcctaacactacttgggactggaggtgctgccaaacaaggtctttcaaacatctgaggtggatgtgatagctccttctgtctccactccccaaacaacccactggcagaaccataggcatgtcccaaataaataattgtttgcactaatgccagaagagaagactcacttacagggattggtttggatggggctcacaggaagactatatgtaaggagggggtgtcaaaagcctcttacaagggggctcccagcatatctcaaaatcttccataactcttacccccgtcccctgcagccaaatgaccctgtgactaacatctgtcaggcagctgacaaacagctattcacgcttgttgagtgggcgaagaggatcccacacttttcctccttgcctctggatgatcaggtcatattgctgcgggcaggtcagtgaccttggatccctttgacttcttgacatttgacccctctttgacttcccgatctttagtgaccccagtggccttaccttgcgtacccagggagccaaacttgctgacctcgccacctcttttctccttctcttccactgatgtgctttgaatcccttggcctgatttctggctcctgacccttgctgccccacccaggctggaatgaactcctcattgcctccttctcacaccgatccattgatgttcgagatggcatcctccttgccacaggtcttcacgtgcaccgcaactcagcccattcagcaggagtaggagccatctttgatcggtcagtggccctcggctaggctggcatgtagatagagggggtggggctataggctggtccgtgtccaaggctggctgagctgtgacctttgagtgacctgcaggtccctctccagggtgctgacagagctagtgtccaaaatgcgtgacatgaggatggacaagacagagcttggctgcctgagggcaatcattctgtttaatccaggtaagaggaggattacctttgtctctcaaggccaaggcaaccttggagcctcctcttctccgagactcctaagttacccagctatccccctcagtaagaccctcaacctggaaatctcccaactggccgaggaaaaacccacatccacggatccatcccacaaacccagtgggccctgcttcccttgccaggcctctggtaaagggcaagcaagtgcagcccaaaaggggcatcctgtggctcacatctgcatggccatcctgatttggtttgtcttcatttctcttcctcctctcttccctgccatcccagatgccaagggcctctccaaccctagtgaggtggaggtcctgcgggagaaagtgtatgcatcactggagacctactgcaaacagaagtaccctgagcagcagggacggtgagatggggctggggggcactgaggctcccttgggggtggggagtgctatgaggatgtgttcagggcccgtgtgcttccagctctcaatccccttctcccacctccaccccaggtttgccaagctgctgctacgtcttcctgccctccggtccattggccttaagtgtctagagcatctgtttttcttcaagctcattggtgacacccccatcgacaccttcctcatggagatgcttgaggctccccatcaactggcctgagctcagacccagacgtggtgcttctcacactggaggagcacacatccaagagggactccaagccctggggcagggtggggggccatgttcccagaaccttgatggggtgagaagtacagggcagaaccaagaacataaaccctccaagggatctgcttgatatcccaagttggaagggaccccagatacctgtgaggactggttgtctctcttcggtggccttgagtctctgaatttgtcgggttctcccatgatttggggtgatttctcaccctctgtccttcccccagcacaaagcactggccttgcctccaggaccttgcttccttctcatcttgcctcattttgcttcccatctgaagagtggaaatggggaactcccccagaggtggatactggggggcaggcctcccaagctgatggacatgagagtagggccctgacaggccttcctcctctcaaacctggcagatgggggcctctctggaagagggaggggccctgtcactgtccagagtctctttttacacttcacctccttctgcagtcagactgaaatataaaaaaggtggtggtggtggtgaaggggctggtggagatgtaggaaccgatctgctatttttaatttcctgtgaggatagagacttgcagttagactcaaagaagtactgtactttcccaggttgactaagaaatgccagtggtggaggtgggtgtttgggaaaggcagggccctgaaatggcctgtccctagggctctccaagcactagccttcccagcttcccgccgccccccctatctcttcctgtctaacttggggaaggggcctgggctgtgaggacagggcccccacaggggatggtttcacgagtgtagtcccggaggccttccctttacagctctcctccagccctgggcacatagcataggctggggacacaggatcctggcctgagaattgaggggaggtggccagcccgcagaggtggggtgctggggctgcatgatttttgccctgcgtcccttctctttggggctcctttcccctctcatacataaaatcgctttcaaattaaaatcgctgttttctggactgaggtgactgtatgaggatgggcagcgccgtttcgggcttggggggggtatccggttgggagctccggacgcgatccctggacgccgccgccgaggttccgtggggcccaagaggtgcacgcagggtgggggacacaaggcaagctttgtcggggagggggggagagggtgtgccaggctgggggcggggccggccggaggaggaagggggggcggtggattctcaaaggcgccttgttcgctcgtgccctctccgcgccagcgggcggcggcgcctcggctcgctgcggccttccctccgggcgcgctgcgggctccgggcagacccggcgccgtgcccgctcgtgggggcgcactgggcgccgagcgctgcgtgcttctcaggcgttgcccgcccggaggcggcccacgtccccgagtgaccccatttccctggacccttccaaccagagtcgactgctcgctcatcttaccactgctccccctctacctcggctctgggactccctaaccgcgtcccccatttgcatctccttggacctgactatcctgtgtttgtcggtgggtcccagtctctggcctcttgctctccgtagaacaccttcccccacatcccccgccccagttcgtcgctatctatagccttgtctataaatacccccgccccggcccggctctgtaattacacggggcggggtgagggaagtaattatggagacctgattatgggtggggtggggactgcgacccccaggcccgcctctccccctcccactgtgccctaaatcccgcccaggcctctgctgaaagggggctctgggcccccaagagggagggaatgggagggagtgtgtgtgactggacgtttgggtcctagaaaaggaaggggctagggaagatattggggttcccgaaaagagaatcttagggtacaggccgttgagacctacaaggggcaggagagagcgagcgatagagggagggttcccgcctccctccccaggtggagactgagggtggggtttcccttcggtggctgtgggcgggcggctggaggcgggggccgggccgggggcgggggcgaggtgggggctctgggcgccagggtggccggggacacacagaagcggcagccaccgaggagggagcagtgccgggagccccgacggcgccttgctgcatggagctgggccgctgacagctgtcgctgcccgcagcctctgacctccctgggaccccggcgtctgaggctcatagtctgctccctgtcttctgtcagcctcagggcatccagcgtctcaggccgacctgggtccctgggacccggcgtttcggcttctcagccatggagcggtgcagccgctgccatcgcctcctcctcctcctacctctggtgctggggctgagcgcggccccaggctgggcaggtaagaggagtcctgatgcctgggtcctcggagtcaggctggagctctgagtgtctctgggaaggggccagctgatgcctggggcagcagcttctgagtctaacaaggagatttggggcttcaaggctacaggctggagggcaggacacctgtgttccttggcatcagggtaggaatctctttgtccttgaagtgactgggggagggcagatagggctgaaaggtggaggaccactttagagaccctgaatggggatggatgcttgtcttgagtccttgggaagagctgaagatggccagggccagcccggtccagtcattgctggtgtggggatggtagacgccaatgttgatgggtgaggcgtgtgtgtcttatggcctttctgtcgatatctctgggataaggggtgagcacctgtgattcagagcagagcccagtaagcccagaaagaaaggaagccttgactacctgtctcattggcctcctggacagggtccccctccccctccctgtgccctcgatgctgccacgcagctggctctggggagcactggggctggctgccaacaggacggccctccggacaggccaggatcaggtgtctgaggccagagccaactctttgcattcctgcctagcccctcctccctctggccagccaggctcccctgggaccctggtgcccacaaccctgatcttttccttctttttcccaggacccagaattcctggcttctaggaaaggggatttgatgtcaaggagggggctgtgagcaccaggtcctcagcaggctggaaaaagcccatttctggagacactgagggctgatttatatttaacttgactattcaattcttctctgctttagagagaaaatggtcagaatggcagcacaaaagatttagattagattacagaaggaagaacttccaagatgtgaggacagtgaagcccagggccaggaaattgaggcgtatggtgcctgtagactctaggagtctttcttagggggaagagggatcctctggcatggaggcagggggatggctaagatgactgtttcttatttcaggggctgagtgggtagagatgggactttagggaaatgagttactgtagaattcctaaggttagagaataaggcatttggggggccagaggtagagtggtggagggtaatggggctaccagtggagagcctgtggggcagatgaggctggatacaggggggcaagtatgtggcctggtgtgggaagatggtgggcggacctgtgggtaggttgtccatatgatcgaattgtactcccttgggatttggtggagggtggggacctccgaggtctccttggcctggggtctctgtgagtctctgtgtctctgtctcactcacccttcgtctctcaaagcggcccttgtgtgttggtgtttgcggagctgccacacgcgcaggggccaggcttaggtgggggttgggggggaaggccgtggtttcccggctcctggactcaaagggccttttctctgcctgcccgccccacctcacccaccccacccagctccactctcctgattgctctggcctctgccctgccccacacttctatgaaagttttgctggaactctctttgggatttttccaatctggagctgatggtgtgaagagtatgtgtataggggagcaagtgagggcaaggtgatcttttgttttccagggagtttgagccctaggaagctgagttctagttagacagggctggtgggctggatcttgaatctttgggtcccaggtggtggggttggtgggggtggtgagacaggattgaggttccaggtgtggggagcttgcctgggttctgggcaagcactctgatgggatccacagatgtgtggctcagggccctggacctgttccggctcctcgtggcagcttcagcccctgccccccattcctccacccctccccaactcatctcctaaaccaggaggcacagactcctgggagagctttcttgtatctttttcctcagcatgctcagggttgttcccaccccatcccccaaacctgggatctgtagctgcttgaagcccctctcatagagctgcttctctgatctcttcctcgtggcccccctgcacctcctgtgtccaggatgatcgattgcccagatgttgtttgtgctggagacgggacagatgccagagacccagagagtgagagatatggaaagagagtgagagcgagggagagaaaaagatggacagaatgtgagagaaatggaggaagacagaagagacagagacagaaagaccaacataaagagacagagagagtaagagagtgagagagacaggagacagatgaagagttgagagaagtattcagagagtacagagagagacagattcataagctaaaagacaaagagagagcgtaagagaggcagatgctaggagagacaggggcaggagtgtcatggggttgaatgccagctgatgctgccctgcctccaggtacctttgcatttcccatccattcaggtactgttcagaagcaagtgagggtgaggtgacctttcccttgcagggagtttgagccctggaaagctgagttctagatcccaaatgtcccctatttatgccctcctgagggcatgtcccttctcctgataatcatggactctcccaggtgcaccccctgtggatgtgctccgggccctgaggttcccctccctccctgatggtgtccggagagcgaaaggcatctgtccagctgatgtggcctaccgagtggcacgacctgcccagctcagtgcacccactcgccagcttttcccaggtatgggtgacatggtggggtaggcctggggggaggtaatgggatggggcctaggatcagacaccaggaggaaaggggttgtggcggctccctttgcctctcactctgtgtgtatctctctcggttactaggaggatttcccaaagatttctctctgctgactgttgtccggacccgccctggtctccaagctcccctcctgactctctacagtgcccagggtgtccgacagctgggcctggagctgggccgacctgtccgcttcctgtatgaagaccagactgggcggcctcaacctccctctcagccagtcttccgaggcctcagcctagcagatggcaagtaagtttgtttgctcctctggtctgcctggcccacactttcaggaggaagtgcccccaaacccctacactctaaactgtgaaacccttgagaccctttgggccacaccacacctacccactgcccaaacttcagtcacttctagtccagagtttgggctttagagtgtacagccttctctgcatgttgaactagcctgtaccttgggcaagttacttaaaatttttgagcctcagtttctacatctgtaaaatagacattaaatagaattggcacaaataagaaagtgacggcatggtgtctggtgcactgtaaacactcaataaatggtagccattgttattactgctcttatcaccattggcttcaactcttatcactgctctccaaccatgttgactcccctacttcagtcagccaagagttcacttgaacctcttccaccatttccaggtggcaccgtgtggctgtggctgtgaagggccagtctgtcaccctcattgttgactgcaagaagcgagtcacccggcctctcccccgaagtgctcgtccagtattggacacccatggagtgatcatctttggtgcccgtattctggatgaagaagtctttgaggtaaccagagcaatcagaggcaggattgacttctggtcccctatcttgtgcccactacccttctggccccagcatgtcatcttcctattccctaggccttcatttttttttcttatgaattttcatttctattttctatcttcagggctactgttctctgcaggagaactagatgcctacctatatggcaggactgtccacagccacttactctgtccttcagtcttcatcagttccctcatccatccatcctttcaacccctctctctcctcacccacccatcaattcactcactcctcagccaacctatgcacacatccaccacccactcccccatctatttactcatctatgcatcacacgcccacctagtcatgcatgaaggcttatatccacccacctgtcaatcctccccctgcttaaccactctccccttcaaaccacccatctatgcttccacccatctatcctcttcaactcctccatccagctattcactcacccacccaccctccccatctacccatgcacacaaggatgcatgcatccattcagccaccatctatccattatccttccaccaactcaaccactccctccttcaatatactcactcacccatctcctgacccacttacttcccatccgaccagtcattcacccacataagttcacccatctacctattcacccatttgcttccatctactctgtaacccctgttcatctgcccaccagtccaccctcccatacgtacatttattcattcactatttatcaatgctcccattcattaatgcatgtaagcccccagccacccatccactcatctatccattcatccatccctccattcacccatccctccattcaccctccaagccagtcaacattttctgaatacctgctggacaaggagaacaaagaagagaatataaagtctctatcacccccaagagctcccaggtgggctctggaaatgagacaacatccctaaattacggtgcaatggagagtatgctaagcaccatgtgtgggcaaaagtaaatgccattggaactcaggaatgggttctccccactccatttctagaggtccagagaattctcatcatcactgccattggaaggccaaggcatctaaagctgagccctgtctctggaagccactgtggaagggctcatgcattgccacaaagcgcagaggggaggcaaagaattctggaaggcctgggatctggtcctgattctgccactctgggtgaccttggggcagtcattgcctttttgggcttttctgaatggaacgtatctgaaaaatgggtagaaagaaagatccttgttctgccaactttcctggttgttatgaaggtcctatgagatcccttgaaaactgaaaagggctatgcatatggaggactgacagtgaccttctcccatcttagggttttacctaggattggcctcctatactctttctcccggatgataccctctgcctctctctgaatctctccgctccatctctctcatgtctttgcagggtgatgtccaggagctggccattgtcccaggggtccaggcagcctatgaatcatgtgaacagaaggagctggaatgcgaggggggccagagggaaagaccccaaaaccaacagcctcacagagcccagagatctccacagcagcaaccatcaagacttcacaggccacaaaatcaggaaccccagagccaggtgagggagctgggagaacccccaagtgcagcacaccccagagagggaagacacccaggcatctctcctccttagttgtcctccccaccctcatctccctattcccatcaccccctcctcctagtcctcattcatcagcctttttattttcatctaaaaataaaaagagttgttatgaagaatttgtggctgggagctgtgttccctgctctgcgtctcctcttccagtcttcctggaactgtgtccctgggttttcccttccttctttgaattaagagattgggagacagtgggagaggggtgagagctggggaggcaggtaggagaggggactgaagccaggagaaagcagtcgggatggtgagaccaaggaggaggaatgggaaggagtagggatggagggaatatcaaaggaggggtgggtctgcagaagtgtgggagtggggaacccagtctctggcccccacctacctggaggggcaggaaaaggggaggtagtagggggaagggaagggaagggaagggaagggagcctgggttggatggggtctggagagttagaatacagataggctctggggtcagtggtctaaagatcagagggtcaatctcttaatcatgtctgcttctgccccacatgtggaaccccttccctctgttccacctctccccacctccctcctgaccctcaacctctcctttcatcttcaactctctgtttcccaactccacacctcccccgtttcccaactcccacaccatctttatatctccctctcccgcccttcaccccacaccttccaattaccccatccccttcctgtctatgcccaccccccaatcccagcccactgagtctctctactatgactacgagcccccctattatgatgtgatgactacggggacaacccctgattatcaggtaaactcaagggaccccttcatttcttccctttctcccagcacccccagcaatcatctcctaggacaccttcctcccctcccccatgccgtggggagaggcacagtgtgtttagattatggggatttacttagttcctgccccctccctgtctctctgctgcagcgccctgggagcccccaccacaggcctcccccttcccctagggggcatcggacccctccctgaaagcctaccccacctgccaagaggtcaatgacctggcaggcgtccccctcaccaggccggaggccctctccaactgcaggctttgacccagcatgggagcagcctcccctttcccgatgcccagctcggagggcagcttcccccactgcctggccccgccccaggaccagaggcagcagttaccggcaggtagagccggggcatccagatctctgcccccaaacctctatgaccttggaggttcactctccccaaactatccccctccaaaggggctgcaagatctgagatttcccccatcccccagcacagacagaccctgccctcgggggacaggtgctgctttgcctcccttctgctaacctttctccttccttcccaccccgtgctactcttctccttctctccttggggtcaggaccccaccccaggtgaagaggaagaaatcctggagtcgagcctcttgccaccccttgaggaggtaactctatgccccaccctcaccccatctgggggcagtgggcacctgtgactcactcccctcagcccatgacgtcttgatggaccagcccctcctctggccggagggttcttcctcatttccatcgatggcctctgtctttgggtcactctagaactatgggcctgcttgactctctaagtccagcccgttcccttcagtatccaggcctccttccatgattctttgattcctgagtgacttcgtctccatgttggtgtctaatggtcttactttctatgtcctatttctgttcccgggagccactttctgtccagtgttgtcaccttttgtcttccctatctctttggtcattcctggactcctttgtctgtcattgtgtccatggactgtccactctgcatccagtcctctggccacccttcattttatccaccacttcttcccactggacccactttctggacatctctccccatttttggcctttgatggcccttccatttatatacaaccactcccatggctattcttgggagaaaaaatccagttctgattaaccttggaatcctttgggctagaagttgtcacaccctggggtccagggtaagcctcctctcagccctaacatccccatctacccctctccctccccatccctgggaaggagcagacagatctccaggtcccccccacagccgacaggttccaggcagaggaatatggggagggtggcacagacccccctgaagggccctacgattacacctatggctatggggatgattatcgtgaggagacagagcttggccctgccctctctgcggagacagcccactcaggagccgtaagtgaaaggagcttctctttcattcttgactaccagtgggagctacaagaattgcttcttgttggtttcgatccttggctgttggctgtgagctatttcccatctgtgacttgtcattttctgttcacatatgatgtatatgttgcttgaaaagattactacctgccttcctttgccaaatgtcttaccttgtgactcttgtggatacttctgaagctcagggcaattgggaattaaaaaaaaaaaatatgtagccacttcctgtctttcttgatcacttcctgtctaactgaagctggatttcataaagcttttggtcaataaactactaccttgtgtccattttcagtgtttctctatcatttcttcttggtgaaataattttttgttcatgtttctaccaaacgatctaacttcctatgtcacctttttttaacctacaggccacttattatctgtcccttcctgccttctccagctgtggcatccatcttggtaactgtgtcaggttggactgcaaaggatttattatttctgttctggctctagtttttcatgctatcttgtgacaatttcctttgacaataagccacttcctgtatgtctcaggtcatttaaatcataaagtttccattgtgggcctcgatatttctcatggctacttcctgtgtgtcctagccttgatgtttgtgttttctggaagaattacccttcactgtcatgattccctttgcaggagtcacattctacctgttttgtgcctttcttttctctctgggtcacggtagccatgttgttgttgaaggttcacttcctggatcctctttccttcactgcttcctgtctgttcagccacttcctgtttgtccagtttgttttctagccatgttggccatgtttaattttcttcggccctggcatcattcttgcttccaagtgtttgtcattttgttctgtgtcccttagttgttctcatatcttccagtattccctgtcagtgggactgcttcctgtctactccgctccgtcttctgcctgccttgcagttctggctgcctcctctaccctgctgccgcatcccatacctcccctctcttgcctcccttgagcttctactgcctttcctttatgctgctctctgggtatttggggttgatatgggatttcagagaattttgaaacggatcattgctgtttggtcagaatgaaggtggagaggggcattctgaagatctttgttggggggagttggggtggtggtgaatgtctcccattctccatgttcttattttgtttctggccctagagttgggaacaggtatctgagtggaggtgtgcaagggagggaactggtgggcttggaatgtacttgagggccagaggaagggcaatgcattcagtggggactgctctccaattttttttttttttgaggcagggtctcactctgtcacccaggttggagtgcagtggcaggatcttggcttactgcaacctctgcctcctgggttcaagcgatcctcctgcctcagcctcccgagtagctggggttacaggcatgtgccactgcgcccggctattttttgtatttttagtagagacggggtttcacatgttggccaggctggtctcaaactactgacctcaaataagctgcctgtttcggccccacaaagtgctgggattacaggcgtgcgccaccgtgcctggcctccaaatttacttagagtatggatgcttaacctttggggcatatttaaacttgtgaaccctttgaaatcctaaataaaattctgtgcataatagcatatactttttcatttctggagaaagactcacagctttcatcagattcttttttttctttgagatggagttttgctcttgttgcctaggctggagtacgatggcataatcttggttcacttcaacctccacctcctgggttcaagctattctcctgcttcagcctcccaagtagctgggattacaggcatgtgccaccacacccagctaattttgtatttttattagagacggggttgcaccatgttggtcaggctggtctcaaactcctgacctcaggtgatctgcctgcctccgcctcccaaagtgctgggattatgggcgtaagccacctcgcccggcctagcttttatcaggttctcatgtgggtccatgccctcctcttaccccaaataaaaatctacccacaatcttagagagattcttttatgatgtattggggtaaatgtgtggaaggtttcttaaggttctagggagggagtttgggagatgggatagggtcctctagaagagggtttgggaaggtgggtggtttaaaatccctgaaaggggccaggtgtggtggctcacgcctgtaattccagcactttaggaggccaaggcaggcggatcacctgaggtcaggactttgagaccagcctggccaacacggtgaaaccccatctctaataaaaatacaaaaattagccgggcatggtggtgcacatctgtaatcccagtgtaatcccagctacttgggaggctgaggcaggagaatcacttgaacctggaaggcagaggttgtagtgagccgagatcgcgccactatacttcagcctgggtgacagagcaagacttcgtctcaaaaaaaaagaaaaacaaacaaacaaacaaaaacactggaagtttctggggaccttcctgaaagaggctctcagggagatggagaggttgtgtttgctgaggtggggctgggaactgggggggcagggaaccctgaggtctccttggcaggcttgaaaggtttgtgaagagggagttttaggggagggctgtggggctttcgggtagggctgaagtggtctcgagaggatctaagaatcaaggttggagttgagatggagaaaggcctagtgtcctggctgctcacgggccccactgggggctacatgtgtgtctccttcaggctgcccatggaccccgagggctgaagggagagaaaggagagcctgcagtgttggaacctgtaagttatgctggtcacagggctgaggcagtggaaataggagaagcaagtggggttgagtgtgctggtcctgtcgcctctgatttttaacctttgactccacagggtatgctcgtggaggggccccctggcccagaaggccctgcggtaagtctagcagtgacctggtggccattcttttcttagaaaccccttctatgtgctcatctgagccttccccacatatgcccaggcctcctcctcagaactcgggagcatccctccaatcaacgcttcccagattcccagatctgttctgcacagaccactcctcagccacaggctgagtgtccacacctgtctgaatgcccacacctgacccctactcttttgttcctcagggattgattggtccccctggcatccaggggaacccaggcccagttggagaccctggagagagggtaagggggtgtcctccatgtgacgggggagcttcgggggagctagtggtatccaagcggggggctaccatgcccagtaatttcctgttaggtggcttgttaatgaatgaggggatggttgtgcttcccagcaaccagaaaggagggcagactctggttggggagggtgctgatctggtttctcaccctcaattttccccatgggggtgggagactcaggaaagagctagagcaatctacacttattctgttgaagaggatatttcaaaatcatgctaagatcttgatgtccccttatctttcaccccatcttttgcattctcttcccactccagggcccccctggccgagcagggctccctggatcagatggggctcctggtcctcctggcacatctctcatgctcccagtgagttgtctcttgggttttggaacatgctgatggggaagacaaggaattgtgtcatgttaccaagaaccagatgggcaggaaagatatggaggagtctctaaagatcatcaaagatcatcactccaaagggattcgtctttggggttggggagatggggcctcattaggtgacctagaacaaaacaccagtcttctaggactgggcagaacagcctatatttcaggagctctagaaccaaaagagagtctttctggaggcagaggctggacagtggaggagggcaggagttgagtttctggaatcctctaagtgattatctggaagccatgggaaatgctggatgggcagaacctggggcaaggggcagaaacgctggagaaagcctctggctgggagggtcccatggctttcttggaagagaacctggaccttctctccttgccctcagttccggtttggcagtggtgggggtgacaagggccctgtggtggcggcccaggaggctcaggcccaggcgatcctgcagcaggcgagggtgagtggggctgcttccctggaaagggaaggctctggggggcactggaagggttggctggagtgagtgtgcggcaggggaggcctgggttggtttggggttaacagggaggttggggagaatcctgggcagtggatgaggatcaggagaggaagggtgaatttggagagtgctggggctgggggttcccactgcctgtccctcagctctgctgtccccctgctcccctagctggcgctccgtggaccccctggccccatgggatacacagggcgccctggacccttggtgagtgagcagggtgctcgggtggaggatgctttaattgtgtgtggggtgtggatagttctggaaaggggcttcttttggggaacactccgaggccactgttggctgtgtccttccttacggccatcctctcttctctctcccagggccaacctgggagccctggcctgaaaggagagtctggagacttaggacctcaggtgaccactttcctccactgcacccccatatctgttcaccagctcagtctccctcactcccctccctatgcctcctaaccccaccccatctctcctctcaccagggccccagaggacctcagggcctcacaggccctcctggcaaggctgggcgaagggtgagtgccccaggggtggatgggtgttgtggggagacagggcctgcagggtaggggactgcggccctgcttgttctgacacttcccttgttctcccagggccgggcaggtgctgatggagcccgagggatgcctggagatcctggagtgaaggtaacaggcttgggcccctccctgaagcctgtagccttcagcccacgctgggctcagttgtttcttggggatgacctagttctccaggcttccccaggatcagcacccccccagctcagggtgcagtagggaggggtgggcttggacagggtcgtgtcctcactctggctatccttcctcctcctagggtgaccgaggttttgatggactcccagggctccctggagagaagggccatagggtgagtacattagtgggtgtgtgttgtaattggggatagatcttctatgggagtgaggataggtagggaagggaggctggaggcttggcaggagctcagtgaaagtaatggatgggctaagtgcagaggttcggtgtctgcctgtgttgggggctctgaagcccctcttatgagttcccccattttgcagggtgatactggtgcccagggccttcctggtccccctggtgaggatggagagagggtaagtgtagtggcaaatgtaggggctgggtgcaggggaggtctagaaggcacagcagctttaccgtgtcctcctcctccagggagatgacggggagattgggcctcgagggctgcctggagagtcggtgagtgtccaagggctggtgctgggggagggggctctggggaagccaggagggggacatgagcactaaagggacacagttttatccattcatgccgtctcctcccctcaccgtgaatgcagggacctcgaggtctccttggccccaaaggcccacctggtattcctggaccccctgtaagtgactccctgacttctgaccctgaggtgaccccttgacctctccaactctccacctttctagctgccttcctgctccctctcctctctcggcatgcttcccattccctgtgagctccccctgctctggccccacactgccctgcctcagcattcgctccccgtgcacagggagggccatgtggcgtggtgccctgagggctctgggcataggaaataaatcttcaagaaaaacaaaaccaaagcccacaagcccacagatccacagtcccacacactgcaggcctgggccagctgtgtttgcttgggtttgggatggattggaggtctgaccgtcacaccctcgggaatggccgggcctctgaaaaccacaggtccctccgggccaccttcaggctgctcacacccatcttgtgtttcagggcgtccgaggcatggatggtccccagggccccaaagggagcttggtgagtgatggataggagatcccacccccattcttatcccccgaggtccctgccagcatcctgttggccgccattttgacccctgcctgcttcctgctcttgccttcttggctatcgctgctgggactcagctccatgctctaaatgtgttctatctccatctccttctgacctgtgcctggtcacctgtttctagggaccccagggagagccaggacctcctggacaacagggcacccctgggacccaggtgagcggtgcccctactccgttccccaattttcatgacttcccttgggcttccacaatgggtgagaccctggaaggatacaggaaattaagggtttcaggcccttgttgctcatgctttgaatccgggaggctggatagaagagaccctgggaggcccctgtgacttctctttccctgtatgtagggtcttcccgggccccagggtgccatcggccctcatggagagaaggtaagtgactaagtgatttggaggacagggggtttagagtggggatgtagggagaaccctaaatgtctgctgggattttgtctgtgtccagacatgacccctctagtgaccctgagcctctttgtcttcctgcagggtcctcaagggaagccagggctccccggcatgcctggctcagacggacccccggtgagtgggaccccacttccgaatccagttccccttggccttccaccccagtgcatgacctcagagctcctttagtgacccttgcactgaaaggtcactggtggtgcttgtccatggacagttggcttcactaggtcacaggaaatttgggttggaagaagtgtagggaggggtgcagagagggtgacaggggccactggtcacattttctatcccttactcccagggtcacccagggaaggaaggtccccctggaaccaaaggaaaccaggtgagatcttccatgtctttatcccctgagggagtctccactcagactctagagcctcaaagtccttgggattcccttgtctcattattcatatggctctgccacattctcaattcctgtgccccgtaaccctacccagacagcaatgttgttacttccatttcttccacccctggaggcccccacagtgacactctgaggcccctttataaatgcctctcttttatcttccagggtccctctggacctcagggacctctaggatacccaggacctcgaggggtcaaggtaactgaccaccaggctgggagacaaagggctgtggtcaggggagctatatggggtcttaggagcttggtccttccaacccacctccatcccctgatcagtctacttctatcccctctctaattctagggtgtggacggaattcggggtctgaagggtcataagggtgagaaggtgagcaccatgcccccagcttcccagcacctcagtcctgccatctcctctcttcactgccttccctccagcctgccccgcactgccgtccagctcccgagcccctgcttccacctatgttctcagaattcccattagaaccccagccctctgtcatctgtggtgccctctcacctctatctttctcagggtgaggatggctttcctgggttcaaaggtgacataggcgtgaaaggtgacagggtgagtagagaccccacactggccccaattccgtgttctactgagcctccactctggagacgccaagcaccggtttattctcctgagtctccagactccacagttccagtgctgtgttcccttgggagcctgacccctacaggatgatagccccatggtctgtgtcatgcctgccctcccatcactacactatccctaccctgtgggcttcctgtcctgcagtggctcctgggacccctctagccctcctcagcctcactgttgcccatttctcctctgggcccagagcccacctccagtccccagcactcacctgtccttccctctcacacagggcgaagttggagtccctggttccaggggagaggatggtcctgaggggccaaagggacgcactggaccgactggagaccctgggcccccagggctcatgggcgagaaggtgatgggatgagggttctgtgcctgggtcctctgggggtgtgggcactggggtcaggatgggcatgggtgcccgccgctgaagtcagccagtcagttgaagatggccatgcattagtcatacttctgtctgtgtaaatggcatctctcagctgctgatgggcagaggggatatataagaagtcagggaaaagtccctgacacgaagagttggacggagacgaagcttagagagtagctgttgcttcaaggcagagtgaacatgcgttcggactccaagcagcaggagttctgagatgagagagtctaatagcagtgaggtctgtggtctccagagctgactctccccagcacttgccctgtgccaggtgctgtgctgatgcgtcctatctatcctatgaggtaggcatgtgacgtactgttttgctgaggttttacagaggcacagggatgcttgttgattacatggccagtgaagggcagagccaggggttatgcccaggcagtctggcttcagaatctctgcttctaagccctgctctgcagctgcctgggcaggatgagccctggagcctctggggaagggggcatgcacgggagtcggggtgtggtgggcaaagggcctgcagggggattggtcttggtgaagcaggagtgggtgctggggtagatcccacctccctcatggggtagatggagctgaggttgacatgtgggaagcagctagatcacagtgaccctgaaggacctgatacctctgtaggcgcaggggaggtgggatcccagcactagaattggggaaaatccactcacgggaaggagagacaacaaaagtcaggcagctgtggggcaagcaatgaggggctagaggctggggtgtgtggggtgtgttgctgggtgggggctggctgttatatgtgagggttgaggtctccaggggtggagtggcatctgggcctcctctcactcctaactgcttcctgtccctccacagggcaagctgggtgttcctggtctgcctggctatcctggacgtcagggacccaaggtgatgccatgccccatacgtgcccctcctcctcttccctcctcctcttcgcttccctgatccctgattccagaaagcacagatgcagggcagtgggggaccccagagggaatttagctgacccccattttacagatgaccgaaagaggcccagagagcagcaggggtttgtccaaggccacttagttcttggcagagctcgggtctccctggccactgcctttgtggcctctcctgaccccctttgccccttcctgaaccctacctttcataacttctcaatttccccccttctccgttctcaccaggggtccctaggatttcctggctttcctggtgccagtggagagaagggagcccgggtaagctggggggtgggagggcgtgagaagggaagggctggagaggtgtagagggggtctgtggggagtctcaccctgggtaatgttctctctgcttcattcccaccagggcctgtcggggaagtcagggcctcggggagaacggggccccacggtgagtgcaggggagagaaacaggtggctcaaggtccagaggggggcatttgggtttctctgacaactcctcttccctctgtagggtccacggggtcagcggggaccccgaggtgccactgggaagtctggagctaaggtgaatggtctctacaaggctccaactgccccctgcccactcagccccagctctccctcaacatcctgacctcggggacagctggaaagcagctcttcccactccttgactctgtttccccccacagggaacatctggtggtgatggcccccatgggccccctggagagagggtgagtgtggatcgaaagtccccttcccctgaggtccccagggagcctgggtgagccagctccctcctcaccccaggaaggaagctgagttcccccactcagcccctgcccccattctcctgactccccccacctctgtctctagggcctccctggacctcagggtcccaacgggtttcctggaccgaaaggacccccggtaagttgaccccgagctctgacccacctcgcttctctgagtccttcttccctacctgctttccagctgagagttcacctgacaactgaaccccttccagggcccccctgggaaggatgggctgccgggacacccaggccaaagaggagaagtggtgagtgatgctcctggctacccattctcagggggctccatcagaccctttaaccccagagctgtctggaaagcccacagggacgttctcctggcagagtggagcctgccctggtgctgctcagacccagggcccagggcttggtgtgatcctgcctgggatccttcctcctgggaatctgccccacagcgcccacacctgctttcactgcattctcctctcttcctcgcctagggtttccaagggaagaccggcccccctggtcctccaggagtggtgggacctcaggtaggtttgtccctaggagagaatggacccctgaccctagcattagtctgtctgcccctcttctctcggtcacacgcccctgccccggtccacctgacccagactcctggggctggggtcaatggtgggggctgggatttcaggcaggggtcctgaacttcagtctgttttggggcgcattctcatcgtcttgtcccccaggagaggcagttccccaacagagacacaaggtggtgtagttactccacatggggcaggagaaaacccagcagtggcccaggaatgccggggaatttcccaggagctttggggcagggataaaggggttctgaaggagtgttttagggtggccagggtgggctcttctggcagacagcgagatgaagggcctgagagcaggaactctctctgcttttgtctagggagcagcaggagaaaccggccctatgggggagagaggtcacccaggccccccggggccccctggagagcagggactacctgggacagctggaaaagaaggaacaaaggtcagtgaggggccaggcagaggggaagtgggggagctggggattggggtgctgaaaggaacaggtagttggagatttgagggggggctggagagctctctgtttaatttgggagcatggctgtggctcatcacctctcctttccttttagggtgaccctggtccccctggggccccagggaaggatggtcctgctggtctgaggggattcccaggagagagaggcctcccaggcactgctgtgagtgtgactcccagacccctgcctgtcacaagcaccaagcatcctgagtccccaccccgaccctgtccctgaccccctccatgtcactccccacttccatcttttggagcctgtcttccctctccagctctcacccttctcttttcgtgaagtctcatcttccccaaatgtctggttcatagggtggacctggtttgaaggggaatgaaggtccgtctggcccccctggccctgcagtgagtctggggttccggaagtggtaggaaggacccgggaaaggggtgggtgaggaagggtgggagtggcattctgataccaccaggggctgtggggctgggcagaggctgtccagggacagccagaatgcatctgattttgggagcttttgggaattgtggtctgaggttctctggtatgggcgggctggtgtctccttcctgggggtctgaccatctgctttctcagggctcccctggggaacgaggtgcagcaggatcagggggacccattggtccgccagggcgcccaggcccgcagggtccccctggagcagcaggagagaaaggtgtcccagtgagtgtgggggtattggggaggggtactggggaggggtctgtgagcctagggttgatgggctatgacaagtttcctgtggggtgtttgagggtgagggtcacctccaggccatgactccttcctcctgtcccacagggtgagaagggccccattggcccgactggccgagatggagtgcagggtcctgtggggcttcctggtcctgctgggcctccaggtgtggctggagaggatggagacaaggtgaggggaccccacagaccccgaccaatcttctttccagatgagatggctgacctgggcatgacccctggcccctgtcatgtcatccttcccactctacccatcacctttctctaatatcttgtctgccttctcctcccagggtgaggtgggggaccccggacagaagggcaccaaagggaacaagggtgaacatgtaagtgtccatgaccttgacttctgacctcctggcctttaacctcagtcccacctgatctctctccctgttttggtgtctctgactctcttctcttacccagggccctcctggaccccctggacccattggtcctgtggggcagcctggagcagcggtgagtgacccctccacccccaccgaaggcccagcagcctctggtcctccctcagctcccctctgcccttgtggctgggagtgggagcaggggctctgggcctggcacccagctgtgtgtggtatttgtggatagagaacacctgacccatgacccatccctgtgtccttgtctctgctttccctcaaccctgcagggagcagatggggagcccggagctcggggaccccagggacactttggagccaaaggtgatgaaggaacaagaggattcaatgggcccccaggacccattggcctacaggtgtgttgggggataggacaggggctgagaggtggggtcaggatggggtgatgttttgccccagactctgccagcagcctgccgatgcagactggagggcctgcggagtctgaggggaggggactgtggggcctatctgcctagggctgtgctttgggtggggtcgtcacctgggcccatgttttgtatacttgcacagggtttgccaggcccctctggggagaagggagaaacaggagatgtgggtcctatggtgagtgtgacccctactgaccctagccaccatactagtcacccccctccctggccagcccccaccccacaccccactgccaactccctggcctggctgctcctcctcctctctccaccattcatgctcagccatctcacttcccttcctcatctgtggtgtatctgtttcccccagggaccacctggccccccaggacctcgaggtccagctggacccaatggcgctgatgtgagtcatctcagcccctgtcccttgagaccaatgtgtctggtcttctgcctccctttcccagaccataaactctagtatcgtctcaggggccaagagaagcccttactcccgggaggcctccactgcctcactgctgtaaccttgggctgctcatctgggactttgcccctgaaatggcacctccatcctaagcaatgacacctatttctgttcctcttccagggcccacaaggtcccccaggaggtgttgggaacctgggtccccctggagagaaggtaactgggaagggggtgagtggaccagtcatggaagtgggggatgggagttggatttctgccaattttgggtggggagacaaaagagaatgagatgtggggaatatcagaattttttggctggggaaggagaggaggtttttgactctaatctctttgcagggggaaccaggagagtcaggatctccagggatccagggcgagccaggtgtcaaggtgagtgacagctccaaggccttactccccagtccccgtcacccgcctcaaccctgcctccacttttcctgtgacctccttgagctggaactcctctgcagcaagagtctctgctccctgccatcctctatgaggcctgatccctggtctgagtgagcccttcttcccctttcttgaagggaaggggactagggaactccccttgtaggcttgactttctgtgtatgtcccattgtcactgaggggagtaggggatggctgaaggtgtctcggaagcagaggctgcatccctgatcttcaagattccctgggccatctgtgtcctccgcttgttctgcagggtccacgcggggaacgtggagagaaaggagagtcggggcagccaggagagccagggccaccagggcctaaaggccccacaggcgatgatggccccaaagggaaccctgtgagtttggggcagtgggggctgagtcctctcaggccctgtgaatgacagacctgggaatatgggtgtgtgtgagggaggatcttggagtgtggggagcccgggagtttgggggatgctctgggaatggggcatccagagggatgcctggggtctaggatccggagagaaagtgagagatgccctgcagttaataccttgaggaattagacaacacgcagtgcacgtggtagggaaaggggtgcaggagacctcttcctggggggtactggtgggcactgcctctagagaggcagtggcgtgtgcctgtgggcgtctgtgctgggtgggcttagggagctgtgaccctgactcttattctccatctggatcagggtcctgttggttttcctggtgaccctggcccccctggagaaggtggccctcgggtgagtcttactcagagaagggaaggggcaaaaagggtggggcttctatgggggggtcctctgtgtggccgctgggcttgggtattgggaagccgggggtatggcagggtgggcaaggggatggggtattgacagttttggaggtgatgccagccaggttgggggcccacctctgaccttgcttcacttctgcagggccaggatggtgctaagggtgaccgaggcgaggatggtgagccaggacagcctgtgagtgcctggtgaccccaccacccccctgagcccaagcctcatcctctttacccctcttctgtgccccactcctgaggggtcccttggctggaggataaacactcagccaccccaattcctctctccctagggatcccctggtcccaccggggagaatggacccccagggccacttggaaagcgagtaagtgaggtggacccctgagaccttgggaggcagtccctgggctgtgtgggtggaggctgggcaatggcaggtgggatgggtggggaggtgcctggtgtctgcattgccctgggtgtgtgtgtgtgcaggagctggtgggtttaagggcgtgtggtatctcattgcccggggcagggtgtgtgtgcaggagctggtgggtttaagggtatgcggtatctcattgcactgggcgagtgtgtgtgcaggagctggtgggttgatgggtgtgcggtatctcgggcatgtttgttcctgggttctggtgtgtatgttttcaccaggggtagtggtggttactgacaaagcagaatggaaactggaggaggggctggccagcttttctgtgggccaggggtgaacctttttagtttctggggcaggagacgggccaccaggtagggtgtgggcaagtggcccttcaccaaatgtacagactacccagtattttcacaactgtcacagctgtatctgttctgcacatctgtgaatcggccctcggcgcgtgtccctgtgtatgcacgtgtgtgtgtgcatgtgtatgtgtgtgtctaggacaggaagggggaagagttgagcctggctgcccacggcctcatgtgctcttccttcccactccacctgcagggtcctgctggctcgcctggttccgaggggcgacaaggagggaagggagccaaggtgagggacaggctgccctagtgctggagcatcccctccgcctcccacccctcagatgcctccgtctccagatgcccaccccatctccacctctcccatgttgggcccttatggggacaggcgttccctgtacttgttcctgcgctaggggctcctagagtttggcccttcctccctgcctcctgcctcccaccttggaccctctgccccctgcccacaccccactcctctgccattcaggacacattccttgtgtgtgtttcagggagatcctggcgctataggtgccccggggaagacaggcccggtgggtcctgcaggcccagcagggaaacctggccctgatggtctgagggggctcccaggctcagtggtgagtcactgcagggaagggctgggctggggtgggagtgaggggccatgggaggggtgcagtgtggggatgctcctcctgacccctgtggccccctcaaatcttcagggtcagcaaggccgacctggagctacaggccaggctgggcccccaggtcctgtggtgagtgactgggattgggctgagtgaggggtgagggcagtgccctgggacagagctgaagccctggaggaagtggaggctgttggggagaacttggtcccacctctcccttaagagaatgacttcccatctctccacagggacccccagggctgcctggtctccggggcgatgctggagccaagggagagaaggtgagtgacagacagacacgtggccaggtgtctctcccatcaccctcgcccctgaaacctgtgggacccaagtgcccactgctctgcttcaggcctccggcagccagtcccagggagtccctgctcagtgagggccactcacggaccctgtgcccggctccctctccatgtcctcttggcccttctcccccatccacatgactcctctttgcattctcttgactgcctgcacccttctctagggccacccaggtctcattggactgattgggcccccgggtgagcagggagagaagggagatcggggacttcctgggcctcagggctcccctgggcagaagggtgagatggtgagtagagggcatggtcccggaagttgtgggggttgagagtggggtggagaggggtggagttggagttgggactcagctgtgggtaagcgagcaggtgctcaggagggtgtgggaggaggacccagttgaaccagccactacccttcctagggtatcccaggagcatccggccccattggtcctggaggtccccccggcctccccgtgagtactgcctctgttcctccacaaaatcccctaaacccctctgctgcccacccacagcctcccaacgacttccatggaactaccagcctaacaagcatagtcctcagggtcccccatttgtcctgtcaccaccagtacctcctccccatacttccagggctctgaatgtcctagtattcccacaagactgcccctcctgtagtaacccgaggctcctgtggacttgggaccttgcctccccgctcctctgagtcctgtccttcccctgcagtgaccgtctctttcttgttcctcatttcacagggacctgctggccccaaaggagccaaaggagccacagtgagtgacccccttcagctctgacctttgctccaccccctctggacttgatgatgtctctccaggacaaatagactccccccaaggagccccttattccagccaggaggctgatttgatctttctgtgaccttgatccatgcttgacctcttgacccctccatgacctgatttattccttgtcagggcccaggcggacccaagggagagaagggtgtgcagggccctccaggacacccggtgagtgaggagtcagggctgctcccagggccgtacccctctgctccactgctgccacacttcaccctcacaccaaccttgtctcttgccctctccatctgtccctgcacccagggtcccccaggcgaggtgatccagccactgcccattcagatgcccaagaagactcggcgctcggtggatggaagccgtctgatgcaggaagatgaggccataccgaccgggggagcccccggcagtcctggggggctggaggagatctttggctcactcgactccctgcgggaggagatcgagcagatgaggcggccaacagggacccaggacagccctgctcgcacctgccaggacctgaagctgtgccacccagagcttcccgatggtcagtgctagcctcagggtgcacaaacatctccccaacagcatgggtactgaacaagcagaatggatgctgggggagtcttggtgaaggtgttgggttaagggtgagggctgaggggctgggaactgcagctatttactaaccaggtcagaagtatcccaatactttagcaactgatctgggtcagtgtgtaccagttggttatcagcccagtgtctgcaggagcagataaacatacatacgtgcacacacctgtttgcacctgaacagatgaatctgtgtgagcgcctgcaaccacatgttgtcccagtatgaggctgtccatactcggacctcctccagtcagagggcctggggttactgttgggggcagaggtgtcacgtgatgaaagtgggctgcagcagtaggatgctgcagtagaatccagcagtagggctagattctagggctctgggagggaggaggaccaggagactaagagcatgatggggagggctgggagggaagagggaatgtgggggcaaagagcatcttggagcctggacctaggtggccctgacctcccctctccctcacccaacaggagagtactgggtcgaccccaaccagggctgtgctcgggatgccttccgagttttctgcaacttcacagcagggggtgagacctgtgtgacgcctagggatgacgtcacgcaggtgagagctggcccctcgctgcccctcccctgccccatagtgcaccccctcagggcttagtggtttctgggttttggtgacagtcccctggctgttgccccccccggagcctgccaccccttctccacttcttagctctttcttcttttccttttcaccggtcctctgatgctcactttgcctttcgtgtacccccagtctcctggctcccactcagcccctcacttctttcccagcccttgccttccagccattccctgctcatgactctggctcagctgtcctggtgccgtagatgctcctgaggcccgtctctgtctccctctgaggccagccctctctctccccctctgaggccagccctctctctccttccagttctcttacgtggactcagagggctccccagtgggtgtggtccagctcaccttcctgcggctgctcagcgtctcagcccaccaggacgtctcctacccctgctctggagcagcccgtgacggtcccctgagactccgtggggccaatgaggatgagctgagcccggagactagcccctatgtcaaagaattcagagatggctgccaggtgggaacaggaagagctgggttgggggctgatctcagactcaggctggaggaaggaggtgggaagaccccttgggcagggcacccagggggagcagggaggagtccgtcctgctggattgtcagggatgcctgagggagctcaaaagccggtgaggaaggtggaaaggatggaagccatcgggtgaggttcacttgggtacaaacaccagcgtcacacaggttaatgcagacgtgtacacagactggcacatggctgcccagcagaggcacacggtgggggagaagcaaacacacacgcatgggcacatagacactgccagtctgtacatcctgagtcaccctgatggggccaaggtgctcagaggagaggtgagcctgggcctgaggttagagggtggggggtgcctccatcctgctcacactttcttccttgtctccccctgaagacacagcaaggccggacggtgctggaggtgcgaacgcctgtgctggagcagctgccagtgctggatgcctccttctcagacctgggagccccaccgaggcggggaggggtgctgctggggcctgtctgcttcatgggataggaccgtctctgtctgatcctgtccattcggaaccaggcccacctggaatcccacaacatcagctctgtgccacctcccaagagggctcctcactatctagggagccctgggccagggcgtggagagccctcagtcggggcaggccaggggaggggtgaagtggttgcctggacaccccacgggaggagtggcatctggggctcttggccctcccacctggagcctgttacccgttagagagctgagacccttatttaaaactcacctcccaatcaccccaaacaaatggaagagaagagaaaggacatggcgtattttgtatttaaaagtaattgtattaattatttaaagtgtggaaagcaaaataacaaaaaagagaaacgccaacaaaaatcagcagatgttgaagacaggggtctcgggggtgggctccggcacccacatcctggagtcaggactttcctcagtgactgtgtgtaggggggtttcagggctgaaccccacctccctcccaccttcctcccacctcacctgtcgcacccactgtgaaagttggaatatgtggtctccctggcctcagggctctgactctgccagggtggggctctctaacccacaggtgttggctgcctggcccatgtgcccactgtctcttccacttggtctgggtttggcaggcactgcctgctacttgagggccaggatgctcccccagggaagaaacggaatagtgtggggtgtgtgcagggctgcatcccgcagatggctggaatattaaaattcttctatattggctggtaaattgccatggccctgagccactgagtatgttcattgccacccctgtccctcccctgggcacccctcactttccctgatcctgcaattaaagggttaatgtgtggcatatggaagggactcccaggaccctgtgcccagcttccatgctgactgatggttaaataatgtgattgtctcctcccaggtgtctgtgtcactgcttgtgttgttatttcagtctcccccgacacccatctgatgcttcctcttcccagctaagtggttaccagaattgtatggcttaatccagataccctgcaagccctgtccagctggggtgtcagggcccagagtttacaaagcctgggtgactagcataaaattacaaacaatgccccagggacaccaggtaggggtttggggtccctatggctccagtttctgacacgggtgtgcgattgcttctgggtgtgggtgggacggtgctacatagcccagtctaatctctgtaaatggggaggctgaggcccacttccaagacatttactgcatctgtacctctggtcaccttatccctccccatcagccttgtgctgtgctgtcatgaagacagggctgtggccaagccctccacactcacgtgtagtcacgtgcatactcgttgagtctttgtgttgggtggagtcgcacagtccctgcctgtgtccccccactcttgactgtcccccacttccctagcctccctctcctgggcactcagaagctctgctccctccgggttaggtgtccaagaagtcctctcttccaaggctggccgcgcagccgggaccccctagctggtgcccatggggcttcacattcccaagcagtattgcctggctcctgcctgtcagggcgctgcattaaaacgtgttccagggcctcatcccaatttcctcttcctcaaccctcttcctcgcgggcgccagaatcaagagtgcgtgcctttgtgtcccggtttcgcatccgcggcctcggggcgggcctggcagggctgcccttcaccccgagcccgagcccgggtaggctgcgcccccgcccctcctccccgtacttcttcctcccagcgtgtccccgacagcatcggcttggccgtcccggtcccaacccattctcgatcgcaggaagccccacgtgggcgtctagacggccactctggcccgggcgggtgcgcggaaaaacttctcgagggtggaagcctctcctcggcgctggcgcctccgcctctgccgccccctagtgcccgcgctgagaacgcggccccgcaccgggtcagctgaataatcccggtccctcagacgcctctcccttgtcacccgctgaaccccctgaccccgaaggccgaatggaggaagaagcccctgatctcatctgtcaggggtccggtggtggataggttttctgggatcacccatgctctccatctcaatatccaaggcccaggggcttcagacacccccttccctccctctcagggtcacattaagagaagatcagatctaaataggtgccccccatctgtttctcccgcacccactcccagcctagccgggctggagttgagatttctttggcgctctttctgcagctggagccaaacccgcagaggtcgccccccgaacagcaagaggactttcccaaaggtcacgtttggtcagatgctctgagttccgagggcttttctgcatggaaacaatgctgtacagtcactcgaataacatttatatacggcggctaagtccccaggccaatccacaaaagcctatttaattacacagtgctgccaaccaaccccatctaacagtttcacgagggagtctgtgccaagttgctgttgaggaggccaggcccacagccttctcttggcagctaacttcttcctcccctccagaaagctagagccaggctctaaggaagggtgggaggggtttaagggcagggctgtagaggcggggttttggggtggggctgggtgtgaacagggcaagtgcccaggcctctgggcctgtgggtagtggcagtgtctccattggctggtgggcaatggaggcttctgggtggggtgggaggagatagggaagagaaaggcagaggctttagggaactactttgggtttgattggggcagaacataaactcttgttggaggggccaaattctgaaaagtattgactgcttatggaaggagttgaacttgaacccaaaaactaagagtcatagatggtgtttgaaaaggcaagtgatatgatggaattgtgcgctggcatggtggcgaaggagaggagggtttgggatggggcgcacagggagatcatgtttttttttgttttttgagacagagtctcgctctgtcgcccaggctggagtacagtggcgccatctcggctcactgcagcccctgcctcccaggttcaagcaattctcctgcctcagcctcccggatagctgggactacaggtgtgcactgccacgcctggctaatttttgtatttttaatagagacggggtttcctcacgttggccagtatggtcttgatctcttgacctcatgatcctcccgcctcggtctcccaactgctggggttacaggcgtgagccaccgcgtgggagatcatgtatagagggagggctgtggggtgggtcctctggcctgtcacacccttggagctgcttttcagctttgggcttatatttttagcatctgtaacttatggttaaaaaataacttgtgaaataaaataaaaataacttacagaccacttatatcactgtacctgggagcaccacatatcctagaaggccatgggctacacagagctgttcttgccctcccttggctgtcttgcatgctgaggtttgtagttttgaatggaccagggcaggtgggggtggagatgagacagaggagggagtgagtcaaaacaaggctctcagcctgggcaacaaagcaaaactccttctctctctctctttctctctttctttgagacagggtctctgtcactcaggctggagtggagtggcaagatcaaggttcactgcagcctcgacctccgtgggttccagccatcctcccacctcagcctcccgagtagctgggtctccaggcatgtaccactagtttttgtattttttgtagagatgggattttgccctgtttcccaggctggtctagaactcctgggctcaagagatctgcccatctcggcctcccaaagtgctgagattacaggtatgagccatcacgcacggccctttttctctacaaaaaaataatttaaaaattagctgggcatggtggcatgtgcctgtagttgcagctactcaggagactcaggcaggaggttcacttgagcccaggagtttgaggttgcagtgagctatgctcttgccactgcactccagcctgggtgacagagagataccccatcacaaacagacaaacaaacaaacaaagggctccagctgcctcagaaataagtttctgtgtagtagctggtggccaccttgttttgtggttgtttagcccattttcctgcaagtgtggtgtggtcagcagggagcatcagggccacactggagcttctttctgtcgagctgtgcagttcacagactgtacatgtgtaggccatagctatgtttctcaactcaaacctagagtaagcagaagtcaccttaagtgcttataactggccttctgttatcttttctttcatcttcagagtagtttcatgggaactggcactgacagcagactgccatctcagtagggaagacctgtcctgctagtccttgaataaagaccaaggatatctacatgggcttctcctttagggctctccccacacggccaggatggtagaatgaacacagctctcagcttcagcgagagccagcatatcaccctgtctggctttccctcagttggcaggagcaggtcaatccattttgtgtacttcgctctggcctctctttttttgtttgttttttgtttttgagatagggtctcactccccttgcctaggctagcatgcagtggtgcaatcatggctcactataggcttgacttcttatgctcaggtgattcttccccctcagcctcctgagtagctgggactacaggcgagtgccaccatgcccagctaattttttgtatttttagtagagacgtggtttctccatgttgccgaggctggtctcaaactcctgggctcaagagatccccctgcctcagcctcctaaagtgctgggattatctggcctctcttttttttttttgagatggagtctcgctgtgtcgcccaggctggagtgcagtggcacaatctcggctcactgcaacctccgcctcccgggttcaagcgattctcctgcctcagattcctgagaagctgggactacaggtgcccgccaccacgcccggctatttttttttatttttagtagagacggggtttcaccgtgttagccaggatggtttcgatctcctgacctcgtgatccacccgcctcggcctcccaaagtgctgggattacaggcgtaagccaccgcgcctggcctatctggcctctctttctattaaaaacgccttactctaggttgaaggacttttggttcctgaggcatggatgatttcccaaagtacaacagataaaagccacactggctgtacagcagctcatgtaaaggtcatgttccaggtcttggccacttcacctataacaggcttggactccggcagagcctcaggccaaagaatgtgggtcatagagatatcttcctcattgtgttcacatctcccccgggcctgggctatattgttgggtcaggtaatcggcctgctctcctcatgaaataaggcatactggtattactaatgctgggagagatttcttataattccatcccttcccagttgtggttatttgtattaatacctgcaggttttccttacttttagaactgcaaatctatagaaacaaacaaaccccaaacccaacaaacatgtttcagagacattagggttagggagaagatgagaaatagcaccctgtttgattggggccctggggagagagggactcagacaaaagagtttgagtggcaggtggtctagttttgggtgggttgagcagacttagggccaaaggtggcactgattctagctggcagcgggcctctctgtatttccaactgagagttctccttcccttagcttgtcagggatgctttgcttctgagttgcacacttaacctcagcatcagtttcttcacctgcaaagtgggataatacttgctctgtaaaggaataattccccctttactgacagcctttgcaatggaaagtgaggggagggacctgacaagctgtcttggttccatctccatgtcttggttattgctgacacacgggtgtctggggcatgctgctgtcccttttccacttttgattttagtgctgttaaaactctatacttactattattattttcttgttacaaatcgcaaaatctgattcagaatagcttaagctccaaaaggaattgattgtaaaaatattgggtttctcaggatttgaagacagagttgactcaatagggaagactgagagcagttctgggaacctcaggggctcatggactttgtccctggagtgtggatactcaccgctcagcagagctgctcttgacctctgtgtctctgggccccatggttcaaagtcatggagagacaatctgattggcttagcttgggttatctctgtccctgagaaccaattttctgtggctgagggcccagtgtaccaaccaagaggtatgtaggatgccttttggtggaggtggaggggtgttgggggcactgtttccagataatgtgggatcctgaggcccccaggaggtgtccgcttgctttcccttcttcgcttctgtctaaacagactcctttgtatgcatttgaccagcgtgatacaaccagccccttcatgatcacatttgctctccctgccacccaatctcctggatgggcaacccaaagtcacattcagcagctgtggccatgacctctctaacctcagaggccgacatctctgccagttcatattttgccactttgtgattcagcacataaactttcatggccgttatatccagggctgcattgtgacttctgcgggccctatgcacttttctaaacaataaatttgaaatcatacttatgattgtgttgatacacaggcaactatattaatattatatattaaaatattttcttcaaccttaattttttttctattttaaaagaaatgtaaacattgccatgggagtattgtgggccctaggcactgtggctactgtacataatggatccatgggccctccttgtgtctttgttaaagattttactggtttactcttaaaatgtaatcacaattctgttaccacaattttaataatcattcctgttgtcatcaaatatccagtcagtgttcagatttccaagattgttttgtaggttttgtgtgtgtgtgtgtgtgtgtgtgtgtttgactcatgatccaaataaagctgatggtcttctaattctttaaagatctacattttccgtaatttatttaaagtcctttttgtttacttttattatggacaaatttaagcatatacaaaagtggtttattattgctttttttttttttgagatggtcttgctctgtcgcccaggctggagcacagtggcacgatctcaactcactgcagcaacatccgcttcccaggctcaagcgatgctccagcctcagcctcccaagtagctgggactacaggcgccagccaccatgcccggctaatttttgtattttttgtagagacagggttttgccatcttgtccaggctggtctccaactcctgagctcaagcaatctgctcatcttggcctcccaaagtgttggaattacaggtgtgacccactgagccttgcctgttattgctatcttaaaaagaattaatacatgttaaaaatctttttcagttctaattttgaatacagtaaatatcaataacaatattgtaagtaaagcttttcagtgttttcagtaatttaaggggtttatactcttaaaacattgagagatttttttcaaattgagatttaaaaaaattgggaatcactaatctagaagaactgttaaaaatagtcatgcccacctgtgctcacccttagagatctggattcaacccatttggggcgtgagcaaagggacttgtgtaaaatttccccaggcaattccaaagtgcaggctgtgttgagaacctctgctctaaactggagagatcccgtaagaaataagcagccaggtgcagtggctcatgcctgtaatcccagcactttgggggaccaaggcaggtggatcacttgaggtcaggagttcgagatcagcctgttcaacatggtgtaaccttgtctctactaaaaatacaaaaaattcgccaggtgtggtggcacgtgtctgtaatcccagctactggggaggctgaggcaggagaatcacttgaaccagggaagcagaggttgcagtgaaccgagatcatgccactgcactccagtctggtagacagaatgagaccctgtctcaaaaaaaaaagaaaagaaaagaaaagaaaaaaaaggaaataagcaaagatggccgggtgtggtgcctcacacctgtaataccagcactttgagaggctgaggcgggcggatcacctgaggttaggagttcgagaccagcctgaccaacatggagaagccccatctctactaaaaatacaaaattagtcgggcatggtggcacatgcctgtaatcccagctacttaggaggctgaggcaggagaatcgcttgaacttgggaggcagaggttgcggtgagccaagatcacggcattgcactccagcctgggcaacaagagtgaaactctgtcaaaaaaaaaaaaaaaaaaaaaaaaaaaaaagaaataagcaaaggcacagagtgggaaaggcaaccaaatgccctaggtctgaaagggcctcctgggacctgctgcatccagcagaaatgcttgcaagtgcaggcccagacaacccaaaaaacaggggctaaaataaacaggaattcatttttctcataagaaatctgaggatgacagcttccagcattggtttgagcatttaatgatgccatcagggttccagactcttcatatgtttacactccaccctcctacaatgttacccttacatattttctttggctaaactataagcacgtttttcataatagctgttccatttacatcactgtcaaacatgacattcaccaataaagaaaagacgcatgagacgtgttcggcaccccttagctgtagaaatgcctgccaccactttgctgtcatgaaagagccattttatggctattgcaaatgctttgaagatactaagacttgtacctggaagctatagatgcagtgtgggaaactgggaagtataattgccactgaattgttattgatttattactgcaaataaatctatgcaggtcccttctgaaattgagatggtcctgaactgaacaaatgtcttattgacattcataacagtggctggtattccttctttatcctgtttgtcagtttttggaattcactgattagataaataaaaagaatctcacttgctccttactaatgagctccacggaggggcatgatgttggaaaaggcaccatgcaaggcatgattactaagaagacaaagggcttgtcggggagctgttagggagggtcctgagccagggcttgggaaaaacatctcatcattttactgtatctgatgcttggaagtgacatgtgctttgcaaactccagtttatgttttgatgactttgtgcttgttctgcttggttaggattagcttcagctgtgagaaacagaaaaccctggaaacaacagtgtctccaccaagatagaagttcatttctctctcacatgcaaccctagggctggtgtagtgtttaagtgtcagagactcaggttccttctatctatctttttcttccaccagcctcaacatagctcttctctctcaggtccaatatgactgcttgaattccagctttcatgtttgtgttccagtcagctgaaggagaaaggggaaataaggacaacccctcttttttttcgttttttcttttttttttttttttgagagggagtcttgctctgtcacccaggctggagtgcagtagtggatctcagctcactgcaacctccgctccccaggttctagcgattctcctgcctcagcctcctgagtagctgggattacaggcatgtgccaccatgcctggctaatttttgtatttttagtagagatggagtttcaccacgttggtcaggctggtctcaaactcctgacctcaagtgatccacccgccttggccaatacctttaggcaaagcattatagcaggagtctaaaaacaaatgggctgttttgttttgctcggaaatctctccccgagggattgttttgttttttgaagtatttgaaggctaagtatctagagcttgtgctcatggagtagctgccccctttcctcgtgaggccaccattagttacctctgcatatatgtctgcctctgaggtgtctggctgtctgaggaggcctctggctttgttagtacaccactaggatatttgcaacgtggatcaagactttttaatttaatttaattttattttatttttttgagatggagtctttctctgtcgcccaggttggagtgcagtggcacaatctcagctcactgcaacctctgcctcccaggttcaagccattcttctgcctcagcctcccaagtagctaggattacaggtgcctgccaccatgcctgttgcgggaagtcagggaccccaaatggagggaccagctggagccgaggcagaggaacataaattatgaagatttcattttaatatggacatttatcacttccctaataatgctcttataatttcttacgcctgtctttacttcaatctctgaacataaattgtgaagatttcatggacatttatcagttcccaagtaatactcttataatttcttatgcctgtcttactttaatctcttaatcctgttatcttcacaagctgaggatgtacgtcacctcagggccactattgtacaaattgactgtaaaacatgtgtgtttgaacaatatgaaatcagtgcaccttgaaaatgaacagaataacagcgattttagggaacaaggaaagacaaccataaggtctgactgcctgtggggttgggctaaaagagccatatttttcttcttgcagagagcctataaacagacgtgcaagtaggagagatatcgctaaattcttttcctagcaaggaatattgataattaatactctgggagaataattgcattcctgggggggaggtctataaacagccgctctgggagtgtctgtcttatgcggttgagataaggactgaaatacgccctggtctcctgcagtaccctcaggctcactagggtggggaaaaaccccaccctggtgaatttgaggtcagaccggttctctgctctcgaaccctgttttctattgtttaagatgtttatcaagacaatatgtgcacagctgaacatagaccctcatcagtaattctaattttgccctttgccttgtgatctttgctttgccttttgccttgtgatctttattgccctttgaagcatgtgatctttgtgacctactccctgtttgtacaccccctccccttttgaagtccttaataaaaagctgctggttttgtggctcaggtggacatcatggacctactgatatgtgatgtcactcctggcggcccagctgtaaaattcctctctttgtactctttctctttatttctcagactggccgacacttagggaaaatagaaagaacctgcattgaaatattaggggtgggttccccagatacatgcccagctaatttttttgtgtttttagtagagacagggttttgccacgttggccaggctggtctcgaattcctgacctcaggtgatccacccgcctcagcatcccaaaatgctgggattacaggcattagccactgcacctggccatctcaagatggttttaatggttagaaatatcagccaattgaattttcagagacatatgccacctaattttgtattcaatacttaagtcattaaactccagtggtctatatacctgtttggcctctaagacagtttggcctctaagtctgtgagcactttctgtgatgatggaaatgttctacatttatgcaatttaatgtggtggccactattcacatgtggctattgagcacttgaaacatggctagtgcactaagaaactcaatttgaaattcaattttaattttatttaatttaaatggccacaggtagctagctagtgctaccatattagacagcacagctctgaggctcttttaaggacccacataattcaatgaagagacccaagtttaatacagatgtgggatatgcagtcttcataattcaaacacgccaattaatttctgttcctctttttcaagttgggggctttcaagcacaaaggcttttacctggtcttgggcagaatttttctccccaaaattttaccaaaatttgtgctcttttatagtctgtttcttttaactcataaaattaataatggaagacagaggtttttgatgcagttgctttaaaaaaaaccaggttcaggccaggcacagtggctcacacttgtaatcccagcactttgggaggctgaggtgggcggatcttctgaggtcaggagtttgagaccagcctggccagcatggtgaaaccccgtctctactaaaaatacaaaaattagctggatatggtggtgcgcatctataatcccagctacttggggctaaggcaggagaattgcttgaacctgggagggggaggttgcagtgagctaagattgcgccactgcactccagcctgggtaacagagtgagactccatctcaaaaataaataaataaataaataaataaacaaataaataaataaataaaatcaggtttactgagatataatttaaatacagtgaaaccatagctgtacagttgtttatgagtttgacaaacatattcattgtgtaactactactacaatgaagataatatttctattaccccaaaaagtttcctcatggacacagcttttttcaagtcactcctggttaatgtgtcattaggataataaaacaatgttaattcttgaattggatttaggctgtttaaattttgctcaatggattgtacctcggaggtgggagaatttactctggcaacttagtaaaggttggggctgccaagtgaaggcagactgttggtgaatataatttattgggattgaaaaggatacatcagccagattgatgcatgaatgctaatcaagggttgatttagcaagatcttctactaaggaaataatatcatgtacagtgctaatcacatttttttcataattgagttaaatatttaaagtgattagaatagtcactgccacttaatactacttcagaattagctgcttttcttaatatcggtgtaattcacaattcacttccaagctccacctcccttttgactggccaaaatgagttgttaaagagtcctactctttatctcagatttctccaacaatgttatcataattgttattttacatattctcctagggatttgtcactatttcatattacgttgttttgtggggaagtggaattctagaggttcttaagagcttttccagctcccacagttgcaatcgtaagttcagataacatgcccaggggcaggagaaatcaccatagaagctttcttggttctctgtatctcccattaatgtttcataaccttccaccttcatactttcccctccacataccctggtattcaaaatagcccttcttattagattgggttttcctaattaaaagttgccattgatgttatatcccaaagtttctggaattcctaaacattgtcctttgtctaaactgttttaacgtgagtgcagagtgtctccagacacaggttggaacagatctggagggaagaacattggggctgtttgtcccatcgtatgagaggatctggttttagggcaaagctctttaagcttggagaatgaatcgaaggtgtggaagtgccctgtttctgtatgttctaagagaaatgagctcttaacgtttataagctttttgtcttggcattccccttcggtttggtccctagcactgttaaattacatgaattgagggtttgccaaaactactgctaaatttgcagcttctgtatctgctaaaattggactatttttgatacttcctttaacctgttttgtaaccttgttaattttctaatgagcccattgtgctgttgggattccgatatccccaaatctaatgagccatgaccatacacgttagctccattcccactgggttctcaaattctttttcatttttccaattctttagcactttctcttataggcggttctgccttaatttgaaattaaatgagaacttcagccccttcgtctcaccagctgctagcattttaagtgctcaagagccacctgtggctagtggcggctcggttggatagatacagaacatttccctaacagcagaacattgtataggacagcactgctcaagggattgctctccgatgatcacacccactttacagatactaggactgtctaactatgtacactttctttcttgcaaatacgatgtcgaaggctttccctcagtttgtaacttttaacaaatattttagaatctttattgatcaatctctcatacatctgtgcaacaaaaatatatgtattcatttaattctttaattgacaaaaattgaatctgtttatggtatacaatatgatgtttatatatatatatatatatatatatatacacacacacacacattgtttaatggctaaatcaagctaattaatatgtgcattaccttacatattttttgtgttgggtgagaacacttaaaattattcacttagcaatattcaagtacacagtacattgttattaactagagtcaccatgttgtacagtacatatcttgaatttattcctcctgtctaactgaaattttgtattcgctgatgttagtttttacaatcaactcattaaaaacataattaggtcacagaggttaggaggttttgaaaaaaacaaaaaaccaaacaaaaaatgtaattaggtttgtttggcaatctagcaatgcttagtgtgcaatgctttttacagccgctgctaaaagcatagcaaaaccacagaaatctatgtgctgccaaaacctatgtatttggtcgtatttattttgacttgtgttttttgtgtgcttaactttttctctaacttttttttgtaaagaatgctggagtattcccaaattgttgacaaattcccttgtttcctgttctaagcccagatttcagtgttggccactaggtggggataggatcctatttctctatagccttaacatgattttgaatttcgatgctttgcaatggataatctggctttttcctcgaaagctttaaaacatttcttttgtgaattcaaaacaacccacagttttaattctctgtagttatccaataaaaaggaataggaaacaagtgctaaattatttacttacagtgtttctgtatggaaatccttacccccaagacagctggcttcacggcttgactcaatggtgagaaatctgagttgactcacagctttctgacagtattctgatggtaccatgttaggataaattcttggccaaacagaaaatcaagggagacctgcttctccttgtcaaactctaaattaatctactagttcttttcataaagcaaaaggtgaaatgttgaggactctgagttggcgatcaatcgaaatgacttttaaactaagtcccttttaatgatgactgaagacaatattacattaatcactgggtattataaacactggtttctcagtcctctgctgatgagatttgcttgtttgtgtatttctttcttaaaatcccaaatagtgaagtctctcttaagcaatggaggcttattcatagtcaaattccttgcatgtaaaattctctgggtataaagtagattgcctccctcttgtttttctttttcttgagacagggtctcactctgttgtctaggctggagtgcagtggcattatcacggctcactgaagcctttacctcccaggctcaagtgttcctcctgcctcagcttcctcagtacctgggactacatttattttttgtttttttttatttttttgtagagacaggattttgccatgttgcccatgctggtcaagctatctgccctccttggcctcccaaagtgctgggattataggcatgagccaccaggcccagcctgctttcttctgttttaccaagatgcttgacagtggatatataaaatgacaacttccctttctcatatgtggcactgagagatgggcagcctctatttcctacaagaaatatagaatagaaaatgtgtaatttctcatggtgttctgcaaaaattaaaaatttcacagaacactcatctgaatccctggtttgtaactttggctttccaaaacttgcctatatgctaatatgattcataaaacatttgtaataataatttcaaaataaaaattttaaaatgaatttagtcctatatcttctataataaccaatatttccagaaaagtaggatttaagagtatcttctgtaccaatatatgactttctttttcttttttttggagacagagtctcactctgttgcccaggctgaagtgcagtggtgtgatttcagctcactgcatcattcagtcggccgaaggcctggctagaacaaaaaggcagaaaaaaggtgaattcactctctctctgttttggagtagggagtcctatattctcctccccttggacatcagatctccaggttctctggccttcacactcagggacttgcaccagcagccccccaggtttttggtcttggactcagagttactccaccagctcccccggttctcagcccttcagacttggactgagccatgctacaggctcccctgtctcagcagcttgcagaaggcctaacgtggaactcctcagcctccatagttgagggagtgaatccccagcataaatccccactcatctatctacctatatatatccaataaattctgtttctctgaagaagtctgacttcttcagcccccaaaggcccccaaaggcccccaaagcccccaaaggccctagtgctggtctgttacgaacttcctggtagacaatattttacacatggcatcccagctccttgtgggaggaattaagtgtgtcctgtgtggctccagggggacaggatccttggaagcctgagcctggtttctcttaggctttgcccagtgtgcctttccctctgctgagtttgctttgcatccttttactgtaatgaatcagagccctgagtaggaccatatgatgagccctgtgagtcctcctagggcatcatctcacctggggtggctctgaggacccctaacatagctcttcctctggcattggtatctggtaatatataaaatcccccaaaactaacttgtcatcataatttatgaccatatttcatcaaaatttcatgtagtcacatccaagaagttcagttaagaaagaaaaactctttaagcttggttaaggctctgccatttttgaaaattcgtattaatctaataatattgtagcaactataagttttcctgataactttttttttttttttgggagtgggggatggagttttgctcttgttgcccaggctggagtgcaatggtgtggtctcagctcactgcaacctccgcctcctgggttcaagcgattctcctgcctcagcctcccaagtagttgggattacaggcgcccaccaccacacccagctaatttttgtatttttagtagagacaggatttcaccatattggtcaggctggtcttgaactcccgacctcaggtgatctgcctgccttggcctcccaaagtgctgggattacaggcgtgagccactgtgcccagcctcctgattactattatataaaatttggacttagtggcacatcataacatgaaatgactggagacaatatgacttctctctcggtattaaacatcaattaagatgaattacaaaaatcatagtaaactgtattctaaaaccaagcctatgtctgaaaaatctggtatacacacatcttttgaaactaaggatgtgttttcactgtacaacctgcaagaggtgatctggtgaacatagagttatggaataagtgtgtatctataacttagtacaaattatatataaagagaacgataatgtattatgcggctgggcatggtggctcacttctgcaatcccagcattttgggaggctgaggtgggtggattacttgagctcaggagtccaagaccagcctggccaacatggtaaaacctcatctctaccaaaaacaaaaaattagctctcaggtactctagagactgagatgggaggatcgcctgaaccctggaggcagaggttgtagtaagctgagatcatgccattgcatttcagcctgggtgatagagtgagactccatctctaaataaataaaataaaataatatattatgctattatacctattatttaaaattgggatcttatgttgatttctacatttgggaagcatattcctaagaactgacctttatcagtcctgtattgtgttcagtggcaaagccaggcttggtgtctgttccatctgggaacgccaggagtaatggtcccactccttgctgctgagggtacagcatgaatttgggcagcaccatgaaataaagcaaatgtaagaagatcctctatcagttgcactcctatttaataagaagacttgatcatcatcaagaagttaatttctccatgcctgggtaaaaccacactgactcatcctgaggctcttcattggacccagtgatcaccaaatccttcaagcataaaatggccatgtgattatctccttcctctcaggatgaaaggcagacaataacatgtgatttatgttcctcaggaaaggacatgctataaactagagaacttgggaaagaggaagtacatgaattaacatgcaggcacatggtggcatgtgcctgtggtgccagctactgggcaggctgagtggcagcattgtttgagcccaggaggtgcaggctatggtgaactgagatcgtactactgcactccagcctggttaacagaatgagaccctgtctgtctttctctctctctctctctctctcacacacacacacacacacacacacacacacacacacagaaaataaaggagaaaaagaaattgttcttaaacataaaatttaaagtggggttctttgtatcaccacattctttttttttttttttttttgagacggagttttgctcttgttgcccaggctggactgcaatggtgtgatctcggctcactgcaacctccacctcctgggttcaagtaattctcctgcctcagcccctcgagtatctgggattacaggcacgtgccaccatgcccagctaatttttgtatttttagtagagatggggtttcaccatgttggccaggctggtctctaactactgacctcagatgatccacccgcctcagcctcccaaagtgctgggattacaggcatgagccaccatgcccagctgactcttttaaatttcattcagctgtgcgcccagcctcaccacatttttatatgaagaagaaataaaataataattactaattaaataattaaatattatttccttctccttatatgtttgaaaacatcaaagattttctccaaaattatcatagctataaaagagtcaatataatatcaaatggtaacaaattaattcctttatgatgcaaagaatcaatgtaaaattgtgaagaaatatcatccattcacagaagtaactacaaataaaaaatcccaacttgcgtaatgtatttcctcattaagaagaaggcgattatgatgtcctaattttcgttttcttcacttacccctacactctgcagtagtcaggggcttcctgggccatgctggtgcctgacaattctcccacttcctcccagcagctctagcatcttccttccccagccacagaggcgtcccttctgatgtattctggttggttgagccccatcaggctgtttcatctccagcctttctccctggacaggggtagggaaaggagtgctggattcggagctcctccgtccctgcgccgcctcattttctccttggggtctggcgcacagtggccattgcaatacaccctaaagactgaaggagccagcgcagagccaggatcctcctgtgagccatggccctggctgccctgcttagcctctcggaacctgggccatcaggggtgagtgcgcaggtgggcgagggagggtgggggctctgggcgcttctggagcccacagatgctgttttctttcctgttctgttttctctgttctttactgttttgcctgaggctctcttttgctttctctcattctaacaataataaccaccatttactgaacaccccctaagtgccaaacctcatgtccttcctcacaacaccattttcagaaaggcttttattaacacaattttagaaatgaaaagactgagactcaaagagattcataaatgtctcctttcccccacgtcctctcgctgaagagagaagagccagggcttggcaacctcctctggagattctctcgtagattgtagaggtcagcttcctcaaataggagtctgcaatgtgggtttttttccttccttccttccttccttttctccctccccctcctcctcttcttcttctttttcttcttcttcttttctctctctctctcctctctctctctcgacagggtcctgctctgttgcccaggctggagtgcaatcacgtgatcacagttcactgcaaccttgaactcccaggctcaagggatcctcacacctcagcctcctgggtaactgggactacaggtgtctgtcactacacctggataattatatatatatatatatatatatatatatatttttccccttagagatggagtctcactgtatttcctaggctggtctcaaactcctggcctcaagtgatcctcctgttcagcctctcacagcactaagattacaggtgtggggcaccacacccagcccgttagtgactctctctcatttcattcagtcttcaacccactgcattctccctttttcttccccatgtctctgatgctgctctcagggtgacctttatacaacccaaaccagtgatcacttctctgtctctatctcttgtgtgtcatgagtgtgtttgtgatgcgtccgctcctctttcctggatgttttctcctgtctgttgtcttttatgaacccacttcctctttgttttcctcctactttctagtcttctccttttcagcctcttttttcatgtctgtgtttgtctggatgcaggagcacacactcagacccttgctattctgactttatgtacttttcttgagtgacctgatccaaatttagaacttcacttataatgagcttccttccagtctgtgtcactaacctagtgtaccctgggacccaaccatttagctcactatcctcagattaactccatttgttcccacagaaattcaaactaagcatgttccaaacagaacatcttaccttctctcgattatttcatttttcattacctttgcagtcactgatgccccagttctccacgttgggtattctggactccttcatatccctacttctggcacatgatttagtgattcctgtggcttttcgtttcattattgccattcaaacccatccgctgtccgcctcaccccatgttcctgcccctgcctacaccctcatgacttcccatctgatggctgaagagtcctattggtcttgctttcaaaccagcctcctccaacccatcctccctactgctgccccctccaaacacaaatctcatcatgttactgtcctgcttaagatgcttcacaacttcctatgtcctgtgggataaaatgcaaacacctgccatcagcacacaaggcaggttacagtgccgcccggcgcacatctcccctggcttcagtcacactgaagaactgcagttctccttgcaacttcatgcctctctgctcctgagcctgcttctcctgctggacatgtcatttcccctttctctgttttgctaacctcaggtaaacactatccctaggaacctttcctaaatctcatttttcaaaaatgccctctttatgctcctgagtggctccagtgtatatcttatggtactcagtggtgatcaaaataacctatggaaataaaagattaaaaaacacaaaataaaatacacatgtagcaatcttcttttctttgtctctttggattttggatttcaagcagcttgaggacaaggcccaggtcttctgagttacatccttagtgcctaaagagtgaaaattcacaatgagtgtttatttaacagaatgaaaattaacatttgaaggtggaatttgctttaaaatgcacggacctttagttttaggctttgtgaatataataaaaacaaagtaacctaaggtgaacaaacgacatctctgtagcacagcttgtatatcatgtttttcatctgttttttctttgggcttcgtttctgtaggtgagtaattctctagcccataaactcagtggaggaccctcaacatccgtgtgtgacactgaacttgccgtcacttgacgcatccaaggagaggactggttttccccgtgttgggcttggaggggaaatccttgcttaggaccaacaaccatgcagtgtcatttcgcctcacttcctcttccaggctggactccatgctctctcatctttattcttttactgtaatagtaatagacttttttgttgtcaaccttttgctgcgtcttatacatcaggtaaagctaagatactatcccataggtgtttcaaatgcaacaacttttatttccaggtagaactacacttgaaaatatacattcaacaagaacattttgtgaatgttatgtgcatgacaacatttttaaatttactatgacttaaaatgaaagtgttttgataaaattatataaggtaccatttttattctaaaagtaagaacataacttgaagtctataaatagcactaagacttactctggaggtcttttcatgtccttcattatttttttccctgaaagatttttatttcatctccctacttccctgcctttctctgtggctatatcagtattttttatgtctggtcctgtaggggttctgagatcctatgggaatggggtagggggactacgcaattttgtttctaccaggagttgtgtgacttgggtgagttactttacttctgtggtcagttccccaatatgtaaaataatttcctctgttaaatcaagtttagcctaaagttgcctccttacatattttaagtttggcctaaaggtttctctgtacatcatgaactataacaagtggaggtgtaaacagaccatagcctacacttgtgccaatcactgagttttggccaatcatatgtagctaactgtttgaactgtgttcaagttaggcaaacattgagctgtaaccaatctggctgtttctgtacctcacttctgttttctgtatgttacttttctttttctgtccataaactttcttccaccacgcagctgtgctggagactttgagcatactctggcttgggaggctgcccgatttgtgaattgtttattgcgcaattaacctcatttaaaatttattcggctgaagtttttcttttatcaggtggtgtcagaagtggggttggaagtagagcttctaatgacccccagaagtgctgagtgacctagcgaggtaccctccaggcccattgtgtctctttctctctcggagcagctggggatcgtggtaagttctcttgcagattccgaagctccacagatttgtgtgttgagctctctgagtttctttgagtaaatttctgatcctgattgggtttgaaagtcgcgaaagaaattgaactgagtccaggatcggattggctccagtaattaactggcttggatccggttagagacctcttacatctaactggatcagaaagaaaccagtattaaatggcaatattgcaaaggtgtaaaatttggcttttggaaattcacagagatttttatgttctactcctttatttttcttgtgcacttaggtaggaaaaaaaatcattggctaagttgaacaagggacctgagagcaaagccagtatttgaggtaaaaaatgtgatactgaatttctgaagaactgagttccttctggattatacatgcataagtattaggccctggaaggagcaacgccttacagaaattgtccaggcgtggtggctcacacctgtaatcccagcactttgggaggccaaggtgggtagattgcttgaggctaggagtttgagaccaggctggccaacactgtgaaaccctgtctgtactaaaaattaaaaaaaaaaattagccaggcatggtggcatgtgcctgtaattccagctactcgggaggctgaggcacaagaatcacttgaacccaggaggtggaggctgccatgagctgagatcataatactgcactccagcctgggtgacagagactgtgtctcaaataaaatttttttttttttttttactaaagataacttgcagtagaacattccaaaagaacaacactgcactgaagtgcatttgaaaatgagggctcccaaattagtctcatgtacagtctcatgtagggatgcctattgatatgcagaagcttctaaaaaatttcaatatttgcatttaaagactttacaaaaaagaattaaaaagcttaaacaactaattgatttaaaaaattaaatctgccttgcgctttttgctgatggctgtgtgtgacaggattaggcatgtacaggatcatgggacatggggaacttttttctccccaaagcaggaaacttgagagctgatgagatggctggaaaagatccttcatgactgacaagcagctgcctgaactttttttttttttgaaacggagtctcactctgtctcccaggctggagtgcggtggcacaatctcagctcactgcaacctccgcctcctgggttcaagtgattcttctgcttcagcctcccgagtagctgggattacgggcgcccaccaccatgcctggctaattttttgtgtttagtagagacagggtttcaccatgttggtcaggctggtctcgaactcctgacttcaggtgatccgcccaccttggcctcccaaagtgctgtgattaaaggcatgagccaccgcacctggccctgaacttttaatttagtgtcactgcaataggtgggtccttctctgtcctccctgaactctttgccttccccaccctgctgcagacaatgcttttctttctctctttctcttctttcctttttctatctttcttttttttttttttttgaaacggagtcttgctatgtcacccagcctggagtgcagtggtgcgatctcggctcactgcaagctctgcctccgagggtcatgccattctcctgcctcagcctccggagtagctgggaccacaggcgcccgccaccacgcctggctaattttttttttttttgtatttttagtagagacggagtttcaccgtcttagccagaatggtctcgatctcctgacctcgtgatctgcccacctcggcctcccaaagtgctgggattacaggcgtgagccaccacgcccggcctcctttttccagctttctattatatcttgtctagagatcacatgttgaaacttctggctggaggccattccaccccactttgaatagattaaagatagcagagccccaccaggggcaagtttaagccttaccagttcaatattgggcactaagcagagtggctaatgtctatgttttgtcacatgtattttgctgtggctggaagggaaaatgttaattcagttccctcatgcaatctcttgggcagcatcttacaaaattgagaggcttttgcctatggttccctgaaatcagaaaagatgattttcctttgtgttgtggcttggcccccaggctatgtgcagctaacagggtcgctagggccactcagaaagagggaacccagaagcctggcatgctggcaaaagggtaagaatttcttagcagtcaggcttctggcctctctctgtgtggaaacggttgtaggagtagtaaaaatcactgtctcctctgcaaagttttaattaatgataaaaaggatttgtgaggctgggtgcggtggctcacgcctgtaatcccagcactttgggtggccgaggcgggtggatcacgaggtcaggagatcaagaccaacctggctaacacagtgaaacccagtctctactaaaaatacaaaaaattagccgggcgaggtggcgggcgcctgtagtcccagctactcgggaggctgaggcaggagaatggcgtgaacccgggaggtggaacttgcagtgagccgagatcgcgccactgtactccagcctgggcgacagcgagactccgtctcaaaaaaaaaaaaaaaaaaaaaaaggtatttgtgagccaatcttaagctgtagcaaacctggtgtactttgtgctatgaattggtctttctgtgtcatttggtcataaaaggggttaaataggataaaatgtgggcctaggactcccagaagtctgctgttcaagccagcctggcaaactggtcagttacaaactgcgggtccctgaaacaaaaaaacactggataaggtttccctgtcatcttgttttatgtcattgggagcttgaccttataatcacatgatggtactttcttttggtctccaccctctggaggacaggaattttggagttcatgtcatagttagctctaaaaattatcttgagcagttaaaagcctttgcaagctgaaaattggctgctcttaggctcctccttctgggaggaacaatggaaaccaccaacggtgtagcttagtggctaagctttgccatcttatgatggcagccaggcagggttcaattccagctcagggaatgagacctgtttggtttgatatctttctgatctttgctatttgctgattctcttcccctcatgaacaacttctgactctcgtcttgaattttcctttctctgagctacgtttggaaattctagagtttgtaaaaattgcttgctacctttttgaaaatacttcgtacacttgtggttaagtcataaccttgttaaagcttattggtttcacctgggagattacctttggtaaagttcaaaagccagacatattggctgtttggcctggctaaagtcaagtaataagagatttaaaatgattattttaggctgggcgcagtggtccatgcctgcaatcccagcgctttgggaggccaaggaggatggatcacctgaagtcaggagtttgagaccagcctgaccaacatggagaaaccctgtctctactaaaaatacaaaaattagctggatgtggtggctcgcactggtagtcccacctattcaggaggctgaggcaggataatcgctttaacctgggaggcggaggttgcagtgagctgagattgaaccactgaactccagcctgggcaagagagcaagactccgtctctaaaaacaatcaaataatttttttttttttttagaaagagcgctatagttaaaagtcagcttaattaaaagtggatatccaagctataggtatgtttaaagggccttttggttttttctcttcttggatctttgtttttttttaattatttttttctttttagccaactgaattgtttttctccattttgtcttcttgccacttttgttgcacacatgagaggacctaaggtaacttctaacagcctgggactccttgggaaaaacagaggaggcactacgatcctgctttgggatataactcggttttcctcctgaaacaccaggaatcaaaagcagatagatccctctcaaaatctaaggctaaatatccttttggggatggccaacagggtgaaacccagtctctactaaaaatacaaaaattagcctggtgcagtgctgcacacctgcagttccagctactggagagtctgaggcatgagaattgcttgagcctggaaggcggagttgcagtgagccgagattgcaccactgcactccaacatgggtgacagagtgatactctgtctcaaaataaaggaaagaaagaaagagagagagagagtggaaggaaggaaggaaggaaggagggaaggaaggaaggtcatttccacagttaattgcttaatgctggtgcaatttctgaaaacttcacaagtaggcacaattctagaatatggtgtcttttaggaggttcctgaaaggatggaaaggaccccaaaaagtattcttgattacaggtttctgataactttagaatcatatcatttttactgggtaagaattcctggagctttttttttttttttgagacggagtctcactctgtcacccaggctggagtgcagtggcacgatcttggctcactgcaagctccgcctcccaggttcacgccattctcctgcttcctggagcttaaaagactgactggtttataaaactgccatcccaagtagaacacaaattaattgaataccaagaaaatactttgcaagtctttcatgctaaatcacccaatactgaaattgtttagatacacaatttgaagaaactctgtggtctaactcaaattacctatgataacccatcagttatcagtgctatgcacctcaattggagaaacaaccgtgtcaagcatggactcatggagaaccaaggtggctgccttatccttcctgagcttttaaagcttttataatttaaagttctgcattccatgactcatcatggaaaagataaaatgatccaagttaaatgcatattggtgtggtgacttctaaattgctaaaatagtttatgaccaatgtttggtttgccaaacccatattcctgggaagacaatcaaagcttcaggtacattctgctacctcatgagccatttaaacatttatgaagagatttcagtcaattgtcattttcaatgcatgttttctggttgtgtaaaagctttcctatacaagtgaactgatgttataacagtagattattatttcacagtgtattttcaccaggtaaagaaagctttttatggttcactgactgaggacaatcaaccccttcacaatctagaacctgaagattggatcttctgagaacatcagagaaagactgcccttgccatccacactgcagtaaacatcagaaccgtgaactttggcttcatgatctcacaactgagaaggatccctccacattcttggaaatatacacccattggaacccgtaaggtaaagctaaccagggaagtttcttcccagaagaagatggtatccttgatgtgaacagctttcaccaagatcacagatcaagacttctctgctatatggaggctcttatctctgagtattttttcccttgctgatgcctctatgaacagtagaagtgaaaagggggtctgttgtgtgcacttatggggtaggtatacttttattggtgaagggttttgcagccagccttatacatggataacctcacaccttgatagatgaaagatgaaggcccagtgtaggtgagaaattttaatggtacataggttgcctcataatcagtcagaaacagaacattgattcactcccctattccacatcatgggttaaagagaacattgccaggaggccttcagtcttctagaagggcatttttccttaggtcctttttaccatgatttggagtaaaacaggcaatgattagaaatgtatccctcatgataggctctaaacagagtctactgtaagggcgatggttacacaacagattttacattcttttgtgaaagttatgcttaatagtaggattgctgtagattacttactggctaaacagagaagtatctgtgcagctgctggcacttgtggcctatggagaaatacatcacgtcaggtgtacagagattcagttgtaggtaattaatgaagagactgcttaaatgagtaaactctttatttagctcattctttgatctatttaattttgggtggtttggtttatggggaccctgtgtaaggagcatactccaaactcttggtattatcctcctgatagtcataatagtagtctacctggtgcactgtattctctcgaaagttttaaatgtttgcatgcagccatctctagaatgtcaaattgtctctcttcaactagaatgacaagattgaaagaaatgtgtgaccatgatggcattttaacctatgaatgatgtgttgagactggaaacccaaaatgatggtaactgagagtggcgctaaggccctaagctttggtcacattctcacctaagtgagaacctgaccaaaaaggggacatttttaaacaaaatcatgggaggccattgttttggactttaatgaaaggctaaatgcactaagccccaacagaccagaccaaaccaaaatggagtcacttgtgctaaatgtgacatgattaaactaagactttaaggaaacacatagatcctaaaacagaccaggttttgtttttttctcctataaacagaacgttccaacaaaaggaggtaacccttacaaaaataaaattaaaataaaataaaataacctgaaatccttgttcccccttacaaaactcattgttctgctatttcccagggggtttcaagaccaaataagtacatttacaatggtgatagtggcatcgatgactaaagttttggtaaatctctaaaaattgagaagattaccaaaagaggaaaattgttaaatcaagtttagcctaaagctgcctccttacatattttaagtttggcctaaaggtttctctgtacatcgtgaactataacaagtggaggtgtaaacagcctacacttgtgtcaatcaccaagtttcggccaatcatatgtagccaactgttcaaactgtgttcaaataaggatacacccagctgtaaccaattgggtgtttctgtacctcacttctgttttctgtatgtcactttcctttttctgtccataaatcttccaccacatgcctgcaatggagtctctgagcctactctgcctcgggaggctgcctgattcacgaatcgttcattgctcaatttgactcttttatattgaattcagctgaagtttttctttttttcttttttctgtttttttttttcgagacagagtctcactccgtcacccagtctggagtgcagtggcgcaatctcagcccactgtaacctcctcctcccggttcaagtgagtctcctgactcagcctcccaagtagctgggactacagacagcatgcctggctaatttttgtatctttagtagagacaaagaggtttagtagagacaaaggggtttcaccatgttggccaggctgatcttgaattcctgacctcaagtgaaccacctgcctcggcctcccaaagtgctgggattacaggcatgagccaccgtgcctggccagaagtttttctttgaacactttttttttttggggaaggagtctcgctctatctcccagactggagtgcagtggcgcgatctcagctcactgcaagttccacctcccgggttcacgccattctcctgcctcagcctcctgagtagctgggactacaggcacctgccaccatgcccggctgattacaggcgtgagccaccgcgcctggccttctttgaacacttctacgtggaagtgtgaggattcaatgaataagttgagcacatggtacatgttcagtgaatattttctgattgtgttcagtgttatcattattggaatgatgtgagggttgtgtaagtcttcagagaccagggaaacaactgaaatcttctatgagtcatttctatcattgtttatccccattccttttcttttcattcatttttgcatttctcattttctgtgttattcccttgcattcttaaaaactttcccattacctgcttgcttcttgtattctgtgtactttcctatgtgttcctcattctgcagtcttcttccccttcaagcagcagaccatgtgtcaacgtatgcagagtttgtgcagacacacagaccctctggggagtatatgtttgaatctgatgaagatgagcagttatttgtgcacctggacaagaaggagatggtctggcatctgtgagagtttattcacactgtgaggggcagaaaatgaagggaccagggatggaaagggatgggggacaaaacactaagctggcagtcctaaccccagtgtgtttattctactccaggtgtcaaactttctcagtcagaagttgacataaggaagtcaccagcttcggtctcctttaaatctccaggcctaactttcttttgaatctctgctcttcaaccacaatgacttatgtaccccaagacccctctacctcctggctgcctatgatcctcctgcacctagcactcagcaaagggtctgacatttagatagcactcaatcaatatgaagaatttaaagtgtagtgacaaggtggtgcccacactggatggtctgtatgctgtggtctcatcagggacctttctctgcactgcatttttcctgagaaatcccagagttcaataaatacctgtgtatgtcagagctagacacatctgatgcctttctgtgactccttatattctcagtttcctaggaatgcatatcctggacttgtaattgtaggatacaggtgcattatgggtgcagatacctcagttttgcatcctgcaagagttagtctgcagattgttcactccactctgggtacggctgacacctacagcatgcgtccaggggacaaaaatggaaggagatgactctgattggatagtgagaggaacacgggtagctctggtaaactgaccagggataggcgcccaggggcacacaaggcccagaagctgcttgtgagaagtcagggagaaggaagccaacaactggatgtatcagagagccactgagtgctcattatgtgtatcaggagttaagaatccatcctctacacgaaaaggaaagcttagaccaaacatgtcttctaccttgatttacagtaacttaatatgaaatgcaaaacacataaaaattttggattgtaaaaaaaagatcagtgtttgaaagagtgccacacctattgctttgcactttcaacaatggggaggcgtttctcactgtatgaattattagctcaggaggtctaggctggtgaatgaatgcagacttagaggataaatgtcgtctacttgatgcattcaaaaggccaagcaacatttggttaaggagtatttgtgatggcatgcaaagaaagtgttgaaagtgtgttagattatatttatcgttttaaattctgatgcttatttaatttcttaacagtaatgctaaaacatgctcttagtaagttatatgttgaatcagtgctatgttaatatgggccctataacacagatttgcagtctgttcaccactgaatagctaagaatagtggtttcagacctaaatggatgaagacaccaaacatgaggatggggcagctcactgaaatatagtaaatccaggagaggtctatgtccaacaggccaccttatacaatagtttggatggattttcatggttttcttctttcttatgtctaaaccacaatgaacttctttattaactcttactgtttgctaaaaccctcaagaaaaaaaagtgatgagacatgacttggtgtatattcttaacagaaaacaatcatgttgggttttgattactcatggcactaaggagaaaggaacttggttggaggccccatgagagaaatcatgcattgaatccaggaacagaatgatagaaactggcttcagtgcaactactcagcagcagcatcctcttgggaggctgggggtctgtcctcgaagcaggtactgggactaaagaaacagaaaatgcttgttccaaggtacattagtcttttaggcaaggtcagcaaccctgtggaggtaaaaaacagtaatcccaggttgctggcccccttcctttaagtcccaggagagaaaaataacggtgagaggctgaatctcaggctgataaagaatagagttgatctgagcttcctttagcaatggagccatgcaatgctctttccccactcaccccagggctgaggttgaaacaggggttcaggcctgagactctcttcctagaggagcctcagtgtcacaatgtacagaaaggacttatgtcctcctgtaactcccccaacaaggaaatcaaagcaggaatattgcctgtttatgcagatccttgttgaactgaaatgaaaggaagaaagagaatgtattcagctactgaaaccacctcagacaattagagaaacgtttccatcatacttccattgtccatcactcaatggacgctctgcaggagcctgagtaactgggacctcatgcatctggctgcattaagatgccaggctgggattcctctctccttagaagctgtctttgtttcctgactatgtgatttcagcaagctctctggcattgcttttttgtctcagcccctcatcatggaagaagggtgagcaagtcaggagaaaggtgggtatcggtcacctcacaggatttctcacctttcttcctccttctgtgcgtgaagatgtccactccacagatgatgagccccagcatgaagcccctggctcctgtcagcatcttgctctgcacagaatcagactgtgcctctaggaagaacaggctcaggttcagagtcacctccagcagctgcacccctgctcatgtcctgtctgagatcctgcagcctgatgcagaatattggcacgaggagagcgaggggaatacaccttgcatgacaggaaactgagaaacagtcactctctcaattctagaacagagggtgtgacctcaacaacagacaaattcagattcaattagttgcctctcaaaataacttagaataagctaaaaggctgaaggaagacaacagttcccatgtgtaacgtcagttcaggaaggtgtctgcatccctagattcttggacaaggagaggaactatgaatccttaggacctcctaggccgggggcaggcaagtctccattgtccccaggttaaaactacactcagtgtcttctatagaagaaaaatggggtcggacatggtcccagggtatagacacataaaaccagatgagtggaccccagagccagagagtcccctgctcttcagaggtggagtctagagggtcatcagaccctcactccactccacggtgacaggactgtccaggctggggtgctccacttggcaggtgtagatgtttccctgctggggggtcatttccagcatctccaggatctggaaggtccagtctccattacggatcaggttggtggacacgaccccagctgtttcctcctgtccattcaggaagcatcggacttgaatgctgtctgggtagaaatctgtcacgtggtagacaagcaggtcgtggtgctgctggggctccttcttggaggggagatgttcaccctaggctggactaggagtggcgaagtggaaaagtggaatagcatttgagattattatttcttagacatgctcattatggaattgatttctagatgttgaaaataggaatgatggccaggcgcagtggcttacacctgtaatcccagcactttgggaggctgaggcaggtggttcgagaccagcctgactaacatggagaaaccctgtctctactaaaaatacaaaattagctgggtgtggtggtgcatgcctgtaatcccagctactcaggaggctgaggcaggagaatcgcttgaaccccggaggcagaggttgcatgagctaagatcccaccattgcactccagcctggacaacaagagtgaaactccatctcaaaaaaaaaaaaaaaaaaaaaaaaaaaagtaggaatgatgtgattaggtcagcagataaggggagtattgaaactatagaaatagtattgagaagaataggaggcacacaaacttaccagtttgtagcacatttaaaagaaatcttatctatcagccactgattaacagcctttggacaaagaatatttaatcagcagcgacactaccttatcttttgagtgctctatcctgatgcaaaagatgaaaaatttgctcaaatgatttcttgaaattaatatatatataaacaacattctcatggtctattaatccaataactatatttcataaatatatcaagaaaataaagtgagcttgacgtgacttattctgcaagtaacctatggattttttggcctcgcctacaaacactcaaaatcgtcctatttacataatatcctgagcccctacatgcttaaatagtagaaatcccatccttttccctaagttcctcctctgcactgtgttgtcacatccattgaggttctcagataaccaggtgttggctgtgccttggcacctctctatttttgccccctctttttctgccgcttacagatcaatttccacgctaccacctgcagcttttcctttggacctcagccactagtacccttttcctccacagaaggcagaaaagggccaactgcccctagagaacagctcaattacaggaatgttccaaatcagagcttctaggtccttatttactctgttcttgagaacacaaatacactgacatgggcctgggccaggtaaatgaacagtctttaactaacccccaacccttactccctatggcatttttgccttgtgtccaggattattcacattcacagcctcttccctcgatgtgattttatttcagaggcacttggctctcgttctacttcaaccacctccataaactcaacatccatcagactggtcagcccatgtctgaatcccaactgtagattaagtggaacttcttccccataggtgtcctgggaagcatgaaagtcatgggatatatggttgtctgttttaaggtcccagaacattccatgagaagcactttgctccatcttctttgacgggagctggtcagcctggagcacatcagagacctccagggggcgctgcagctaaggcttgatacccagtggcctcatttctgacccagttcaaatatatttcaccaaggcaaggattcctgtttgttctagttgcctttgctcagttaattctagatttgggtgggcatccacactggaatggccagaacttctatcctcctctttcctccgtctcccctacccctgtgtttttctttattctaattcatgcccttattatgatttccctggacaataatagcagtttcttatctcatctccctgcctctggttcctgctctctccaactgacttcacagtcttctgaaaacagatttccagccttgtcattttttttttttttttttttttttttttttgagacggagtctcgctctgtcgcccaggccggactgcggactgcagtggcgcaatctcggctcactgcaagctccgcctcccgggctcacgccattctcctgcctcagcctcccgagtagctgggactacaggcgcccgccaccgcgcccggctaattttttgtatttttagtagagacggggtttcaccttgttagccaggatggtctcgatctcctgacctcatgatccacccgcctcggcctcccaaagtgctgggattacaggcgtgagccaccgcgcccggcctccagccttgtcattttaagatttctgaagattttcaaaaatcttcagtgttctgacacttatctggttacatgggaatcacttgggagctgattaaaaacacagatctccagacctccccacctagggaatctgaatctctaggcagtgagtctgggggaatctttgcttcacagaggatctcaaggagagtcttctgagcagtgaggttggggacctgctggcctagaggagaaatccacactccttagtctggcatcagggggtctctgcaatctggcagctgattaccatgaaagaaaatgctgccatgtcgctgatttgggcctctatgaagtcttagtgacagattttgcaaattgtattacttttatttggacacctctctcaaccaattacgccagcaactttcctaaacccatgtcactctcctcatccttactcaatgtgtgcatcttctccctcttatcagatgaccttcctttcactttcccaggaacagagggatcaatggtgtgacctcctgagcctcctcctcctcccccttcattctttcctctttctctaggacaaaggtccttgtttagtgagaggctcactggtgctttttggtctccttcacgggatttctctcttggccaaattaatacacgagttattaagagattatttttaggcagctaaaaagggtaaaagttctgggtggaattttcctttaataaaaagcagccccaagccattttttctctaacagaaagcagcctgaaaactcaggcatagatatgcaaactagaagcttttatgtaaatgctggcagctgttcctggaagtcaggtaattcaatatggctattctcaccctctttcccttctacgtttacaggtgtcaaggcagcctccaggttaaagcacgtgtacaggtatcatggccgccaccaggtggaggccgcatttgtataataaaatactagggtgggaaggccagtctttttgcaggttatgtaaatgacacacctggtcaaaccaatcccctgagccctatgtaaatcaatcactgcctcctcaagcctctgtacaaaaccaattgctttccaccagaaacaggagaccctctcttgggtgacctgccttatcagcattaggaagcttttcctctcactcctcttttctattaaactttccgctcctaaacccactccttgtgtgtgtccgtgctgtgaattcttttcggctatggtaaagaaccagggtatataccctagacagtggagctgtttcattttgggagctcatccgggatccaaatcagaatggaagatagaaacatcggagtggtgagtatagagcaaacgtcaaatctgttctttaatctcaagaatctcttcataccagtttcctttcatggagaacttcaccatcgcatgaggctgggaaagtcttggggcaactgaaaatttctggccagggcacaccctggtgttattcaaaggcttctggactgaacgcagcctccgacagcctgtccaggtgttggtaatggatctccagctaacccgttgcaaaattttcctttcctttgtatccgtggtcactatgtctcctgtcctctctctctgtgtgtgcaatttgcgggaagttttacagttcagggaaacactcctgttagggaagatcggcaaatgccacaggcagtaactgttactctctatcctctctggcgagcacatggtagtgctaagccaacagcaccacctagtggaaataaaaatcctcttcatcaggcaccttgtcggttttttaccgtaacactgccgcttccaaattctttcgtgccgctagaaaagcctcttctgtgaacgagaaagcactgtcttcaacagtttggagtaaaatgtcctctgtagtgaaattttagttctgatactgtctcatcagcaggaaaaacagccattaggttcctacgttcatttccgtctccaattaggatagtacttaattagcaaggggattttaggttcggaagttaaccagagccattttgctaagggtaaatgtcttagcatgggccgtaatggcaggcaatctagcacactgcctccgttaaaggagcctacccaaagatgacatagtctctctggagatccatttttctgggagccaggcagatcacacaaatttaggacgtcaaaggggatcacataaggtggataagctaaggttgtgtgggtaaagtatggttaatcccatcacttagtttatccagttccacggcttggaggaccacgcctacaaccatgggtggtacatttaacacgttgccaggacccaggaaccaaggagagaaaacagtagggaggacacttccactgtcttctcctccaccctgggtcacaatgaaagaatggggacgaaaggatacttttattctcacttctttttctagatgggtgacagaccagcttcagcttgcacccctctggagtgcactctgaaacactggaactcctttaacctcaggactttgaagagaaaagtgactcattttcttttgcacaagggcatggcttttttactaaacctttgcaagcactgtaagatcagcccagctttttaaataggcatatcaggtaggcctatagaaaataatcccccagaattagaaaggcaatttccaagggaaccatctgagaattccccttatttagggttgccaatatgggggaagtaaagagaaatcagactgttgctgtgtctatgtagaaaaaggaagacataagaaactccattttgatctgtactaaggaaaattcttctgccttgacatgctgttaatctgtaaccctagccccaaccctgtgctcgcagaaaacctgtgctgtattgactcaaggtttaatggatttagggctgtgcagggtgtgctttgttaaaaatgtgtttgtaggcagtatgcttggtgaaagtcattgccattctccagtctcgagtacccagggacacaatgcactgtggaaggccgcagggacctctgcccaagaaagcctgggtattgtccaagggttccccccactgagagagacagcctgagacatggccttgtgggaagggacctgacctgaccgtccccaaggctgattcccataaagggtctgtgctgaggaggattagtgaaagaggaaggcctctttgcagttgagataagaggaaggcatctgtctcctgctcatccctgggaatggaatgtctcggtgtaaaacccgatcatacattctatttactgagataggagaaagccgccttatggctggaggtgagacatgctggtggtaatactgctgtttactgcactgagatgtttgtgtaaagtcaaacataaatctggcctatgtgcacatccaggcacagcacctttccttaaacttatttatgacacagagtcctttgctcacatgttttcctgctgaccctctccccaccattaccctatagtcctgccacatccccctcactgagatgatagagatagtgatcaataaatactgagggaactcagagaccagagccggcacaggtcctctgtatgctgagcactggtcccttgggcccactgttctttctctatactttgtctctgtgtcttatttcttttctcagtctctcgtcccacctgatgagaaatacccacaggtgtggaggggctggcccccttcaacctcaagctcccttttcattacaggaccttaggcaaacaaaggaagacttatgctaattttctgataaccccaataggtatatagaagctgtccagaatttaactcaggtgtttcacctcacatggaaggatgttatgctgctcctaaaccaaactctaaccgcagttgaaaagcaggcagctctgcaggcagcagataattttggagatgagcaacatatctcctataatacaccaaaagggaagaaaagagatagggaaagtgaaaaaaataacagaaacaccattcccagtaggaagggaagcagttcctctcgacaaccccaactgggaccccagtagctctgcaaatgaatagaaatggaagcatttttaaaatatgcatattagagggtctatgaagaactaaggcctgacctcttaattcctctcaactgtctatgatagaccaaaagccagatgggaatcctgcagcttttatggaaaggctgagagaggcactaatagagcacacttccttagcccctaattcagtcaagggatggctcattgtaaagacaagtttattacacaggcagctcttgatattagaaggaaactgtagaagcacgctataggaccagatagcaccttggggaacctcctgagggtggccacttataatagggaccaggaggaggccccccagaaagagagaaagctcaggagaaagacagaggctctagtagcagctttgcaagcttgcaaagtccaagatttctgaggtgcatccgctagttgctatcagtgtggcaagccagggcattttaaaaaggagtgcccaaacagcaagaggaagccacctcaaccctatccagcctgtggtggagaccactggaaatcaaactgcccccggagacggaggtcactggagtcagaaccagtctcacagatggtccagcaggactgatgggtcctggggctcaaaccccagctccagtggctcaaactgccattacagcacgggagccaccaggtgattctggaaattgaaggaaggaaactagacctccttctaaacactcgagccagtctctctctcttttctcctctttaatccaggcctctcttcttcccatagcgtgagtgtaaggggtgtctcaggaaaaactctattccaatatttttctcaacctcctaattgcagttaggaggacctattgtttacacatgcttccaagccattgccacggtggctctactagtcaaaaaagcctccaaattaaccctaggaaataatttaactgtttacaccccacataatgtagcaggattactgtcctctaggggagactttagctaacaaacagcaggtaaagcaagaaatacataaggcaggacaagcaatagtcactctaatgtctctccccagacacaagcactcaattagctgaactaatagttcttgcaagtgcacttaaattaagcaggggaaagatagctaacatttccactgactccaagtatgctttcttagttctccatgctcatgctgctatttaaaaggaaagacattcttttaccactaaagcatctcctataaaatatcaccaggaaattaacaggttattatcctcagttttcctttcacgaaaaatagcagtaatgtattatagggaacatcaaagaggaacagatgaagtagccaaaggaaataggttagctgagcagggagctaagcaggcggcagggaagcctcaaggcattaacacacttcaagcccttttaatctcggaagcctccataaaagaaattaaacctcagtattcccctgcagaaataaaataagccacttcttaagggtattccagccctgaggatgacaaactccatttactggcctccagtcaatggaaagtccttaaaatccttcaccaagctttttcacataggaaaggataaaacttatcatcagtgtgctcagagattgttttcaggcagaaaacctctaagttgtttaaaaatgtaacctctctagctcacttccaacagaaattgacacaactagccaaaggccaaccccaggaaattggaccaccagaaaatttggtattggtgaaaactcatctctctctccttccctaagccaggctgggaagggccctacacaattcttctttcaacccccccagcagtaaaagttacaagtatcaacctctgaatatatcacactcaagtcaaagcctgaaaagctgagggagcaacctttgacagcccagaggaacatcctgaatatcaatgtggagatatagaagatcttaagctgaaaatcataaaaggtaagtaaatgagtgagggctactcgccttagcgccaatcctacctcaccaggtactctttatcatttctacctttcctctcaaaattcactgctcagtattagaacttttttttaatgcatatttgcagagagattttaattatacatgggactgcatttgttactttgtaaatccccaaagggaaacattatatcttggcaagtaaagttttaaatggaaattatttactacgtcacttttgtgggaattgttatcatcatgctgttatttgcaatagaactgtatactgtggcacccacaatgtggaattctggttgtaaaattctaattcctgtaatattttgcctaattatcatctttatgacagaattaataattgcaggaaggatttggtcaagtttgttttgcttatagcaggagtaatagttacagacaagaagtaagcatgaaaattttactatcactaagtttgataggacttttttattgaagattggtaaatggtgcactctaagctatggaaagaaggttacaaataaagggattttatataagaaaggatcttgtatagtaaattcttgtcctaaaaggaaatgactggttgtttaagacaagtcagaaagttgagtacattgtaagagggtctgtgaaagtcatgaaagaatttaataattaagaaatttaataattaaaggaaaggaattgccaagattaacaccaaagttattttagccacccaataacgtttttctcccaatcatatcataagttataaagaatggcctaaaccaaaaattatgccctaatagcaagtcaagggggaaacatgttttctcaaaggaaatgatgcttttatattaacgtttctggtaatgtacagcgacatctagtggagacaaaccagtattacaatccattggtgtaacaggtatcaaactctactgccatagttacagtctataggtggtaatcttaatactcatatggtaaccctatattttaaaccttcttgtaaaatttatctctttttgcctagaagcaatcaaacttcaaatggtgctgcaaacaaagccacacatggacatgccattcttccaagaagccttagatcaacctcaggaggagccccaactgcagccccccgacacgacgccccttttcagcaggaagtagccagaaagaatcgtcgtccaacaccccctaacagcagttatggttacgtctcctgagggaggaaataatacaggagttattaagaaattatttttaggcagctagaaagggtaaaaattctcagtggaattttcctttaataaaaagcagccccaaaccatttcttctctaacaggaagcagcctgaaaactcaggcatagatatgcaaactagaagcttttatatgtaaatgctggcagctgtacctggaagtcaggtacatccaatatggcggttcccactctcttttccttgtcaccacgtttacaggtgtcatggcagcctccaggtaaaaccacatgtacaggtatcctgtccaccaccagttggagaccgtatttgaataataaaagactagggtgggagagtcagtcttttcgtgggctatgtaaatgacacaactggtcaaaccaattccctgagcgctgtgtaaatcaatcaccgcctcctcaacctctgtacaaaaccgactgcattccaccacaaaccgcagaccctcttttgggcaacccactttctcagcatgaggaaggattttttctctcttttcttttctattaaactttccactcccaaacccactcctcacgtgtgtctgtgtcgtgaattttctcggccatgacaaagaaccagggtatataccccagacaatggagccgttacaaaatcacaatgtccagttcccaggatccagtcctatgctcctcccgatgggtcccctctccctgggtggtctcatgtaggccaggcccctcccccgctgcaaactcttctctcacctgcccatcagaccacccatctggcccctcaagcacctcaaacccggccatctccctgcaggtttcttctctgcgtggcctgctgtgccatctccaccttcatctccacaccgcacccctgcacgatgtcctgtggcctcgtttcccctcaccccacatgcagtcagctgccaggcctgatgaagtcccaggggtctctctccctccatcctcctcactccatgctcagcccaatctccgtgctcaccctcctgacaagctccggttgggctcctcccatcaatccccagctcaaagtccccttccctcctggcactaaggtcccttaggccggcccggctgcccctactccctgggcccggcccccacggctgccccaggagccctggttcacagccctcacctcggcgctgccggatgagtggctccatcagctcgtacttgtgtctgcacaccttgtccacctcggctcgcttccgttccataaagtccttctggctattgaagtactcgcctatgggccgccccagctccatcactgctaggaactcccccactgcgctgtcaaaatgcacgtattcctcccggttgtagatgagcccgtccacaacgcgctgagtcccattgaacgcatagcattcctgccgttcctggtagacggaattctctgtgaagagcagggagagatgggggtggtgccactcccaacaacacccccctcctcaatattagctatttcctcaaatcttcccactcaggactggatttaaaaatacgatttttcctactaccgagttctgtggtcctaggcaggtcacagactccaggcatcagttttctcaccacgcagcgagaggatttactgaaaagaatgagactcactgctcagggtggctgagtgatcattagggaggggcgcacgctgaagggaaagcagccctttctgctggcggctggaggaggagggggagacatttcacgtctcagggaggagcagaatctcatcagggggagggtccctcaggcagagaaacagtagggataggaggagttggggacctggagggcagagctgctccccctacccaactccctgcctgacatttccatcctgatgtcagtcctggctcaaatgccacctcctccaggaagccctccattccttctttccctttctacagctaggttctctctcttctctgccagtttcttgagaatacctcaagttgctcttgctgttctgcatgctggacatctctgcatgctggaaatgcagaaatgcatttccatgcattcctccctccctccctcccttccttccttccttctttcttctttcaatggagtatccttccctccctcccttccttccttcctttcttcctttctttctacagagtatcactctgtcgcccaggctggagtccagtggtgcaatctcagctcactgcaacctccgcctccagggttcaagcagttttcctgcctcagcctcctgagtagctgggattacaggtgtgcgccaccacgcccagataatttttgtatgtttagtagagatggggtttcaccatgttggccaggctggtctcgaactcctgacctcgagatctgccctcctcggccacccaaagtgctgagattacaggcgtgagccaccgcacttggccagaaatgcatttcttaaaacttcttgaagtttgccataagaaagactatatagcctgaaagttaattttcactttcaaacatggctgtagaaagacttgatctcaaaacacctggaaaatatcttaatcaatgaaaatcaccagaacaaccagaaagctaagtgactggactcctttaggtttagaggcattgatggtgtcatttttacataaagaaatgtggcagccttttccacctgcagttcttcctagggagcaccatgggcagtgtccggcaggtgcatgtgtatacagaagtagcaatgacccagcacatgtctgagcctcttcagacctgatctgttcctggtcacagtggggaaattaaggtgctgtgatttgcacacagcaaattaaggtttgttttggacacctgtttcatgttctacaaattagcaactcacgtttattaacctcacccctcatagaagatgcacagctggtggcagaggacagtagagaaggggtggggctgggcacagcggcaccgtagactcggcctgtagtttccagtgtcatctctaaggcaagatcccaggactttagatatcttaacccgcctcttctcctagtgccttgggctccagccccctaaacacccaggcccatcgcccccagcccctttagtctgcccctccctattcaagtgccccactctgagtgtgtatcctctcattttaatttgttagtctttgttcctcttcctcattcaccaataagagaacatgttttagtcaagagtaagaatttcaaaactttcttttgcaacatttaactttttaaagggaattttacctgcaaagcagatataagtgatgagtatatggtggaatttgaaacttcattttttttttgtatatggaaaactttatcctgtttgcggaatctttgacaattgagttacccagagcacaatttgaaaaccaatgatctgagtgaattcatctctcactcagaataggttatattaaaatataattgcagaaagatataatggagtccatatgggtaagaaaaggaacatgtcacaggtagggattccagttcttgcactacgactgattactaagttatcttggacaagaaacaaccttctaactctccatttcttgaaaggcaaaatagtaataatactatttaccttgcaaaactgctgtgagaaccaaatgagctcacatatgtcaaatacatagtattgtatctgttactgctcaatctatcttagttcccttcctcttttcactgtgtgacctgtttgaataggagcaaaattctaaataaatgcatatgaaagaggaactggatcagcttatgagctactggaaaacctcaagaagactttccatgggcaccagatcttaatggttgaaaagtgtttgttggctgtttgctgactggatcttcccccctctcttttgtgctagggattttatttgaggagttagattaacagaagagtgagtacccaaagactcaaatgcacaatgagcttctgcaagtgcccagctggtgtgacaggaggactcgggagtgtggccctaatgcttggggtagtgggtggggtgaggtaagaaactcagctgtaaaaagcccattttaaagcttgtcagagacaaaagggtgttgcctccaggagtctggttttttcacctgccgtgtcatctcctgctccctaggtgttgtctcagccaagcttttctccaccctcccctctctcactttaagccacagcctggctccttgtgcctttcctcttgtgctctgaggatggagatgtgtgtggagttggaagagcatgcagggaattgtggaattggccctgcccactctacccactcccctatggctccagctctccctccagcaggttttgattggacattcattctacacggggagctctggtaacccaccctcggttcctgtcacatggctccactgcctcatctcatttcccctaccaacctcaacccagtctttgcccgtccacctgttcactgcccaccatcatcacgctccctcctgtgctttctgctaccccgcaccttgagggtttccatggcgtttcccaacaccacccctcatcctacaaacaactctgcctatggacactgttgctatggacctcctgctggacactgttcagtgtcaccagcgctgctccagcctcctctctccccaacctcacccctctccagttcccagggctgagccattctgctggttagttctcagcacccctgtgactacaagtgcagtttgtccaccctttcccggacaatgaacctgaggtaataggtgaggggctttggggtttgaggggctgtcctcaggagattcgaatactgttaccctggaaaatgaggaggtgacatgagaacagcactttctaggggtgtcctaggtggatgtggaagggtctcagagggagggtctatgcagaaaggtggaagtcagtggaaaactaagacacctactctgcagtccttcctctcagggtgttggtgtaaatttggaccagaaaagtaagaacatcctgagagaaaaacaatgggtcatagaagccataatattacaccagccacaaggagacagcaggagacagaggtttttcccttggttactgctttcttggctgtctgataacctacactcaatctctttcacgtactcacacatccttatctcattcttctgacaggtttcaacccatcctaatactctaaccaagttctggggaggctgggagaaataccttcaacaagagtgcctttaggggctcgaacctgtccccctccctcccatcttgccttcattgtccagggagcattggctcctgctccaccctggagaatgagaggcattctctgtgagcactgaatcctcagtgatacttgtagtctggacacaccagctaagggctctctgcctgagtcccctcaaggttggatgcagatgtgagcacacccaggagtctgcacttgccaacctctctctctgaaaccttgtctgtccaaggttatcctgaacctcttggccccatttccccatagacaagcaacttgacccctgagcacctccccttatttactgtgtccatgttcctggagagagaatagacctggtggatagcaacctatcctataggaggtgagtttgattctccagctgtgatagaaggacactaggccgtggcaggagccccacatgctgtctcagagtctggttccataaagagaaagtcccctaggaattgttccctgagccagaccctccaggaatagcagctctgctcttacctggagtggccctgctctggaccacagatatgagcagcaccatcagtaatgctgtcagagccactgtccaggggccccctgaaacctgcaggatcatcatggagttggaaaaggttggcagaatgaagagagctgcagtcaggaaaacaagaactcattaaagggagctcctgtctgaaatattagagaccatgaacccaagcagtcttctgtgaccctaggattggacagactctgagaaaagaaccaatgggcactgagctttgtatgagtcattgctcactgggcagaaagttagtattaaagatctgacaatatagagccagtgatgctgttacgaggacagatggagaacactgacactcattttaaccagtcagagtcatgagttttggggagatgatgtgttttctttgctctgaaggtgatctcagatattctgctggcccacctacagggattatcatttccccaattctgccacacctcacacacccacaggacatggcctggtgtggaagaaatgctatctcaatgtgtaaaaggtcattcagtggcatgatttagagagattagagtatccatcccagaactgaaaatgaggcctggagtctgttttgcctttgttcaaggccgtgcttcagattagtgcacattcatattttcttcctcccacatgtctgtgagtcctgagatgtgcgggggatactggctccttccataggactgtcatcagggtcagcagggctcagtctaggggccttacacctgggagcatggacacaccacctacactaccatggaagtatgcagcttgaaggacactgcctgtcttggacttcagttctttgtcttcagtatggggatgatatgacctgcctttacagcagggctcttaaggtcaaattagatcaacggatctgtaattgctttggaaaaatgagactaaaaattatacagtgaatgaggaagaaatgaaaaaatgtcataaaagaccctacattttccaaaacatctgattctgtggtgtttatactgaatagtttcataaactttcaaagaatattactccttaatttaaagacctataaatgtgatcctgtacgacctcctaatctaataaagaaaatgtaaaagtggcatcatttgtttatataaatgttaaaatgtaaatagaagactagcatgtaaaattcaagagaagaaaataattatgcagtagaaggggccagtataggattgcggggaaaaagctcacgttccctgccatagtcaccaagacagcatggtactggtataaaaataggcacatagacaaagggaacagaatagagaacccagggataaacccaaatacttacagccaactgatcttcgacaaagcgaacaaaaacatatggtggggaaagacacccttttcaacaaatggtgctgggataattggctatccacatgtaggagaatgaaaccgggtcctcatctctcaccttatacaaaaatcaactcaagatggattaaggacttaaacctgagacctgagactataaaagctctagaagatcacattggaaaaacccttctagacattggcttaggcagggatttcatggccaagaacccaaaagcaaattcaataaaaacaaagataaatagttgagacttaattaaactaaagagcttttgcacggcaaaaggaaccatcagcagaataaacagacaacccacagagtagtcacaatctatacatctgacaaaggactaatatccagaatctacaatgaacacaggcaaatcagtaagaaaaaacaaacaaccccatcaaaaagtgggctaaggacatgcatagacaattctcaaaagaagatatacaaatggccaagaaacataagaaaatgctcaacatcactaatgatcagggaaatgcaaatcaaaaccacaatgtgataccaccttactcctgcaagaatggccatgataaaaaaataaaaaaacagtagatattggtgtggatgtggtgatcagggaactcttctaccctgctggtgggaatgtaaactagtacagccactgtggaaaacagtgaggagattccttaaagaactaaaagtggaactataatttgatc

SEQ ID NO: 35 - DNA, 661 bp, human
tacctggtaagagttggtttctttttatttggaaataactatggtcaactttcattaatgtggaagttgactgataatgttaagagtcaaagtgaaaaagaaagccgatacccatattcaacagtgctttcagttacaacattttttgaatttccttctctaaaacattcacagcagaggtgtccatttcacttgcctgtttcacctctagataccaccacagttgctggggtcatgtgaggtaaaggaatatatccaattgactgttagcccaacatgactatctgcaattctgatagaaaagctactttcagccatgtcttacaaagtgcttaaagctggacagaagactttaaataaagctaaaatcgcttattgcctgagttgaacaagttacaaaccacagctcttcctcctcccctgctgtctcaccaccaattagccttcttgtgaggacaacattagaaatggaactggtttttgagcccagaaacataaaaatttaagtgttcttagcccagcgtggtggctcatgcctgtaattccagcactcttggaggccgaggtggggtgatcacaaggtcaggatttaagaccagcctggcaaccatgggaaactcttgctctactaaaatcccaaaaatagcctggccttgtgggc

SEQ ID NO: 36 - DNA, 3227 bp, human
gtttgaggccggccagtcgtgactgggcggcaatgaggtcagtgactgccgggagtcctgcaggggcggggcggcgccaagcgcagggagcccggctgagtggcagccagattgaagatggatacgtgacaatcccagggaccgctgcactgacttcatttccttagacaagacacagtgtagggcccggcccggttggccccaggactcctttggaatatagctgtggacaatgaatcctgcgagcgatgggggcacatcagagagcatttttgacctggactatgcatcctgggggatccgctccacgctgatggtcgcggctttgtcttctacttgggcgtctttgtggtctgccaccagctgtcctcttccctgaatgccacttaccgttctttggtggccagagagaaggtcttctgggacctggcggccacgcgtgcagtctttggtgttcagagcacagccgcaggcctgtgggctctgctgggggaccctgtgctgcatgccgacaaggcgcgtggccagcagaactggtgctggtttcacatcacgacagcaacgggattcttttgctttgaaaatgttgcagtccacctgtccaacttgatcttccggacatttgacttgtttctggttatccaccatctctttgcctttcttgggtttcttggctgcttggtcaatctccaagttggccactatctagctatgaccacgttgctcctggagatgagtacgccctttacctgcgtttcctggatgctcttaaaggcgggctggtccgagtctctgttttggaagctcaaccagtggctgatgattcacatgtttcactgccgcatggttctaacctaccacatgtggtgggtgtgtttctggcactgggacggcctggtcagcaacctgtatctgcctcatttgacactgttccttgtcggactggctctgcttacgctaatcattaatccatattggacccataagaagactcagcagcttctcaatccggtggactggaacttcgcacagccagaagccaagagcaggccagaaggcaacgggcagctgctgcggaagaagaggccatagctgctccagccggggctccggggcggcagcagagctggcacaccgattctgggaagccccgcgaatgatggcttttgaattaatgaggcagtgaatgttttgtgtttacttctaagggaaatactaactttctttcgcattagtattaattttgaagtagctacaaagtatttttaagaaattataattttatgactgtctggcaggctctgtcagtttagccgcgccggaccgtgtcaagcatctaggagaggagtccatggtgtccaggcatcggggcgtcacacctgttgaggagtggggtggctttgaatgctggaaatggcttcatagtgaagtgcctcccacagggcgggtgggtcagcgttgactctttccactgcacactcatatgccgtgtgtcttattcagaagtcacattcttttcagttggagagaattgggctaagatagaaaataacatgatttgttccttattaaagtttcccagcgtatgaaattctaagctgggtggggtggctcacgcccgacgtaatcccagcacgttgggaggccgaggcaggtggatcacttgaggccaggagttcgagaccagcctggtcaagatggtgaaaccccatctctactaaaattacaaaaattagccgggtgtcgtggcacacacctgtaatcccagctatttgggaggccaaggcaggagaattgcctgaacccgggaggcggaggttgcagtgagctgagatcgcaccactgcactccagcactccagcctgggtgacagagcaagactctctctcataaaaaaaaaaaaaaaaaaaattctagaccgaagtgtacggcagtgccatctggtggcaagtggtacaaagacaacgctgaggggtagtgactcctgtagcagcagagacgccttggagtttaacccccaccaaccagagcgtgggctggtaaagatgaaatcttgcaagttttttttttttttaaatcatggtacctgttttaaaatgagaagttattattcatactgtgttgctcatttgacaaaataaggtaaggatttcataatcaggttgtctgggtttcagagctgtttttaattgactgagttacctacaggcacccaatctagaccctaattctgtggttggtgttcctctgtagatttcaagacaagtagtgatgtcattttctccctgagctgtagggtggacctggacccctcagagatgctcgactgcagggtcctggggtccctagagtggtcctgcagcagcccctggcaatgagctgcctgatgatgagtcccaaggtacattgttctactgttttatacccaggtagaagttacacagcaatttagcaatgttgttaacagacatacagaattgtgtgtgtgtgtgtgtgtgtgtgtgtgtgtgtgagtgttttgatataaacgtgcaattaaaattcaaagtttattttttatcacaagcacctttgaaatgtacgtggaaaacgtcggctgctcgtgagaatcatgtgtgacttacgttccttatgttttatggaactctcggaacagtgttttgtttttgtttagtttttgttttcgtttttttggacggagcctggctttggctcccaggctggagtccagtggtgtgatctcggctcactgcaacctccacctcccgggttccagggattctcctgcctcaccctttcaagtagctgggattcaggtacctctaccatgcctggctgattttttgtatttttagtagagacagggtttcaccatgttggccaggctggtctctgactcctgactaaaagtgatctgcccacctcggcctcccaaagttctgggattacgggcgtgagccacttcacccggcccggagcggtgtattttgattgctggtgctttaactatattgaccaaggtgtatagtaacctatttatcacattttacgaagcaattttaatcttaaacacatccagcaaatggtgtccctgctgaaacttttaactggaaacctcacacctggcaggcagcactctctgcagccgtgcccttgtggagtcagcaggagctggtgtttttcagccagacagaggcataataaagtgactgatgtttacgaaaaaa

SEQ ID NO: 37 - DNA, 3080 bp, human
gggggcggggccttgccgctgcggaggccggagacgcggcggcgctggacgcggaggcgctgggcgcacggcgcggagccggccggagctcgaggccggcggcggcgggagagcgacccgggcggcctcgtagcggggccccggatccccgagtggcggccggagcctcgaaaagagattctcagcgctgattttgagatgatgggcttgggaaacgggcgtcgcagcatgaagtcgccgcccctcgtgctggccgccctggtggcctgcatcatcgtcttgggcttcaactactggattgcgagctcccggagcgtggacctccagacacggatcatggagctggaaggcagggtccgcagggcggctgcagagagaggcgccgtggagctgaagaagaacgagttccagggagagctggagaagcagcgggagcagcttgacaaaatccagtccagccacaacttccagctggagagcgtcaacaagctgtaccaggacgaaaaggcggttttggtgaataacatcaccacaggtgagaggctcatccgagtgctgcaagaccagttaaagaccctgcagaggaattacggcaggctgcagcaggatgtcctccagtttcagaagaaccagaccaacctggagaggaagttctcctacgacctgagccagtgcatcaatcagatgaaggaggtgaaggaacagtgtgaggagcgaatagaagaggtcaccaaaaaggggaatgaagctgtagcttccagagacctgagtgaaaacaacgaccagagacagcagctccaagccctcagtgagcctcagcccaggctgcaggcagcaggcctgccacacacagaggtgccacaagggaagggaaacgtgcttggtaacagcaagtcccagacaccagcccccagttccgaagtggttttggattcaaagagacaagttgagaaagaggaaaccaatgagatccaggtggtgaatgaggagcctcagagggacaggctgccgcaggagccaggccgggagcaggtggtggaagacagacctgtaggtggaagaggcttcgggggagccggagaactgggccagaccccacaggtgcaggctgccctgtcagtgagccaggaaaatccagagatggagggccctgagcgagaccagcttgtcatccccgacggacaggaggaggagcaggaagctgccggggaagggagaaaccagcagaaactgagaggagaagatgactacaacatggatgaaaatgaagcagaatctgagacagacaagcaagcagccctggcagggaatgacagaaacatagatgtttttaatgttgaagatcagaaaagagacaccataaatttacttgatcagcgtgaaaagcggaatcatacactctgaattgaactggaatcacatatttcacaacagggccgaagagatgactataaaatgttcatgagggactgaatactgaaaactgtgaaatgtactaaataaaatgtacatctgaagatgattattgtgaaattttagtatgcactttgtgtaggaaaaaatggaatggtcttttaaacagcttttggggggtactttggaagtgtctaataaggtgtcacaatttttggtagtaggtatttcgtgagaagttcaacaccaaaactggaacatagttctccttcaagtgttggcgacagcggggcttcctgattctggaatataactttgtgtaaattaacagccacctatagaagagtccatctgctgtgaaggagagacagagaactctgggttccgtcgtcctgtccacgtgctgtaccaagtgctggtgccagcctgttacctgttctcactgaaaagtctggctaatgctcttgtgtagtcacttctgattctgacaatcaatcaatcaatggcctagagcactgactgttaacacaaacgtcactagcaaagtagcaacagctttaagtctaaatacaaagctgttctgtgtgagaattttttaaaaggctacttgtataataacccttgtcatttttaatgtacaaaacgctattaagtggcttagaatttgaacatttgtggtctttatttactttgcttcgtgtgtgggcaaagcaacatcttccctaaatatatattaccaagaaaagcaagaagcagattaggtttttgacaaaacaaacaggccaaaagggggctgacctggagcagagcatggtgagaggcaaggcatgagagggcaagtttgttgtggacagatctgtgcctactttattactggagtaaaagaaaacaaagttcattgatgtcgaaggatatatacagtgttagaaattaggactgtttagaaaaacaggaatacaatggttgtttttatcatagtgtacacatttagcttgtggtaaatgactcacaaaactgattttaaaatcaagttaatgtgaattttgaaaattactacttaatcctaattcacaataacaatggcattaaggtttgacttgagttggttcttagtattatttatggtaaataggctcttaccacttgcaaataactggccacatcattaatgactgacttcccagtaaggctctctaaggggtaagtaggaggatccacaggatttgagatgctaaggccccagagatcgtttgatccaaccctcttattttcagaggggaaaatggggcctagaagttacagagcatctagctggtgcgctggcacccctggcctcacacagactcccgagtagctgggactacaggcacacagtcactgaagcaggccctgtttgcaattcacgttgccacctccaacttaaacattcttcatatgtgatgtccttagtcactaaggttaaactttcccacccagaaaaggcaacttagataaaatcttagagtactttcatactcttctaagtcctcttccagcctcactttgagtcctccttggggttgataggaattttctcttgctttctcaataaagtctctattcatctcatgtttaatttgtacgcatagaattgctgagaaataaaatgttctgttcaacttataaaa

SEQ ID NO: 38 - DNA, 1078 bp, human
gtgtcgcgcggcgcggtggcctcggtcggtaccctgggcgcggacgagctgcctcattagtattcgtacccacgaggcggcgcacggggccctcggggacagcgagcgtcgcgccatggcttatcactcgggctacggagcccacggctccaagcacagggcccgggcagccccggatccccctcccctcttcgatgacacaagcggtggttattccagccagcccgggggatacccagccacaggagcagacgtggccttcagtgtcaaccacttgcttggggacccaatggccaatgtggctatggcctatggcagctccatcgcatcccatgggaaggacatggtgcacaaggagctgcaccgttttgtgtctgtgagcaaactcaagtatttttttgctgtggacacagcctacgtggccaagaagctagggctgctggtcttcccctacacacaccagaactgggaagtgcagtacagtcgtgatgctcctctgcccccccggcaagacctcaacgcccctgacctctatatccccacgaaggccttcattacttacgtgctcctggctgggatggcactgggcattcagaaaaggttctccccggaggtgctgggcctgtgtgcaagcacagcgctggtgtgggtggtgatggaggtgctggccctgctcctgggcctctacctggccaccgtgcgcagtgacctgagcacctttcacctgctggcctacagtggctacaaatacgtgggaatgatcctcagtgtgctcacggggctgctgttcggcagcgatggctactacgtggcgctggcctggacctcatcggcgctcatgtacttcattgtgcgctctttgcggacagcagccctgggccccgacagcatggggggccccgtcccccggcagcgtctccagctctacctgactctgggagctgcagccttccagcccctcatcatatactggctgactttccacctggtccggtgaccccctggccccagatggcactgagtttttcattcattgaagatttgatttccttgaaaaaaaaaaaaaaaaaaaaaaaaa

SEQ ID NO: 39 - DNA, 1145 bp, human
attctctccccagcttgctgagccctttgctcccctggcgactgcctggacagtcagcaaggaattgtctcccagtgcattttgccctcctggctgccaactctggctgctaaagcggctgccacctgctgcagtctacacagcttcgggaagaggaaaggaacctcagaccttccagatcgcttcctctcgcaacaaactatttgtcgcaggaataaagatggctgctgaaccagtagaagacaattgcatcaactttgtggcaatgaaatttattgacaatacgctttactttatagctgaagatgatgaaaacctggaatcagattactttggcaagcttgaatctaaattatcagtcataagaaatttgaatgaccaagttctcttcattgaccaaggaaatcggcctctatttgaagatatgactgattctgactgtagagataatgcaccccggaccatatttattataagtatgtataaagatagccagcctagaggtatggctgtaactatctctgtgaagtgtgagaaaatttcaactctctcctgtgagaacaaaattatttcctttaaggaaatgaatcctcctgataacatcaaggatacaaaaagtgacatcatattctttcagagaagtgtcccaggacatgataataagatgcaatttgaatcttcatcatacgaaggatactttctagcttgtgaaaaagagagagacctttttaaactcattttgaaaaaagaggatgaattgggggatagatctataatgttcactgttcaaaacgaagactagctattaaaatttcatgccgggcgcagtggctcacgcctgtaatcccagccctttgggaggctgaggcgggcagatcaccagaggtcaggtgttcaagaccagcctgaccaacatggtgaaacctcatctctactaaaaatacaaaaaattagctgagtgtagtgacgcatgccctcaatcccagctactcaagaggctgaggcaggagaatcacttgcactccggaggtagaggttgtggtgagccgagattgcaccattgcgctctagcctgggcaacaacagcaaaactccatctcaaaaaataaaataaataaataaacaaataaaaaattcataatgtg

SEQ ID NO: 40 - DNA, 1840 bp, human
ggaagaagaggctctcccgcgggagcccttgaggaccaagtttgcggccacttctgcaggcgtcccttcttagctctcgcccgcccctttctgcagcctaggcggcccgggttctcttctcttcctcgcgcgcccagccgcctcggttcccggcgaccatggtgacgatggaggagctgcgggagatggactgcagtgtgctcaaaaggctgatgaaccgggacgagaatggcggcggcgcgggcggcagcggcagccacggcaccctggggctgccgagcggcggcaagtgcctgctgctggactgcagaccgttcctggcgcacagcgcgggctacatcctaggttcggtcaacgtgcgctgtaacaccatcgtgcggcggcgggctaagggctccgtgagcctggagcagatcctgcccgccgaggaggaggtacgcgcccgcttgcgctccggcctctactcggcggtcatcgtctacgacgagcgcagcccgcgcgccgagagcctccgcgaggacagcaccgtgtcgctggtggtgcaggcgctgcgccgcaacgccgagcgcaccgacatctgcctgctcaaaggcggctatgagaggttttcctccgagtacccagaattctgttctaaaaccaaggccctggcagccatcccacccccggttccccccagcgccacagagcccttggacctgggctgcagctcctgtgggaccccactacacgaccaggggggtcctgtggagatccttcccttcctctacctcggcagtgcctaccatgctgcccggagagacatgctggacgccctgggcatcacggctctgttgaatgtctcctcggactgcccaaaccactttgaaggacactatcagtacaagtgcatcccagtggaagataaccacaaggccgacatcagctcctggttcatggaagccatagagtacatcgatgccgtgaaggactgccgtgggcgcgtgctggtgcactgccaggcgggcatctcgcggtcggccaccatctgcctggcctacctgatgatgaagaaacgggtgaggctggaggaggccttcgagttcgttaagcagcgccgcagcatcatctcgcccaacttcagcttcatggggcagctgctgcagttcgagtcccaggtgctggccacgtcctgtgctgcggaggctgctagcccctcgggacccctgcgggagcggggcaagacccccgccacccccacctcgcagttcgtcttcagctttccggtctccgtgggcgtgcactcggcccccagcagcctgccctacctgcacagccccatcaccacctctcccagctgttagagccgccctgggggccccagaaccagagctggctcccagcaagggtaggacgggccgcatgcgggcagaaagttgggactgagcagctgggagcaggcgaccgagctccttccccatcatttctccttggccaacgacgaggccagccagaatggcaataaggactccgaatacataataaaagcaaacagaacactccaacttagagcaataacggctgccgcagcagccagggaagaccttggtttggtttatgtgtcagtttcacttttccgatagaaatttcttacctcatttttttaagcagtaaggcttgaagtgatgaaacccacagatcctagcaaatgtgcccaaccagctttactaaagggggaggaagggagggcaaagggatgagaagacaagtttcccagaagtgcctggttctgtgtacttgtccctttgttgtcgttgttgtagttaaaggaatttcattttttaaaaaaaaaaaaaaaaaa

SEQ ID NO: 41 - DNA, 2263 bp, human
aggaagtagggagcggggtggcaggggggggacccgccgcggctgctgccaccgccgccaccaccgcctctgctcgtggcgtgggaaaggaggtgtgagtcccgggcgcgagccgcggcggcgccgctgcgggagggtcggcggtgggaaggcgatggcggatttagataaactcaacatcgacagcattatccaacggctgctggaagtgagagggtccaagcctggtaagaatgtccagcttcaggagaatgaaatcagaggactgtgcttaaagtctcgtgaaatctttctcagtcagcctatcctactagaacttgaagcaccactcaaaatatgtggtgacatccatggacaatactatgatttgctgcgactttttgagtacggtggtttcccaccagaaagcaactacctgtttcttggggactatgtggacaggggaaagcagtcattggagacgatctgcctcttactggcctacaaaataaaatatcctgagaatttttttcttctcagagggaaccatgaatgtgccagcatcaacagaatttatggattttatgatgaatgtaaaagaagatacaacattaaactatggaaaactttcacagactgttttaactgtttaccgatagcagccatcgtggatgagaagatattctgctgtcatggaggtttatcaccagatcttcaatctatggagcagattcggcgaattatgcgaccaactgatgtaccagatcaaggtcttctttgtgatcttttgtggtctgaccccgataaagatgtcttaggctggggtgaaaatgacagaggagtgtccttcacatttggtgcagaagtggttgcaaaatttctccataagcatgatttggatcttatatgtagagcccatcaggtggttgaagatggatatgaattttttgcaaagaggcagttggtcactctgttttctgcgcccaattattgcggagagtttgacaatgcaggtgccatgatgagtgtggatgaaacactaatgtgttcttttcagattttaaagcctgcagagaaaaagaagccaaatgccacgagacctgtaacgcctccaaggggtatgatcacaaagcaagcaaagaaatagatgtcgttttgacactgcctagtcgggacttgtaacatagagtatataaccttcatttttaagactgtaatgtgtactggtcagcttgctcagatagatctgtgtttgtgggggcccttccttccatttttgatttagtgaatggcatttgctggttataacagcaaatgaaagactcttcactccaaaaagaaaagtgttttgttttttaattctctgttccttttgcaaacaattttaatgatggtgttaaagctgtacaccccaggacagtttatcctgtctgaggagtaagtgtacaattgatcttttttaattcagtacaacccataatcatgtaaatgctcattttctttaggacataaagagagccctagggtgctctgaatctgtacatgttcttgtcataaaatgcatactgttgatacaaaccactgtgaacattttttatttgagaattttgtttcaaagggattgctttttcctctcattgtcttgttatgtacaaactagtttttatagctatcaacattaggagtaactttcaaccttgccagcatcactggtatgatgtatatttaattaaagcacacttttccccgaccgtatacttaaaatgacaaagccattcttttaaatatttgtgactctttcctaaagccaaagtttctgttgaattatgttttgacacacccctaagtacaaggtggtatggttgtatacacatgctgccttcttggggattcaaaaacaggtttttgattttgaatagcaattagtgatatagtgctgtttaagctactaacgataaaaggtaataacattttatacaatttccatatagtctattcattaagtaatctttttacagttgcatcaggcctgaacccgtccattcagaaagcttcaaattatagaaacaatactgttctatacgagtgaccgattatgctttctttggcctacattctttattctgcggtgaagttgaggcttataagttaaaacaaaggaactaacttactgtccaccagtttatacagaactcacagtacctatgacttttttaaactaagatctgttaaaaaagaaatctgtttcaacagatgaccgtgtacaataccgtgtggtgaaaat

SEQ ID NO: 42 - DNA, 6755 bp, human
gagagaagcctgatatgtaacccaggcgggtgggagcctcagtctgtcgggctgaggtctggcatctacaaagcctcttggccgtgttctgaacttgaagcctggaggagttctctgctcagcacagccaaggaacagaattagaagaaaaggaaccctggcctgaggcaggtgacaaacattaccaccccagctgtgcacgatgcagcagatgcaaccagatgttcacagaaggagaggaaatgtatcttcaaggctccaccgtttggcatcccgactgtaagcaatctacgaagaccgaggaaaagctgcggcctaccaggacatcctcggaaagtatttattctaggccaggctccagtattcctggctcaccaggtcatactatctatgcaaaagtagacaatgagatcctggattacaaggatttagcagccattccgaaggtcaaggcaatttatgacattgaacgtccagatcttattacctatgagcctttctacacttcgggctatgatgacaaacaggagagacagagccttggagagtctccgaggactttgtctcctactccatcagcagaagggtaccaggatgttcgggatcggatgatccatcggtccacgagccagggctccatcaactcccctgtgtacagccgccacagctacactccaaccacgtcccgctctccccagcatttccacagacctggcaatgagccgtccagcggccggaactcccctctcccttaccggccagacagccgccctctaactccaacttacgctcaggcccctaaacatttccatgttccagatcaaggaatcaacatttaccgaaagccacccatctacaaacagcatgctgccttggcagcccagagcaagtcctcagaagatatcatcaagttttccaagttcccagcagcccaggcaccagaccccagcgagacaccagagattgagacggaccactggcctggtcccccctcatttgctgtcataggacctgacatgaaacgcagatctagtggcagagaggaagatgatgaggaacttctgagacgtcggcagcttcaagaagagcaattaatgaagcttaactcaggcctgggacagttgatcttgaaagaagagatggagaaagagagccgggaaaggtcatctctgttagccagtcgctacgattctcccatcaactcagcttcacatattccatcatctaaaactgcatctctccctggctatggaagaaatgggcttcaccggcctgtttctaccgacttcgctcagtataacagctatggggatgtcagcgggggagtgcgagattaccagacactcccagatggccacatgcctgcaatgagaatggaccgaggagtgtctatgcccaacatgttggaaccaaagatatttccatatgaaatgctcatggtgaccaacagagggcgaaacaaaatcctcagagaggtggacagaaccaggctggagcgccacttagcccctgaagtgtttcgggaaatctttggaatgtccatacaggagtttgacaggttacctctttggagacgcaacgacatgaagaaaaaagcaaaactcttctaagtcccactcgtgaatggcaattagagaaaggactgacagtggcggtgccccataggatgtcatattgaggcccaaacttgattggagaatttgcaaactaccgtcgctcagcaacaccaaaaagagaaagtctggttaaaacaccatgagtcaaatgtcgggccagccaacagtaacacttgccaagaagcatggcgtagaaatttctatgttccgaaacacagagtagtgtccacatgtgaccttttcacattaccttatgtatacaacaggagctgcgttgttttcttctttttcttttcctttatctgttgtccaacaacagtgctgactgtccggataagagctggcaagtgcccttaggatgccgcatgggaaaaatcggttatcataatttcaaagtataaatatatttattatgtagcgctgcggctaagaaggaagagtgaggggtctgtccatggggtggcagtgatttcacacccgcctttcttgaatggcttcgtgttactcagccgtgccctggcaggaatggaactccatcagggaacagggcagctctgtttgaatggggtgaaaaacagcaaaattaattcttagcaagctccttgttctctactgttcaccgggacctggctggcaaatgacttagctattagtatatttaaaaactgttcaaaagcaaaagagaaggaacaggaatgaaaggaaaccttcccttaactctttctgcttcccatagcaggtctggttcctgctccccgcctaggacaggagctgtctggagctccacctttgcaaagaaaggaaaaacacaaattgcccccagattggcccagtggcatgccattaggtgattttgcgtaggggccatatcattcatggccaaaaaaaaccaaaaagcaaaaaacaaaaacaagtcccatgtcctactgactctagttcttccagtcgttatcctctgctgtctctctggcatcctatgaatcagatcaagcccatgctgttgttggtttttaaggtttctttcaataataggctaaggaaagacatgtttttctcttttaaatctctgcaactccaaagtagactccttcgaacgtatttaatttggccttttcagctttcttcttgcccagctctgtcgaattcatgggtgtgtgtgcacatgttggtttcactcacccagagtaattttgtgagcatgcatgtgctttttaatttctgcttgaatgtttgtcctgctgtgttgcagcttctaaagacattgtcaccgagtgtgtgtgactcaaagatcaagagtgaggctaaactgcgaccccaacatcacttccttcatggaaaggtgttggcgctgatgtagctcatggaaggatcagactgggaaccacagaggaaggatgaggtgggatgtggaggtcagagcatctcacgagagtccctcaggctctgcaacactagattcgaatatgagcctagggtttccccaacacgtgcgtgcatatgaaaccacatcatgattcccgacgtgagttagatgagggagagtttgtgtaactccacacccaggagggtaagccactttattatcatgctttggggctgtgcttaagaatttaagctctgttttaaagccgaagaagaaaacatttgaatttaccttcatatgccactaagaacaaaagctctgaagttcttcctgctggcaccaaatattaggattacaagctgtctctctgacaccagtggtaaaaaagcaagtagaatagtttctgagtctgtgttgcagcctctgacacgtggctcactgctgtcatttggggggtggaatgaacagtcactgttctatagcatcctcctgtgctcttacctaaggaatctaaattgtcctagccttccattgctatgcactgaatgaggactactctcgtgtgtgtgtgtgtgtgtgtgtgtgtgtgtgtgtgtgtgtgtgtgtgttggatctactaacatgtttagcaaatggctctgacagggtggccatgagcagatgagaccagctcttgcttggttggaagccacactgcagtttgcagtttagcctttggtgtctcagtggcctcttgcttattccatgctttttctatcccatcctctctctgtgccctcctgcaatggcagccttggcacaatgcctgggtgtcctcatccttgctttactctggaaagtgtggggatggagaggatggtgcatttccagtgcttccttacctgtcaataacaatggtggccacctgggtcccagcacttggctggtggagagatttttttcattatggagctaataaagggtccctacgttttacattgattaatgaattatcacccagacatgtgcagatgaaatgtcaatataagaaaaatcaaggtatttgggaaacatgggcataatcaggtagttttgctaaaattcctcttccttggtctacataaagaagacatgggtctgtcatagaatggggacattagactgaaaactgggacaacccccttccttattttatagagactaggagttaaggggcctgcctaagattgcagtgataaagaactgaaccctaaatcagctcccactaatgaaaccctgctttccttgtatcttttaaactcagttctgcatccaacttgagaagaaaaaaagcttctttccatatcaagtaccatatgagttcatttaaactgcaccttgcagaaatgcattgccagaaagcaccagtagcctcctatctgcaagcagagtagtgctctgctctggggaggggtcattggaaaccataatgcagagtgggccccctactccatttcccagcaaaaggctccagctggagggatgggttgtggggcaacctggttcctgctaactgccagattgaatgtgtgggctagaatgcctgcgcatttagttaaactgggctcagcatgcttgtcctcaaaatgtccatcctggtcacagcacacaagatggctattggtctgcttttaccctaccctgtactatacatgaaaattccagttattaacacactcaaactggtggagcttgttcaccctaggaaggggattgtatatatggcaggcttccctggtgccgatgtaaagggctacatttgggaacatttgacttccttgggactcttaagtgcatactgatggcatgaagtaaaaggggcctcaatgatgataggaaaatcagttcttttaaaatttcttcaagaaaatccaggctatcacatagtctttctgtgtgacttattaggagataggaagagcattgggaaacttgcacagctagctatgcatctacattttggtttggggttagttatgaaatgttcttaatatgacgtgttcaataacttcacataaacttcctgttctccaaaacctcaaagagatagagttaatgagttgttgtttttttttaaatgggggtagttttctatctgtcatgggctctagcatctactccgctacccaattctgtcatctccaagctgagtttcctcttctgaggcagaggctggagcagttctttttcagttctcatcctctccatcccaatccagtatatcaatcaactctaactcggagacgtctagctggcaatgtttctaaaactttcactggatttctttagacattgaagcaaacatttttttctaagaattgcttctcagatgatgatatcaaatgtatatgcttttgcaagtttgaaaagttcaaattaaccacttttgactaggtaagtctttctaaaaaccatttaaagctaactgggtcttagcatcctcctgtgtatggaagagacaggtgaccgctccaggttgggtgctcacagaacccttttcctgactctcatggaagatggtggaaggaaaatagactgtctcatcaaccctcctgtgtcctctgaagcaatctcagtttttattaaccacctcttctgtgtttctggtagctatttaacctgtatttaatctgtacttcctatgccagcctcaattttatttgatttttaaaattattctcttctaaccaatgaagtgtttgtcagtatgccccaaagcttgctcttttgtgctcccttttgaataactttctatccagaaaaagagattatttgggacttgagatttgcagtgataccaacttatagcaatgatgtactttaagggaactacccaactatgttgtgatagaagaaagagaaaccttcactttggcattttttttaatcactgtttatttttctgtttgcggcccaggaagcagtgggaggtggtggcagatatgctttgcatatggattgttatgtttttatttgggcaagtttaatcatggaaaactcaaaaagaaggggggaaatggtcagtttaagccaaaagaaactttctaaacaatgtataggtacacagcaaaattaaacaaatccaacaatttctgaagcttagtgtaattgagtggtggttgttattcaataaaattattcccaaaagtgtttctcctaagagtgcagttcccatgagtcacttcctgaacccattgaccaaaggtggacagagacaatcctgtagaccttgacattcagaaagatgtgagctgcttactgatcatatatgcatacgtttctttacagcagaggaaaccattgtccacaaaactgatgttcttttggggttttatgtacagacttgtccaatcatgtgtgtggttcctgcgagttgctgatgactccgcattgaagctctctgagttctttgattttaagttgggtttatggaattttttcaaatgttggaaggcgtgtggttcttcctgccctccctccccttttggaaatatgaaagcaaatgtttagaagaattccttttgaaaagctgtgtcgtgttccctgtgaaactgagcaggtgtgtgttggcgcgctaagtgccacatgcttgtgtgtagaggaggaggtggccctgccggctccgcgctgctgtgcctgtgatccctacctgctccccgctcctgttgccagcagcactcactgcactcctttgtcatatactctgcatcactgtcatactcacaacttcgtgaataaagttgtgtgctttattcgtc

SEQ ID NO: 43 - DNA, 1607 bp, human
cctaattctcctgaggctgagggagggtggagggtctcaaggcaacgctggccccacgacggagtgccaggagcactaacagtacccttagcttgctttcctcctccctcctttttattttcaagttcctttttatttctccttgcgtaacaaccttcttcccttctgcaccactgcccgtacccttacccgccccgccacctccttgctaccccactcttgaaaccacagctgttggcagggtccccagctcatgccagcctcatctcctttcttgctagcccccaaagggcctccaggcaacatggggggcccagtcagagagccggcactctcagttgccctctggttgagttggggggcagctctgggggccgtggcttgtgccatggctctgctgacccaacaaacagagctgcagagcctcaggagagaggtgagccggctgcaggggacaggaggcccctcccagaatggggaagggtatccctggcagagtctcccggagcagagttccgatgccctggaagcctgggagaatggggagagatcccggaaaaggagagcagtgctcacccaaaaacagaagaagcagcactctgtcctgcacctggttcccattaacgccacctccaaggatgactccgatgtgacagaggtgatgtggcaaccagctcttaggcgtgggagaggcctacaggcccaaggatatggtgtccgaatccaggatgctggagtttatctgctgtatagccaggtcctgtttcaagacgtgactttcaccatgggtcaggtggtgtctcgagaaggccaaggaaggcaggagactctattccgatgtataagaagtatgccctcccacccggaccgggcctacaacagctgctatagcgcaggtgtcttccatttacaccaaggggatattctgagtgtcataattccccgggcaagggcgaaacttaacctctctccacatggaaccttcctgggactttgattttacggatatcttgcttctgttccccatggagctccgaattcttgcgtgtgtgtagatgaggggcgggggacgggcgccaggcattgttcagacctggtcggggcccactggaagcatccagaacagcaccaccatctagcggccgctcgagggaagcacccgccggttggccgaagtccacgaagccgccctctgctagggaaaacccctggttctccatgccacacctctctccaggtgccctctgcctcttcaccccacaagaagccttatcctacgtccttctctccatctatcggaccccagtttccatcactatctccagagatgtagctattatgcgcccgtctacagggggtgcccgacgatgacggtgccttcgcagtcaatttactcttcgggtcccaaggtttggctttcacgcgctccattgccccggcgtggcaggccattccaaggccttccgggctggaactggtgtcggaggagcctcgggtgtatcgtacgccctggtgttggtgttgcctcactcctctgagctcttctttctgatcaagccctgcttaaagttaaataaaatagaatgaatgataaaaaaaaaaaaaaa

SEQ ID NO: 44 - DNA, 5067 bp, human
ctcctccccatcctctccctctgtccctctgtccctctgaccctgcactgtcccagcaccatgggacccacctcaggtcccagcctgctgctcctgctactaacccacctccccctggctctggggagtcccatgtactctatcatcacccccaacatcttgcggctggagagcgaggagaccatggtgctggaggcccacgacgcgcaaggggatgttccagtcactgttactgtccacgacttcccaggcaaaaaactagtgctgtccagtgagaagactgtgctgacccctgccaccaaccacatgggcaacgtcaccttcacgatcccagccaacagggagttcaagtcagaaaaggggcgcaacaagttcgtgaccgtgcaggccaccttcgggacccaagtggtggagaaggtggtgctggtcagcctgcagagcgggtacctcttcatccagacagacaagaccatctacacccctggctccacagttctctatcggatcttcaccgtcaaccacaagctgctacccgtgggccggacggtcatggtcaacattgagaacccggaaggcatcccggtcaagcaggactccttgtcttctcagaaccagcttggcgtcttgcccttgtcttgggacattccggaactcgtcaacatgggccagtggaagatccgagcctactatgaaaactcaccacagcaggtcttctccactgagtttgaggtgaaggagtacgtgctgcccagtttcgaggtcatagtggagcctacagagaaattctactacatctataacgagaagggcctggaggtcaccatcaccgccaggttcctctacgggaagaaagtggagggaactgcctttgtcatcttcgggatccaggatggcgaacagaggatttccctgcctgaatccctcaagcgcattccgattgaggatggctcgggggaggttgtgctgagccggaaggtactgctggacggggtgcagaacctccgagcagaagacctggtggggaagtctttgtacgtgtctgccaccgtcatcttgcactcaggcagtgacatggtgcaggcagagcgcagcgggatccccatcgtgacctctccctaccagatccacttcaccaagacacccaagtacttcaaaccaggaatgccctttgacctcatggtgttcgtgacgaaccctgatggctctccagcctaccgagtccccgtggcagtccagggcgaggacactgtgcagtctctaacccagggagatggcgtggccaaactcagcatcaacacacaccccagccagaagcccttgagcatcacggtgcgcacgaagaagcaggagctctcggaggcagagcaggctaccaggaccatgcaggctctgccctacagcaccgtgggcaactccaacaattacctgcatctctcagtgctacgtacagagctcagacccggggagaccctcaacgtcaacttcctcctgcgaatggaccgcgcccacgaggccaagatccgctactacacctacctgatcatgaacaagggcaggctgttgaaggcgggacgccaggtgcgagagcccggccaggacctggtggtgctgcccctgtccatcaccaccgacttcatcccttccttccgcctggtggcgtactacacgctgatcggtgccagcggccagagggaggtggtggccgactccgtgtgggtggacgtcaaggactcctgcgtgggctcgctggtggtaaaaagcggccagtcagaagaccggcagcctgtacctgggcagcagatgaccctgaagatagagggtgaccacggggcccgggtggtactggtggccgtggacaagggcgtgttcgtgctgaataagaagaacaaactgacgcagagtaagatctgggacgtggtggagaaggcagacatcggctgcaccccgggcagtgggaaggattacgccggtgtcttctccgacgcagggctgaccttcacgagcagcagtggccagcagaccgcccagagggcagaacttcagtgcccgcagccagccgcccgccgacgccgttccgtgcagctcacggagaagcgaatggacaaagtcggcaagtaccccaaggagctgcgcaagtgctgcgaggacggcatgcgggagaaccccatgaggttctcgtgccagcgccggacccgtttcatctccctgggcgaggcgtgcaagaaggtcttcctggactgctgcaactacatcacagagctgcggcggcagcacgcgcgggccagccacctgggcctggccaggagtaacctggatgaggacatcattgcagaagagaacatcgtttcccgaagtgagttcccagagagctggctgtggaacgttgaggacttgaaagagccaccgaaaaatggaatctctacgaagctcatgaatatatttttgaaagactccatcaccacgtgggagattctggctgtcagcatgtcggacaagaaagggatctgtgtggcagaccccttcgaggtcacagtaatgcaggacttcttcatcgacctgcggctaccctactctgttgttcgaaacgagcaggtggaaatccgagccgttctctacaattaccggcagaaccaagagctcaaggtgagggtggaactactccacaatccagccttctgcagcctggccaccaccaagaggcgtcaccagcagaccgtaaccatcccccccaagtcctcgttgtccgttccatatgtcatcgtgccgctaaagaccggcctgcaggaagtggaagtcaaggctgccgtctaccatcatttcatcagtgacggtgtcaggaagtccctgaaggtcgtgccggaaggaatcagaatgaacaaaactgtggctgttcgcaccctggatccagaacgcctgggccgtgaaggagtgcagaaagaggacatcccacctgcagacctcagtgaccaagtcccggacaccgagtctgagaccagaattctcctgcaagggaccccagtggcccagatgacagaggatgccgtcgacgcggaacggctgaagcacctcattgtgaccccctcgggctgcggggaacagaacatgatcggcatgacgcccacggtcatcgctgtgcattacctggatgaaacggagcagtgggagaagttcggcctagagaagcggcagggggccttggagctcatcaagaaggggtacacccagcagctggccttcagacaacccagctctgcctttgcggccttcgtgaaacgggcacccagcacctggctgaccgcctacgtggtcaaggtcttctctctggctgtcaacctcatcgccatcgactcccaagtcctctgcggggctgttaaatggctgatcctggagaagcagaagcccgacggggtcttccaggaggatgcgcccgtgatacaccaagaaatgattggtggattacggaacaacaacgagaaagacatggccctcacggcctttgttctcatctcgctgcaggaggctaaagatatttgcgaggagcaggtcaacagcctgccaggcagcatcactaaagcaggagacttccttgaagccaactacatgaacctacagagatcctacactgtggccattgctggctatgctctggcccagatgggcaggctgaaggggcctcttcttaacaaatttctgaccacagccaaagataagaaccgctgggaggaccctggtaagcagctctacaacgtggaggccacatcctatgccctcttggccctactgcagctaaaagactttgactttgtgcctcccgtcgtgcgttggctcaatgaacagagatactacggtggtggctatggctctacccaggccaccttcatggtgttccaagccttggctcaataccaaaaggacgcccctgaccaccaggaactgaaccttgatgtgtccctccaactgcccagccgcagctccaagatcacccaccgtatccactgggaatctgccagcctcctgcgatcagaagagaccaaggaaaatgagggtttcacagtcacagctgaaggaaaaggccaaggcaccttgtcggtggtgacaatgtaccatgctaaggccaaagatcaactcacctgtaataaattcgacctcaaggtcaccataaaaccagcaccggaaacagaaaagaggcctcaggatgccaagaacactatgatccttgagatctgtaccaggtaccggggagaccaggatgccactatgtctatattggacatatccatgatgactggctttgctccagacacagatgacctgaagcagctggccaatggtgttgacagatacatctccaagtatgagctggacaaagccttctccgataggaacaccctcatcatctacctggacaaggtctcacactctgaggatgactgtctagctttcaaagttcaccaatactttaatgtagagcttatccagcctggagcagtcaaggtctacgcctattacaacctggaggaaagctgtacccggttctaccatccggaaaaggaggatggaaagctgaacaagctctgccgtgatgaactgtgccgctgtgctgaggagaattgcttcatacaaaagtcggatgacaaggtcaccctggaagaacggctggacaaggcctgtgagccaggagtggactatgtgtacaagacccgactggtcaaggttcagctgtccaatgactttgacgagtacatcatggccattgagcagaccatcaagtcaggctcggatgaggtgcaggttggacagcagcgcacgttcatcagccccatcaagtgcagagaagccctgaagctggaggagaagaaacactacctcatgtggggtctctcctccgatttctggggagagaagcccaacctcagctacatcatcgggaaggacacttgggtggagcactggcctgaggaggacgaatgccaagacgaagagaaccagaaacaatgccaggacctcggcgccttcaccgagagcatggttgtctttgggtgccccaactgaccacacccccattcc

SEQ ID NO: 45 - DNA, 3887 bp, human
gcgtttgaatctggtccgagcgcgggaaacggcgggtccccgagcccagggttacaaaataaatgccatttgaacagtgccatctgtcatggaaaaacgtgagacgtttgtacaagccgtgtctaaggagctggttggagagtttttgcaatttgttcaacttgataaagaggcctctgatcctttcagcctaaatgaattactagatgaattatcaaggaaacagaaagaagaattatggcaaaggctgaagaatttattgacagatgtgttgttagaaagcccagtggatgggtggcaggtagtggaagcccagggtgaagacaatatggaaaccgaacatggctcaaaaatgagaaaaagcatagaaataatttatgcaattacatctgtgattcttgcttctgtgtctgtaataaatgaaagtgagaactacgaagccctactggaatgtgttattatattaaatggtattttatatgcattacctgagtctgaacgaaaactacagagttctattcaggatttgtgtgttacctggtgggagaaaggcctgcctgccaaggaagacacaggaaagacagcctttgtcatgttactaaggaggagtctggagactaagacaggtgcagacgtatgtcggctttggcgtatccatcaagctttatattgctttgattatgatttggaggaaagtggagaaattaaagatatgttacttgagtgcttcataaatattaattatatcaagaaagaagagggaagaagatttcttagttgtctcttcaactggaatatcaacttcatcaaaatgatccacgggaccattaaaaaccagttacagggattacaaaagtctttgatggtatacattgcagaaatttatttcagagcttggaaaaaggcttcagggaaaatactggaggcgattgaaaatgattgcatccaggacttcatgttccacgggatacaccttccgaggaggtctccagtgcattccaaagtgcgggaggttctgagttactttcaccatcaaaagaaagttcggcagggagtggaagagatgctttatagattatataagcccatcctttggagaggattaaaggccagaaactctgaagttcgatcaaatgctgcattgttgtttgttgaagcatttcctattagggatccaaaccttcatgctattgaaatggatagtgaaatccagaaacagtttgaagagctctatagccttttagaagatccttacccgatggtccgttccacagggatccttggtgtttgtaaaataacttctaagtactgggaaatgatgcccccgaccattcttattgacctcctgaagaaggtgactggggaactggcatttgacacgagctcagctgatgttcgttgttctgtctttaagtgtctgccaatgattttggacaacaaactgagccacccattgttagagcagctccttccagctctcagatacagtctccacgacaattcggagaaagtgagggtagcttttgtggacatgctgttgaagatcaaagctgtgagggctgctaagttttggaaaatatgtcccatggagcacattctggttcgtctggaaactgattctcgacctgtgtctcggcgcctggtgagcctcatctttaattctttcctgcctgtgaatcagccggaggaggtctggtgcgagcgctgtgtcaccctggtgcagatgaaccacgccgctgccaggaggttctatcagtacgcccacgaacacaccgcctgcaccaacatagcaaagctgattcacgttattcgtcattgcttaaatgcctgtatccagagggcagtgagagagcctccagaggacgaggaggaagaggacggaagggagaaggagaatgtgactgttctggacaaaacactgtcagtaaacgatgttgcatgcatggcaggtttactagaaatcattgtgattctctggaaaagtattgacagaagtatggaaaataataaagaggccaaactttacacgattaacaagtttgcctctgtgcttccagagtatctgaaagtatttaaggatgatcgctgcaagatccctttattcatgctaatgtcctttatgccggcctctgctgtccccccattcagctgtggtgtgatttccacgctgagaagccgggaggagggcgctgtggacaagagctactgcactttgttggattgcctctgctcctgggggcaggtggggcacattctggagcttgttgacaactggctgcccacagagcatgcccaggccaagagcaacacagcttctaaaggtagggtgcagatccatgacacacgcccagtcaaacctgaattggcattggtctacattgagtatctgctgactcatccaaagaaccgcgagtgcttgctctctgctcctcggaagaaacttaaccatcttttgaaagcccttgaaacgtcaaaggcagatctggagtcacttctgcagacaccgggtgggaagcctcgtggcttcagtgaagcagctgccccgcgagcctttggtctccactgtcgcctgagcatccatcttcagcacaagttctgctcagaaggaaaggtgtatttgtccatgttggaagacactggcttttggttagaaagcaaaattttatcttttattcaagatcaagaagaagactacctgaagcttcatagggtcatttatcagcaaattatccagacctacctgactgtgtgtaaagatgttgttatggtaggccttggtgaccatcagtttcagatgcaactcttacagcggagtcttggaatcatgcaaacagtgaagggatttttttatgtttcattacttcttgacattctgaaagagataactggaagttccttgattcagaaaacagattcagatgaagaagttgcaatgctgttggacacagtccagaaagtatttcagaaaatgttggaatgtattgcacggagcttcaggaagcagccggaagaaggcctgcggctgctttattctgttcagaggcctcttcatgagttcattactgctgttcagtctcggcacacagacacccctgtgcaccggggtgtactttctactctgatcgctgggcctgtggttgagataagtcaccagctacggaaggtttctgacgtagaagagcttacccctccagagcatctttctgatcttccaccattttcaaggtgtttaataggaataataataaagtcttcgaatgtggtcaggtcatttttggatgaattaaaggcatgtgtggcttctaatgatattgaaggcattgtgtgcctcacggctgctgtgcatattatcctggttattaatgcaggtaaacataaaagctcaaaagtgagggaggttgcagccactgttcacagaaaactaaagacattcatggaaattactttggaagaggatagcattgaaagatttctctatgaatcatcatcaagaactctgggagaacttttgaattcataaccaagccaacatctccagacatgtaaaaatagggaaaagtgattcaaattgaaatgcctgtgtattttcctattgtttttaatgttaataacccatataatagggaaagggtgggatttttttgtgggaatgtgggaaggtgggggttatggaggagataactcaaaacttcttcaattttgcctagtgcctgcgtaaataatatatttaatataaaggactccaggtatgaatggtgtagaaatccatgattccaagaaaaaacacttttctagcaaacctggttgtttttaaaatgacttttatatatgtaatattgcttggaaactatgagtaataaagcaatgacaacaaaaaaaaaaaaaaa

SEQ ID NO: 46 - DNA, 5745 bp, human
cacagggcctgcagagaggggacccaccgggcaggatgcactggcttccagcaggccacgacatcaacggtgccctggagccctccaacatagacaccagcatcctggaggagtacatcagcaaggaggatgcctccgacctctgcttccctgacatctctgctccagccagctcggcctcctactcccacgggcagcctgcgatgcctggctccagcggggtccaccacctgagcccccctgggggtggaccctccccggggcgccatggtcccctcccacccccgggctacggcaccccgctgaactgcaacaacaacaacggcatgggcgctgcccccaagcccttcccggggggcaccgggccccccatcaaggctgagcccaaggctccctatgccccaggcacactgccggactctcccccagactcgggctccgaggcctactccccccagcaggtgaatgagccccacctcctgcgcacgataacccctgagacactgtgccacgtgggagtgccctcccgcctggagcatccgcccccacctccagcccacttgccaggccccccgccacccccaccacccccacctcactaccctgtcctgcagcgggatctgtacatgaaggccgagcccccgatcccccactacgctgccatggggcaggggctggtgcccactgatcttcaccacacccagcagtcccagatgctgcaccagctcctgcagcagcacggagctgagctccctacacacccctccaagaagaggaagcactctgaatcccccccgagcaccctcaatgcccagatgctgaatggaatgatcaaacaggagcctgggaccgtgacagccctgcctctgcaccccactcgagccccatcgccaccctggcctccccagggtccgctctccccgggccctggttccttgcctctcagcattgcccgtgtccagacaccgccttggcacccgccaggtgccccctccccaggcctcctgcaggacagtgacagcctcagtggctcctacctggaccccaactaccagtccatcaagtggcagcctcatcagcagaacaagtgggcgaccctgtacgatgctaactacaaggagctgcccatgctcacctaccgcgtggatgcggacaagggcttcaacttttcggtgggcgacgacgcctttgtgtgccagaagaagaaccacttccaggtgacagtgtacatcggcatgctgggcgagcccaagtacgtcaagacgcccgagggcctcaagcccctcgactgcttctatctgaagctgcacggagtgaagctggaggccctgaaccagtccattaacatcgagcagtcccagtcagaccggagcaagcggcccttcaacccggtcacggtcaatctgccccctgagcaggtcacgaaggtgactgtggggcggctgcacttcagcgagaccaccgctaacaacatgcgtaagaagggcaagcccaacccggaccagaggtacttcatgctggtggtggccctccaggctcatgcacagaaccagaactacacgctggccgcccagatctcagagcgcatcattgtgcgggcctccaacccaggccagttcgagagcgacagcgatgtgttgtggcagcgggcacaggtgcccgacaccgtcttccaccacggccgcgtgggcatcaacacagaccggccggatgaggcgctggttgtgcacgggaatgtcaaggtcatgggctcgcttatgcacccctccgacctgcgcgccaaggaacacgtgcaggaggtggacaccaccgagcaattgaagaggatctcgcgcatgcggctggtgcactacagatacaagcccgagttcgccgccagcgcgggcatcgaggccaccgcgccagagacaggtgtcatcgctcaggaggtgaaggagatcttgcctgaggctgtgaaagacaccggagacatggtctttgccaatgggaaaaccatagagaacttcctggtggtgaacaaggagcgcatcttcatggagaacgtaggggccgtgaaggagctgtgcaagctgacagacaacctggagacgcgcattgatgagctggagcgctggagccacaagctggccaagctgcggcggctcgacagcctcaagtccaccggcagctcgggcgccttcagccatgcagggagccagttcagtcgggcgggcagcgtcccccacaagaagaggccccccaaggtggccagcaagtcatcgtccgtggttccggaccaggcctgcatcagccagcgcttcctgcagggaaccatcattgccctggtggtggtcatggccttcagcgtggtgtccatgtccacactgtacgtgctgagcctgcgcacagaggaggacctggtagacactgatggcaggtccagccagagctttgggaccacgcagctccgacagtcccccttgaccacggggctaccaggcatacagccctctttgctgctggtgaccaccagcctcaccagctcggccccaggttctgctgtccgcaccttggacatgtgttccagccacccctgccctgtcatctgctgttcctcacccactaccaaccctaccactggtcctagtcttggccccagctttaaccctggccatgttctcagcccaagtcccagccccagcaccaaccgctcaggccccagccagatggcccttctgccagtcaccaacatcagagccaagtcctggggtctttcagtcaatggcattggccactccaagcatcacaagagtctggagcctctggccagccctgcagtccccttccctggggggcagggcaaagccaagaacagtcccagccttggtttccatggccgggcccgccgaggggccctccagtccagcgtgggccctgctgagcccacctgggcccagggccagtcagagccagtgccctccctgacctccatccaggtgctggagaattcgatgtccatcacctcccagtactgtgctccaggggatgcctgcaggcctgggaacttcacctaccacatccctgtcagtagtggcaccccactgcacctcagcctgactctgcagatgaactcctcctcccccgtgtctgtggtgctgtgcagcctgaggtcaaaggaggaaccatgtgaggaggggagccttccacagagtctccacacccaccaggacacccagggcacctctcaccggtggccaataaccatcctgtccttccgtgaattcacctaccacttccgggtggcactgctgggtcaggccaactgcagttcagaggctctcgcccagccagccacagactaccacttccacttctaccgcctgtgtgactgagctgccctcctgaggcagcaccacaccagggaccaggggtgcccaggcaccccccaacactggatgcaatggtgttacactggagcccgctgcaggccagctctgctgttcactggccctacccgagactggtgaaactggaagtcttcacactggagttgctgttccagctggtcgcccctcacggcacagagggaacctgagagccagagacttcttgggccttcctgcctgccaccccctaggggccaggacaggaccagtttacctctttccagatatggtggttggagggctggttcaggtgccctggagggaaggggaagcctgtggccctgatttgttcagagcccattctcccttgcctccccttttgagactggagccaacccttttggagagaggacctgcccacctttgagatcagcagggggctcggatccagccctaagagacttgggtggacccccatgagtcaatggagggcagacggctctcccccttaaagctgttccctgggggatggcttggtagtggactttctggggtttgcctgttacgccagactcggacttctaagctttaagtgtggcccaggaggtttcttctccctgggagggcttggctcccaagaagtcccagggcagccgaggccagccctgcctgggttggagaaactgactttgtgccttaagtctactcagtgcctggtgaagccaccctcagcccttcacaggcctgaaccagtaggggccagtgggccaggtaagccctagagccttgaaccaggaatatccaggaagaggaaattccctttgagcccccagatggtattgcagcttcactgcctgcgttcctgggagcgtctggagctcacagtgatcagtgaccacatcattctctctgagcagaggagcaggaatccctcaagcagcagcctggtcttggctggtgggcagatgcaaatagcttttgctgttattaatgaagtaattactaaatgcacttaaaccagggcaggaaggaatggaaggatggagctagaaagctcagagtgggccagagcaggggtgtgacacttgcaaagacagggctctgactctgatccctcccagggagcctccgacacccatcccactcccaaccaccaagaccctgggttagggaagaagttgtatcttaagtgccaccttcaagtttcttagtggtgcctggtgcattccgaggctacatccaggctcatggaaggagtgtagtattcatttagccatgtctgccatgggtccagaaatgggaaagggaattgctgtccttgccctgtggtatgctgccacctctttgggaagcaggccttgcccctgtcccaccactcattctcagctttgaatgggaggcctttctatagtggaggcctttccttgaagcctatgaactgcaggcccccttttgccattgatctcaaagcacttgtcctcaggatagggaagagcagggggatgcaggaatagcagggatagcttgctcccagccccctccccaatttggttccgttgacataggaattttacgattcccaaaccatgcaggggctgagccttccttatgatgactttgttctccctcccactgggggaatcctccctatgccttaaaactgccgagccccactccatgtaataggattcctgggcttcctcaatgggggttcatgttcttggactgcgggccctcagtccttaactggaaagtgaccgtccactgccccatggagcccatctggacacagcacagccccaaaaccgttagcagctggctctgtttccaagcctggggaggggttcctcagtgcaggagttggggacaggctggggatccaagctgcttgagggggtcaaccttggaccaaagttgccttaagcctgtggtaaaagggcttcagggaaggtaagtgggccacctgctggaagctgccagctgcccggctggcaatggtgtgagtgtcttggccctgtccctgccctggggtccagcaggtcatccctcccttcttctctctcctttggcgtttgttcctgtagtcactgggctaatctccccctagcttcaagctgtacatagggcctcccagtgcaaatcctcctgcccataccgtgcacccttagaagcctgcgtgtgcatagagcgccccctacttcccagttaactcccagttcttctccctgagcttggtatttgtcatgtgccaactctgactctgaggtgggcagtgagggaagcagccccgggcctgcttgcttcctgtccccgaaatgttcgtttcttctgaagtaaatatacatatataaataaatgtataaatactgctttgtatctg

SEQ ID NO: 47 - DNA, 5261 bp, human
ctcaggcacgtctagattttcccacttacataggtcttgttaagaaaccagtcctgccttcttgccactcgtgtctttcgatggctcccttcccgaagtcccgctgcctctaagcggagtgttaagcggggctctcggaagccggtggaccgagatttccccgggtggggcgggcgcggtgtagcggcccgcgggctgacttgctcccggctgtcccccggccccagcgaccatgccccgtaaaggcacccagccctccactgcccggcgcagagaggaagggccgccgccgccgtcccctgacggcgccagcagcgacgcggagcctgagccgccgtccggccgcacggagagcccagccaccgccgcagagactgcaagtgaggaacttgataatagaagtttagaagagattttgaacagcattcctcctcccccgcctccagcaatgaccaatgaagctggagctcctcggcttatgataactcatattgtaaaccagaacttcaaatcctatgctggggagaaaattctgggacctttccataagcgcttttcctgtattatcgggccaaatggcagtggcaaatccaatgttattgattctatgctttttgtgtttggctatcgagcacaaaaaataagatctaaaaaactctcagtattaatacataattctgatgaacacaaggacattcagagttgtacagtagaagttcattttcaaaagataattgataaggaaggggatgattatgaagtcattcctaacagtaatttctatgtatccagaacggcctgcagagataatacttctgtctatcacataagtggaaagaaaaagacatttaaggatgttggaaatcttcttcgaagccatggaattgacttggaccataatagatttttaattttacagggtgaagttgaacaaattgctatgatgaaaccaaaaggccagactgaacacgatgagggtatgcttgaatatttagaagatataattggttgtggacggctaaatgaacctattaaagtcttgtgtcggagagttgaaatattaaatgaacacagaggagataagttaaacagggtaaagatggtggaaaaggaaaaggatgccttagaaggagagaaaaacatagctatcgaatttcttaccttggaaaatgaaatatttagaaaaaagaatcatgtttgtcaatattatatttatgagttgcagaaacgaattgctgaaatggaaactcaaaaggaaaaaattcatgaagataccaaagaaattaatgagaagagcaatatactatcaaatgaaatgaaagctaagaataaagatgtaaaagatacagaaaagaaactgaataaaattacaaaatttattgaggagaataaagaaaaatttacacagctagatttggaagatgttcaagttagagaaaagttaaaacatgccacgagtaaagccaaaaaactggagaaacaacttcaaaaagataaagaaaaggttgaagaatttaaaagtatacctgccaagagtaacaatatcattaatgaaacaacaaccagaaacaatgccctcgagaaggaaaaagagaaagaagaaaaaaaattaaaggaagttatggatagccttaaacaggaaacacaagggcttcagaaagaaaaagaaagtcgagagaaagaacttatgggtttcagcaaatcggtaaatgaagcacgttcaaagatggatgtagcccagtcagaacttgatatctatctcagtcgtcataatactgcagtgtctcaattaactaaggctaaggaagctctaattgcagcttctgagactctcaaagaaaggaaagctgcaatcagagatatagaaggaaaactccctcaaactgaacaagaattaaaggagaaagaaaaagaacttcaaaaacttacacaagaagaaacaaactttaaaagtttggttcatgatctctttcaaaaagttgaagaagcaaagagctcattagcaatgaatcgaagtagggggaaagtccttgatgcaataattcaagaaaaaaaatctggcaggattccaggaatatatggaagattgggggacttaggagccattgatgaaaaatacgacgtggctatatcatcctgttgtcatgcactggactacattgttgttgattctattgatatagcccaagaatgtgtaaacttccttaaaagacaaaatattggagttgcaacctttataggtttagataagatggctgtatgggcgaaaaagatgaccgaaattcaaactcctgaaaatactcctcgtttatttgatttagtaaaagtaaaagatgagaaaattcgccaagctttttattttgctttacgagataccttagtagctgacaacttggatcaagccacaagagtagcatatcaaaaagatagaagatggagagtggtaactttacagggacaaatcatagaacagtcaggtacaatgactggtggtggaagcaaagtaatgaaaggaagaatgggttcctcacttgttattgaaatctctgaagaagaggtaaacaaaatggaatcacagttgcaaaacgactctaaaaaagcaatgcaaatccaagaacagaaagtacaacttgaagaaagagtagttaagttacggcatagtgaacgagaaatgaggaacacactagaaaaatttactgcaagcatccagcgtttaatagagcaagaagaatatttgaatgtccaagttaaggaacttgaagctaatgtacttgctacagcccctgacaaaaaaaagcagaaattgctagaagaaaacgttagtgctttcaaaacagaatatgatgctgtggctgagaaagctggtaaagtagaagctgaggttaaacgcttacacaataccatcgtagaaatcaataatcataaactcaaggcccaacaagacaaacttgataaaataaataagcaattagatgaatgtgcttctgctattactaaagcccaagtagcaatcaagactgctgacagaaaccttcaaaaggcacaagactctgtcttgcgtacagagaaagaaataaaagatactgagaaagaggtggatgacctaacagcagagctgaaaagtcttgaggacaaagcagcagaggtcgtaaagaatacaaatgctgcagaggaatccttaccagagatccagaaagaacatcgcaatctgcttcaagaattaaaagttattcaagaaaatgaacatgctcttcaaaaagatgcacttagtattaagttgaaacttgaacaaatagatggtcacattgctgaacataattctaaaataaaatattggcacaaagagatttcaaaaatatcactgcatcctatagaagataatcctattgaagagatttcggttctaagcccagaggatcttgaagcgatcaagaatccagattctataacaaatcaaattgcacttttggaagcccggtgtcatgaaatgaaaccaaacctcggtgccatcgcagagtataaaaagaaggaagaattgtatttgcaacgggtagcagaattggacaaaattacttatgaaagagacagttttagacaggcatatgaagatcttcggaaacaaaggcttaatgaatttatggcaggtttttatataataacaaataaattaaaggaaaattaccaaatgcttactttgggaggggacgccgaactcgagcttgtagacagcttggatcctttctctgaaggaatcatgttcagtgttcgaccacctaagaaaagttggaaaaagatcttcaacctttcgggaggagagaaaacacttagttcattggctttagtatttgctcttcaccactacaagcccactcccctttacttcatggatgagattgatgcagcccttgattttaaaaatgtgtccattgttgcattttatatatatgaacaaacaaaaaatgcacagttcataataatttctcttcgaaataatatgtttgagatttcggatagacttattggaatttacaagacatacaacataacaaaaagtgttgctgtaaatccaaaagaaattgcatctaagggactttgttgaactttatgctgaagattcttcaagttgattcagtgtattactgatttttttctatttgtaaaggattatgagttgtataaaatacatactccctaaactagatcatgaaactggtttctgttttatgcagttgtcatttgtaaagtctaataaaatattctctataattgcttctagattacaaaaatatgacaatcttgtaagtagcagactatggagaaaaatgagttacctggagggtcaggtaacttgccaaactaaaaagtatgttagttgaggcaaagtcctaagcaaggttgtgctatcaaggctcagcataccttcgtgggcctttgatttaccaacactggaaatgcctgccaactaatcttggatagattctttaaggcattccacttagcttgccagttgagacaatcaccacagttattacccaaatactatgaacatatttttgtaaaccagtcattctgaattatagtgatgagaatttaaatatatgcttttctagaatttgatgtttgaccatttatgacttaattaccagagagccagtaaattaggacagtgtttcaacaagcctaggctatctcgtaagttgaaaaatatcccactatagttgcttcatgagtatgaagtaagatggcctctgatttacactggttcaatttacaaattttcaactttatgataggtttatccgggtactaaatgcatttcaacttgatagtttcaacttatgataggtttaccaggatgtagtcccactgttgaggagcatctatttaggggttaattactttagtaataagtggaaagtaagataccttgagtaatgtttgcctataaaattgtcagcgtatttttacactattggctcaagaatgttataatgctaagggacataagttggcaaccacttggtttttggaaggactttcggtattgtattagaagtctgccctagctgttaaatttctgggtatttatcctaaggaattaattaaagagttaattgttcctttcttcagtgggccattgttttagatatttaaaaaatccaacagtttctatcataatgtaactgtaaaaatgtaaacacattattagcatggacttttaaataaagatttaaagaaagcaaaaaaaaaaaaaaaaaaaa

SEQ ID NO: 48 - DNA, 924 bp, human
atgctgaatacaacctcagtcaccgaatttctcctcttgggagtgacagacattcaagaactgcagccttttctcttcgtggttttcctcaccatctacttcatcagtgtgactgggaatggagccgttctgatgattgtcatctccgatcctagactccattcccttatgtatttcttcctgggaaacctgtcctacctggatatctgttactctacggtgacactgccaaaaatgctgcagaactttctctctacacacaaagcaatttctttcttgggatgcataagccagcttcatttcttccactccctgggcagcacggagtccatgttgttcgccgtgatggcatttgacctctctgtggctatctgcaagccacttcgctacactgtcatcatgaaccctcagctctgtacccagatggccatcacaatctgggtcattggttttttccatgccctgctgcactccgtaatgacttctcgcttgaacttctgtggttccaaccgtatccatcattttctctgtgatattaagccattgctaaagctggcctgtgggaacactgagcttaatcagtggctactcagtactgtcacggggacaattgccatgggccccttctttctgacacttctctcctatttctacattatcacttatctcttcttcaagacccgttcttgtagcatgctctgtaaagcactgtccacttgtgcctcccacttcatggtagttattcttttctatgcacctgttcttttcacctatatccatcctgcgttagagagcttcatggaccaggaccggattgttgccatcatgtacactgtggtcactcctgtactaaacccactgatctatactttgaggaacaaggaagtgaagggggccttgggtagagtgatcagaaggctttga

SEQ ID NO: 49 - DNA, 2496 bp, human
cgagtgcatctggaatacgcagagtcagtaagaccatggctacgtcctcgatgtctaagggttgctttgtttttaagccaaactccaaaaagagaaagatctctctgccaatagaggactattttaacaaagggaaaaatgagcctgaggacagtaagcttcgattcgaaacttatcagttgatatggcagcagatgaaatctgaaaatgagcgactacaagaggaattaaataaaaacttgtttgacaatctgattgaatttctgcaaaaatcacattctggattccagaagaattcaagagacttgggcggtcaaataaaactcagagaaattccaactgctgctcttgttcttggtgtgaatgtcacagatcatgatttgacattcggaagtctaacagaggcccttcagaataatgtcacaccatatgtagtctcattgcaagctaaagattgtccagatatgaaacattttttgcaaaagttgatctcacagttgatggactgctgtgtagatataaaatccaaagaggaggaaagtgttcacgtcacccaaagaaagacacattattcaatggattcactttccagttggtatatgactgtcacacagaagacggacccaaaaatgctaagcaaaaaaaggactacttctagccaatggcagtctcctcctgttgtcgttatcttgaaggatatggaaagctttgccacaaaagtactacaagacttcataattatcagcagtcaacatctccatgaatttccactaatactcatttttggaatagccacatctcctattatcatccaccgattgcttcctcatgcagtatcatctctattgtgcatagaactgttccaatctttgtcttgtaaggagcacctgactacggtactcgataagctacttcttacaactcagtttccctttaaaataaatgaaaaagtattacaggttctgaccaacatctttttgtatcatgatttctcagttcaaaactttataaaaggacttcagctttctctattagagcatttctattcccagcccttaagtgtcctgtgctgtaatcttccagaagccaaaagaagaataaattttttatcaaataatcaatgtgaaaacatccgacgtctaccatcttttaggaggtacgtggaaaagcaagcttcagaaaagcaagttgcgctcttgaccaatgagagatatttgaaggaggaaacacaattattactagaaaacctgcatgtttatcatatgaattacttcctggttttgagatgtcttcataagttcacctcttctcttcccaagtatccactaggtcgacagatcagagagttgtactgtacatgtttagaaaagaacatatgggattcagaggagtatgcatcagtcttgcagctgctgaggatgttggcaaaggatgaactgatgaccatacttgagaaatgtttcaaggtttttaagtcttattgtgaaaaccaccttggcagcacagctaagagaatagaggagttcctggcccagtttcagagcctcgatgaaaccaaagaggaagaagatgcttctgggtcacagccaaaggggcttcagaagacagacctctatcatcttcagaagtccttattggaaatgaaggagttaagaagaagtaagaagcaaaccaaatttgaagtactcagagaaaatgttgtgaacttcattgactgtctagtgagagaataccttctgcctcctgagacacagcctctccatgaggtggtgtacttcagtgctgcccatgcccttcgtgagcatttaaatgctgctccgcgaattgccctccatactgcactcaacaatccttactattatctcaagaatgaagcactgaaaagcgaagaaggctgcattccgaatatcgccccagacatctgcatagcatacaaactgcacctagagtgtagcaggctcatcaacctcgtggactggtcagaggcttttgcaacagttgtgacagctgctgaaaaaatggatgcaaattctgcaacctcagaagaaatgaatgaaattatccatgctcggtttattagagctgtttctgaactagaacttttaggatttataaaacctaccaaacagaagactgaccatgtggcaagactaacatggggaggctgctagaaagcaaataagcaaagccagaactatcacatttagcttaagagaaaaaggtgaccagtcatatttacatatattagaggagcctgttttgttgagaagataaatgtgtaacccccattgatgtttaaccagaaaagtacattgctaaccccaaacaggcatgtatcaaaacacctgtggagtactttagactccaacaaataataatgtaactaaaactgctcacacattttactgtactttccaaagtcattactaaattgtgagtaaatcattcttgaacttagagtatgtaaatgtaataaattccgttatccaggagtat

SEQ ID NO: 50 - DNA, 4916 bp, human
agaattcggagcgcggaagagccagagctgcgagcgcctggagctggatctctctccggtcgcgcacgccgaggccagtagggagagaagatggtggttctccgcagcagcttggagctgcacaaccactccgcggcctcggccacgggctccttggacctgtccagtgacttcctcagtctggagcacatcggccggaggcggctccgctcggccggcgcggcgcagaagaaacccgcggcgaccacagccaaagcgggcgatgggtcatcagttaaggaagttgaaacctaccaccggacacgtgctttaagatctttgagaaaagatgcacagaattcttcagattctagttttgagaagaatgtggaaataacggagcaacttgctaatggcaggcattttacaaggcagttggccagacagcaggctgataaaaaaaaagaagagcacagagaagacaaagtgattccagttactcggtcattgagggctagaaacatcgttcaaagtacagaacacttacatgaagataatggtgatgttgaagtgcgtcgaagttgtaggattagaagtcgttatagtggtgtaaaccagtccatgctgtttgacaaacttataactaacactgctgaagctgtacttcaaaaaatggatgacatgaagaagatgcgtagacagcgaatgagagaacttgaagacttgggagtgtttaatgaaacagaagaaagcaatcttaatatgtacacaagaggaaaacagaaagatattcaaagaactgatgaagaaacaactgataatcaagaaggcagtgtggagtcatctgaagagggtgaagaccaagaacatgaagatgatggtgaagatgaagatgatgaagatgatgatgatgatgacgatgatgatgatgatgatgatgatgaagatgatgaagatgaagaagatggagaagaagagaatcagaagcgatattatcttagacagagaaaagctactgtttactatcaggctccattggaaaaacctcgtcaccagagaaagcccaacatattttatagtggcccagcttctcctgcaagaccaagataccgattatcttccgcaggaccaagaagtccttactgtaaacgaatgaacaggcgaaggcatgcaatccacagtagtgactcgacttcatcttcctcctctgaagatgaacagcactttgagaggcggaggaaaaggagtcgtaatagggctatcaataggtgcctcccactaaattttcggaaagatgaattaaaaggcatttataaagatcgaatgaaaattggagcaagccttgccgatgttgatccaatgcaactagattcttcagtacgatttgatagtgttggtggcctgtctaatcatatagcagctctaaaagagatggtggtgtttccattactttatccagaagtctttgaaaaatttaaaattcaacccccaagaggttgtttgttttatgggccacctggaactggaaagactctggttgccagagcacttgccaatgagtgcagtcaaggggataaaagagtagcatttttcatgaggaaaggtgctgattgtctaagtaaatgggtaggagaatctgaaagacagctacgattgctgtttgatcaggcctatcagatgcgcccatcaattattttttttgacgaaattgatggtctggctccagtacggtcaagcaggcaagatcagattcacagttctattgtttccaccctgctagctcttatggatggattggacagcagaggggaaattgtggtcattggtgctacgaacaggctagattctatagatcctgctttacgaaggcctggtcgctttgatagagaattcctctttagcctgcctgataaagaggctcgaaaagagattctaaagattcacaccagggattggaatcccaaaccactggacacatttttagaagagctagcagaaaactgtgttggatactgtggagcagatattaaatcaatatgtgctgaagctgctttatgtgctttacgacgacgctacccacagatctataccactagtgagaaactgcagttggatctctcttcaattaatatctcagctaaggatttcgaggtagctatgcaaaagatgataccagcctcccaaagagctgtgacatcacctgggcaggcactgtccaccgttgtgaaaccactcctgcaaaacactgttgacaagattttagaagccctgcagagagtatttccacatgcagaattcagaacaaataaaacattagactcagatatttcttgtcctctgctagaaagtgacttggcttacagtgatgatgatgttccatcagtttatgaaaatggactttctcagaaatcttctcataaggcaaaagacaattttaattttcttcatttgaatagaaatgcttgttaccaacctatgtcttttcgaccaagaatattgatagtaggagaaccaggatttgggcaaggttctcacttggcaccagctgtcattcatgctttggaaaagtttactgtatatacattagacattcctgttctttttggagttagtactacatcccctgaagaaacatgtgcccaggtgattcgtgaagctaagagaacagcaccaagtatagtgtatgttcctcatatccacgtgtggtgggaaatagttggaccgacacttaaagccacatttaccacattattacagaatattccttcatttgctccagttttactacttgcaacttctgacaaaccccattccgctttgccagaagaggtgcaagaattgtttatccgtgattatggagagatttttaatgtccagttaccggataaagaagaacggacaaaattttttgaagatttaattctaaaacaagctgctaagcctcctatatcaaaaaagaaagcagttttgcaggctttggaggtactcccagtagcaccaccacctgagccaagatcactgacagcagaagaagtgaaacgactagaagaacaagaagaagatacatttagagaactgaggattttcttaagaaatgttacacataggcttgctattgacaagcgattccgagtgtttactaagcctgttgaccctgatgaggttcctgattatgtcactgtaataaagcaaccaatggacctttcatctgtaatcagtaaaattgatctacacaagtatctgactgtgaaagactatttgagagatattgatctaatctgtagtaatgccttagaatacaatccagatagagatcctggagatcgtcttattaggcatagagcctgtgctttaagagatactgcctatgccataattaaagaagaacttgatgaagactttgagcagctctgtgaagaaattcaggaatctagaaagaaaagaggttgtagctcctccaaatatgccccgtcttactaccatgtgatgccaaagcaaaattccactcttgttggtgataaaagatcagacccagagcagaatgaaaagctaaagacaccgagtactcctgtggcttgcagcactcctgctcagttgaagaggaaaattcgcaaaaagtcaaactggtacttaggcaccataaaaaagcgaaggaagatttcacaggcaaaggatgatagccagaatgccatagatcacaaaattgagagtgatacagaggaaactcaagacacaagtgtagatcataatgagaccggaaacacaggagagtcttcggtggaagaaaatgaaaaacagcaaaatgcctctgaaagcaaactggaattgagaaataattcaaatacttgtaatatagagaatgagcttgaagactctaggaagactacagcatgtacagaattgagagacaagattgcttgtaatggagatgcttctagctctcagataatacatatttctgatgaaaatgaaggaaaagaaatgtgtgttctgcgaatgactcgagctagacgttcccaggtagaacagcagcagctcatcactgttgaaaaggctttggcaattctttctcagcctacaccctcacttgttgtggatcatgagcgattaaaaaatcttttgaagactgttgttaaaaaaagtcaaaactacaacatatttcagttggaaaatttgtatgcagtaatcagccaatgtatttatcggcatcgcaaggaccatgataaaacatcacttattcagaaaatggagcaagaggtagaaaacttcagttgttccagatgatgatgtcatggtatcgagtattctttatattcagttcctatttaagtcatttttgtcatgtccgcctaattgatgtagtatgaaaccctgcatctttaaggaaaagattaaaatagtaaaataaaagtatttaaactttcctgatatttatgtacatattaagataaatgtcatgtgtaagataactgataaatattggaactttgctagaacaagaccctgtagtaatagtaataatagttgaagtttggccaactcttaataaagttattttggtaactaatgttttatggcacttaagaataattagcagcgttaaattttgtttgtattaagcacttttaattttatccttcctaaaaatagtttattgtatctgacaagaaacttacttaaccattgtgtccttcccatcttttttgtcatctttgttttcttcaaatgccctcctcccatctgccttgagattcccttgtcttcacttaaaagccagagtgcaagtcatgatttgcgggagggctcttgaaccacttctggctgcaccacaattctgtacttgagtatcacagtcattgtttttgagacaaacatttttataattctaatttgggttaataaagattttaaatatttaaaaaaaaaaaaaaaaaaaa

SEQ ID NO: 51 - DNA, 4183 bp, human
atgagaccacagtcataagagtaaaacaaaaattctgatggaaataaatgcttatttggtaggaagcaaaaagcacaaaaacaagattgtctttctttttctttttctttttaaatgagaaggatccacccttgtgggaaaggaactctcaaaagacagcagtgggcctgacatcttgggcactttccaggttcttgggaggaagggagaaaagaagcagagagagagaagaaatatctactatttttatatacggtgtattcctctcatatcttaaagaaaagctttatgtagaaaaaaagattcatgacgcagcttatgtaagaataaacttgtggaatggaattagttgcagggaggaggtctgatttcgttcagttctcacagtgatctagtgtctgacttttctagggaatccaatcaataaaattgaagactggctgcaggattgcggatactctgaagaaggattttctgaggaagcaggacagtttatctacaatggcttctgcagccatgggaccagctttgaagatgacttgaccctgggagcggaggccacactactggccgccaatggcaaactcttctccaggagtttcctcgagacagccaggccttgccagctacttgatcttggctgtagtttggcttccagcagcatgactggggggaccaacaagactagttcaagcatctcagaaattctggacaaagtgcaagaagatgcagaagatgtcctcttcagtctgggctttggccaagaggatcacaaagacacttctcggatacccgcccgatttttcaccaccccctctcaggccaagggcattgatttccagctcttcctgaagtcccaggtgcggaggattgaaatggaagacccctgcctcatgctggccagcaggtttaagcaggtgcaaacactggctgtcactgctgatgccttcttctgtctctactcctatgtgtctaagacacctgtccaaaagttcacaccatcccacatgttctggaattgcaaccatccaactgatgtgccatccatcaggattctgtcccgagagcctgaaccccagtcacccagagaccgccttcggaaagccatctccaagatgtgtctgtacacatgcccccgagaccggccaccaccaccccacaacacccccaaaaggaacagtttggaccaagttgtgttggaagtgatggacaaagtgaaagaagagaagcagttcctccagcaagactctgatttggggcaattctcacaggaagatcctgtgccccctgctgagggtaagaagttgcccacttctccctatccatgtgtcttctgctgtgaagaggaaacacagcagaggatgtccacagtgctagcaccatcccaaactctggattcgaaccccaaggtaccctgttgcacacattctctgcccatagaagatccacagtggagcacagacccagctcagataaggagagagctgtgtagtctaccagccaccaatacggaaacccatccagccaaggatgagactttctggaaaagaaagagcagagcaagaaagagcctcttccagaagaatctcatgggcaggaaggttaagtccttggacctgtccatcacccagcagaaatggaagcagagtgtagacagaccagaactgcgtcggtctttaagccagcagccacaggacacttttgacttggaggaggtgcaaagcaacagtgaggaggagcagagtcagagtcgctggcccagcagacccaggcacccccaccaccaccagacttttgctggcaaagactcgtgaagctttctccaccaccacaacagcatgtggtctgacagaattggcttcaaagaagaaactaaaagccacctcttgcaggaaactgcactgccctcaacatccagcccctactccttggtcctccaaacccccaaactctgcaggagaaaggcagcatggtacatggggaggaagaccagactgagcgatttggaatccacatcctaatgctgccacaagctgcatgcacaaagaccttaggcacatctcttcatttctctgtacactggtttctctactatgtgtgtattaaaatatataatgtggatgatagtaaactgaacaaagccttaattttctcccaagctttgacattgccaagggcagttaggagacttcaggatcaagtttaggggacaagtttttttctaatactttcaaaaggcccaagtgaagagaggaaggacacctcactttctggctctaaaagcatggtacatctcacactaggattccctctgcacctctgttcatccctgtattcctaactaaattccagaaaatcttccacaacactggagtcctatttttcttcttgagacctcctttgaagaaatcaacttggagactcctccttaaatccagtttctttctctacccaaaattttgcatctttatagaaaaagagataagtaggattcagagtctcttctcctagccattgtggggggaaaaaatgctatttctgttggcagaaaacaatacaaatattttttttaaatcatggcatgtttttaaagttaactaacagctgtagtttgctttggaagttaaatgttttgtcaataaacagggcttgagcacatgcagtgctctcttaagcagtatgtggtttcagagcatcttggacacatttctcccctgagaaaatattagtattaatagaatgggagaagtaagatgcacgcatttccagaagtatattatttggctgcctaataaattgtgagacaaaagaaaaaaaaaaaacaggaagagagggacttgagggaatttgagggaaatcataaccaaaagtttaaaggaaaaaattacaccagacatgataaacagaagttgattttgttctggtgctattgccagcattaggggagaaggcacaaactgagtctgatacgtagtgaggcagagacaaaagggctagggtggaaaaatttttaaaccatctgtgtttactaattccccataccaaaagaacaaaatgaactttctcctatttcatgataaaagatagtttcacaatttggaaaaagccccccactccaagtttcctacccttcccacccacagaaaccaggagacaggggaactatcttccttgatgttaacatttcaaagaggtggttctcaggtcctagagaaagatattcctgagttgtaaaactgggaagaggctggaagaagatttagatctcaagaaatagaaagaactacagcaaattttctaaagcaaatattcttaagggaggtcaagggcctgaagtcaggaagaaacctatttggtcaagctgaggggaaagttaaggctatcttgtcacaagagaacagaaatatctgacaatatttcatttaaagaactgtattagtccattctcacactgctataaataaatacctgagactgggtaatttatacagaaaagaggtttaattggctcacagttccgcaggctgtacaggaagcatggcaacatcagtttctggggaggcctcagggagctttaactcatggtggaaagcaaagtgggagcaggcatcttacctggtatgagcaggaggaacaaaggaaaggggaggtgctaacacttttaaatgaccagatcttgtaagaactcactcattatacagtaccaaggggagatggtgctaaacgattaatgaaaactctacccagtaatccaatcaccacccaccaggccccacctccaacaatggggattacagtttgacatgagatttgggtgaggacacagatccaaactgtcaggcctctgagcccaagctaagccatcatatcccctgtgacctgcatgtatacatccagatggcctgaagcaagtggagaatcacaaaagaagtgaaaatggcctgttgctgccttaacagatgacattactttgtgaaattccttcttctggctcagaagctcccccactgagcaccttgtgacccccacccctgcctgccagagaataatcccctttgactgtaattttccactacctacctaaatcctataaaacagccccacccctatctccctttgctgactctctttttggactcagcctgcctgcacccaggtgattaaaaagctttattgctcacac

SEQ ID NO: 52 - DNA, 2505 bp, human
gctgagcgccggaggagcgtaggcagggcagcgctggcgccagcggcgacaggagccgcgcgaccggcaaaaatacacgggaggccgtcgccgaaaagagtccgcggtcctctctcgtaaacacactctcctccaccggcgcctccccctccgctctgcgcgccgcccggctgggcgcccgaggccgctccgactgctatgtgaccgcgaggctgcgggaggaaggggacagggaagaagaggctctcccgcgggagcccttgaggaccaagtttgcggccacttctgcaggcgtcccttcttagctctcgcctgcccctttctgcagcctaggcggcccaggttctcttctcttcctcgcgcgcccagccgcctcggttcccggcgaccatggtgacgatggaggagctgcgggagatggactgcagtgtgctcaaaaggctgatgaaccgggacgagaatggcggcggcgcgggcggcagcggcagccacggcaccctggggctgccgagcggcggcaagtgcctgctgctggactgcagaccgttcctggcgcacagcgcgggctacatcctaggttcggtcaacgtgcgctgtaacaccatcgtgcggcggcgggctaagggctccgtgagcctggagcagatcctgcccgccgaggaggaggtacgcgcccgcttgcgctccggcctctactcggcggtcatcgtctacgacgagcgcagcccgcgcgccgagagcctccgcgaggacagcaccgtgtcgctggtggtgcaggcgctgcgccgcaacgccgagcgcaccgacatctgcctgctcaaaggcggctatgagaggttttcctccgagtacccagaattctgttctaaaaccaaggccctggcagccatcccacccccggttccccccagcgccacagagcccttggacctgggctgcagctcctgtgggaccccactacacgaccaggggggtcctgtggagatccttcccttcctctacctcggcagtgcctaccatgctgcccggagagacatgctggacgccctgggcatcacggctctgttgaatgtctcctcggactgcccaaaccactttgaaggacactatcagtacaagtgcatcccagtggaagataaccacaaggccgacatcagctcctggttcatggaagccatagagtacatcgatgccgtgaaggactgccgtgggcgcgtgctggtgcactgccaggcgggcatctcgcggtcggccaccatctgcctggcctacctgatgatgaagaaacgggtgaggctggaggaggccttcgagttcgttaagcagcgccgcagcatcatctcgcccaacttcagcttcatggggcagctgctgcagttcgagtcccaggtgctggccacgtcctgtgctgcggaggctgctagcccctcgggacccctgcgggagcggggcaagacccccgccacccccacctcgcagttcgtcttcagctttccggtctccgtgggcgtgcactcggcccccagcagcctgccctacctgcacagccccatcaccacctctcccagctgttagagccgccctgggggccccagaaccagagctggctcccagcaagggtaggacgggccgcatgcggcagaaagttgggactgagcagctgggagcaggcgaccgagctccttccccatcatttctccttggccaacgacgaggccagccagaatggcaataaggactccgaatacataataaaagcaaacagaacactccaacttagagcaataaccggtgccgcagcagccagggaagaccttggtttggtttatgtgtcagtttcacttttccgatagaaatttcttacctcatttttttaagcagtaaggcttgaagtgatgaaacccacagatcctagcaaatgtgcccaaccagctttactaaagggggaggaagggagggcaaagggatgagaagacaagtttcccagaagtgcctggttctgggtacttgtccctttgttgtcgttgttgtagttaaaggaatttcatttttaaaagaaatcttcgaaggtgtggttttcatttctcagtcaccaacagatgaataattatgcttaataataaagtatttattaagactttcttcagagtatgaaagtacaaaaagtctagttacagtggatttagaatatatttatgttgatgtcaaacagctgagcaccgtagcatgcagatgtcaaggcagttaggaagtaaatggtgtcttgtagatatgtgcaaggtagcatgatgagcaacttgagtttgttgccactgagaagcaggcgggttgggtgggaggaggaagaaagggaagaattaggtttgaattgctttttaaaaaaaaaagaaaagaaaaagacagcatctcactatgttgccaaggctcatcttgagaagcaggcgggttgggtgggaggaggaagaaagggaagaattaggtttgaattgcttttttaaaaaaaaa

SEQ ID NO: 53 - DNA, 2204 bp, human
gagcggtgcggaggctctgctcggatcgaggtctgcagcgcagcttcgggagcatgagtgctgcagtgactgcagggaagctggcacgggcaccggccgaccctgggaaagccggggtccccggagttgcagctcccggagctccggcggcggctccaccggcgaaagagatcccggaggtcctagtggacccacgcagccggcggcgctatgtgcggggccgctttttgggcaagggcggctttgccaagtgcttcgagatctcggacgcggacaccaaggaggtgttcgcgggcaagattgtgcctaagtctctgctgctcaagccgcaccagagggagaagatgtccatggaaatatccattcaccgcagcctcgcccaccagcacgtcgtaggattccacggctttttcgaggacaacgacttcgtgttcgtggtgttggagctctgccgccggaggtctctcctggagctgcacaagaggaggaaagccctgactgagcctgaggcccgatactacctacggcaaattgtgcttggctgccagtacctgcaccgaaaccgagttattcatcgagacctcaagctgggcaaccttttcctgaatgaagatctggaggtgaaaataggggattttggactggcaaccaaagtcgaatatgacggggagaggaagaagaccctgtgtgggactcctaattacatagctcccgaggtgctgagcaagaaagggcacagtttcgaggtggatgtgtggtccattgggtgtatcatgtataccttgttagtgggcaaaccaccttttgagacttcttgcctaaaagagacctacctccggatcaagaagaatgaatacagtattcccaagcacatcaaccccgtggccgcctccctcatccagaagatgcttcagacagatcccactgcccgcccaaccattaacgagctgcttaatgacgagttctttacttctggctatatccctgcccgtctccccatcacctgcctgaccattccaccaaggttttcgattgctcccagcagcctggaccccagcaaccggaagcccctcacagtcctcaataaaggcttggagaaccccctgcctgagcgtccccgggaaaaagaagaaccagtggttcgagagacaggtgaggtggtcgactgccacctcagtgacatgctgcagcagctgcacagtgtcaatgcctccaagccctcggagcgtgggctggtcaggcaagaggaggctgaggatcctgcctgcatccccatcttctgggtcagcaagtgggtggactattcggacaagtacggccttgggtatcagctctgtgataacagcgtgggggtgctcttcaatgactcaacacgcctcatcctctacaatgatggtgacagcctgcagtacatagagcgtgacggcactgagtcctacctcaccgtgagttcccatcccaactccttgatgaagaagatcaccctccttaaatatttccgcaattacatgagcgagcacttgctgaaggcaggtgccaacatcacgccgcgcgaaggtgatgagctcgcccggctgccctacctacggacctggttccgcacccgcagcgccatcatcctgcacctcagcaacggcagcgtgcagatcaacttcttccaggatcacaccaagctcatcttgtgcccactgatggcagccgtgacctacatcgacgagaagcgggacttccgcacataccgcctgagtctcctggaggagtacggctgctgcaaggagctggccagccggctccgctacgcccgcactatggtggacaagctgctgagctcacgctcggccagcaaccgtctcaaggcctcctaatagctgccctcccctccggactggtgccctcctcactcccacctgcatctggggcccatactggttggctcccgcggtgccatgtctgcagtgtgccccccagccccggtggctgggcagagctgcatcatccttgcaggtgggggttgctgtgtaagttatttttgtacatgttcgggtgtgggttctacagccttgtccccctccccctcaaccccaccatatgaattgtacagaatatttctattgaattcggaactgtcctttccttggctttatgcacattaaacagatgtgaatattcaaaaaaaaaaaaaaaaaaaaaaaaaaaaaaaaaaa

SEQ ID NO: 54 - DNA, 2530 bp, human
aaaacaggagctgattcgagctggcagagctgggccatggaaccggtagagacctggacccccggaaaggtggcaacttggctgagaggtcttgacgactccctgcaggactatccctttgaggactggcagctgcctggcaagaacctgctccagctctgcccccaaagcctcgaggctctggctgtgcggtctctgggacaccaggagctcatcctgggcggggtggaacagctccaggccctgagctccaggctacagacagagaacctgcaaagcctgacagagggacttctgggggcaacccatgacttccagagcatagtccaaggctgcctgggggactgtgccaagacccctattgatgtcctctgtgcagctgtggagctgttgcatgaagctgacgccctcctcttctggctcagcaggtacctcttctcccacttaaatgatttctcagcatgccaggagatccgagacttgttggaggagctgagccaggtcttgcatgaggatggtccagcggctgagaaggagggcacagtcctgaggatctgcagccacgtggctgggatctgccacaacatcctggtctgctgccccaaggagctgctggaacagaaggccgtgctcgagcaggtgcagctggacagtccattgggcctagaaattcacaccaccagcaattgccagcactttgtgtcccaagtggacacccaggttcccactgactcccgactgcagatccagcctggagacgaggttgtccagatcaacgagcaggtggtggtgggatggccccgtaagaacatggtgagggaactgctgcgggagccagccggactcagcttagtgctgaagaagatcccgataccggagacccccccacagacgccccctcaggtcctggactccccgcaccagaggagcccatcactgtctctggccccactgtctcccagggccccatctgaagacgtctttgcctttgacctgtcttcaaaccccagtcccggacccagccctgcctggacagactctgcctcccttggccctgagcccctgcccatccccccggaacccccagccatactcccagcaggggtagcagggactccagggctccctgaatcccctgacaagagtcctgttggtcggaagaaatcaaaaggcctggcgacccggctgagccgccggcgggtgtcatgccgtgagctgggccggccggactgtgacggctggctcctgttgcgaaaggcaccgggcggcttcatgggcccgcgctggcgccgccgctggtttgtgctcaagggacacacgctctactggtaccgccagccccaggatgagaaggctgagggcctcatcaatgtctccaactatagtctggaaagtggacatgatcagaagaagaaatatgtgtttcagctcacccatgatgtgtacaaacccttcatcttcgctgctgataccctgacagatctgagcatgtgggtgcgtcatctcattacctgcatctccaagtaccagtctccaggccgggcccccccaccccgagaggaagactgctacagtgagaccgaagcagaggacccggacgatgaggctgggtcccactcagcctcgcccagccctgctcaagctgggagtcccctccatggagacacatcacctgcagccacccccacacagcgcagcccacggacctcctttggctctctgacagacagcagtgaagaggcactggaaggaatggtacgggggctgaggcagggtggcgtgtccctcctaggccagccacagcccctgacccaggaacagtggcggagctctttcatgcggcgcaaccgagaccctcagctcaatgagcgagtgcaccgtgtgcgggcgctacagagcacactcaaggcaaagctgcaggagctgcaggtcctagaagaagtgctgggtgaccctgagctgacaggagagaagttccgccagtggaaggagcagaaccgggagctgtactcagagggcctgggggcctggggagtggcacaggctgaaggcagctcccacatcttgacctctgactccacagaacagtcccccaactccctgccctctgaccctgaagagcactcccatctctgccccctgacctcagagagcagcctccgacctcctgacctctgaccctggccagcactctagctcctgacctttgacccgagggccacctcaaccccagcttctgacgtgtccaggacagagcatccctggattctgttcagggtgggaagtagtactgctagtcatggtctcaccccgagctgacccctctgcctgggctttgtgccaccctctcccttgccaaagaagaaactctccccccaaatcctccaacctctggggccacagccctgcccctccagttccttggcagttctcccccaaaccaggtctgtacaggtgttctttattttacatgagggctactttccaaccaaataaagtcaatttttctaagaaaaaaaaaaaaaaaaaa